(19) United States
(12) Patent Application Publication
(10) Pub. No.: US 2026/0108554 A1
(43) Pub. Date: Apr. 23, 2026

(54) METHODS FOR REPROGRAMMING AND GENE EDITING CELLS

(71) Applicant: Factor Bioscience Inc., Cambridge, MA (US)

(72) Inventors: Matthew ANGEL, Cambridge, MA (US); Christopher ROHDE, Cambridge, MA (US); Ian HAY, Cambridge, MA (US); Ismet Caglar TANRIKULU, Cambridge, MA (US); Tae Yun KIM, Cambridge, MA (US); Abigail BLATCHFORD, Cambridge, MA (US); Mackenzie PARMENTER, Cambridge, MA (US)

(21) Appl. No.: 18/861,162

(22) PCT Filed: May 1, 2023
(86) PCT No.: PCT/US2023/066464

§ 371 Date: Oct. 28, 2024

Related U.S. Application Data
(60) Provisional application No. 63/337,166, filed on May 1, 2022.
(60) Provisional application No. 63/342,144, filed on May 15, 2022.

(51) Int. Cl. A61K 35/15; A61K 40/31; C07K 14/54; C07K 14/55; C07K 14/74; C12N 9/22; C12N 15/11; C12N 15/62; C12N 15/88; C12N 15/90

(52) U.S. Cl. CPC A61K 35/15; A61K 40/31; C07K 14/5418; C07K 14/5428; C07K 14/5434; C07K 14/5443; C07K 14/55; C07K 14/70539; C12N 9/222; C12N 15/111; C12N 15/625; C12N 15/88; C12N 15/907; C07K 2319/02; C12N 2310/20; C12N 2510/00

(57) ABSTRACT

The present disclosure provides improved methods for reprogramming and gene editing cells, including manufacturing a population of cells comprising cells of the lymphoid lineage and/or cells of the myeloid lineage.

### CROSS-REFERENCE TO RELATED APPLICATION

This application is a National Stage Entry of International Application No. PCT/US2023/066464, which claims the benefit of U.S. Provisional Patent Application No. 63/337,166, filed May 1, 2022, and U.S. Provisional Patent Application No. 63/342,144, filed May 15, 2022, each of which is entirely incorporated herein by reference.

### SEQUENCE LISTING

The instant application contains a Sequence Listing which has been submitted in XML format via Patent Center and is hereby incorporated by reference in its entirety. Said XML copy, created on May 1, 2023, is named 61057-719_601.xml and is 81,727 bytes in size.

### BACKGROUND

Methods for reprogramming differentiated cells into pluripotent cells and methods for gene-editing cells have progressed greatly over recent years. However, there remain unmet needs for improved methods for reprogramming and gene editing cells.

### SUMMARY

Accordingly, the present disclosure provides improved methods for reprogramming and gene editing cells.

An aspect of the present disclosure is a method for treating a cancer. The method comprising administering to a subject in need a therapeutically-effective amount of a first pharmaceutical composition comprising one or both of a population of isolated lymphoid lineage cells and a population of isolated myeloid lineage cells.

In numerous embodiments, one or more of the isolated lymphoid lineage cells and/or one or more of the isolated myeloid lineage cells comprise a genomic modification that expresses a chimeric antigen receptor (CAR), e.g., a CAR which comprises an antigen binding region that binds to one or more antigens expressed by a cancer cell. In some cases, the antigen binding region binds to one or more tumor antigens. In various cases, the CAR comprises an antigen binding region that binds to ROR1. In embodiments, one or more of the isolated lymphoid lineage cells and/or one or more of the isolated myeloid lineage cells comprise a genomic modification that expresses or over expresses a cytokine. In various embodiments, the method further comprises administering to the subject in need a synthetic mRNA encoding a gene-editing protein and a single-stranded or double-stranded repair template which encodes a chimeric antigen receptor (CAR). In some cases, the gene-editing protein creates a single-stranded break or a double-stranded break in the genomic DNA of a cell in the subject and the single-stranded or double-stranded repair template which encodes the CAR inserts into the break. In these embodiments, the cell in the subject expresses the CAR.

In numerous embodiments, the method further comprises administering to the subject in need a synthetic mRNA encoding a gene-editing protein and a single-stranded or double-stranded repair template which encodes a cytokine. In some cases, the gene-editing protein creates a single-stranded break or a double-stranded break in the genomic DNA of a cell in the subject and the single-stranded or double-stranded repair template which encodes the cytokine inserts into the break. In these embodiments, the cell in the subject expresses or over expresses the cytokine.

In various embodiments, the isolated lymphoid lineage cells are manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes; wherein steps (5) and (6) occur in an adherent culturing vessel.

In embodiments, the isolated myeloid lineage cells are manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a myeloid progenitor medium; and (6) culturing the cells of step (5) in a macrophage cell medium under conditions to obtain a population of cells enriched for macrophages; wherein steps (5) and (6) occur in a bioreactor.

Another aspect of the present disclosure is a plurality of compositions for use in any herein-disclosed method for treating a cancer.

Yet another aspect of the present disclosure is a method for killing a cancer cell or for inhibiting the proliferation of a cancer cell. The method comprising contacting the cancer cell with a population of isolated lymphoid lineage cells and a population of isolated myeloid lineage cells.

In some embodiments, contacting the cancer cell with a population of isolated lymphoid lineage cells and a population of isolated myeloid lineage cells occurs in vitro.

In various embodiments, contacting the cancer cell with a population of isolated lymphoid lineage cells and a population of isolated myeloid lineage cells occurs in vivo.

Yet a further aspect of the present disclosure is a plurality of compositions for use in any herein-disclosed method for killing a cancer cell or for inhibiting the proliferation of a cancer cell.

In an aspect, the present disclosure provides a method for manufacturing a plurality of population of cells comprising a population of isolated lymphoid lineage cells and a population of isolated myeloid lineage cells for treating a cancer, for killing a cancer cell, and/or for inhibiting the proliferation of a cancer cell. The method a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor.

An aspect of the present disclosure is a method for manufacturing a population of cells that is enriched for cytotoxic lymphocytes. The method comprises steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes. In this aspect, steps (5) and (6) occur in an adherent culturing vessel. When CD34+ cells are selected, the embryoid bodies may be first chemically and/or mechanically dissociated.

Another aspect of the present disclosure is a method for killing a cancer cell. The method comprising steps of: (1) obtaining a herein-disclosed cytotoxic lymphocyte and (2) contacting cytotoxic lymphocyte with the cancer. In some cases, the cancer cell is in vivo.

Yet another aspect of the present disclosure is a method for treating a cancer patient in need thereof. The method comprising a step of administering to the cancer patient a therapeutically-effective amounts of a herein-disclosed cytotoxic lymphocyte.

In an aspect, the present disclosure provides a pharmaceutical composition comprising a herein-disclosed cytotoxic lymphocyte and a pharmaceutically acceptable carrier or excipient.

In another aspect, the present disclosure provides a composition comprising a cell comprising a genetically engineered disruption in a beta-2-microglobulin (B2M) gene, wherein the cell is a cytotoxic lymphocyte from a lymphoid lineage cell, e.g., an NK cell, or the cell is from a myeloid lineage, e.g., a macrophage, or the cell is a mesenchymal stromal/stem cell, or the cell is a hematopoietic stem cell.

In yet another aspect, the present disclosure provides a pharmaceutical composition comprising an isolated NK cell of any herein-disclosed aspect or embodiment.

In yet another aspect, the present disclosure provides a pharmaceutical composition comprising an isolated myeloid cell of any herein-disclosed aspect or embodiment, e.g., a macrophage.

In yet another aspect, the present disclosure provides a pharmaceutical composition comprising an isolated mesenchymal stromal/stem cell of any herein-disclosed aspect or embodiment.

An aspect of the present disclosure is a method of making an engineered cell comprising a disruption in a beta-2-microglobulin (B2M) gene. The method comprising steps of (a) reprogramming a somatic cell to an iPS cell, the reprogramming comprising contacting the iPS cell with a ribonucleic acid (RNA) encoding one or more reprogramming factors; (b) disrupting a B2M gene in the iPS cell, the disrupting comprising gene-editing the cell by contacting the cell with RNA encoding one or more gene-editing proteins; and (c) differentiating the iPS cell into a differentiated cell. In this aspect, the differentiated cell is a cytotoxic lymphocyte from a lymphoid cell lineage or is from a myeloid cell lineage, e.g., a macrophage.

Another aspect of the present disclosure is a method of treating cancer. The method comprising steps of obtaining an isolated cell comprising a genetically engineered disruption in a B2M gene and administering the isolated cell to a subject in need thereof. In this aspect, the cytotoxic lymphocyte is a lymphoid cell or a CAR-myeloid cell or a CAR-mesenchymal stromal/stem cell.

Yet another aspect of the present disclosure is a composition comprising an isolated cytotoxic lymphocyte comprising a gene edit in a CD16a gene, wherein the cytotoxic lymphocyte is a lymphoid lineage cell, e.g., an NK cell.

An aspect of the present disclosure is a method for producing macrophages from an induced a pluripotent stem cell (iPSC). The method comprises steps of: (1) obtaining an iPSC; (2) culturing the iPSC in a first medium for about three days; (3) culturing the iPSC in a second for about four days; (4) culturing the iPSC in a monocyte differentiating medium for at least seven days, thereby obtaining monocytes (5) isolating the monocytes; (6) culturing the monocytes for about four days; (7) culturing the monocytes in the presence of M-CSF for three to four days, thereby obtaining macrophages; and (8) harvesting the macrophages. In some embodiments, the macrophages are further contacted with interferon gamma (IFN-γ) and/or lipopolysaccharide (LPS) to obtain M1 macrophages and/or the macrophages are further contacted with IL-4 to obtain M2 macrophages.

In embodiments, the macrophages, e.g., the M1 and M2 macrophages, are capable of killing cancer cells.

In embodiments, the iPSC was reprogrammed from a differentiated or non-pluripotent cell.

In embodiments, the iPSC or a progenitor cell was gene-edited. In some cases, the iPSC or the progenitor cell was gene-edited to knockout the beta-2 microglobulin (B2M) gene.

In embodiments, the gene-editing comprises transfection of a repair template.

In embodiments, the repair template includes the coding sequence for one or more of HLA class I histocompatibility antigen, alpha chains (HLAs).

In embodiments, the repair template comprises a TTAGGG motif for decreasing synthetic oligodeoxynucleotides (ODNs)-related activation of pro-inflammatory responses and/or the cell is transfected with a TTAGGG motif separate from the repair template.

In embodiments, the differentiated or non-pluripotent cell was contacted with resveratrol before reprogramming.

In embodiments, the iPSC was contacted with resveratrol before gene-editing and/or the iPSC was contacted with resveratrol after gene-editing.

Another aspect is an isolated macrophage obtained by a herein disclosed method.

Yet another aspect is pharmaceutical composition comprising a herein disclosed isolated macrophage of and a pharmaceutically-acceptable excipient.

In an aspect, the disclosure provides an isolated M1 macrophage and/or an isolated M2 macrophage obtained by a herein disclosed method.

In yet another aspect, the disclosure provides a pharmaceutical composition comprising a herein disclosed isolated M1 macrophage and/or an isolated M2 macrophage and a pharmaceutically-acceptable excipient.

In another aspect, the disclosure provides method for treating a cancer comprising in vivo administering to a subject in need a herein disclosed pharmaceutical composition.

An aspect of the present disclosure is a method for decreasing synthetic oligodeoxynucleotides (ODNs)-related activation of pro-inflammatory responses, the method comprising transfecting a cell with an ODN comprising a TTAGGG motif.

In embodiments, the ODN is a double stranded ODN (dsODN) and comprise a repair template.

In embodiments, the TTAGGG motif is attached to the 5′ and/or the 3′ end of the repair template.

In embodiments, the ODN is a single stranded ODN (ssODN) and does not comprise a repair template.

In embodiments, the cell is transfected with a synthetic nucleic acid encoding a gene-editing protein along with a repair template.

Another aspect is an isolated cell obtained by a method relating to an ODN comprising a TTAGGG motif.

Yet another aspect is a method for enhancing the efficiency of gene-editing, the method comprising contacting a cell with resveratrol before gene-editing.

In embodiments, contacting the cell with resveratrol arrests the cell in S/G2 phase.

In embodiments, the cell is further contacted with resveratrol after gene-editing.

In embodiments, gene-editing comprise transfection of a synthetic nucleic acid encoding a gene-editing protein.

In an aspect, the present disclosure provides a method for enhancing the efficiency of gene-editing, the method comprising contacting a cell that has been gene-edited with resveratrol.

In embodiments, the gene-editing comprise transfection of a synthetic nucleic acid encoding a gene-editing protein.

Any aspect or embodiment herein may be combined with any other aspect or embodiment as disclosed herein.

### BRIEF DESCRIPTION OF THE DRAWINGS

FIG. 1A shows a non-limiting schematic of the mRNA-based reprogramming and gene-editing, followed by differentiation of the present disclosure. FIG. 1B illustrates differentiated cells killing cancer cells.

FIG. 2 shows the design of the gene-editing scheme for beta-2-microglobulin (B2M); shown are the following sequences: TCATCCATCCGACATTGA (SEQ ID NO: 1), AGTTGACTTACTGAAG (SEQ ID NO: 2), AATGGAGAGAGAATTGAA (SEQ ID NO: 3).

FIG. 3 shows an RNA gel demonstrating gene-editing of B2M.

FIG. 4 shows a sequencing experiment that shows the 14 base pair deletion from a gene-edited B2M; shown are the following sequences from bottom to top: ACATTGAAGAATGGAG (SEQ ID NO: 4), ACATTGAAGTTGACTTACTGAAGAATGGAG (SEQ ID NO: 5), and TGAATTGCTATGTGTCTGGGTTTCATCCATCCGACATTGAAGTTGACTTACTGAAGAAT GGAGAGAGAATTGAAAAAGTGGAGCATTCAGACTTGT (SEQ ID NO: 6).

FIG. 5 shows RNA levels of B2M with or without IFN gamma activation (“IFNY”; two left bars are the B2M knockout, and the two right bars are naïve cells).

FIG. 6 shows a sequencing experiment that demonstrates heterozygosity of CD16a (at G147D dbSNP:rs443082, Y158H dbSNP:rs396716, and F176V dbSNP:rs396991); shown are the following sequences from top to bottom: GKGRKYFHHNSDFHIPKATLKDS (SEQ ID NO: 7), GKDRKYFHHNSDFYIPKATLKDS (SEQ ID NO: 8), KDSGSYFCRGLFGSKNVSSETVN (SEQ ID NO: 9), and KDSGSYFCRGLVGSKNVSSETVN (SEQ ID NO: 10).

FIG. 7A-7B shows images of control (PMBC-isolated) NK cells in co-culture with K-562 tumor cells, demonstrating NK Cell cytotoxicity of tumor cell (note immunothrombosis or “clumping”).

FIG. 8A-8B shows images of the gene edited and differentiated cells of the present disclosure (e.g., B2M knockout NK cells) in co-culture with K-562 tumor cells, demonstrating NK Cell cytotoxicity of tumor cell (note immunothrombosis or “clumping”).

FIG. 9A-FIG. 9H show results of the cytokine release assay with the Luminex MAGPIX. Unless indicated (i.e., “+IL2, IL15”), conditions are without added IL-2 or IL-15. Further, ratio of cells is indicated (1:1 or 3:1). As elsewhere herein, PBMC-NK are control NK cells. FIG. 9A shows interferon gamma. FIG. 9B shows IL-2. FIG. 9C shows IL-7. FIG. 9D shows IL-13. FIG. 9E shows MIP-1a. FIG. 9F shows MIP-1b. FIG. 9G shows TNFα. FIG. 9H shows GM-CSF.

FIG. 10A-FIG. 10D show flow cytometry data for a gene edited and differentiated cells of the present disclosure (e.g., B2M knockout NK cells) as described in the Examples.

FIG. 11A shows the structure for the B2M-HLA-E repair template. FIG. 11B shows an ideal target site for the B2M-HLA-A repair template is shown (SEQ ID NO: 11: MSRSVALAVLALLSLSGLEAIQ; and SEQ ID NO: 12 ATGTCTCGCTCCGTGGCCTTAGCTGTGCTCGCGCTACTCTCTCTTTCTGGCCTGGAGGC TATCCAGCgtgagtctctcctaccctcccgctc). FIG. 11C shows additional target binding sites (SEQ ID NO: 11 and SEQ ID NO: 12 are again shown). FIG. 11D shows a gel with sizes of two lines having the B2M-HLA-E repair template inserted. FIG. 11E includes graphs showing the intensities of signal and ratios thereof from the bands shown in FIG. 11D. FIG. 11F shows a gel with sizes of two lines having the B2M-HLA-E repair template inserted. FIG. 11G includes graphs showing the intensities of signal and ratios thereof from the bands shown in FIG. 11F. FIG. 11H shows relevant sequences in the B2M-HLA-E repair template.

FIG. 12A and FIG. 12B, show target site sequences and repair templates for replacing the phenylalanine (F) at position 158 of CD16a with a valine (V). Relevant sequences are shown in these figures.

FIG. 13 is a graphical representation of different protocols in the differentiation of cytotoxic lymphocytes.

FIG. 14 is an illustrative flow cytometry fluorescence map used in data analysis of cytotoxicity assays.

FIG. 15 are graphs showing percentages of cancer killed in 24 hours. The left data for each graph are cells that were not activated and the right data for each graph are cells that were activated with IL-15 and IL-2.

FIG. 16 are graphs showing the ability of cytotoxic lymphocytes to kill K692 cancer cells and their inability to kill NK-resistant cancer cells.

FIG. 17 is a scatter plot showing two distinct populations of cells.

FIG. 18 are scatter plots for cells derived from Protocol 2, 3, or 4 as illustrated in FIG. 13.

FIG. 19 is a cartoon showing methods for manufacturing mixed population iPS-cell derived lymphoid lineage cells and myeloid lineage cells for enhanced tumor cell killing.

FIG. 20 shows an overview of scalable iPSC differentiation into lymphoid and myeloid cells.

FIG. 21 includes photomicrographs showing morphology of iPSC to macrophage progenitor cells in the T75 and Bioreactor.

FIG. 22 includes graphs showing the sum of the viable macrophage progenitor cells harvested from the T75 (left) and bioreactor (right) throughout the culture period. * Indicates when a large harvest (>50% of total cells) was performed.

FIG. 23 is a graph assessing baseline macrophage cytotoxicity.

FIG. 24 includes florescent photomicrographs of mRNA transfection of macrophages.

FIG. 25 includes photomicrographs showing morphology of NK cells on the final day of the differentiation protocol (left) versus 24 hours post thaw (right).

FIG. 26 is a graph showing the effects of cryopreservation on the cytotoxicity of NK cells.

FIG. 27 includes a graph (left) and photomicrographs (right) of data from an isolated huPBMC mixed cell type cytotoxicity assay.

FIG. 28 includes data from cytotoxicity assay cytokine release heat maps with iPS derived macrophages and NK cells. * Indicates below level of detection. ** Indicates sample read N/A.

FIG. 29 includes photomicrographs showing iPSC derived immune cell clustering during a cytotoxicity assay after 24 hours.

FIG. 30 is a cartoon showing methods for rapid prototyping of macrophage gene-editing strategies for cancer immunotherapies.

FIG. 31 is a timeline showing efficient differentiation of iPSCs into macrophages.

FIG. 32 is a photomicrograph showing iPSC-macrophages displaying macrophage-like morphology following differentiation.

FIG. 33 is a schematic of an mRNA encoding a ROR1-CAR.

FIG. 34A includes florescent photomicrographs and FIG. 34B includes graphs of data from GFP-encoding mRNA transfected into macrophages.

FIG. 35A includes florescent photomicrographs and FIG. 35B includes graphs of data from macrophages transfected with mRNA encoding an ROR1-CAR.

FIG. 36 includes photomicrographs of data from a Zymosan bead phagocytosis assay.

FIG. 37 includes photomicrographs of data from a CD3ζ phosphorylation assay.

FIG. 38 includes photomicrographs (left) and a graph (right) of data from an SKOV3 cytotoxicity assay.

FIG. 39 is a cartoon showing a protocol for insertion of transgene.

FIG. 40 is a schematic of the structure of a ROR1-CAR transgene.

FIG. 41 is a gel showing insertion of a ROR1-CAR transgene into iPSC lines.

FIG. 42 is a cartoon showing methods for reprogramming fibroblasts into induced pluripotent stem cells (iPSCs), which are then differentiated into monocytes which are further differentiated into cancer killing macrophages.

FIG. 43A to FIG. 43C show general steps for the process of differentiating iPSCs to macrophage.

FIG. 43A shows iPSC to monocyte differentiation, FIG. 43B shows CD14+ magnetic bead positive selection, and FIG. 43C shows monocyte to macrophage differentiation.

FIG. 44 shows progressing of cells from an iPSC colony (top left), on day 3 mesoderm (top right), on day 7 hematopoietic stem cell (bottom left), and on day 14 monocyte (bottom right).

FIG. 45A to FIG. 45C are flow cytograms showing peaks marked by CD14 (FIG. 45A), CD45 (FIG. 45B), and CD163 (FIG. 45C) for cryopreserved PBMC-monocytes and for cryopreserved iPSC-monocytes flowed directly after thawing.

FIG. 46 shows monocyte cultures at day zero (left image) and at day 4 and upon activation with M-CSF (right image).

FIG. 47 shows macrophage cultures at day zero (left image) and at day 3 after (right image).

FIG. 48 is a graph showing ELISA on iPSC-Macrophage supernatants (1M cells/mL).

FIG. 49 shows the process for testing the cancer-cell killing ability of iPSC-macrophages of the present disclosure.

FIG. 50 is a flow cytometry scatter plot showing iPSC-derived macrophages killing of U2OS cancer cells in vitro.

FIG. 51 is a chart showing that iPSC-derived macrophages killed 45% of U2OS cancer cells in vitro.

FIG. 52 is a flow cytometry scatter plot showing iPSC-derived macrophages killing of MA011sk cells or donor fibroblasts in vitro.

FIG. 53 is a chart showing that iPSC-derived macrophages do not kill MA011sk cells or donor fibroblasts in vitro.

FIG. 54 is a cartoon showing methods for reducing an immune response by including a TTAGGG motif in an dsODN.

FIG. 55A to FIG. 55C shows that iMSC electroporated with a synthetic nucleic acid encoding a gene-editing protein and with a repair template comprising the code for GFP, expressed GFP 24 hours after electroporation (FIG. 55A), 72 hours after electroporation (FIG. 55B), and 28 days hours after electroporation and by passage 4 (FIG. 55C).

FIG. 56 is a blot showing results from gene-editing iMSCs with the gene-editing protein alone (lane 3) or with the gene-editing protein, the A151 oligo, dsODN repair template, and U11 (lane 4).

FIG. 57 are flow cytometry scatter plots for iMSCs that were gene edited along with the A151 ODN.

FIG. 58 is a cartoon showing the process of contacting Resveratrol with a cell in advance of gene editing.

FIG. 59 are graphs showing numbers of cells in S/G2 that were not pretreated with Resveratrol (left graph) and numbers of cells in S/G2 that were pretreated with Resveratrol (right graph).

FIG. 60 is a gel showing that Resveratrol pretreated fibroblasts have increased gene-editing efficiency.

FIG. 61 is a gel showing that that Resveratrol treatment after electroporation with gene-editing nucleic acids increased 1 kb insertion 1.6-fold in iPSC.

FIG. 62 is a gel showing that NU7441 (a DNA-PKs inhibitor that is known to inhibit NHEJ mediated DNA repair pathway) have increased gene-editing efficiency.

FIG. 63 is a gel showing that a maximum 32% of 1 kb insertion rate was observed using ssDNA repair template in iPSCs.

### DETAILED DESCRIPTION

### Methods for Manufacturing Cytotoxic Lymphocytes

An aspect of the present disclosure is a method for manufacturing a population of cells that is enriched for cytotoxic lymphocytes. The method comprises steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes. In this aspect, steps (5) and (6) occur in an adherent culturing vessel. When CD34+ cells are selected, the embryoid bodies may be first chemically and/or mechanically dissociated.

In embodiments, the stem cell is an induced pluripotent stem (iPSC).

In some embodiments, the stem cell has a wild-type genome or has a genetically engineered disruption in a beta-2-microglobulin (B2M) gene. In some cases, the stem cell has a biallelic disruption in a B2M gene.

In some cases, mRNA-reprogrammed iPSC lines with a biallelic knockouts of the beta-2 microglobulin (B2M) gene, a key component of MHC class I molecules, are obtained using an mRNA-encoded chromatin context-sensitive gene-editing endonuclease. The B2M-knockout iPSCs may be differentiated using a novel, fully suspension process that replaces specialized micropatterned culture vessels with a spheroid culture step. Additional details regarding B2M knockout iPSCs useful in the present disclosure are described in PCT/US2022/019020, the contents of which are incorporated herein by reference in its entirety.

In various embodiments, the bioreactor is suited for culturing shear-sensitive cells and/or does not require use of anti-foaming agents or shear protectants, e.g., a vertical wheel bioreactor such as a PBS Biotech vertical-wheel bioreactor.

In embodiments, the medium in step (2) is serum-free and feeder-free culture medium, e.g., an mTeSR™ medium.

In some embodiments, the medium in step (6) is a serum-free and feeder-free culture medium, e.g., a StemDiff™ NK medium.

In various embodiments, the adherent culturing vessel is a multi-well plate or a cell culturing flask.

In embodiments, the method provides from about 10-fold to about 100-fold more cytotoxic lymphocytes than obtained by a method in which each of the culturing steps comprise adherent culturing vessels; obtained by a method in which step (2) comprises a spheroid suspension culture and steps (3), (5), and (6) occur in adherent culturing vessels; and/or obtained by a method in which steps (5) and (6) occur in bioreactor.

In some embodiments, the cytotoxic lymphocytes are enriched for CD56+ cells, for CD16+ cells, NKG2D+ cells, CD226+ Cells, NKp46+ cells, NKp44+ cells, CD244+ cells, and/or CD94+ cells.

In various embodiments, the method provides from about 5-fold to about 30-fold more CD16+ cells than obtained by a method in which step (2) comprises a spheroid suspension culture and steps (3), (5), and (6) occur in adherent culturing vessels and/or obtained by a method in which steps (5) and (6) occur in bioreactor.

In embodiments, the method provides from about 5-fold to about 25-fold more NDG2D+ cells than obtained by a method in which step (2) comprises a spheroid suspension culture and steps (3), (5), and (6) occur in adherent culturing vessels and/or obtained by a method in which steps (5) and (6) occur in bioreactor.

In some embodiments, the method provides from about 2-fold to about 30-fold more NKp44+ cells than obtained by a method in which step (2) comprises a spheroid suspension culture and steps (3), (5), and (6) occur in adherent culturing vessels and/or obtained by a method in which steps (5) and (6) occur in bioreactor.

In various embodiments, the method provides from about 2-fold to about 8-fold more CD94+ cells than obtained by a method in which step (2) comprises a spheroid suspension culture and steps (3), (5), and (6) occur in adherent culturing vessels and/or obtained by a method in which steps (5) and (6) occur in bioreactor.

In embodiments, the method provides from about 2-fold more NKp46 cells than obtained by a method in which step (2) comprises a spheroid suspension culture and steps (3), (5), and (6) occur in adherent culturing vessels.

In some embodiments, the cytotoxic lymphocyte targets and kills cancer cells, e.g., a K562 cancer cell. In various embodiments, the cytotoxic lymphocyte targets and kills cancer cells without requiring IL-15 and/or without requiring IL-2 activation. In embodiments, the cytotoxic lymphocyte targets and kills at least 70% of cancer cells in a population within about 4 hours. In some embodiments, the cytotoxic lymphocyte targets and kills at least 80% of cancer cells in a population within about 24 hours.

In various embodiments, the cytotoxic lymphocyte has reduced cytotoxicity to an NK-resistant cancer cell, e.g., a NAMALWA cell.

In embodiments, the cytotoxic lymphocyte is a Natural Killer (NK) cell. In some cases, the NK cell is a mature NK cell.

In some embodiments, the cytotoxic lymphocyte is a Natural killer T (NKT) cell.

In various embodiments, the cytotoxic lymphocyte is a delta-gamma T cell.

In embodiments, the iPSC was reprogrammed from a somatic cell comprising contacting the somatic cell with one or more ribonucleic acids (RNAs), wherein each RNA encodes one or more reprogramming factors.

### Cytotoxic Lymphocytes

In embodiments, the present cytotoxic lymphocyte is of the lymphoid cell lineage or the myeloid cell lineage.

In some cases, the lymphoid cell is a T cell, e.g., a cytotoxic T cell or gamma-delta T cell.

In some cases, the lymphoid cell is an NK cell, e.g., an NK-T cell. The NK cell may be a human cell. In some cases, the myeloid cell is a macrophage, e.g., an M1 macrophage or an M2 macrophage.

In various embodiment, the cytotoxic lymphocyte is reprogrammed from a stem cell, e.g., an iPSC, and differentiated into the cytotoxic lymphocyte.

In embodiments, the cytotoxic lymphocyte has a disruption in its beta-2-microglobulin (B2M) gene. In embodiments, the cytotoxic lymphocyte has a disruption in its beta-2-microglobulin (B2M) gene and expresses a fusion protein comprising a B2M polypeptide and an HLA polypeptide (e.g., an HLA-A, HLA-B, HLA-C, HLA-E, HLA-F and HLA-G polypeptide).

In embodiments, the cytotoxic lymphocyte is gene edited to express a high affinity variant of CD16a (See, FIG. 12A and FIG. 12B).

In embodiments, the myeloid lineage cell is a cell derived from, or derivable from, a common myeloid progenitor cell. In embodiments, the myeloid cell is a megakaryocyte, erythrocyte, mast cell, or myeloblast. In embodiments, the myeloid cell is a cell derived from, or derivable from, a myeloblast. In embodiments, the myeloid cell is a basophil, neutrophil, eosinophil, or monocyte. In embodiments, the myeloid cell is a cell derived from, or derivable from a monocyte. In embodiments, the myeloid cell is a macrophage. In embodiments, the myeloid cell is a dendritic cell.

In embodiments, the cytotoxic lymphocyte is an NK cell. In embodiments, the NK cell is a human cell. In embodiments, the NK cell is derived from somatic cell of a subject. In embodiments, the NK cell is derived from allogeneic or autologous cells. In embodiments, the NK cell is derived from an induced pluripotent stem (iPS) cell. In embodiments, the iPS is derived from reprogramming a somatic cell to an iPS cell, the reprogramming comprising contacting the iPS cell with a ribonucleic acid (RNA) encoding one or more reprogramming factors, optionally selected from Oct4, Sox2, cMyc, and Klf4. In embodiments, the iPS cell is derived from allogeneic or autologous cells. In embodiments, the NK cell expresses one or more of CD56 and CD16.

In embodiments, the NK cell expresses CD16a, which optionally binds an antibody/antigen complex on a tumor cell and/or wherein the CD16a is optionally a high affinity variant, optionally homozygous or heterozygous for F158V (See, FIG. 12A and FIG. 12B).

In embodiments, the NK cell does not express CD3.

In embodiments, the NK cell is CD56^{bright} CD16^{dim/-}. In embodiments, the NK cell is CD56^{dim} CD16+. In embodiments, the NK cell is a NK^{tolerant} cell, optionally comprising CD56^{bright} NK cells or CD27− CD11b− NK cells. In embodiments, the NK cell is a NK^{cytotoxic}, optionally comprising CD56^{dim} NK cells or CD11b+ CD27− NK cells. In embodiments, the NK cell is a NK^{regulatory}, optionally comprising CD56^{bright} NK cells or CD27+ NK cells. In embodiments, the NK cell is a natural killer T (NKT) cell. In embodiments, the NK cell secretes one or more cytokines selected from interferon-gamma (IFN-g), tumor necrosis factor-alpha (TNF-a), tumor necrosis factor-beta (TNF-b), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b).

In embodiments, the present cytotoxic lymphocyte has reduced or eliminated cytotoxic lymphocyte fratricide, e.g., NK-cell fratricide. For instance, in embodiments, the present engineered NK cells surprisingly do not engage in NK cytotoxicity and therefore are able to survive despite disruptions, e.g., in beta-2-microglobulin (B2M).

In embodiments, the present cytotoxic lymphocyte is capable of self-activating. In embodiments, the present cytotoxic lymphocyte is capable of activating without the need for extracellular signals (e.g., cytokines), including signals that may be provided exogenously. In embodiments, the present cytotoxic lymphocyte does not require ex vivo stimulation for activity. In embodiments, the present cytotoxic lymphocyte is capable of self-activating in the absence of an interleukin, optionally selected from IL-2 and IL-15.

In embodiments, the present cytotoxic lymphocyte is capable of inducing tumor cell cytotoxicity. In embodiments, the present cytotoxic lymphocyte is capable of inducing tumor cell cytotoxicity in the absence of an interleukin, optionally selected from IL-2 and IL-15. Assays for assessing tumor cell cytotoxicity include in vivo anti-cancer response evaluation, as well as microscopic evaluation, e.g., a calcein acetoxymethyl (AM) staining-based microscopic method (See EXAMPLES and Chava et al. J Vis Exp. 2020 Feb. 22; (156): 10.3791/60714, the entire contents of which are incorporated by reference). Further, a colorimetric lactic dehydrogenase (LDH) measurement-based NK cell-mediated cytotoxicity assay may be employed (see Chava et al. J Vis Exp. 2020 Feb. 22; (156): 10.3791/60714, the entire contents of which are incorporated by reference).

Scalable, Mixed Population iPS-Cell Derived Cytotoxic Lymphocytes and Myeloid Cells

Induced pluripotent stem cell (iPSC) therapies have the potential to treat a wide variety of devastating diseases. iPSC-derived lymphocytes (e.g., T cells and NK cells) engineered to express targeting molecules such as chimeric antigen receptors (CARs) have shown clinical promise to treat hematological malignancies. More recently, iPS cell-derived myeloid cells are being developed to treat both hematological malignancies and solid tumors due to the ability of these cells to infiltrate and modulate the tumor microenvironment. Despite preliminary success, several challenges still remain, including poor infiltration of cytotoxic lymphocytes into solid tumors and insufficient cytotoxicity of myeloid cells.

As is known in the art, an animal's immune system comprises a wide variety of immune cell types capable of contributing to an anti-cancer effect. And, in vivo, one type of immune cell promotes the cancer-killing ability of a second type of immune cell. Notably, NK cells are expert in killing cancer cells but rarely not infiltrate solid tumors alone and require recruitment by macrophages which have already infiltrated the solid tumor and, on the other hand, macrophages are less adept at killing cancer cells but expert in infiltrating solid tumors and secreting cytokines that recruit cancer killing cells. Thus, each type of immune cell has its function which work in cooperation with the other cell types to attack and kill cancer cells. Nonetheless, many cell-based cancer therapeutics in clinical trials employ one type of immune cell rather than a plurality of immune cell types as existent in vivo. Without wishing to be bound by theory, a multi-cell-type therapy comprising both lymphocyte and myeloid cells may work synergistically, enhancing cytotoxicity and efficacy.

This disclosure, e.g., in Examples 8 and 9, describes a scalable bioreactor-based process for parallel differentiation of mRNA reprogrammed iPSC into both CD14+ (>95% positive) macrophages and CD56bright/CD16dim NK cells. This process yielded 1×10⁶ myeloid cells/ml and 3×10⁵ lymphoid cells/ml, and is amenable to scaling to clinically relevant doses. In vitro, the lymphoid and myeloid cells showed synergistic tumor cell killing of SKOV3 ovarian cancer cells (combined: 15.6%; macrophage alone=2.2% (p<0.01); NK alone=7.5% (p<0.05); E:T=5:1). The combined cells showed increased expression of TNFα and demonstrated enhanced clustering and tumor cell engagement. To further improve the macrophages' ability to target and infiltrate solid tumors, macrophages were transfected with mRNA encoding a humanized ROR1-CAR protein. mRNA transfection increased cytotoxicity towards SKOV3 cells by 6-fold.

In conclusion, the present disclosure provides a scalable platform for generating iPSC-derived multi-cell-type therapies comprising both lymphoid and myeloid cells. These cells act synergistically to kill tumor cells in vitro. And, by closely mimicking natural cellular immunity, multi-cell-type cell therapies represent a new class of cell therapies that may play an important role in the development of new medicines for treating cancer.

An aspect of the present disclosure is a method for treating a cancer. The method comprising administering to a subject in need a therapeutically-effective amount of a first pharmaceutical composition comprising one or both of a population of isolated lymphoid lineage cells and a population of isolated myeloid lineage cells.

In embodiments, the first pharmaceutical composition comprises the population of isolated lymphoid lineage cells and wherein the subject in need is administered a therapeutically-effective amount of a second pharmaceutical composition comprising a population of isolated myeloid lineage cells.

In some embodiments, the first pharmaceutical composition comprises the population of isolated myeloid lineage cells and wherein the subject in need is administered a therapeutically-effective amount of a second pharmaceutical composition comprising a population of isolated lymphoid lineage cells. In some cases, the first pharmaceutical composition and the second pharmaceutical composition are administered simultaneously or sequentially. The first pharmaceutical composition and the second pharmaceutical composition may be administered sequentially with the first pharmaceutical composition administered before the second pharmaceutical composition or the first pharmaceutical composition and the second pharmaceutical composition may be administered sequentially with the second pharmaceutical composition administered before the first pharmaceutical composition.

In various embodiments, the first pharmaceutical composition comprises both the population of isolated lymphoid lineage cells and the population of isolated myeloid lineage cells.

In numerous embodiments, one or more of the isolated lymphoid lineage cells and/or one or more of the isolated myeloid lineage cells comprise a genomic modification that expresses a chimeric antigen receptor (CAR), e.g., a CAR which comprises an antigen binding region that binds to one or more antigens expressed by a cancer cell. In some cases, the antigen binding region binds to one or more tumor antigens. In various cases, the CAR comprises an antigen binding region that binds to ROR1. In embodiments, one or more of the isolated lymphoid lineage cells and/or one or more of the isolated myeloid lineage cells comprise a genomic modification that expresses or over expresses a cytokine. In some embodiments, one or more of the isolated lymphoid lineage cells and/or one or more of the isolated myeloid lineage cells comprise a genomic modification which disrupts the beta-2-microglobulin (B2M) gene, optionally, wherein the cells express a fusion protein comprising a B2M polypeptide and an HLA polypeptide (e.g., an HLA-A, HLA-B, HLA-C, HLA-E, HLA-F and HLA-G polypeptide).

In various embodiments, the method further comprises administering to the subject in need a synthetic mRNA encoding a gene-editing protein (e.g., a temperature-sensitive gene-editing protein) and a single-stranded or double-stranded repair template which encodes a chimeric antigen receptor (CAR). In some cases, the gene-editing protein creates a single-stranded break or a double-stranded break in the genomic DNA of a cell in the subject and the single-stranded or double-stranded repair template which encodes the CAR inserts into the break. In these embodiments, the cell in the subject expresses the CAR.

In numerous embodiments, the method further comprises administering to the subject in need a synthetic mRNA encoding a gene-editing protein (e.g., a temperature-sensitive gene-editing protein) and a single-stranded or double-stranded repair template which encodes a cytokine. In some cases, the gene-editing protein creates a single-stranded break or a double-stranded break in the genomic DNA of a cell in the subject and the single-stranded or double-stranded repair template which encodes the cytokine inserts into the break. In these embodiments, the cell in the subject expresses or over expresses the cytokine.

When the synthetic mRNA and/or the repair template is administered to a subject, the synthetic mRNA and/or the repair is combined with a lipid system comprising a compound of Formula (IV).

In various cases, transfection of a cell with synthetic nucleic acids for gene-editing may be facilitated by use of the ToRNAdo™ Nucleic-Acid Delivery System. This system relates to new lipids that find use, inter alia, in improved delivery of biological payloads, e.g., nucleic acids, to cells. The system relates to use of a compound of Formula (IV)

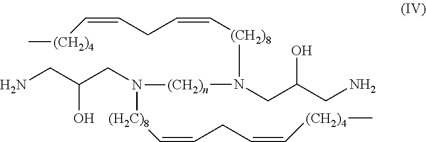

where n is 1, 2, 3, 4, 5, 6, 7, 8, 9, 10, 11, 12, 13, 14, or 15. Further description of ToRNAdo™ Nucleic-Acid Delivery System is found in one or both of U.S. Pat. No. 10,501,404 and WO2021003462. The entire contents of which are incorporated by reference in their entirety.

In embodiments, the cell in the subject (e.g., which expresses the CAR and/or cytokine) is of the lymphoid lineage or is of the myeloid lineage.

In some embodiments, the isolated lymphoid lineage cell and/or the isolated myeloid lineage cell is derived from an induced pluripotent stem cell (iPSC). In some cases, the isolated lymphoid lineage cell and the isolated myeloid lineage cell is derived from the same iPSC. The iPSC comprises a genomic modification that expresses a chimeric antigen receptor (CAR) and/or the iPSC comprises a genomic modification that expresses or over expresses a cytokine. In various cases, the iPSC comprises a genomic modification which disrupts the beta-2-microglobulin (B2M) gene.

In various embodiments, the isolated lymphoid lineage cells are manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes; wherein steps (5) and (6) occur in an adherent culturing vessel.

In numerous embodiments, the isolated lymphoid lineage cells comprise cytotoxic lymphocytes. In some cases, the isolated lymphoid lineage cells comprising cytotoxic lymphocytes are enriched for CD56+ cells, for CD16+ cells, NKG2D+ cells, CD226+ Cells, NKp46+ cells, NKp44+ cells, CD244+ cells, and/or CD94+ cells. In these embodiments, the cytotoxic lymphocyte targets and kills cancer cells and, in some cases, the cytotoxic lymphocyte targets and kills cancer cells without requiring IL-15 and/or without requiring IL-2 activation. The cytotoxic lymphocyte has reduced cytotoxicity to an NK-resistant cancer cell. In various cases, the cytotoxic lymphocyte is a Natural Killer (NK) cell, e.g., a mature NK cell, or is a cytotoxic T cell. In these embodiments, the cytotoxic lymphocyte is a Natural killer T (NKT) cell. In some cases, the NK cell expresses CD16a and/or the NK cell does not express CD3, and/or the NK cell is CD56bright CD16dim/−. In many cases, the NK cell secretes one or more cytokines selected from interferon-gamma (IFNγ), tumor necrosis factor-alpha (TNFα), tumor necrosis factor-beta (TNFβ), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b). The cytotoxic lymphocyte may be a delta-gamma T cell. In numerous cases, the cytotoxic lymphocyte is further engineered to express a chimeric antigen receptor (CAR) and/or is further engineered to express or overexpress a cytokine.

In embodiments, the isolated myeloid lineage cells are manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a myeloid progenitor medium; and (6) culturing the cells of step (5) in a macrophage cell medium under conditions to obtain a population of cells enriched for macrophages; wherein steps (5) and (6) occur in a bioreactor.

In some embodiments, the isolated myeloid lineage cells comprise a megakaryocyte, erythrocyte, mast cell, myeloblast, dendritic cell, basophil, neutrophil, eosinophil, monocyte, or macrophage.

In various embodiments, the isolated myeloid lineage cells express one or more of CD11b, CD13, CD14, CD33, CD45, CD80, CD163, CD206, and SIRPα, e.g., in amounts that are similar to amounts expressed by PBMC-derived cells.

In numerous embodiments, the isolated myeloid lineage cells have increased expression of CD80 and/or CD206, which is indicative of an activated state.

In embodiments, the isolated myeloid lineage cell is a macrophage. In some cases, the macrophage expresses one or more of CD11b, CD68, CD80, CD86, CD163, CD206, and SIRPα in amounts that are similar to amounts expressed by PBMC-derived cells and/or secretes one or more of TNFα, IL-12p70, and IL-10 in amounts that are similar to amounts expressed by PBMC-derived cells. In various cases, the macrophage expresses one or more of CD34, CD44, CD45, CD73, and CD90. In these embodiments, the method further comprises a step of differentiating the macrophages into M1 and/or M2 macrophages, e.g., by exposure to MCSF. And, the method may further comprise a step of polarizing the M1 macrophages with interferon gamma (IFN-γ) and/or lipopolysaccharide (LPS) and/or treating the M2 macrophages with IL-4. In these cases, the macrophages comprise M1 macrophages and/or M2 macrophages. The M1 macrophages and/or M2 macrophages secrete one or more of TNFα, IL-12p70, and IL-10 in amounts that are similar to amounts expressed by PBMC-derived cells.

In some embodiments, the isolated myeloid lineage cells kill cancer cells and/or promote cancer cell killing by cytotoxic lymphocytes.

In various embodiments, the isolated myeloid lineage cell is further engineered to express a chimeric antigen receptor (CAR).

In numerous embodiments, the isolated myeloid lineage cell is further engineered to express or overexpress a cytokine. In cases when CD34+ cells are selected, the embryoid bodies are first chemically and/or mechanically dissociated.

In embodiments, the stem cell is an induced pluripotent stem (iPSC). In some cases, the stem cell stem has a wild-type genome or has a genetically engineered disruption in a beta-2-microglobulin (B2M) gene, e.g., a biallelic disruption in a B2M gene. In various cases, the stem cell expresses a fusion protein comprising a B2M polypeptide and an HLA polypeptide (e.g., an HLA-A, HLA-B, HLA-C, HLA-E, HLA-F and HLA-G polypeptide).

In some embodiments, the iPSC was reprogrammed from a somatic cell and the method further comprises contacting the somatic cell with one or more ribonucleic acids (RNAs), wherein each RNA encodes one or more reprogramming factors. The one or more reprogramming factors may be selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In some cases, the somatic cell is selected from fibroblasts, keratinocytes, melanocyte blood cells, bone marrow cells, adipose cells, and tissue-resident progenitor cells.

In embodiments where the stem cell is an iPSC, the iPSC is further engineered to express a chimeric antigen receptor (CAR) and/or the iPSC is further engineered to express or overexpress a cytokine. In various embodiments, the isolated lymphoid lineage cells and the isolated myeloid lineage cells are manufactured by a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor. In cases when CD34+ cells are selected, the embryoid bodies are first chemically and/or mechanically dissociated. In various cases, the stem cell is an induced pluripotent stem (iPSC).

In embodiments, the method of manufacturing provides at least 1×10⁶ myeloid lineage cells/ml and at least 3×10⁵ lymphoid lineage cells/ml.

In some embodiments, the method of manufacturing provides both CD14+ (>95% positive) macrophages and CD56^{bright}/CD16^{dim} NK cells.

In various embodiments, the method of manufacturing is amenable to scaling to clinically relevant doses.

In embodiments, the population of isolated lymphoid lineage cells and the population of isolated myeloid lineage cells act synergistically to kill cancer cells.

In numerous embodiments, the administering is intravenous, intraarterial, intratumoral, or injected in the vicinity of a tumor.

In embodiments, the cancer is a blood cancer.

In some embodiments, the cancer is a solid tumor.

In various embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Another aspect of the present disclosure is a plurality of compositions for use in any herein-disclosed method for treating a cancer.

Yet another aspect of the present disclosure is a method for killing a cancer cell or for inhibiting the proliferation of a cancer cell. The method comprising contacting the cancer cell with a population of isolated lymphoid lineage cells and a population of isolated myeloid lineage cells.

In numerous embodiments, the cancer cell is contacted with the population of isolated lymphoid lineage cells and the population of isolated myeloid lineage cells simultaneously.

In embodiments, the cancer cell is contacted with the population of isolated lymphoid lineage cells before being contacted with the population of isolated myeloid lineage cells or the cancer cell is contacted with the population of isolated lymphoid lineage cells after being contacted with the population of isolated myeloid lineage cells.

In some embodiments, wherein one or more of the isolated lymphoid lineage cells and/or one or more of the isolated myeloid lineage cells comprise a genomic modification that expresses a chimeric antigen receptor (CAR). In some cases, the CAR comprises an antigen binding region that binds to one or more antigens expressed by a cancer cell. In various cases, the antigen binding region binds to one or more tumor antigens. In these embodiments, the CAR may comprise an antigen binding region that binds to ROR1.

In various embodiments, one or more of the isolated lymphoid lineage cells and/or one or more of the isolated myeloid lineage cells comprise a genomic modification that expresses or over expresses a cytokine.

In numerous embodiments, one or more of the isolated lymphoid lineage cells and/or one or more of the isolated myeloid lineage cells comprise a genomic modification which disrupts the beta-2-microglobulin (B2M) gene, optionally, wherein the cells express a fusion protein comprising a B2M polypeptide and an HLA polypeptide (e.g., an HLA-A, HLA-B, HLA-C, HLA-E, HLA-F and HLA-G polypeptide).

In embodiments, the isolated lymphoid lineage cells comprise cytotoxic lymphocytes. In some cases, the isolated lymphoid lineage cells comprising cytotoxic lymphocytes are enriched for CD56+ cells, for CD16+ cells, NKG2D+ cells, CD226+ Cells, NKp46+ cells, NKp44+ cells, CD244+ cells, and/or CD94+ cells. In various cases, the cytotoxic lymphocyte targets and kills cancer cells, e.g., the cytotoxic lymphocyte targets and kills cancer cells without requiring IL-15 and/or without requiring IL-2 activation. The cytotoxic lymphocyte has reduced cytotoxicity to an NK-resistant cancer cell. In some cases, the cytotoxic lymphocyte is a Natural Killer (NK) cell, e.g., a mature NK cell, or is a cytotoxic T cell. The cytotoxic lymphocyte may be a Natural killer T (NKT) cell. In these embodiments, the NK cell expresses CD16a and/or the NK cell does not express CD3 and/or the NK cell is CD56bright CD16dim/−. In some cases, the NK cell secretes one or more cytokines selected from interferon-gamma (IFNγ), tumor necrosis factor-alpha (TNFα), tumor necrosis factor-beta (TNFβ), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b). In other case, the cytotoxic lymphocyte is a delta-gamma T cell. In these embodiments, the cytotoxic lymphocyte is further engineered to express a chimeric antigen receptor (CAR) and/or is further engineered to express or overexpress a cytokine.

In some embodiments, the isolated myeloid lineage cells comprise a megakaryocyte, erythrocyte, mast cell, myeloblast, dendritic cell, basophil, neutrophil, eosinophil, monocyte, or macrophage.

In various embodiments, the isolated myeloid lineage cells express one or more of CD11b, CD13, CD14, CD33, CD45, CD80, CD163, CD206, and SIRPα, e.g., in amounts that are similar to amounts expressed by PBMC-derived cells.

In numerous embodiments, the isolated myeloid lineage cells have increased expression of CD80 and/or CD206, which is indicative of an activated state.

In embodiments, the isolated myeloid lineage cell is a macrophage. In some cases, the macrophage expresses one or more of CD11b, CD68, CD80, CD86, CD163, CD206, and SIRPα in amounts that are similar to amounts expressed by PBMC-derived cells and/or secretes one or more of TNFα, IL-12p70, and IL-10 in amounts that are similar to amounts expressed by PBMC-derived cells. The macrophage may express one or more of CD34, CD44, CD45, CD73, and CD90. In various cases, the macrophages comprise M1 macrophages and/or M2 macrophages. The M1 macrophages and/or M2 macrophages may secrete one or more of TNFα, IL-12p70, and IL-10 in amounts that are similar to amounts expressed by PBMC-derived cells.

In some embodiments, the isolated myeloid lineage cells kill cancer cells and/or promote cancer cell killing by cytotoxic lymphocytes.

In various embodiments, the isolated myeloid lineage cell is further engineered to express a chimeric antigen receptor (CAR).

In numerous embodiments, the isolated myeloid lineage cell is further engineered to express or overexpress a cytokine.

In embodiments, the isolated lymphoid lineage cells and the isolated myeloid lineage cells are manufactured by a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor. In cases when CD34+ cells are selected, the embryoid bodies are first chemically and/or mechanically dissociated. In these embodiments, the stem cell may be an induced pluripotent stem (iPSC). In some cases, the isolated lymphoid lineage cell and the isolated myeloid lineage cell are derived from the same iPSC. In various cases, the iPSC comprises a genomic modification that expresses a chimeric antigen receptor (CAR) and/or comprises a genomic modification that expresses or over expresses a cytokine. The iPSC may comprise a genomic modification which disrupts the beta-2-microglobulin (B2M) gene, e.g., a biallelic disruption in a B2M gene; in these cases, the iPSC expresses a fusion protein comprising a B2M polypeptide and an HLA polypeptide (e.g., an HLA-A, HLA-B, HLA-C, HLA-E, HLA-F and HLA-G polypeptide).

In embodiments, the iPSC was reprogrammed from a somatic cell, and the method further comprises contacting the somatic cell with one or more ribonucleic acids (RNAs), wherein each RNA encodes one or more reprogramming factors. The one or more reprogramming factors may be selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In some cases, the somatic cell is selected from fibroblasts, keratinocytes, melanocyte blood cells, bone marrow cells, adipose cells, and tissue-resident progenitor cells.

In some embodiments, contacting the cancer cell with a population of isolated lymphoid lineage cells and a population of isolated myeloid lineage cells occurs in vitro.

In various embodiments, contacting the cancer cell with a population of isolated lymphoid lineage cells and a population of isolated myeloid lineage cells occurs in vivo.

Yet a further aspect of the present disclosure is a plurality of compositions for use in any herein-disclosed method for killing a cancer cell or for inhibiting the proliferation of a cancer cell.

In an aspect, the present disclosure provides a method for manufacturing a plurality of population of cells comprising a population of isolated lymphoid lineage cells and a population of isolated myeloid lineage cells for treating a cancer, for killing a cancer cell, and/or for inhibiting the proliferation of a cancer cell. The method a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor.

In numerous embodiments, when CD34+ cells are selected, the embryoid bodies are first chemically and/or mechanically dissociated.

In embodiments, the stem cell is an induced pluripotent stem (iPSC). In some cases, the isolated lymphoid lineage cell and the isolated myeloid lineage cell are derived from the same iPSC. The iPSC may comprise a genomic modification that expresses a chimeric antigen receptor (CAR) and/or a genomic modification that expresses or over expresses a cytokine. In various cases, the iPSC comprises a genomic modification which disrupts the beta-2-microglobulin (B2M) gene, e.g., a biallelic disruption in a B2M gene. In these cases, the iPSC expresses a fusion protein comprising a B2M polypeptide and an HLA polypeptide (e.g., an HLA-A, HLA-B, HLA-C, HLA-E, HLA-F and HLA-G polypeptide).

In some embodiments, the iPSC was reprogrammed from a somatic cell, and the method further comprises contacting the somatic cell with one or more ribonucleic acids (RNAs), wherein each RNA encodes one or more reprogramming factors. The one or more reprogramming factors may be selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In some case, the somatic cell is selected from fibroblasts, keratinocytes, melanocyte blood cells, bone marrow cells, adipose cells, and tissue-resident progenitor cells.

In various embodiments, one or more of the isolated lymphoid lineage cells and/or one or more of the isolated myeloid lineage cells comprise a genomic modification that expresses a chimeric antigen receptor (CAR), e.g., which comprises an antigen binding region that binds to one or more antigens expressed by a cancer cell. In some case, the antigen binding region binds to one or more tumor antigens. In these embodiments, the CAR may comprise an antigen binding region that binds to ROR1. In numerous embodiments, one or more of the isolated lymphoid lineage cells and/or one or more of the isolated myeloid lineage cells comprise a genomic modification that expresses or over expresses a cytokine.

In embodiments, one or more of the isolated lymphoid lineage cells and/or one or more of the isolated myeloid lineage cells comprise a genomic modification which disrupts the beta-2-microglobulin (B2M) gene. In some cases, the cells express a fusion protein comprising a B2M polypeptide and an HLA polypeptide (e.g., an HLA-A, HLA-B, HLA-C, HLA-E, HLA-F and HLA-G polypeptide).

In some embodiments, the isolated lymphoid lineage cells comprise cytotoxic lymphocytes. In some cases, the isolated lymphoid lineage cells comprising cytotoxic lymphocytes are enriched for CD56+ cells, for CD16+ cells, NKG2D+ cells, CD226+ Cells, NKp46+ cells, NKp44+ cells, CD244+ cells, and/or CD94+ cells. The cytotoxic lymphocyte targets and kills cancer cells. In various cases, the cytotoxic lymphocyte targets and kills cancer cells without requiring IL-15 and/or without requiring IL-2 activation. The cytotoxic lymphocyte may have reduced cytotoxicity to an NK-resistant cancer cell. The cytotoxic lymphocyte may be a Natural Killer (NK) cell, e.g., a mature NK cell, or is a cytotoxic T cell The cytotoxic lymphocyte may be a Natural killer T (NKT) cell. The the NK cell may express CD16a and/or the NK cell does not express CD3 and/or the NK cell is CD56bright CD16dim/−. In various cases, the NK cell secretes one or more cytokines selected from interferon-gamma (IFNγ), tumor necrosis factor-alpha (TNFα), tumor necrosis factor-beta (TNFβ), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b). The cytotoxic lymphocyte may be a delta-gamma T cell. In some cases, the cytotoxic lymphocyte is further engineered to express a chimeric antigen receptor (CAR) and/or is further engineered to express or overexpress a cytokine.

In various embodiments, the isolated myeloid lineage cells comprise a megakaryocyte, erythrocyte, mast cell, myeloblast, dendritic cell, basophil, neutrophil, eosinophil, monocyte, or macrophage.

In numerous embodiments, the isolated myeloid lineage cells express one or more of CD11b, CD13, CD14, CD33, CD45, CD80, CD163, CD206, and SIRPα, e.g., in amounts that are similar to amounts expressed by PBMC-derived cells.

In embodiments, the isolated myeloid lineage cells have increased expression of CD80 and/or CD206, which is indicative of an activated state.

In some embodiments, the isolated myeloid lineage cell is a macrophage. In some cases, the macrophage expresses one or more of CD11b, CD68, CD80, CD86, CD163, CD206, and SIRPα in amounts that are similar to amounts expressed by PBMC-derived cells and/or secretes one or more of TNFα, IL-12p70, and IL-10 in amounts that are similar to amounts expressed by PBMC-derived cells. In various cases, the macrophage expresses one or more of CD34, CD44, CD45, CD73, and CD90. In these embodiments, the method may further comprise a step of differentiating the macrophages into M1 and/or M2 macrophages, e.g., by exposure to MCSF. The method may also further comprise a step of polarizing the M1 macrophages with interferon gamma (IFN-γ) and/or lipopolysaccharide (LPS) and/or treating the M2 macrophages with IL-4. In various cases, the macrophages comprise M1 macrophages and/or M2 macrophages; the M1 macrophages and/or M2 macrophages may secrete one or more of TNFα, IL-12p70, and IL-10 in amounts that are similar to amounts expressed by PBMC-derived cells.

In various embodiments, the isolated myeloid lineage cells kill cancer cells and/or promote cancer cell killing by cytotoxic lymphocytes.

In numerous embodiments, the isolated myeloid lineage cell is further engineered to express a chimeric antigen receptor (CAR).

In embodiments, the isolated myeloid lineage cell is further engineered to express or overexpress a cytokine.

In some embodiments, the iPSC was contacted with resveratrol before reprogramming.

In various embodiments, one or more culturing steps comprise a medium which is serum-free culture medium and/or feeder-free culture medium. In some cases, the serum-free culture medium and/or feeder-free culture medium is an mTeSR™ medium and/or the serum-free culture medium and/or feeder-free culture medium is a StemDiff™ NK medium.

In numerous embodiments, the adherent culturing vessel is a multi-well plate or a cell culturing flask. In embodiments, the method of manufacturing provides at least 1×10⁶ myeloid lineage cells/ml and at least 3×10⁵ lymphoid lineage cells/ml.

In some embodiments, the method of manufacturing provides both CD14+ (>95% positive) macrophages and CD56^{bright}/CD16^{dim} NK cells.

In various embodiments, the method of manufacturing is amenable to scaling to clinically relevant doses.

In embodiments, the population of isolated lymphoid lineage cells and the population of isolated myeloid lineage cells act synergistically to kill cancer cells.

In embodiments, the cancer is a blood cancer.

In some embodiments, the cancer is a solid tumor.

In various embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In another aspect, the present disclosure provides a plurality population of cells comprising a population of isolated lymphoid lineage cells and a population of isolated myeloid lineage cells for treating a cancer, for killing a cancer cell, and/or for inhibiting the proliferation of a cancer cell which were manufactured by any herein-disclosed method.

### Chimeric Antigen Receptor (CAR)-Bearing Cytotoxic Lymphocytes

In embodiments, the present cytotoxic lymphocytes are engineered with chimeric antigen receptors (CARs), e.g., the present cytotoxic lymphocytes are CAR-NK cells, CAR-T cells, CAR-myeloid cells, or CAR-mesenchymal stromal/stem cells.

In embodiments, the cytotoxic lymphocyte, optionally NK cell or T cell, is genetically modified to express a recombinant chimeric antigen receptor (CAR) comprising an intracellular signaling domain, a transmembrane domain, and an extracellular domain comprising an antigen binding region. In embodiments, the intracellular signaling domain comprises at least one immune receptor tyrosine-based activation motif (ITAM)-containing domain.

In embodiments, the intracellular signaling domain is from one of CD3-zeta, CD28, CD27, CD134 (OX40), and CD137 (4-1BB).

In embodiments, the transmembrane domain is from one of CD28 or a CD8.

In embodiments, the antigen binding region binds one antigen. In embodiments, the binding region binds two antigens.

In embodiments, the extracellular domain comprising an antigen binding region comprises: (a) a natural ligand or receptor, or fragment thereof, or (b) an immunoglobulin domain, optionally a single-chain variable fragment (scFv). In embodiments, the extracellular domain comprising an antigen binding region comprises two of (a) a natural ligand or receptor, or fragment thereof, or (b) an immunoglobulin domain, optionally a single-chain variable fragment (scFv). In embodiments, the extracellular domain comprising an antigen binding region comprises one of each of: (a) a natural ligand or receptor, or fragment thereof, and (b) an immunoglobulin domain, optionally a single-chain variable fragment (scFv).

In embodiments, the antigen binding region binds a tumor antigen.

In embodiments, the antigen binding region comprises one or more of (i) CD94/NKG2a, which optionally binds HLA-E on a tumor cell; (ii) CD96, which optionally binds CD155 on a tumor cell; (iii) TIGIT, which optionally binds CD155 or CD112 on a tumor cell; (iv) DNAM-1, which optionally binds CD155 or CD112 on a tumor cell; (v) KIR, which optionally binds HLA class I on a tumor cell; (vi) NKG2D, which optionally binds NKG2D-L on a tumor cell; (vii) CD16 (e.g., CD16a or CD16b), which optionally binds an antibody/antigen complex on a tumor cell and/or wherein the CD16a is optionally a high affinity variant, optionally homozygous or heterozygous for F158V; (viii) NKp30, which optionally binds B7-H6 on a tumor cell; (ix) NKp44; and (x) NKp46.

In embodiments, the antigen binding region comprises an immunoglobulin domain, optionally an scFv directed against HLA-E, CD155, CD112 HLA class I, NKG2D-L, or B7-H6, as well as any variant thereof.

In embodiments, the antigen binding region binds an antigen, e.g., a tumor antigen, selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, as well as any variant thereof. In embodiments, an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, as well as any variant thereof can be used as a single-target CAR, dual-target CAR, mAb, or any combination of any of those.

In embodiments, the antigen binding region binds two antigen, e.g., two tumor antigens, the antigens being: (a) an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2, as well as any variant thereof and (b) an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2, as well as any variant thereof.

In embodiments, the antigen binding region binds two antigen, the antigens being: (a) an antigen selected from CD16, CD64, CD78, CD96, CLL1, CD116, CD117, CD71, CD45, CD71, CD123 and CD138, a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvlll), CD19, CD20, CD30, CD40, disialoganglioside GD2, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, CD22, insulin growth factor (IGFl)-1, IGF-I I, IGFI receptor, mesothelin, a major histocompatibility complex (MHC) molecule presenting a tumor-specific peptide epitope, 5T4, RORl, Nkp30, N KG2D, tumor stromal antigens, the extra domain A (EDA) and extra domain B (EDB) of fibronectin and the Al domain of tenascin-C (TnC Al) and fibroblast associated protein (FAP); a lineage-specific or tissue specific antigen such as CD3, CD4, CD8, CD24, CD25, CD33, CD34, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF17), multiple myeloma or lymphoblastic leukemia antigen, such as one selected from TNFRSF17, SLAMF7, GPRC5D, FKBP11, KAMP3, ITGA8, and FCRL5, a virus-specific surface antigen such as an HIV-specific antigen (such as HIV gpl20); an EBV-specific antigen, a CMV-specific antigen, a HPV-specific antigen, a Lasse Virus-specific antigen, an Influenza Virus-specific antigen, as well as any variant thereof and (b) an antigen selected from CD16, CD64, CD78, CD96, CLL1, CD116, CD117, CD71, CD45, CD71, CD123 and CD138, a tumor-associated surface antigen, such as ErbB2 (HER2/neu), carcinoembryonic antigen (CEA), epithelial cell adhesion molecule (EpCAM), epidermal growth factor receptor (EGFR), EGFR variantI II (EGFRvl ll), CD19, CD20, CD30, CD40, disialoganglioside GD2, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, CD22, insulin growth factor (IGFl)-l, IGF-I I, IGFI receptor, mesothelin, a major histocompatibility complex (MHC) molecule presenting a tumor-specific peptide epitope, 5T4, RORl, Nkp30, N KG2D, tumor stromal antigens, the extra domain A (EDA) and extra domain B (EDB) of fibronectin and the Al domain of tenascin-C (TnC Al) and fibroblast associated protein (FAP); a lineage-specific or tissue specific antigen such as CD3, CD4, CD8, CD24, CD25, CD33, CD34, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF17), multiple myeloma or lymphoblastic leukemia antigen, such as one selected from TNFRSF17, SLAMF7, GPRC5D, FKBP11, KAMP3, ITGA8, and FCRL5, a virus-specific surface antigen such as an HIV-specific antigen (such as HIV gpl20); an EBV-specific antigen, a CMV-specific antigen, a HPV-specific antigen, a Lasse Virus-specific antigen, an Influenza Virus-specific antigen, as well as any variant thereof.

In embodiments, the extracellular domain of the recombinant CAR comprises the extracellular domain of an NK cell activating receptor or a scFv.

In embodiments, the NK cell comprises a gene-edit in one or more of IL-7, CCL17, CCR4, IL-6, IL-6R, IL-12, IL-15, NKG2A, NKG2D, KIR, TRAIL, TRAC, PD1, and HPK1.

In embodiments, the gene-edit in one or more of IL-7, CCL17, CCR4, IL-6, IL-6R, IL-12, IL-15, NKG2A, NKG2D, KIR, TRAIL, TRAC, PD1, and HPK1 is caused by contacting the cell with RNA encoding one or more gene-editing proteins. In embodiments, the gene-edit of causes a reduction or elimination of expression and/or activity of IL-6, NKG2A, NKG2D, KIR, TRAC, PD1, and/or HPK1. In embodiments, the gene-edit causes an increase of expression and/or activity of IL-7, CCL17, CCR4, IL-6R, IL-12, IL-15, and/or TRAIL.

In embodiments, the cytotoxic lymphocyte, e.g., a T cell, NK cell, further comprises one or more recombinant genes capable of encoding a suicide gene product. In embodiments, the suicide gene product comprises a protein selected from the group consisting of thymidine kinase and an apoptotic signaling protein.

Any cytotoxic lymphocyte disclosed herein (e.g., manufactured by a method disclosed herein, comprising a gene edit (e.g., in B2M), expressing a high affinity CD16a receptor, and/or expressing a fusion protein comprising B2M polypeptide and an HLA polypeptide) can be further genetically engineered to express a CAR.

mRNA Cell Engineering Enables Rapid Prototyping of Macrophage Gene-Editing Strategies for Cancer Immunotherapy Applications

Macrophages' ability to infiltrate solid tumors and engage in both direct killing of cancer cells and recruitment of other immune cells has made them a promising target for development of next-generation cancer immunotherapies. The innate ability of macrophages to ingest foreign genetic material also facilitates their engineering with formulated nucleic acids, including mRNA. The oncoantigen tyrosine-protein kinase transmembrane receptor ROR1 has garnered interest for its minimal expression in healthy adult cells and overexpression in many malignancies, including solid tumors associated with ovarian, lung, and triple-negative breast cancer.

In this disclosure, e.g., in Example 10, an mRNA-based platform for rapid prototyping of macrophage engineering approaches is described. Here is shown mRNA delivery to peripheral blood mononuclear cell (PBMC) and iPS cell-derived macrophages for gene editing prototyping and functional assessment of encoded proteins. To develop this platform, macrophages were transfected with unmodified or 5′-methoxyuridine (5-moU)-containing mRNA encoding green fluorescent protein (GFP). Both mRNAs resulted in more than 95% of cells displaying GFP within 4 hours. ROR1-targeting CAR with a CD3 zeta activation domain and 4-1BB costimulatory domain was designed.

Transfecting mRNA encoding the ROR1-CAR yielded 70% CAR-expressing cells, as measured using PE-labelled ROR1. The ROR1 affinity of rabbit and humanized binding domains was analyzed and the humanized binding domain displayed a 2.5-fold increase in affinity as measured by flow cytometry using PE-labelled ROR1. The human receptor domain, but not the rabbit domain, demonstrated activation when bound to ROR1 as assessed by immunofluorescence of CD3 zeta phosphorylation. The mRNA-encoded ROR1-CAR's functionality was assayed by measuring killing of ROR1-expressing SKOV-3 ovarian cancer cells. Both the rabbit and humanized ROR1 domains of the CAR displayed significantly increased cytotoxicity towards SKOV-3 cells when compared with un-transfected macrophages after a 24-hour co-culture at a 5:1 effector-to-target ratio (p<0.01). The ROR1-CAR sequence was inserted into the AAVS1 safe harbor locus of iPSCs under the control of an SFC promotor, isolated5 biallelic-inserted lines, and the resulting cells were differentiated into macrophages. These results demonstrated an mRNA-based platform for rapid prototyping of macrophage engineering approaches. Transfection of macrophages with mRNA encoding a chimeric antigen receptor (CAR) targeting ROR1 resulted in functional expression in vitro, facilitating optimization of the antibody and co-stimulatory domains to improve protein binding affinity and immune activation. This platform thus enables the assessment and validation of novel macrophage gene editing strategies and is being explored for the development of macrophage-engineering therapies for solid tumor applications.

Indeed, this method permits screening of a library of CAR constructs in vitro to determine which constructs are readily expressed and which are most functional, e.g., in targeting and/or killing cancer cells. Once a CAR construct has been identified as superior, cells ex vivo may be gene-edited to express the construct and the edited cells may be administered into a subject in need and/or the cells of the subject in need may be gene-edited in vivo such that the construct genetically modify cells within the subject.

An aspect of the present disclosure is a method for screening constructs capable of being expressed in an in vivo cell and for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; and (3) identifying cells that have been transformed and capable of expressing the fusion protein for the ability of its fusion protein to recognize and/or bind to a cancer cell. Another aspect of the present disclosure is method for screening constructs capable of being expressed in an ex vivo cell and for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) culturing the cell capable of expressing the fusion protein which recognizes and/or binds to a cancer cell until a therapeutic amount of the cell is manufactured.

A further aspect of the present disclosure is a method for screening constructs capable of being expressed in an ex vivo cell and for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) contacting an ex vivo cell with the synthetic mRNA encoding the gene-editing protein and the repair template encoding the fusion protein which was identified in step (3) as having the ability recognize and/or bind to a cancer cell; and (5) culturing the cell of step (4) until a therapeutic amount of the cell is manufactured.

An additional aspect of the present disclosure is a method for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; and (4) administering to a subject in need the synthetic mRNA encoding the gene-editing protein and the repair template encoding the fusion protein which has the ability recognize and/or bind to a cancer cell.

In an aspect, the present disclosure provides a method for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) culturing the cell capable of expressing the fusion protein which recognizes and/or binds to a cancer cell until a therapeutic amount of the cell is manufactured; and (5) administering a therapeutically-effective amount of the cells of step (4) to a subject in need. In another aspect, the present disclosure provides a method for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) contacting an ex vivo cell with the synthetic mRNA encoding the gene-editing protein and the repair template encoding the fusion protein which was identified in step (3) as having the ability recognize and/or bind to a cancer cell; (5) culturing the cell of step (4) until a therapeutic amount of the cell is manufactured; and (6) administering a therapeutically-effective amount of the cells of step (4) to a subject in need.

In embodiments, the fusion protein that recognizes and/or binds to a cancer cell is a chimeric antigen receptor (CAR).

In embodiments, the CAR comprises an intracellular signaling domain, a transmembrane domain, and an extracellular domain comprising an antigen binding region.

In embodiments, the intracellular signaling domain comprises at least one immunoreceptor tyrosine-based activation motif (ITAM)-containing domain.

In embodiments, the intracellular signaling domain is from one of CD3-zeta, CD28, CD27, CD134 (OX40), and CD137 (4-1BB).

In embodiments, the transmembrane domain is from one of CD28 or a CD8.

In embodiments, the antigen binding region binds one antigen.

In embodiments, the antigen binding region binds two antigens.

In embodiments, the extracellular domain comprising an antigen binding region comprises: (a) C natural ligand or receptor, or fragment thereof, or (b) an immunoglobulin domain, optionally a single-chain variable fragment (scFv).

In embodiments, the extracellular domain comprising an antigen binding region comprises two of (a) a natural ligand or receptor, or fragment thereof, or (b) an immunoglobulin domain, optionally a single-chain variable fragment (scFv).

In embodiments, the extracellular domain comprising an antigen binding region comprises one of each of: (a) a natural ligand or receptor, or fragment thereof, and (b) an immunoglobulin domain, optionally a single-chain variable fragment (scFv).

In embodiments, the antigen binding region binds a tumor antigen.

In embodiments, the antigen binding region comprises one or more of: a. CD94/NKG2a, which optionally binds HLA-E on a tumor cell; b. CD96, which optionally binds CD155 on a tumor cell; c. TIGIT, which optionally binds CD155 or CD112 on a tumor cell; d. DNAM-1, which optionally binds CD155 or CD112 on a tumor cell; e. KIR, which optionally binds HLA class I on a tumor cell; f. NKG2D, which optionally binds NKG2D-L on a tumor cell; g. CD16a, which optionally binds an antibody/antigen complex on a tumor cell and/or wherein the CD16a is optionally a high affinity variant, optionally homozygous or heterozygous for F158V; h. NKp30, which optionally binds B7-H6 on a tumor cell; i. NKp44; and j. NKp46.

In embodiments, the antigen binding region comprises an immunoglobulin domain, optionally an scFv directed against HLA-E, CD155, CD112 HLA class I, NKG2D-L, or B7-H6.

In embodiments, the antigen binding region binds an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2.

In embodiments, the antigen binding region binds two antigens, the antigens being: a. an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2 and b. an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2.

In embodiments, the extracellular domain of the recombinant CAR comprises the extracellular domain of an NK cell activating receptor or a scFv.

In embodiments, the cell type is of the lymphoid cell lineage or the myeloid cell lineage. In some cases, the lymphoid lineage cell is a T cell, e.g., a cytotoxic T cell or gamma-delta T cell, or an NK cell, e.g., an NK-T cell. In various cases, the myeloid lineage cell is a macrophage, e.g., an M1 macrophage or an M2 macrophage.

In embodiments, after gene editing, the cell is a CAR-T cell, CAR-NK cell, a CAR-myeloid cell, or a CAR-mesenchymal stromal/stem cell.

When gene-editing is performed in vivo, transfection may be facilitated by use of the ToRNAdo™ Nucleic-Acid Delivery System, e.g., which relates to use of a compound of Formula (IV)

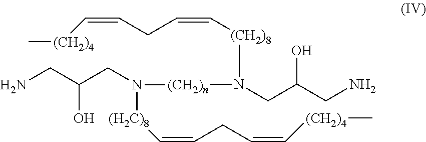

where n is 1, 2, 3, 4, 5, 6, 7, 8, 9, 10, 11, 12, 13, 14, or 15. Further description of ToRNAdo™ Nucleic-Acid Delivery System is found in one or both of U.S. Pat. No. 10,501,404 and WO2021003462. The entire contents of which are incorporated by reference in their entirety.

FIG. 30 is a cartoon showing methods for rapid prototyping of macrophage gene-editing strategies for cancer immunotherapies.

### Immune Silenced, Yet Self-Activating, Proliferative, and Anti-Tumoral Cells

Autologous engineered cell therapies such as autologous chimeric antigen receptor T-cell (CAR-T) therapies have revolutionized the treatment of hematologic cancers, however they are limited by manufacturing time and variability, the requirement for lymphodepletion, and side effects related to cytokine release. Allogeneic cell therapies derived from gene-edited induced pluripotent stem cells (iPSCs) are being developed to address the challenges associated with autologous engineered cell therapies. These “off-the-shelf” cell therapies contain specific edits designed to reduce immune rejection and to confer enhanced therapeutic properties and greater safety. However, efficient, footprint-free, biallelic targeting of defined loci in iPSCs remains technically challenging with current gene-editing approaches.

Further, while induced pluripotent stem cells (iPSCs) readily differentiate into a wide variety of cell types both in vitro and in vivo, the development of directed differentiation protocols that reliably yield pure populations of functional cells has proved challenging, in particular when differentiating into cell of the lymphoid or myeloid lineage. Generating functional cytotoxic lymphocytes from iPSCs is of particular interest to support the development of off-the-shelf engineered cell therapies for immune-oncology applications.

What is needed is improved compositions and methods for generating cellular therapies that can be engineered and produced in a practical manner.

The present disclosure is based, in part, on the discovery that cytotoxic lymphocytes, of the lymphoid cell lineage, e.g., T cells, NK cells, or cells of the myeloid lineage, e.g., macrophages, or mesenchymal stromal/stem cells, or hematopoietic stem cells can be gene-edited and differentiated, using mRNA- and iPS-based methods, to yield therapeutic cells that are immune silenced, yet self-activating, proliferative, and anti-tumoral.

Cytotoxic lymphocytes, including T cells and NK cells, are being developed as allogeneic, “off-the-shelf”, cell therapies for the treatment of hematological and solid tumors. Allogenic lymphocyte therapies face challenges, however, including limited expansion potential and limited in vivo persistence due to host immune rejection.

The resulting lymphocytes were characterized for surface markers via flow cytometry and incubated with cancer cells to assess tumor cell engagement and cytotoxicity. Notably, consistently higher yields of lymphocytes were obtained from the B2M-knockout iPSC line relative to a parental, wild-type iPSC line. Both wild-type and B2M-knockout lymphocytes cells killed 75-90% of K562 cells after 24 hours (effector to target (E:T) ratio of 5:1). Interestingly, cytotoxic lymphocytes derived from B2M-knockout iPSCs exhibited greater K562 cell killing with the addition of IL15 and IL2, while killing by wild-type cells was not controlled by these activating cytokines. Cancer cell killing activity was maintained through cryopreservation, albeit at a reduced level (15-40% reduction in activity). Accordingly, B2M-knockout iPSCs of the present disclosure are an ideal source of cytotoxic lymphocytes for the development of “off-the-shelf” allogeneic cell therapies for the treatment of cancer and without substantial host immune rejection.

In one aspect, there is provided cell comprising a genetically engineered disruption in a beta-2-microglobulin (B2M) gene, e.g., a loss of function, optionally in both alleles, of the B2M gene, wherein the cell is a cytotoxic lymphocyte from a lymphoid lineage cell or the cell is a myeloid lineage cell. In some cases, the lymphoid lineage cell is a T cell, e.g., a cytotoxic T cell or gamma-delta T cell; an NK cell; or an NK-T cell. In some cases, the myeloid lineage cell is a macrophage, e.g., an M1 macrophage or an M2 macrophage. In embodiments the cytotoxic lymphocyte is a NK cell.

The present cytotoxic lymphocyte is sometimes referred to herein as an “engineered cytotoxic lymphocyte”.

An aspect of the present disclosure is a method for manufacturing a population of cells that is enriched for cytotoxic lymphocytes. The method comprises steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes. In this aspect, steps (5) and (6) occur in an adherent culturing vessel. When CD34+ cells are selected, the embryoid bodies may be first chemically and/or mechanically dissociated. In embodiments, the stem cell is an induced pluripotent stem (iPSC). In this aspect, the stem cell has a genetically engineered disruption in a beta-2-microglobulin (B2M) gene. In some cases, the stem cell has a biallelic disruption in a B2M gene.

In another aspect, there is provided a method of making an engineered cell comprising a disrupted B2M gene, the method, comprising (a) reprogramming a somatic cell to an iPS cell, the reprogramming comprising contacting the iPS cell with a ribonucleic acid (RNA) encoding one or more reprogramming factors; (b) disrupting a B2M gene in the iPS cell, the disrupting comprising gene-editing the cell by contacting the cell with RNA encoding one or more gene-editing proteins; and (c) differentiating the iPS cell into a cytotoxic lymphocyte, e.g., a cell of the lymphoid cell lineage or into a cell of the myeloid cell lineage. In some cases, the lymphoid lineage cell is a T cell, e.g., a cytotoxic T cell or gamma-delta T cell; an NK cell; or an NK-T cell. In some cases, the myeloid lineage cell is a macrophage, e.g., an M1 macrophage or an M2 macrophage.

An aspect of the present disclosure is a method for killing a cancer cell. The method comprising steps of: (1) obtaining a herein-disclosed cytotoxic lymphocyte which was derived from a stem cell has a genetically engineered disruption in a beta-2-microglobulin (B2M) gene, e.g., a biallelic B2M knockout, and (2) contacting cytotoxic lymphocyte with the cancer. In some cases, the cancer cell is in vivo.

Yet another aspect of the present disclosure is a method for treating a cancer patient in need thereof. The method comprising a step of administering to the cancer patient a therapeutically-effective amounts of a herein-disclosed cytotoxic lymphocyte which was derived from a stem cell has a genetically engineered disruption in a beta-2-microglobulin (B2M) gene, e.g., a biallelic B2M knockout.

In some cases, mRNA-reprogrammed iPSC lines with a biallelic knockouts of the beta-2 microglobulin (B2M) gene, a key component of MHC class I molecules, are obtained using an mRNA-encoded chromatin context-sensitive gene-editing endonuclease. The B2M-knockout iPSCs may be differentiated using a novel, fully suspension process that replaces specialized micropatterned culture vessels with a spheroid culture step. Additional details regarding B2M knockout iPSCs useful in the present disclosure are described in PCT/US2022/019020, the contents of which are incorporated herein by reference in its entirety.

In another aspect, there is provided a method of treating cancer, comprising (a) obtaining an isolated cytotoxic lymphocyte comprising a genetically engineered disruption in a B2M gene; and (b) administering the isolated cytotoxic lymphocyte to a subject in need thereof, wherein the cytotoxic lymphocyte is selected from a lymphoid cell or a myeloid cell. In some cases, the lymphoid lineage cell is a T cell, e.g., a cytotoxic T cell or gamma-delta T cell; an NK cell; or an NK-T cell. In some cases, the myeloid lineage cell is a macrophage, e.g., an M1 macrophage or an M2 macrophage.

### Immune Silencing

In embodiments, the present cytotoxic lymphocyte is engineered to evade recognition and/or clearance by a host immune system. In embodiments, the present cytotoxic lymphocyte is a stealth cytotoxic lymphocyte. In embodiments, the present cytotoxic lymphocyte is not substantially recognized by an immune system upon administration to a subject.

In embodiments, the present cytotoxic lymphocyte has reduced or eliminated susceptibility to cell killing by T cells as compared to a cytotoxic lymphocyte which does not comprise a genetically engineered disruption in the B2M gene. In embodiments, the present cytotoxic lymphocyte has reduced or eliminated susceptibility to cell killing by other cytotoxic lymphocytes as compared to another cytotoxic lymphocyte which comprises a genetically engineered disruption in the B2M gene. In embodiments, the present cytotoxic lymphocyte is characterized in that the expression of B2M is reduced or inhibited. In embodiments, the present cytotoxic lymphocyte is characterized in that the function of B2M is reduced or inhibited.

In embodiments, the present cytotoxic lymphocyte is characterized in that the expression of MHC class I is reduced or inhibited. In embodiments, the present cytotoxic lymphocyte is characterized in that the function of MHC class I is reduced or inhibited.

In embodiments, the B2M gene is a human B2M gene (e.g., NCBI Reference Sequence: NG_012920). The sequence of the B2M gene of various embodiments is provided in the EXAMPLES section herein. B2M, is the light chain of MHC class I molecules, and as such an integral part of the major histocompatibility complex. In humans, B2M is encoded by the b2m gene which is located on chromosome 15. The human protein is composed of 119 amino acids and has a molecular weight of 11.8 kilodaltons (e.g., UniProtKB—P61769). The amino acid sequence of human beta-2-microglobulin (B2M) is:

(SEQ ID NO: 13)
MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDM.

In embodiments, the present cytotoxic lymphocyte has genetically engineered disruptions of all substantially all copies of the B2M gene. In embodiments, the present cytotoxic lymphocyte has a loss of function of the B2M gene. In embodiments, the present cytotoxic lymphocyte has a loss of function of both alleles of the B2M gene.

In embodiments, the genetically engineered disruption of the B2M gene is in exon 3 of human B2M. In embodiments, the genetically engineered disruption of the B2M gene is a deletion. In embodiments, the deletion is about 10 to about 20 nucleotides. In embodiments, the deletion is near nucleotides 500 to 550 of the human B2M gene. In embodiments, the deletion is of the sequence TTGACTTACTGAAG (SEQ ID NO: 14), or a functional equivalent thereof.

In embodiments, the present cytotoxic lymphocyte has downregulated MHC class I expression and/or activity.

In embodiments, the genetically engineered disruption of B2M comprises a gene-edit and the gene-edit is caused by contacting the cell with RNA encoding one or more gene-editing proteins.

In embodiments, the present cytotoxic lymphocyte is engineered to be further immune silenced, e.g., in addition to B2M (MHC Class I) disruption. In embodiments, the present cytotoxic lymphocyte is engineered to be disrupted at the human MHC II transactivator (CIITA) gene (NCBI Reference Sequence: NG_009628.1).

In embodiments, the present cytotoxic lymphocyte has downregulated MHC class II expression and/or activity.

In embodiments, the present cytotoxic lymphocyte is characterized in that the expression of CIITA is reduced or inhibited. In embodiments, the present cytotoxic lymphocyte is characterized in that the function of CIITA is reduced or inhibited.

In embodiments, the present cytotoxic lymphocyte is characterized in that the expression of MHC class II is reduced or inhibited. In embodiments, the present cytotoxic lymphocyte is characterized in that the function of MHC class II is reduced or inhibited.

In embodiments, the genetically engineered disruption of CIITA comprises a gene-edit and the gene-edit is caused by contacting the cell with RNA encoding one or more gene-editing proteins.

In embodiments, the present cytotoxic lymphocyte is characterized in that the expression of B2M and CIITA is reduced or inhibited. In embodiments, the present cytotoxic lymphocyte is characterized in that the function of B2M and CIITA is reduced or inhibited.

In embodiments, the present cytotoxic lymphocyte is characterized in that the expression of MHC class I and MHC class II are reduced or inhibited. In embodiments, the present cytotoxic lymphocyte is characterized in that the function of MHC class I and MHC class II are reduced or inhibited.

In embodiments, the genetically engineered disruption of B2M and CIITA comprises a gene-edit and the gene-edit is caused by contacting the cell with RNA encoding one or more gene-editing proteins. In embodiments, the present cytotoxic lymphocyte comprises a genetically engineered alteration in one or more genes selected from HLA-A, HLA-B, HLA-C, HLA-E, HLA-F and HLA-G.

In embodiments, the cytotoxic lymphocyte expresses a fusion protein comprising a B2M polypeptide and a HLA-A, HLA-B, HLA-C, HLA-E, HLA-F and HLA-G polypeptide. The fusion protein may be expressed by insertion of a repair template into a single or double strand break of the B2M gene; in some cases, the repair template comprises the coding sequence for B2M and the HLA gene. Notably, the fusion protein replaces endogenous B2M and HLA pairs expressed by a cytotoxic lymphocyte, thereby reducing the likelihood that the cytotoxic lymphocyte will be reduced or eliminated by a host cytotoxic lymphocyte.

In embodiments, the present cytotoxic lymphocyte does not comprise a genetically engineered alteration in one or more genes selected from HLA-A, HLA-B, HLA-C, HLA-E, HLA-F and HLA-G.

In embodiments, the genetically engineered alteration is a genetically engineered reduction or elimination in expression and/or activity of one or more genes selected from HLA-A, HLA-B, HLA-C, HLA-E, HLA-F and HLA-G.

In embodiments, the genetically engineered alteration is a genetically engineered increase in expression and/or activity of one or more genes selected from HLA-A, HLA-B, HLA-C, HLA-E, HLA-F and HLA-G.

In embodiments, the genetically engineered disruption of B2M is combined with a genetically engineered expression of a fusion between B2M or a fragment thereof and one or more genes and/or fragments thereof selected from HLA-A, HLA-B, HLA-C, HLA-E, HLA-F and HLA-G.

In embodiments, the B2M or fragment thereof and one or more genes and/or fragments thereof are separated by a linker region. In embodiments the linker is (G₄S)₃.

In embodiments, the genetically engineered alteration is a genetically engineered increase in expression and/or activity of one or more genes selected from IL-2, IL-15, IL-21. In embodiments, the IL-15 contains the N72D mutation. In embodiments, the IL-15 is fused to the cytokine binding domain of IL-15Rα.

In embodiments, the present cytotoxic lymphocyte is characterized in that the expression of negative regulators of IL-15 signaling are reduced or inhibited. In embodiments, the negative regulator of IL-15 signaling is the CISH protein. In embodiments, the reduction or inhibition of negative regulators of IL-15 signaling is achieved by genetically engineered disruption of the CISH gene. The Cytokine-inducible SH2-containing protein (CISH) gene is found at gene ID: NG_023194.1.

In embodiments, the genetically engineered disruption of CISH comprises a gene-edit and the gene-edit is caused by contacting the cell with RNA encoding one or more gene-editing proteins.

### Methods of Making B2M Knockout Cells

In aspects, the present disclosure provides a method of making an engineered cell comprising a disrupted B2M gene, the method comprising: (a) reprogramming a somatic cell to an iPS cell, the reprogramming comprising contacting the iPS cell with a ribonucleic acid (RNA) encoding one or more reprogramming factors; (b) disrupting a beta-2-microglobulin (B2M) gene in the iPS cell, the disrupting comprising gene-editing the cell by contacting the cell with RNA encoding one or more gene-editing proteins; and (c) differentiating the iPS cell into a cytotoxic lymphocyte, e.g., a cell of the lymphoid cell lineage, or into a cell of the myeloid cell lineage, or a mesenchymal stem/stromal cell, or a hematopoietic stem cell. In some cases, the lymphoid lineage cell is a T cell, e.g., a cytotoxic T cell or gamma-delta T cell; an NK cell; or an NK-T cell. In some cases, the myeloid lineage cell is a macrophage, e.g., an M1 macrophage or an M2 macrophage.

In embodiments, the method further comprises disrupting a CIITA gene in the iPS cell, the disrupting comprising gene-editing the cell by contacting the cell with RNA encoding one or more gene-editing proteins.

In embodiments, the cytotoxic lymphocyte is an NK cell.

In embodiments, the somatic cell is a fibroblast or keratinocyte.

In embodiments, the method provides an increased proliferation rate of iPS cells as compared to the rate of iPS cells without a disruption of the B2M gene.

In embodiments, the method provides an increased proliferation rate of differentiating cells along a lymphoid lineage cell as compared to the rate of iPS cells without a disruption of the B2M gene.

In embodiments, the method provides an increased expansion of differentiating cells along a lymphoid lineage cell as compared to the rate of iPS cells without a disruption of the B2M gene.

In embodiments, the differentiating comprises embryoid body-based hematopoietic commitment. In embodiments, the differentiating comprises enrichment of CD34+ cells. In embodiments, the differentiating comprises differentiating into CD5+/CD7+ common lymphoid progenitors.

In embodiments, the method yields CD56^{dim} CD16+ NK cells.

In embodiments, the RNA is associated with one or more lipid selected from and/or Formulae I-XVI.

Differentiation of iPSCs into Monocytes and Macrophages

In one aspect, the present disclosure relates to induced pluripotent stem cell (iPSC)-derived monocytes that can be differentiated into functional M1 and M2 macrophages with enhanced cytokine secretion and tumor cell-killing activity.

Although cancer immunotherapy has advanced rapidly over the past two decades, with several autologous chimeric antigen receptor (CAR)-T cell therapies approved for the treatment of hematologic cancers, CAR-T cells have shown limited activity against solid tumors, in part due to the immunosuppressive nature of the tumor microenvironment preventing CAR-T cell infiltration. This has led to investigation of other immune cells as alternatives to T-cell-based therapies, including monocytes and monocyte-derived macrophages, which exhibit innate tumor-infiltration properties. Disclosed herein is a new and useful process for differentiating pluripotent stem cells along a myeloid lineage, and generated populations of cells with characteristics of monocytes and M1 and M2 macrophages, including cytokine secretion and tumor cell-killing activity. mRNA-reprogrammed human induced pluripotent stem cells (iPSCs) were differentiated into monocytes using a 28-day monolayer protocol. Beginning on day 14, cells were harvested every 3-4 days. CD14+ isolation yielded >95% CD14+ cells with an average yield of 4.1×10⁴ cells per cm² per harvest. iPSC-derived monocytes were compared to peripheral blood mononuclear cell (PBMC)-derived monocytes for expression of key hematopoietic and myeloid-lineage markers CD11b, CD14, CD33, CD45, CD80, CD163, CD206, and SIRPα. iPSC-derived monocytes showed similar expression of CD11b, CD14, CD33, CD45, and CD163 compared to PBMC-derived monocytes, and increased expression of markers indicative of an activated state: CD80 and CD206. Compared to PBMC-derived monocytes, iPSC-derived monocytes showed both higher viability in culture and superior recovery from cryopreservation. iPSC-derived monocytes were further differentiated into macrophages by exposure to MCSF for 3-4 days, and were assessed for their ability to polarize, secrete pro- and anti-inflammatory cytokines, and for cytotoxic activity when co-cultured with cancer cells. M1 macrophages were polarized with interferon gamma (IFN-γ, 50 ng/mL) and lipopolysaccharide (LPS, 10 ng/mL) for 48 hours, while M2 macrophages were treated with IL-4 (10 ng/mL) for 48 hours. iPSC-derived monocytes differentiated into macrophages with >90% efficacy, as assessed by cell adherence, morphology, and surface marker expression (CD14, CD45, CD163). M1 and M2 polarized iPSC-derived macrophages secreted similar levels of TNFα, IL-12p70, and IL-10 compared to PBMC-derived macrophages. iPSC-derived macrophages killed 45% of U2OS cancer cells in vitro after 24 hours at an E:T ratio of 5:1. Disclosed herein is a process for differentiating mRNA-reprogrammed iPSCs into cytotoxic macrophages. The mRNA reprogramming and differentiation processes are virus-free and DNA-free, avoiding any potential risk of vector integration. This disclosure provides proof of concept that mRNA-reprogrammed iPSCs represent a viable source of macrophages for the development of therapies to treat various indications, including solid tumors. The process is illustrated in FIG. 42.

Monocytes are a type of leukocyte, or white blood cell. They are the largest type of leukocyte and can differentiate into macrophages and conventional dendritic cells. As a part of the vertebrate innate immune system monocytes also influence adaptive immune responses and exert tissue repair functions. There are at least three subclasses of monocytes in human blood based on their phenotypic receptors: The classical monocyte is characterized by high level expression of the CD14 cell surface receptor (CD14++ CD16− monocyte), the non-classical monocyte shows low level expression of CD14 and additional co-expression of the CD16 receptor (CD14+CD16++ monocyte), and the intermediate monocyte expresses high levels of CD14 and low levels of CD16 (CD14++CD16+ monocytes).

Monocytes are mechanically active cells and migrate from blood to an inflammatory site to perform their functions. In general, monocytes and their macrophage and dendritic cell progeny serve three main functions in the immune system: these are phagocytosis, antigen presentation, and cytokine production. Phagocytosis is the process of uptake of microbes and particles followed by digestion and destruction of this material. Monocytes can perform phagocytosis using intermediary (opsonising) proteins such as antibodies or complement that coat the pathogen, as well as by binding to the microbe directly via pattern-recognition receptors that recognize pathogens. Monocytes are also capable of killing infected host cells via antibody-dependent cell-mediated cytotoxicity. Vacuolization may be present in a cell that has recently phagocytized foreign matter.

Macrophages engulf and digest pathogens, such as cancer cells, microbes, cellular debris, and foreign substances, which do not have proteins that are specific to healthy body cells on their surface, via phagocytosis.

Macrophage that encourages inflammation are called M1 macrophages, whereas those that decrease inflammation and encourage tissue repair are called M2 macrophages. M1 macrophages are classically activated, typically by IFN-γ or lipopolysaccharide (LPS), and produce proinflammatory cytokines, phagocytize microbes, and initiate an immune response. M1 macrophages produce nitric oxide (NO) or reactive oxygen intermediates (ROI) to protect against bacteria and viruses. M2 macrophages are alternatively activated by exposure to certain cytokines such as IL-4, IL-10, or IL-13. M2 macrophages will produce either polyamines to induce proliferation or proline to induce collagen production. These macrophages are associated with wound healing and tissue repair. iPSCs obtained by a herein-disclosed method may be differentiated into a monocyte which can be further differentiated into a macrophage, e.g., an M1 or M2 macrophage.

The general steps for this process comprise (1) iPSC to monocyte differentiation, (2) CD14+ magnetic bead positive selection, and (3) monocyte to macrophage differentiation. These general steps are, respectively, shown in FIG. 43A, FIG. 43B, and FIG. 43C.

As shown in FIG. 43A, embryonic stem cells (ES cells) may be used as starting material for the process rather than iPSCs.

Beginning on day 14, monocyte cells can be harvested and continue to be harvested every 3-4 days. Additional details regarding differentiation of iPSCs to monocytes is found at the World Wide Web (www) stemcell.com/stemdiff-monocyte-kit.html, the contents of which are incorporated herein by reference in its entirety.

As shown in FIG. 43B, CD14+ cells, which comprise monocytes and macrophages, are separated using suitable reagents.

Finally, as shown in FIG. 43C, monocytes are cultured under conditions such that they differentiate into macrophages, e.g., by activation with macrophage colony-stimulating factor (M-CSF). Additional details regarding differentiation of monocytes to macrophage is found at the World Wide Web (www) stemcell.com/immunocult-sf-macrophage-medium.html, the contents of which are incorporated herein by reference in its entirety.

M1 macrophages can be then polarized into M1 macrophages with interferon gamma (IFN-γ, 50 ng/mL) and lipopolysaccharide (LPS, 10 ng/mL) for 48 hours, whereas macrophages are treated with IL-4 (10 ng/mL) for 48 hours to generate M2 macrophages.

Mature differentiated cells can be reprogrammed and dedifferentiated into embryonic-like cells, with embryonic stem cell-like properties. Fibroblast cells can be reversed into pluripotency via, for example, retroviral transduction of certain transcription factors or transfection of synthetic nucleic acids encoding transcription factors, resulting in iPSCs. In some embodiments, iPSCs are generated from various tissues, including fibroblasts, keratinocytes, melanocyte blood cells, bone marrow cells, adipose cells, and tissue-resident progenitor cells. In some embodiments, iPSCs are generated via transfection of synthetic nucleic acids encoding the transcription factors Oct4 protein, Sox2 protein, Klf4 protein, c-Myc protein, l-Myc protein, TERT protein, Nanog protein, Lin28 protein, Glis1 protein, Utf1 protein, Aicda protein and biologically active fragments, analogues, variants and family-members thereof. In some embodiments, iPSCs are generated via transfection of synthetic nucleic acids encoding miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

The generation of iPSCs depends on the transduction of specific transcription factors into the somatic cell genome via vectors for its reprogramming.

In various cases, transfection of a cell with synthetic nucleic acids for reprogramming may be facilitated by use of the ToRNAdo™ Nucleic-Acid Delivery System. This system relates to new lipids that find use, inter alia, in improved delivery of biological payloads, e.g., nucleic acids, to cells. The system relates to use of a compound of Formula (IV)

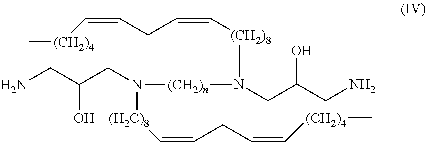

where n is 1, 2, 3, 4, 5, 6, 7, 8, 9, 10, 11, 12, 13, 14, or 15. Further description of ToRNAdo™ Nucleic-Acid Delivery System is found in one or both of U.S. Pat. No. 10,501,404 and WO2021003462. The entire contents of which are incorporated by reference in their entirety.

In some embodiments, following iPSC generation, cells are assessed via pluripotency assays, including morphological and histological analysis, and certain gene expression profiles, proving their ability to differentiate into tissues derived from the three germ layers and teratoma formation. In some embodiments, teratoma assays involve injection of iPSCs into immunocompromised experimental animals and subsequent formed tissue analysis to assess teratoma formation.

In embodiments, the iPSC is derived from a human. In embodiments, the iPSC is derived from a subject who is not intended to receive the therapy. In embodiments, the iPSC is allogeneic to a patient intended to receive the therapy. In embodiments, the iPSC is from a master cell bank.

Stem cells have the ability to self-renew and differentiate into multiple cell types and so have applications in regenerative medicine.

In embodiments, the iPSCs, the monocytes, and/or the macrophage is further gene-edited, as disclosed herein. In some embodiments, the iPSC was gene-edited contemporaneously with being reprogrammed, e.g., from a fibroblast. In various embodiments, the iPSC was gene-edited before being reprogrammed, e.g., from a fibroblast. In other embodiments, the iPSC was gene-edited after being reprogrammed, e.g., from a fibroblast.

In some aspects of the present disclosure, a macrophage that is administered to a patient, e.g., for killing cancer cell, was ultimately derived from the patient. That is to say, a keratinocyte (as an example) is obtained from the patient and this keratinocyte is reprogrammed into an IPSC which is differentiated into a monocyte and further differentiated into an M1 or M2 macrophage. By starting with the patient's own cells, the macrophage administered back to the patient could be considered to be autologous.

The use of autologous macrophages in therapeutic applications is safe because the cells will not elicit an immune response. However, it may be difficult to obtain a large amount of bone marrow or adipose tissue from the subject. Autologous macrophages may also have reduced therapeutic efficacy resulting in poor clinical outcomes. Additionally, if macrophages are needed urgently, there may not be time to extract and expand autologous macrophages from a subject.

Thus, the use of allogeneic macrophages is an attractive alternative because donors can be prescreened for having cells with a high therapeutic potential. Allogeneic macrophages can be prepared on a clinical scale, assayed for therapeutic potential after production and stored in usable clinical doses that can be used readily for urgent therapeutic applications. In some embodiments, the present macrophages are allogeneic.

Thus, obtaining macrophages by reprogramming iPSCs, which may be obtained by reprogramming human somatic cells, is an attractive alternative. In some embodiments, iPSCs are obtained monocytes are generated via cell reprogramming with non-immunogenic messenger RNA (mRNA) encoding one or more reprogramming factors in a defined, animal-component-free process. The monocytes are further differentiated into macrophages. See, e.g., FIG. 42.

In some embodiments, monocytes and/or macrophages are checked for safety using one or more of bacterial and fungal tests, Mycoplasma test, adventitious viral agent test, and tumorigenicity assay (karyotype analysis, teratoma formation assay, soft agar assay, comparative genomic hybridization (CGH), fluorescence in situ hybridization (FISH), and polymerase chain reaction (PCR)).

In embodiments, the monocyte or macrophage has been altered to reduce expression of MHC molecules. In embodiments, the alteration is enabled by gene editing. In embodiments, the MHC molecules are MHC class I molecules. In embodiments, the expression of MHC class I molecules is reduced by gene editing the B2M gene. In some cases, the gene editing occurs in an iPSC cell that is a progenitor of the monocyte or macrophage.

The Immunosuppressive TTAGGG Motif Improves Homology-Directed Insertion of DNA Sequences in Human Primary and Induced Pluripotent Stem (iPS) Cells

In another aspect, the present disclosure relates to use of TTAGGG motif for decreasing synthetic oligodeoxynucleotides (ODNs)-related activation of pro-inflammatory responses; with decreasing the pro-inflammatory responses leading to higher transgene insertion efficiency.

Double stranded synthetic oligodeoxynucleotides (dsODNs) have been used as repair templates in gene-editing applications to insert transgenic sequences into defined genomic loci, albeit with low efficiency. Cells engineered in this way are of interest for many therapeutic applications, including allogeneic NK and T cells engineered to express stealthing proteins, cytokines, and chimeric antigen receptors (CARs) for the treatment of a variety of cancers. To increase the efficiency of integration, gene-editing proteins can be co-expressed to create a double-strand break at the target locus. However, recognition of dsODNs by pattern recognition receptors activates signaling cascades resulting in the production of cytokines, including type I interferons such as IFIT1-3 and IFN-β. This immune response can lead to cell cycle arrest, differentiation, and apoptosis and may contribute to low insertion efficiency observed in primary and iPS cells. It has been shown in human immune cells that co-delivery of a short ODN comprising the immunosuppressive motif, TTAGGG, which is found in mammalian telomeric DNA, inhibits the activation of the damage-associate molecular pattern (DAMP) pathway in response to cytosolic DNA. This ODN competitively binds to inflammasomes and reduces the secretion of proinflammatory cytokines. The present disclosure relates to dsODNs comprising the TTAGGG motif for decreasing dsODN-related activation of a pro-inflammatory response in human cells, with decreasing the pro-inflammatory responses leading to higher transgene insertion efficiency. As disclosed herein, the TTAGGG motif is incorporated either at the 5′ end of dsODNs or delivered separately on a short single-stranded ODN (A151). Human primary fibroblasts, iMSCs and iPSCs were electroporated with a dsODN encoding a GFP reporter and containing an SfoI restriction site. Upregulation of pro-inflammatory markers including IFIT1-3, was measured by RT-PCR. A 29-fold higher expression of IFIT1 and IFIT3 was observed in cells electroporated with dsODNs than in untreated controls. Interestingly, including TTAGGG motifs at the 5′-ends of the dsODNs limited the upregulation of IFIT1 and IFIT3 to 10- and 15-fold, respectively, while co-delivery of the TTAGGG motif prevented their upregulation altogether. A gene-editing endonuclease targeting the AAVS1 safe-harbor locus on chromosome 19 was then used to investigate the impact of the TTAGGG motif on the insertion of transgenes at this site. The TTAGGG motif (whether incorporated in the dsODN or co-transfected in the form of the A151 ODN) resulted in approximately 50% higher viability and approximately 50% more GFP-positive cells than when the motif was not present. This disclosure provides proof of concept that the herein-disclosed immunosuppressive sequences increases ODN insertion efficiency and improves cell viability and is therefore a powerful tool for therapeutic knock-in applications, including the generation of knock-in iPS cell lines.

The process of including a TTAGGG motif in an dsODN for reducing an immune response is illustrated in FIG. 54. As shown in FIG. 54, the TTAGGG motif, found in telomeric DNA, is incorporated onto the 5′ end of a double stranded repair template in primary, iMS and iPS cells. The motif is recognized by, and competitively binds to Pattern Recognition receptors in the cytoplasm of the cells to lessen the immune response mounted against the double stranded repair templates.

The TTAGGG motif may be provided at the 5′ end of a dsODN that serves as a repair template. The TTAGGG motif may be provided at the 3′ end of a dsODN that serves as a repair template. The TTAGGG motif may be provided at both the 3′ end and the 5′ end of a dsODN that serves as a repair template. The dsODN that serves as a repair template may comprise 1, 2, 3, 4, 5, 6, 7, 8, 9, 10 or more repeats of the TTAGGG motif.

As disclosed-herein, the TTAGGG motif may be provided to a cell separately from the dsODN on a short single-stranded synthetic oligodeoxynucleotides (e.g., A151). The A151 ssODN comprise four repeats of the TTAGGG motif and the sequence of TTAGGGTTAGGGTTAGGGTTAGGG (SEQ ID NO: 15). In other cases, a single-stranded synthetic oligodeoxynucleotide may comprise 1, 2, 3, 4, 5, 6, 7, 8, 9, 10 or more repeats of the TTAGGG motif. In some case, the TTAGGG motif may be provided to a cell separately from the dsODN on a short double-stranded synthetic oligodeoxynucleotides. In embodiments, the double-stranded synthetic oligodeoxynucleotide may comprise 1, 2, 3, 4, 5, 6, 7, 8, 9, 10 or more repeats of the TTAGGG motif.

In some embodiments, the cells that are transfected with a TTAGG-containing ODN (either as a repair template or as separate ODN) are skin cells, pluripotent stem cells, embryonic stem cells, iPSCs, MSCs (including iMSCs), mesenchymal stromal/stem cells, hematopoietic cells, hematopoietic stem cells, lymphocytes, β-cells, T-cells (including CAR-T), NK cell (including CAR-NK), monocytes, macrophages (including CAR-myeloid cells and CAR-mesenchymal stromal/stem cells), retinal pigmented epithelial cells, hematopoietic cells, a hematopoietic stem cells, myeloid cells, tumor-infiltrating lymphocytes, marrow-infiltrating lymphocytes, a peripheral blood lymphocytes, cardiac cells, airway epithelial cells, neural stem cells, neurons, glial cells, bone cells, blood cells, and dental pulp stem cells.

In embodiments, gene-editing a cell comprises contacting the cell with synthetic nucleic acid encoding one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a (Cpf1), Cas13a, Cas14, CasX, CasY, a Class 1 Cas protein, a Class 2 Cas protein, MAD7, and a gene-editing protein comprising a repeat sequence comprising LTPvQVVAIAwxyz (SEQ ID NO: 16), or a natural or engineered variant, family member, orthologue, fragment or fusion construct thereof.

In embodiments, the gene-editing protein comprises (i) a DNA-binding domain comprising a plurality of repeat sequences and at least one of the repeat sequences comprises the amino acid sequence: LTPvQVVAIAwxyzGHGG (SEQ ID NO: 17) or LTPvQVVAIAwxyzGTHG (SEQ ID NO: 18) and is from 36 to 39 amino acids long, wherein: “v” is Q, D or E, “w” is S or N, “x” is H, N, or I, “y” is D, A, I, N, G, H, K, S, or null, and “z” is GGKQALETVQRLLPVLCQD (SEQ ID NO: 19) or GGKQALETVQRLLPVLCQA (SEQ ID NO: 20) and (ii) a nuclease domain comprising a catalytic domain of a nuclease. In some embodiments, a gene-editing protein comprises a C-terminal GTHG (SEQ ID NO: 21) produces more efficient editing at the target locus than TALENs at 33° C. GTHG (SEQ ID NO: 21). In various embodiments, a gene-editing protein comprises a C-terminal GTHG (SEQ ID NO: 21) produces more efficient editing at the target locus than TALENs at 37° C.

In embodiments, the gene-editing protein comprises (i) a DNA-binding domain comprising a plurality of repeat sequences and at least one of the repeat sequences comprises the amino acid sequence: LTPvQVVAIAwxyzα (SEQ ID NO: 22) and is from 36 to 39 amino acids long, wherein: v is Q, D or E, w is S or N, x is I, H, N, or I, y is D, A, I, N, H, K, S, G or null, z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), α is four consecutive amino acids; and (ii) a nuclease domain comprising a catalytic domain of a nuclease. In embodiments, α is selected from GHGG (SEQ ID NO: 31), HGSG (SEQ ID NO: 32), HGGG (SEQ ID NO: 33), GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60). In some embodiments, a gene-editing protein comprises a C-terminal GTHG produces more efficient editing at the target locus than TALENs at 33° C. GTHG (SEQ ID NO: 21). In various embodiments, a gene-editing protein comprises a C-terminal GTHG (SEQ ID NO: 21) produces more efficient editing at the target locus than TALENs at 37° C.

In some cases, a cell is contacted with a demethylating agent during the process of gene-editing. In embodiments, the demethylating agent is selected from 5-azacitidine and 5-aza-2′-deoxycitidine (decitabine).

In some embodiments, the gene-editing protein comprises: (a) the DNA-binding domain comprising a plurality of repeat sequences and at least one of the repeat sequences comprises a repeat variable di-residue (RVD) at residue 12 or 13; and (b) the nuclease domain comprising a catalytic domain, the catalytic domain comprising a hybrid of the catalytic domains of Fokl and Stsl, comprising the α1, α2, α3, α4, α5, α6, β1, β2, β3, β4, β5, and β6 domains of Fokl with at least one of the domains of Fokl being substituted in whole or in part with the α1, α2, α3, α4, α5, α6, β1, β2, β3, β4, β5, and β6 domains of Stsl and optionally comprising at least one mutation.

In various embodiments, the cell is transfected (e.g., contacted) with a synthetic nucleic acid encoding the gene-editing protein at about 30° C. to about 35° C., e.g., without limitation about 33° C. In embodiments, the contacting occurs at about 30° C. In some embodiments, the contacting occurs at about 31° C. In some embodiments, the contacting occurs at about 32° C. In some embodiments, the contacting occurs at about 33° C. In some embodiments, the contacting occurs at about 34° C. In some embodiments, the contacting occurs at about 35° C. In embodiments, the gene-editing protein is functionally temperature-switchable. In embodiments, the method further comprises the step of (c) culturing the contacted cell at about 30° C. to about 35° C. In embodiments, the method further comprises the step of (c) culturing the contacted cell at about 30° C. In embodiments, the method further comprises the step of (c) culturing the contacted cell at about 31° C. In embodiments, the method further comprises the step of (c) culturing the contacted cell at about 32° C. In embodiments, the method further comprises the step of (c) culturing the contacted cell at about 33° C. In embodiments, the method further comprises the step of (c) culturing the contacted cell at about 34° C. In embodiments, the method further comprises the step of (c) culturing the contacted cell at about 35° C.

In embodiments, the gene-edited cell, as disclosed herein, is also reprogrammed, as disclosed herein. In some embodiments, the gene-editing is contemporaneously with reprogramming. In various embodiments, the gene-editing is before the reprogramming. In other embodiments, the gene-editing is after the reprogramming. In cases when a cell is reprogramming, the cell may be a differentiated or a non-pluripotent cell. In some cases, the differentiated or a non-pluripotent cell is a skin cell (e.g., a fibroblast or a keratinocyte).

### Resveratrol

In yet another aspect, the present disclosure relates to use of Resveratrol treatment prior to transfection, e.g., with a synthetic nucleic acid encoding a gene-editing protein, and/or after transfection, e.g., with a synthetic nucleic acid encoding a gene-editing protein.

Gene editing technology, which enables the precision modification of DNA in living cells, is being developed for the treatment of various diseases, including genetic diseases and cancer. Gene editing commonly employs sequence-specific endonucleases to create double strand breaks in genomic DNA and relies on the cell's DNA repair mechanisms to apply the desired changes. Precise sequence modifications, such as single-base changes, rely on the homology directed repair (HDR) mechanism. Despite its essential role in gene repair, HDR occurs at a very low frequency in many cells compared to other repair mechanisms. The present disclosure discloses the impact of resveratrol, a small molecule extracted from grape skin, that promotes the expression of key HDR factors and induces cell cycle arrest at S phase in porcine fetal fibroblasts, on single-base editing efficiency in primary human fibroblasts. Following treatment with resveratrol, fibroblasts was co-transfected with mRNA encoding a chromatin context-sensitive gene-editing protein targeting the AAVS1 safe-harbor locus, and a single-stranded DNA repair template designed to introduce a SfoI restriction-enzyme site through a G-to-C mutation. Single-base editing efficiency was determined by restriction fragment length polymorphism (RFLP) analysis. Resveratrol treatment prior to transfection increased the S and G2-phase population 2.3-fold and increased HDR efficiency 2-fold compared to untreated cells. Application of resveratrol after transfection (i.e., no cell cycle synchronization) yielded further improvement in single-base editing efficiency (>2-fold), suggesting that the effects of resveratrol on HDR are not confined to cell-cycle control. This disclosure provides proof of concept that Resveratrol treatment provides a straightforward method for improving HDR efficiency in primary human fibroblasts and serves as a useful tool in the development of HDR-based gene-editing therapies.

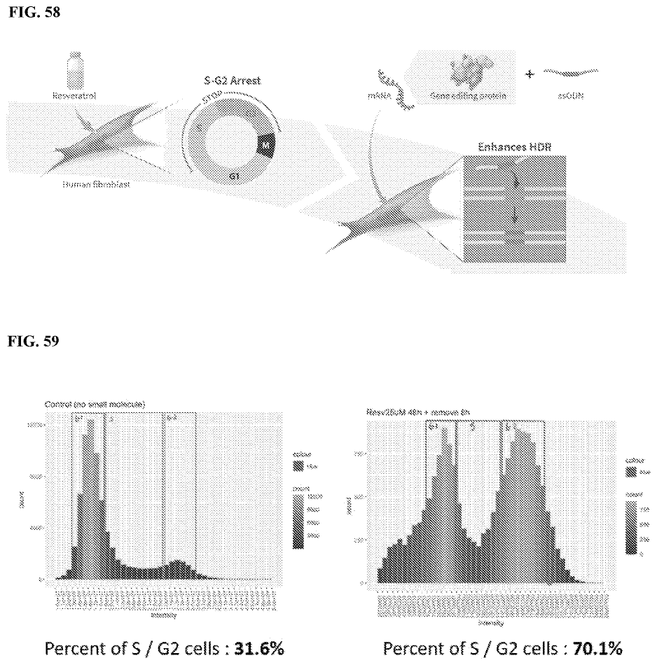

The process of contacting Resveratrol with a cell in advance of gene editing is illustrated in FIG. 58. As shown in FIG. 58, Resveratrol arrests the cell in S or G2 phase and enhances the efficiency of subsequent gene-editing.

Notably, Resveratrol treatment before transfecting with a gene-editing machinery (e.g., a synthetic nucleic acid encoding a gene-editing protein) appears to arrest the majority of cells in S/G2; this pre-treatment enhances the efficiency of subsequent gene-editing. Also, Resveratrol treatment after transfecting with a gene-editing machinery (e.g., a synthetic nucleic acid encoding a gene-editing protein) enhances the efficiency of ongoing gene-editing. Therefore, in embodiments, cells may be pre-treated with Resveratrol and/or post-treated with Resveratrol.

Any of the herein-disclosed gene-editing methods may comprise pre-treatment with Resveratrol and/or post-treated with Resveratrol.

### Reprogramming Methods

In embodiments, the present disclosure relates to RNA-based modifications, e.g., reprogramming and/or gene-editing. In some embodiments, an RNA molecule encodes a gene-editing protein. In some embodiments, an RNA molecule encodes a reprogramming factor.

In embodiments, the RNA is mRNA. In embodiments, the RNA is modified mRNA. In embodiments, the modified mRNA comprises one or more non-canonical nucleotides.

In various embodiments, the present invention relates to the reprogramming of iPSCs to Monocytes, which can then be further differentiated into M1 and/or M2 macrophages, using non-viral, RNA-based means. iPSCs, namely pluripotent or less differentiated cells, can be reprogrammed from non-pluripotent or differentiated cells, including fibroblasts, keratinocytes, melanocyte blood cells, bone marrow cells, adipose cells, and tissue-resident progenitor cells.

In some embodiments, the method for reprogramming a non-pluripotent cell comprises: (a) providing a non-pluripotent cell; (b) culturing the non-pluripotent cell; and (c) transfecting the non-pluripotent cell with one or more synthetic RNA molecules, wherein the one or more synthetic RNA molecules include at least one RNA molecule encoding one or more reprogramming factors selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof; wherein the transfecting results in the cell expressing the one or more reprogramming factors to result in the cell being reprogrammed; and wherein step (c) occurs in the presence of a medium containing ingredients that support reprogramming of the differentiated cell to a less differentiated state.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof; wherein the transfecting results in the cell expressing the one or more reprogramming factors to result in the cell being reprogrammed to a less differentiated state; and wherein step (c) occurs in the presence of a medium containing ingredients that support reprogramming of the differentiated cell to a less differentiated state.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors; wherein the transfecting results in the cell expressing the one or more reprogramming factors; and wherein step (c) is performed at least twice and the amount of one or more synthetic RNA molecules transfected in one or more later transfections is greater than the amount transfected in one or more earlier transfections to result in the cell being reprogrammed to a less differentiated state and occurs in the presence of a medium containing ingredients that support reprogramming of the differentiated cell to a less differentiated state.

In some embodiments, the method for reprogramming a non-pluripotent cell, comprises: (a) providing a non-pluripotent cell; (b) culturing the non-pluripotent cell; and (c) transfecting the non-pluripotent cell with one or more synthetic RNA molecules, wherein the one or more synthetic RNA molecules include at least one RNA molecule encoding one or more reprogramming factors; wherein the transfecting results in the cell expressing the one or more reprogramming factors to result in the cell being reprogrammed; and wherein step (c) is performed without using irradiated human neonatal fibroblast feeder cells and occurs in the presence of a medium containing ingredients that support reprogramming of the cell.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors; wherein the transfecting results in the cell expressing the one or more reprogramming factors to result in the cell being reprogrammed to a less differentiated state; and wherein step (c) is performed without using irradiated human neonatal fibroblast feeder cells and occurs in the presence of a medium containing ingredients that support reprogramming of the cell to a less differentiated state.

In some embodiments, the method for reprogramming a non-pluripotent cell, comprises: (a) providing a non-pluripotent cell; (b) culturing the non-pluripotent cell; (c) transfecting the non-pluripotent cell with one or more synthetic RNA molecules, wherein the one or more synthetic RNA molecules include at least one RNA molecule encoding one or more reprogramming factors and wherein the transfecting results in the cell expressing the one or more reprogramming factors; and (d) repeating step (c) at least twice during 5 consecutive days, wherein the amount of one or more synthetic RNA molecules transfected in one or more later transfections is greater than the amount transfected in one or more earlier transfections, to result in the non-pluripotent cell being reprogrammed, wherein steps (c) and (d) occur in the presence of a medium containing ingredients that support reprogramming of the non-pluripotent cell.

In some embodiments, the method for reprogramming a differentiated cell to a less differentiated state, comprises: (a) providing a differentiated cell; (b) culturing the differentiated cell; (c) transfecting the differentiated cell with one or more synthetic RNA molecules, wherein the one or more synthetic RNA molecules include at least one RNA molecule encoding one or more reprogramming factors and wherein the transfecting results in the cell expressing the one or more reprogramming factors; and (d) repeating step (c) at least twice during 5 consecutive days, wherein the amount of one or more synthetic RNA molecules transfected in one or more later transfections is greater than the amount transfected in one or more earlier transfections, to result in the cell being reprogrammed to a less differentiated state, wherein steps (c) and (d) occur in the presence of a medium containing ingredients that support reprogramming of the differentiated cell to a less differentiated state.

In some embodiments, the method for reprogramming a non-pluripotent cell comprises: (a) providing a non-pluripotent cell, the non-pluripotent cell being derived from a biopsy of a human subject; (b) culturing the non-pluripotent cell; and (c) transfecting the non-pluripotent cell with a synthetic RNA molecule, wherein: the synthetic RNA molecule encodes one or more reprogramming factor(s) selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, the transfecting results in the non-pluripotent cell expressing the one or more reprogramming factor(s) which reprograms the non-pluripotent cell; and step (c) is performed without using irradiated human neonatal fibroblast feeder cells and occurs in the presence of a medium containing ingredients that support reprogramming of the non-pluripotent cell.

In some embodiments, the method for reprogramming a cell to a less differentiated state, comprises: (a) providing a non-pluripotent cell; (b) culturing the cell; and (c) transfecting the cell with a synthetic RNA molecule, wherein: the RNA molecule encodes one or more reprogramming factor(s) selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, the transfecting results in the cell expressing the one or more reprogramming factor(s) which reprograms the cell to a less differentiated state, and step (c) is performed without using irradiated human neonatal fibroblast feeder cells and occurs in the presence of a medium containing ingredients that support reprogramming of the cell to a less differentiated state.

In some embodiments, the method for reprogramming a cell to a less differentiated state, comprises: (a) providing a non-pluripotent cell; (b) culturing the cell in a medium containing ingredients that support reprogramming of the cell to a less differentiated state; and (c) transfecting the cell with a synthetic RNA molecule, wherein: the RNA molecule encodes one or more reprogramming factor(s) selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, the transfecting results in the cell expressing the one or more reprogramming factor(s) which reprograms the cell to a less differentiated state, and step (c) is performed without using irradiated human neonatal fibroblast feeder cells and occurs in the presence of a feeder cell conditioned medium.

In some embodiments, the method for reprogramming a cell to a less differentiated state comprises: (a) providing a non-pluripotent cell; (b) culturing the cell in a medium containing albumin and ingredients that support reprogramming of the cell to a less differentiated state, wherein the albumin is treated with an ion-exchange resin or charcoal; (c) transfecting the cell with a synthetic RNA molecule, wherein the RNA molecule encoding one or more reprogramming factor(s) selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, wherein the transfecting results in the cell expressing the one or more reprogramming factor(s) which reprograms the cell to a less differentiated state.

In some embodiments, the method for reprogramming a cell to a less differentiated state, comprises: (a) culturing a differentiated cell with a reprogramming medium; (b) transfecting the cell with one or more synthetic RNA molecules, wherein the one or more synthetic RNA molecules include at least one RNA molecule encoding one or more reprogramming factors and wherein the transfecting results in the cell expressing the one or more reprogramming factors; and (c) repeating step (b) at least twice during 5 consecutive days, wherein the amount of one or more synthetic RNA molecules transfected in one or more later transfections is greater than the amount transfected in one or more earlier transfections, to result in the cell being reprogrammed to a less differentiated state, wherein steps (a)-(c) are performed without using feeder cells and occur in the presence of a feeder cell conditioned medium.

In some embodiments, the method for reprogramming a cell to a less differentiated state, comprises: a. culturing a differentiated cell with a reprogramming medium containing albumin, wherein the albumin is treated with an ion-exchange resin or charcoal; b. transfecting the cell with one or more synthetic RNA molecules, wherein the one or more synthetic RNA molecules includes at least one RNA molecule encoding one or more reprogramming factors and wherein the transfecting results in the cell expressing the one or more reprogramming factors; and c. repeating step (b) at least twice during 5 consecutive days to result in the cell being reprogrammed to a less differentiated state.

In some embodiments, the method for reprogramming a cell to a less differentiated state, comprises: a. culturing a differentiated cell with a reprogramming medium containing albumin, wherein the albumin is treated with sodium octanoate; brought to a temperature of at least about 40° C.; and treated with an ion-exchange resin or charcoal; b. transfecting the cell with one or more synthetic RNA molecules, wherein the one or more synthetic RNA molecules includes at least one RNA molecule encoding one or more reprogramming transcription factors and wherein the transfecting results in the cell expressing the one or more synthetic RNA molecules; and c. repeating step (b) at least twice during about 5 consecutive days to result in the cell being reprogrammed to a less differentiated state. In embodiments, the reprogramming is non-viral. In embodiments, the reprogramming factor is one or more of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

In some embodiments, iPSCs are obtained and Monocytes, which can then be further differentiated into M1 and/or M2 macrophages, are generated via cell reprogramming with non-immunogenic messenger RNA (mRNA) encoding one or more reprogramming factors in a defined, animal component-free process. In some embodiments the process is immunosuppressant-free. In some embodiments, the process is animal component-free. In some embodiments, the process is defined. In some embodiments, iPSCs are generated from adult human dermal fibroblasts using a high-efficiency, immunosuppressant-free mRNA-based protocol, whereupon iPSCs are differentiated into monocytes using a 28-day monolayer protocol. Beginning on day 14, cells can be harvested every 3-4 days. CD14+ isolation yielded >95% CD14+ cells with an average yield of 4.1×10⁴ cells per cm2 per harvest. iPSC-derived monocytes were compared to peripheral blood mononuclear cell (PBMC)-derived monocytes for expression of key hematopoietic and myeloid-lineage markers CD11b, CD14, CD33, CD45, CD80, CD163, CD206, and SIRPα. In some embodiments, the differentiated monocytes are characterized by downregulation of Nanog and Oct4 and/or changes in expression of CD11b, CD14, CD33, CD45, CD80, CD163, CD206, and SIRPα, e.g., relative to the source cells. In some embodiments, rtPCR analysis is used to characterize monocytes. In some embodiments, monocytes are further differentiated into M1 and/or M2 macrophages. iPSC-derived monocytes can be further differentiated into macrophages by exposure to MCSF for 3-4 days. The macrophages can be assessed for their ability to polarize, secrete pro- and anti-inflammatory cytokines, and for cytotoxic activity when co-cultured with cancer cells. M1 macrophages can be polarized with interferon gamma (IFN-γ, 50 ng/mL) and lipopolysaccharide (LPS, 10 ng/mL) for 48 hours, whereas M2 macrophages can be treated with IL-4 (10 ng/mL) for 48 hours. The efficiency of iPSC-derived monocytes differentiation into macrophages can be assessed by cell adherence, morphology, and surface marker expression (CD14, CD45, CD163). The ability of M1 and M2 polarized iPSC-derived macrophages to secrete TNFα, IL-12p70, and IL-10 can be assayed and compared to PBMC-derived macrophages. Finally, the ability of M1 and M2 polarized iPSC-derived macrophages to kill cancer cells can be assayed.

Cells can be reprogrammed by exposing them to specific extracellular cues and/or by ectopic expression of specific proteins, microRNAs, etc. While several reprogramming methods have been previously described, most that rely on ectopic expression require the introduction of exogenous DNA, which can carry mutation risks. DNA-free reprogramming methods based on direct delivery of reprogramming proteins have been reported. However, these methods are too inefficient and unreliable for commercial use. In addition, RNA-based reprogramming methods have been described (see, e.g., Angel. MIT Thesis. 2008. 1-56; Angel et al. PLoS ONE. 2010. 5, 107; Warren et al. Cell Stem Cell. 2010. 7, 618-630; Angel. MIT Thesis. 2011. 1-89; and Lee et al., Cell. 2012. 151, 547-558; the contents of all of which are hereby incorporated by reference). However, existing RNA-based reprogramming methods are slow, unreliable, and inefficient when performed on adult cells, require many transfections (resulting in significant expense and opportunity for error), can reprogram only a limited number of cell types, can reprogram cells to only a limited number of cell types, require the use of immunosuppressants, and require the use of multiple human-derived components, including blood-derived HSA and human fibroblast feeders. The many drawbacks of previously disclosed RNA-based reprogramming methods make them undesirable for research, therapeutic or cosmetic use.

In some embodiments, reprogramming is performed by transfecting cells with one or more nucleic acids encoding one or more reprogramming factors, including, but not limited to Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In one embodiment, the cell is a human skin cell, and the human skin cell is reprogrammed to a pluripotent stem cell. In another embodiment, the cell is a human skin cell, and the human skin cell is reprogrammed to a glucose-responsive insulin-producing cell. Examples of other cells that can be reprogrammed and other cells to which a cell can be reprogrammed include, but are not limited to skin cells, pluripotent stem cells, MSCs, mesenchymal stromal/stem cells, β-cells, retinal pigmented epithelial cells, hematopoietic cells, hematopoietic stem cells, cardiac cells, airway epithelial cells, neural stem cells, neurons, glial cells, bone cells, blood cells, and dental pulp stem cells. In one embodiment, the cell is contacted with a medium that supports the reprogrammed cell. In one embodiment, the medium also supports the cell.

Importantly, infecting skin cells with viruses encoding Oct4, Sox2, Klf4, and c-Myc, combined with culturing the cells in a medium that supports the growth of cardiomyocytes, has been reported to cause reprogramming of the skin cells to cardiomyocytes, without first reprogramming the skin cells to pluripotent stem cells (See Efs et al Nat Cell Biol. 2011; 13:215-22, the contents of which are hereby incorporated by reference). In certain situations, direct reprogramming (reprogramming one somatic cell to another somatic cell without first reprogramming the somatic cell to a pluripotent stem cell, also known as “transdifferentiation”) may be desirable, in part because culturing pluripotent stem cells can be time-consuming and expensive, the additional handling involved in establishing and characterizing a stable pluripotent stem cell line can carry an increased risk of contamination, and the additional time in culture associated with first producing pluripotent stem cells can carry an increased risk of genomic instability and the acquisition of mutations, including point mutations, copy-number variations, and karyotypic abnormalities.

In embodiments, fewer total transfections may be required to reprogram a cell according to the methods of the present invention than according to other methods. Certain embodiments are therefore directed to a method for reprogramming a cell, wherein from about 1 to about 12 transfections are performed during about 20 consecutive days, or from about 4 to about 10 transfections are performed during about 15 consecutive days, or from about 4 to about 8 transfections are performed during about 10 consecutive days. It is recognized that when a cell is contacted with a medium containing nucleic acid molecules, the cell may likely come into contact with and/or internalize more than one nucleic acid molecule either simultaneously or at different times. A cell can therefore be contacted with a nucleic acid more than once, e.g., repeatedly, even when a cell is contacted only once with a medium containing nucleic acids.

Of note, nucleic acids can contain one or more non-canonical or “modified” residues as described herein. For instance, any of the non-canonical nucleotides described herein can be used in the present reprogramming methods. In one embodiment, pseudouridine-5′-triphosphate can be substituted for uridine-5′-triphosphate in an in vitro-transcription reaction to yield synthetic RNA, wherein up to 100% of the uridine residues of the synthetic RNA may be replaced with pseudouridine residues. In vitro-transcription can yield RNA with residual immunogenicity, even when pseudouridine and 5-methylcytidine are completely substituted for uridine and cytidine, respectively (see, e.g., Angel. Reprogramming Human Somatic Cells to Pluripotency Using RNA [Doctoral Thesis]. Cambridge, MA: MIT; 2011, the contents of which are hereby incorporated by reference). For this reason, it is common to add an immunosuppressant to the transfection medium when transfecting cells with RNA. In certain situations, adding an immunosuppressant to the transfection medium may not be desirable, in part because the recombinant immunosuppressant most commonly used for this purpose, B18R, can be expensive and difficult to manufacture. In one embodiment, the immunosuppressant is B18R or a biologically active fragment, analogue, variant or family-member thereof or dexamethasone or a derivative thereof. In one embodiment, the transfection medium does not contain an immunosuppressant, and the nucleic-acid dose is chosen to prevent excessive toxicity. In another embodiment, the nucleic-acid dose is less than about 1 mg/cm² of tissue or less than about 1 mg/100,000 cells or less than about 10 mg/kg.

In various cases, transfection of a cell with synthetic nucleic acids for reprogramming the cell may be facilitated by use of the ToRNAdo™ Nucleic-Acid Delivery System. This system relates to new lipids that find use, inter alia, in improved delivery of biological payloads, e.g., nucleic acids, to cells. The system relates to use of a compound of Formula (IV):

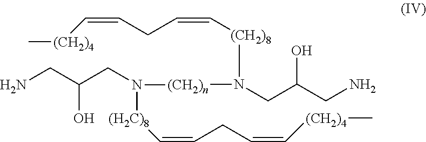

where n is 1, 2, 3, 4, 5, 6, 7, 8, 9, 10, 11, 12, 13, 14, or 15. Further description of ToRNAdo™ Nucleic-Acid Delivery System is found in one or both of U.S. Pat. No. 10,501,404 and WO2021003462. The entire contents of which are incorporated by reference in their entirety.

In any of the herein-disclosed aspects or embodiments, a synthetic RNA molecule may be in the form of a circular RNA (circRNA). The circRNA are manufactured by methods do not require a linear oligonucleotide (splint) to pre-orient the two reacting ends of a linear RNA to assist in ligation to yield a circRNA, the circRNA are manufactured by methods that do not require ribozymes to yield a circRNA, and/or the circRNA are manufactured by methods that do not require HPLC-based purification, e.g., post-ligation. A nucleic acid that can be manufactured into a circRNA has the structure: 5′-X-Y-A-IRES-B-CDS-C-Y′-Z 3′. Here, Y and Y′ each independently comprise one or more nucleotides and Y and Y′ are substantially complementary; X and Z each independently comprise one or more nucleotides and X and Z are not substantially complementary; IRES comprises an internal ribosome entry site; CDS comprises a coding sequence; and A, B, and C are each independently a spacer comprising one or more nucleotides or null. The CDS of a circRNA may encode one or more proteins of interest, the protein of interest being one or more reprogramming factors, optionally selected from Oct4, Sox2, Klf4, c-Myc, l-Myc, Tert, Nanog, Lin28, Glis1, Utf1, Aicda, miR200 micro-RNA, miR291 micro-RNA, miR294 micro-RNA and miR295 micro-RNA, miR302 micro-RNA, miR367 micro-RNA, miR369 micro-RNA, or a natural or engineered variant, family member, orthologue, fragment or fusion construct thereof. In some cases, the CDS encodes two, three, four, five, six, seven, eight, nine, ten, eleven, or more reprogramming factor(s). Additional details regarding circRNAs useful in the present disclosure are described in PCT/US2022/026564, the contents of which are incorporated herein by reference in its entirety.

Reprogrammed cells produced according to certain embodiments of the present invention are suitable for therapeutic and/or cosmetic applications as they do not contain undesirable exogenous DNA sequences, and they are not exposed to animal-derived or human-derived products, which may be undefined, and which may contain toxic and/or pathogenic contaminants. Furthermore, the high speed, efficiency, and reliability of certain embodiments of the present invention may reduce the risk of acquisition and accumulation of mutations and other chromosomal abnormalities. Certain embodiments of the present invention can thus be used to generate cells that have a safety profile adequate for use in therapeutic and/or cosmetic applications. For example, reprogramming cells using RNA and the medium of the present invention, wherein the medium does not contain animal or human-derived components, can yield cells that have not been exposed to allogeneic material. Certain embodiments are therefore directed to a reprogrammed cell that has a desirable safety profile. In one embodiment, the reprogrammed cell has a normal karyotype. In another embodiment, the reprogrammed cell has fewer than about 5 copy-number variations (CNVs) relative to the patient genome, such as fewer than about 3 copy-number variations relative to the patient genome, or no copy-number variations relative to the patient genome. In yet another embodiment, the reprogrammed cell has a normal karyotype and fewer than about 100 single nucleotide variants in coding regions relative to the patient genome, or fewer than about 50 single nucleotide variants in coding regions relative to the patient genome, or fewer than about 10 single nucleotide variants in coding regions relative to the patient genome.

Endotoxins and nucleases can co-purify and/or become associated with other proteins, such as serum albumin. Recombinant proteins, in particular, can often have high levels of associated endotoxins and nucleases, due in part to the lysis of cells that can take place during their production. Endotoxins and nucleases can be reduced, removed, replaced or otherwise inactivated by many of the methods of the present invention, including, for example, by acetylation, by addition of a stabilizer such as sodium octanoate, followed by heat treatment, by the addition of nuclease inhibitors to the albumin solution and/or medium, by crystallization, by contacting with one or more ion-exchange resins, by contacting with charcoal, by preparative electrophoresis or by affinity chromatography. In embodiments, partially or completely reducing, removing, replacing, or otherwise inactivating endotoxins and/or nucleases from a medium and/or from one or more components of a medium is provided and this can increase the efficiency with which cells can be transfected and reprogrammed. Certain embodiments are therefore directed to a method for transfecting a cell with one or more nucleic acids, wherein the transfection medium is treated to partially or completely reduce, remove, replace or otherwise inactivate one or more endotoxins and/or nucleases. Other embodiments are directed to a medium that causes minimal degradation of nucleic acids. In one embodiment, the medium contains less than about 1 EU/mL, or less than about 0.1 EU/mL, or less than about 0.01 EU/mL.

In certain situations, protein-based lipid carriers such as serum albumin can be replaced with non-protein-based lipid carriers such as methyl-beta-cyclodextrin. The medium of the present invention can also be used without a lipid carrier, for example, when transfection is performed using a method that may not require or may not benefit from the presence of a lipid carrier, for example, using one or more lipid-based transfection reagents, polymer-based transfection reagents or peptide-based transfection reagents or using electroporation. Many protein-associated molecules, such as metals, can be highly toxic to cells in vivo. This toxicity can cause decreased viability, as well as the acquisition of mutations. Certain embodiments thus have the additional benefit of producing cells that are free from toxic molecules.

The associated-molecule component of a protein can be measured by suspending the protein in solution and measuring the conductivity of the solution. Certain embodiments are therefore directed to a medium that contains a protein, wherein about a 10% solution of the protein in water has a conductivity of less than about 500 μmho/cm. In one embodiment, the solution has a conductivity of less than about 50 μmho/cm. In another embodiment, less than about 0.65% of the dry weight of the protein comprises lipids and/or less than about 0.35% of the dry weight of the protein comprises free fatty acids.

Certain embodiments are therefore directed to a method for transfecting a cell with a nucleic acid, wherein the cell is transfected more than once, and wherein the amount of nucleic acid delivered to the cell is different for two of the transfections. In one embodiment, the cell proliferates between two of the transfections, and the amount of nucleic acid delivered to the cell is greater for the second of the two transfections than for the first of the two transfections. In another embodiment, the cell is transfected more than twice, and the amount of nucleic acid delivered to the cell is greater for the second of three transfections than for the first of the same three transfections, and the amount of nucleic acid delivered to the cells is greater for the third of the same three transfections than for the second of the same three transfections. In yet another embodiment, the cell is transfected more than once, and the maximum amount of nucleic acid delivered to the cell during each transfection is sufficiently low to yield at least about 80% viability for at least two consecutive transfections.

In embodiments, there are provided methods in which modulating the amount of nucleic acid delivered to a population of proliferating cells in a series of transfections can result in both an increased effect of the nucleic acid and increased viability of the cells. In embodiments, when cells are contacted with one or more nucleic acids encoding one or more reprogramming factors in a series of transfections, the efficiency of reprogramming can be increased when the amount of nucleic acid delivered in later transfections is greater than the amount of nucleic acid delivered in earlier transfections, for at least part of the series of transfections. Certain embodiments are therefore directed to a method for reprogramming a cell, wherein one or more nucleic acids is repeatedly delivered to the cell in a series of transfections, and the amount of the nucleic acid delivered to the cell is greater for at least one later transfection than for at least one earlier transfection. In one embodiment, the cell is transfected from about 2 to about 10 times, or from about 3 to about 8 times, or from about 4 to about 6 times. In another embodiment, the one or more nucleic acids includes at least one RNA molecule, the cell is transfected from about 2 to about 10 times, and the amount of nucleic acid delivered to the cell in each transfection is the same as or greater than the amount of nucleic acid delivered to the cell in the most recent previous transfection. In yet another embodiment, the amount of nucleic acid delivered to the cell in the first transfection is from about 20 ng/cm² to about 250 ng/cm², or from 100 ng/cm² to 600 ng/cm². In yet another embodiment, the cell is transfected about 5 times at intervals of from about 12 to about 48 hours, and the amount of nucleic acid delivered to the cell is about 25 ng/cm² for the first transfection, about 50 ng/cm² for the second transfection, about 100 ng/cm² for the third transfection, about 200 ng/cm² for the fourth transfection, and about 400 ng/cm² for the fifth transfection. In yet another embodiment, the cell is further transfected at least once after the fifth transfection, and the amount of nucleic acid delivered to the cell is about 400 ng/cm².

Several molecules can be added to media by conditioning. Certain embodiments are therefore directed to a medium that is supplemented with one or more molecules that are present in a conditioned medium. In one embodiment, the medium is supplemented with Wnt1, Wnt2, Wnt3, Wnt3a or a biologically active fragment, analogue, variant, agonist, or family-member thereof. In another embodiment, the medium is supplemented with TGF-β or a biologically active fragment, analogue, variant, agonist, or family-member thereof. In yet another embodiment, a cell is reprogrammed according to the method of the present invention, wherein the medium is not supplemented with TGF-β for from about 1 to about 5 days and is then supplemented with TGF-β for at least about 2 days. In yet another embodiment, the medium is supplemented with IL-6, IL-6R or a biologically active fragment, analogue, variant, agonist, or family-member thereof. In yet another embodiment, the medium is supplemented with a sphingolipid or a fatty acid. In still another embodiment, the sphingolipid is lysophosphatidic acid, lysosphingomyelin, sphingosine-1-phosphate or a biologically active analogue, variant or derivative thereof.

In addition to mitotically inactivating cells, under certain conditions, irradiation can change the gene expression of cells, causing cells to produce less of certain proteins and more of certain other proteins than non-irradiated cells, for example, members of the Wnt family of proteins. In addition, certain members of the Wnt family of proteins can promote the growth and transformation of cells. In embodiments, the efficiency of reprogramming can be greatly increased by contacting a cell with a medium that is conditioned using irradiated feeders instead of mitomycin-c-treated feeders. In embodiments, the increase in reprogramming efficiency observed when using irradiated feeders is caused in part by Wnt proteins that are secreted by the feeders. Certain embodiments are therefore directed to a method for reprogramming a cell, wherein the cell is contacted with Wnt1, Wnt2, Wnt3, Wnt3a or a biologically active fragment, analogue, variant, family-member or agonist thereof, including agonists of downstream targets of Wnt proteins, and/or agents that mimic one or more of the biological effects of Wnt proteins, for example, 2-amino-4-[3,4-(methylenedioxy)benzylamino]-6-(3-methoxyphenyl)pyrimidine.

Because of the low efficiency of many DNA-based reprogramming methods, these methods may be difficult or impossible to use with cells derived from patient samples, which may contain only a small number of cells. In contrast, the high efficiency of certain embodiments of the present invention can allow reliable reprogramming of a small number of cells, including single cells. Certain embodiments are directed to a method for reprogramming a small number of cells. Other embodiments are directed to a method for reprogramming a single cell. In one embodiment, the cell is contacted with one or more enzymes. In another embodiment, the enzyme is collagenase. In yet another embodiment, the collagenase is animal-component free. In one embodiment, the collagenase is present at a concentration of from about 0.1 mg/mL to about 10 mg/mL, or from about 0.5 mg/mL to about 5 mg/mL. In another embodiment, the cell is a blood cell. In yet another embodiment, the cell is contacted with a medium containing one or more proteins that is derived from the patient's blood. In still another embodiment, the cell is contacted with a medium comprising: DMEM/F12+2 mM L-alanyl-L-glutamine+from about 5% to about 25% patient-derived serum, or from about 10% to about 20% patient-derived serum, or about 20% patient-derived serum.

In embodiments, transfecting cells with a mixture of RNA encoding Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof using the medium of the present invention can cause the rate of proliferation of the cells to increase. When the amount of RNA delivered to the cells is too low to ensure that all of the cells are transfected, only a fraction of the cells may show an increased proliferation rate. In certain situations, such as when generating a personalized therapeutic, increasing the proliferation rate of cells may be desirable, in part because doing so can reduce the time necessary to generate the therapeutic, and therefore can reduce the cost of the therapeutic. Certain embodiments are therefore directed to a method for transfecting a cell with a mixture of RNA encoding Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In one embodiment, the cell exhibits an increased proliferation rate. In another embodiment, the cell is reprogrammed.

While detailed examples are provided herein for the production of specific types of cells and for the production of therapeutics comprising specific types of cells, it is recognized that the methods of the present invention can be used to produce many other types of cells, and to produce therapeutics comprising one or more of many other types of cells, for example, by reprogramming a cell according to the methods of the present invention, and culturing the cell under conditions that mimic one or more aspects of development by providing conditions that resemble the conditions present in the cellular microenvironment during development.

Other embodiments are directed to a method for reprogramming a cell. In one embodiment, the cell is reprogrammed by contacting the cell with one or more nucleic acids. In one embodiment, the cell is contacted with a plurality of nucleic acids encoding at least one of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In another embodiment, the cell is contacted with a plurality of nucleic acids encoding a plurality of proteins including: Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

Illustrative subjects or patients refers to any vertebrate including, without limitation, humans and other primates (e.g., chimpanzees and other apes and monkey species), farm animals (e.g., cattle, sheep, pigs, goats, and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In some embodiments, the subject is a human.

In some embodiments, a synthetic RNA molecule is used to reprogram iPSCs into monocytes, which can then be further differentiated into M1 and/or M2 macrophages. In embodiments, the synthetic RNA molecule is mRNA. In embodiments, the synthetic RNA molecule is in vitro transcribed. In embodiments, the synthetic RNA is a circRNA.

In embodiments, the RNA is mRNA. In embodiments, the RNA is modified mRNA. In embodiments, the modified mRNA comprises one or more non-canonical nucleotides. In some embodiments, non-canonical nucleotides are incorporated into RNA to increase the efficiency with which the RNA can be translated into protein, and can decrease the toxicity of the RNA. In embodiments, the RNA molecule comprises one or more non-canonical nucleotides. In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 20 nucleotides to about 250 nucleotides.

Certain embodiments are directed to a nucleic acid comprising a 5′-cap structure selected from Cap 0, Cap 1, Cap 2, and Cap 3 or a derivative thereof. In one embodiment, the nucleic acid comprises one or more UTRs. In another embodiment, the one or more UTRs increase the stability of the nucleic acid. In a further embodiment, the one or more UTRs comprise an alpha-globin or beta-globin 5′-UTR. In a still further embodiment, the one or more UTRs comprise an alpha-globin or beta-globin 3′-UTR. In a still further embodiment, the RNA molecule comprises an alpha-globin or beta-globin 5′-UTR and an alpha-globin or beta-globin 3′-UTR. In one embodiment, the 5′-UTR comprises a Kozak sequence that is substantially similar to the Kozak consensus sequence. In another embodiment, the nucleic acid comprises a 3′-poly(A) tail. In a further embodiment, the 3′-poly(A) tail is between about 20 nt and about 250 nt or between about 120 nt and about 150 nt long. In a further embodiment, the 3′-poly(A) tail is about 20 nt, or about 30 nt, or about 40 nt, or about 50 nt, or about 60 nt, or about 70 nt, or about 80 nt, or about 90 nt, or about 100 nt, or about 110 nt, or about 120 nt, or about 130 nt, or about 140 nt, or about 150 nt, or about 160 nt, or about 170 nt, or about 180 nt, or about 190 nt, or about 200 nt, or about 210 nt, or about 220 nt, or about 230 nt, or about 240 nt, or about 250 nt long.

In some embodiments, the RNA comprises a tail composed of a plurality of adenines with one or more guanines.

In embodiments, the RNA comprises (a) a sequence encoding a protein, and (b) a tail region comprising deoxyadenosine nucleotides and one or more other nucleotides.

In embodiments, the one or more other nucleotides comprises deoxyguanosine residues. In embodiments, the tail region comprises about 1%, about 2%, about 5%, about 10%, about 15%, about 20%, about 25%, about 30%, about 35%, about 40%, about 45%, or about 50% deoxyguanosine residues. In embodiments, the tail region comprises more than 50% deoxyguanosine residues.

In embodiments, the one or more other nucleotides comprises deoxycytidine residues. In embodiments, the tail region comprises about 1%, about 2%, about 5%, about 10%, about 15%, about 20%, about 25%, about 30%, about 35%, about 40%, about 45%, or about 50% deoxycytidine residues. In embodiments, the tail region comprises more than 50% deoxycytidine residues.

In embodiments, the one or more other nucleotides comprises deoxythymidine residues. In embodiments, the tail region comprises about 1%, about 2%, about 5%, about 10%, about 15%, about 20%, about 25%, about 30%, about 35%, about 40%, about 45%, or about 50% deoxythymidine residues. In embodiments, the tail region comprises more than 50% deoxythymidine residues.

In embodiments, the one or more other nucleotides comprise deoxyguanosine residues and deoxycytidine residues. In embodiments, the tail region comprises about 99%, about 98%, about 95%, about 90%, about 85%, about 80%, about 75%, about 70%, about 65%, about 60%, about 55%, or about 50% deoxyadenosine residues. In embodiments, the tail region comprises fewer than 50% deoxyadenosine residues.

In embodiments, the one or more other nucleotides comprises guanosine residues.

In embodiments, the tail region comprises about 1%, about 2%, about 5%, about 10%, about 15%, about 20%, about 25%, about 30%, about 35%, about 40%, about 45%, or about 50% guanosine residues. In embodiments, the tail region comprises more than 50% guanosine residues.

In embodiments, the one or more other nucleotides comprises cytidine residues. In embodiments, the tail region comprises about 1%, about 2%, about 5%, about 10%, about 15%, about 20%, about 25%, about 30%, about 35%, about 40%, about 45%, or about 50% cytidine residues. In embodiments, the tail region comprises more than 50% cytidine residues.

In embodiments, the one or more other nucleotides comprises uridine residues. In embodiments, the tail region comprises about 1%, about 2%, about 5%, about 10%, about 15%, about 20%, about 25%, about 30%, about 35%, about 40%, about 45%, or about 50% uridine residues. In embodiments, the tail region comprises more than 50% uridine residues.

In embodiments, the one or more other nucleotides comprise guanosine residues and cytidine residues. In embodiments, the tail region comprises about 99%, about 98%, about 95%, about 90%, about 85%, about 80%, about 75%, about 70%, about 65%, about 60%, about 55%, or about 50% adenosine residues.

In embodiments, the tail region comprises fewer than 50% adenosine residues.

In embodiments, the tail is (A)₁₅₀ (SEQ ID NO: 61). In embodiments, the tail is (A₃₉G)₃(A)₃₀ (SEQ ID NO: 62). In embodiments, the tail is (A₁₉G)₇(A)₁₀ (SEQ ID NO: 63). In embodiments, the tail is (A₉G)₁₅ (SEQ ID NO: 64).

In embodiments, the length of the tail region is between about 80 nucleotides and about 120 nucleotides, about 120 nucleotides and about 160 nucleotides, about 160 nucleotides and about 200 nucleotides, about 200 nucleotides and about 240 nucleotides, about 240 nucleotides and about 280 nucleotides, or about 280 nucleotides and about 320 nucleotides.

In embodiments, the length of the tail region is greater than 320 nucleotides.

In embodiments, the RNA comprises a 5′ cap structure. In embodiments, the RNA 5′-UTR comprises a Kozak consensus sequence. In embodiments, the RNA 5′-UTR comprises a sequence that increases RNA stability in vivo, and the 5′-UTR may comprise an alpha-globin or beta-globin 5′-UTR.

In embodiments, the RNA 3′-UTR comprises a sequence that increases RNA stability in vivo, and the 3′-UTR may comprise an alpha-globin or beta-globin 3′-UTR. In embodiments, the RNA comprises a 3′ poly(A) tail. In embodiments, the RNA 3′ poly(A) tail is from about 20 nucleotides to about 250 nucleotides in length.

In embodiments, the RNA is from about 200 nucleotides to about 5000 nucleotides in length.

In embodiments, the RNA is prepared by in vitro transcription. In embodiments, the RNA is synthetic.

In some embodiments, the synthetic RNA comprises about 200 nucleotides to about 5000 nucleotides.

In some embodiments, the synthetic RNA comprises from about 500 to about 2000 nucleotides, or about 500 to about 1500 nucleotides, or about 500 to about 1000 nucleotides.

Further description of reprogramming is found in one or more of WO/2013/086008, WO/2014/071219, WO/2015/117021, WO/2016/131052, WO/2018/035377, WO/2019/191341, WO/2021/003462, WO2021/231549, or WO2021/222389. The entire contents of which are incorporated by reference in their entirety.

### Gene-Editing

In any herein disclosed aspect or embodiment, a cell is gene-edited.

In embodiments, gene-editing a cell comprises contacting the cell with a synthetic nucleic acid encoding one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a(Cpf1), Cas13a, Cas14, CasX, CasY, a Class 1 Cas protein, a Class 2 Cas protein, MAD7, and a gene-editing protein comprising a repeat sequence comprising LTPvQVVAIAwxyz (SEQ ID NO: 16), or a natural or engineered variant, family member, orthologue, fragment or fusion construct thereof.

In embodiments, the gene-editing protein comprises: (i) a DNA-binding domain comprising a plurality of repeat sequences and (ii) the nuclease domain comprising a catalytic domain of a nuclease. In embodiments, the at least one of the repeat sequences comprises the amino acid sequence: LTPvQVVAIAwxyzα (SEQ ID NO: 22) and is optionally between 36 and 39 amino acids long, where:
v is Q, D or E,
w is S or N,
x is I, H, N, or I,
y is D, A, I, N, H, K, S, G, or null,
z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), and
α is four consecutive amino acids.

In embodiments, α comprises at least one glycine (G) residue. In embodiments, α comprises at least one histidine (H) residue. In embodiments, α comprises at least one histidine (H) residue at any one of positions 33, 34, or 35. In embodiments, α comprises at least one aspartic acid (D) residue. In embodiments, α comprises at least one, or two, or three of a glycine (G) residue, a histidine (H) residue, and an aspartic acid (D) residue.

In embodiments, α comprises one or more hydrophilic residues, optionally selected from: a polar and positively charged hydrophilic amino acid, optionally selected from arginine (R) and lysine (K); a polar and neutral of charge hydrophilic amino acid, optionally selected from asparagine (N), glutamine (Q), serine (S), threonine (T), proline (P), and cysteine (C); a polar and negatively charged hydrophilic amino acid, optionally selected from aspartate (D) and glutamate (E), and an aromatic, polar and positively charged hydrophilic amino acid, optionally selected from histidine (H).

In some embodiments, α comprises one or more polar and positively charged hydrophilic amino acids selected from arginine (R) and lysine (K). In some embodiments, α comprises one or more polar and neutral of charge hydrophilic amino acids selected from asparagine (N), glutamine (Q), serine (S), threonine (T), proline (P), and cysteine (C). In some embodiments, α comprises one or more polar and negatively charged hydrophilic amino acids selected from aspartate (D) and glutamate (E). In some embodiments, α comprises one or more aromatic, polar and positively charged hydrophilic amino acids selected from histidine (H).

In embodiments, α comprises one or more hydrophobic residues, optionally selected from: a hydrophobic, aliphatic amino acid, optionally selected from glycine (G), alanine (A), leucine (L), isoleucine (I), methionine (M), and valine (V), and a hydrophobic, aromatic amino acid, optionally selected from phenylalanine (F), tryptophan (W), and tyrosine (Y). In some embodiments, α comprises one or more hydrophobic, aliphatic amino acids selected from glycine (G), alanine (A), leucine (L), isoleucine (I), methionine (M), and valine (V). In some embodiments, α comprises one or more aromatic amino acids selected from phenylalanine (F), tryptophan (W), and tyrosine (Y). In embodiments, the DNA-binding domain comprises about 15, or about, 16, or about 17, or about 18, or about 18.5 repeat sequences.

In embodiments, α is selected from GHGG (SEQ ID NO: 31), HGSG (SEQ ID NO: 32), HGGG (SEQ ID NO: 33), from GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 4260

In embodiments, the gene-editing protein has a DNA binding domain having at least one repeat of LTPEQVVAIAS*RVD*GGKQALETVQRLLPVLCQAGHGG (SEQ ID NO: 65; the “*RVD*” corresponds to the dinucleotide “xy” of SEQ ID NO: 22).

In embodiments, the repeat sequence is 33 or 34 amino acids long. In embodiments, the repeat sequence is 36-39 amino acids long. In some embodiments, the repeat sequence is 36 amino acids long. In some embodiments, the repeat sequence is 37 amino acids long. In some embodiments, the repeat sequence is 38 amino acids long. In some embodiments, the repeat sequence is 39 amino acids long.

In embodiments, the gene-editing protein comprises (i) a DNA-binding domain comprising a plurality of repeat sequences and at least one of the repeat sequences comprises the amino acid sequence: LTPvQVVAIAwxyzGHGG (SEQ ID NO: 17) or LTPvQVVAIAwxyzGTHG (SEQ ID NO: 18) and is from 36 to 39 amino acids long, wherein: “v” is Q, D or E, “w” is S or N, “x” is H, N, or I, “y” is D, A, I, N, G, H, K, S, or null, and “z” is GGKQALETVQRLLPVLCQD (SEQ ID NO: 19) or GGKQALETVQRLLPVLCQA (SEQ ID NO: 20) and (ii) a nuclease domain comprising a catalytic domain of a nuclease. In some embodiments, a gene-editing protein comprises a C-terminal GTHG (SEQ ID NO: 21) produces more efficient editing at the target locus than TALENs at 33° C. GTHG (SEQ ID NO: 21). In various embodiments, a gene-editing protein comprises a C-terminal GTHG (SEQ ID NO: 21) produces more efficient editing at the target locus than TALENs at 37° C.

In embodiments, the gene-editing protein comprises (i) a DNA-binding domain comprising a plurality of repeat sequences and at least one of the repeat sequences comprises the amino acid sequence: LTPvQVVAIAwxyzα (SEQ ID NO: 22) and is from 36 to 39 amino acids long, wherein: v is Q, D or E, w is S or N, x is I, H, N, or I, y is D, A, I, N, H, K, S, G or null, z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), a is four consecutive amino acids; and (ii) a nuclease domain comprising a catalytic domain of a nuclease. In embodiments, a is selected from GHGG (SEQ ID NO: 31), HGSG (SEQ ID NO: 32), HGGG (SEQ ID NO: 33), GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60). In some embodiments, a gene-editing protein comprises a C-terminal GTHG (SEQ ID NO: 21) produces more efficient editing at the target locus than TALENs at 33° C. GTHG (SEQ ID NO: 21). In various embodiments, a gene-editing protein comprises a C-terminal GTHG (SEQ ID NO: 21) produces more efficient editing at the target locus than TALENs at 37° C. Certain embodiments are directed to a nucleic acid molecule encoding a non-naturally occurring fusion protein comprising a first region that recognizes a predetermined nucleotide sequence and a second region with endonuclease activity, wherein the first region contains an artificial TAL effector repeat domain comprising one or more repeat units about 36 amino acids in length which differ from each other by no more than seven amino acids, and wherein the repeat domain is engineered for recognition of the predetermined nucleotide sequence. In one embodiment, the first region contains the amino acid sequence: LTPXQVVAIAS (SEQ ID NO: 29) where X can be either E or Q. In another embodiment, the amino acid sequence LTPXQVVAIAS (SEQ ID NO: 29) of the encoded non-naturally occurring fusion protein is immediately followed by an amino acid sequence selected from: HD, NG, NS, NI, NN, and N. In a further embodiment, the fusion protein comprises restriction endonuclease activity.

In embodiments, the gene-editing protein comprises (i) a DNA-binding domain comprising a plurality of repeat sequences and at least one of the repeat sequences comprises the amino acid sequence: LTPvQVVAIAwxyzHG, (SEQ ID NO: 30) wherein “v” is D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In another embodiment, the repeat sequence comprises: LTPvQVVAIAwxyzHG, (SEQ ID NO: 30) wherein “v” is D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwxyzHG, (SEQ ID NO: 30) wherein “v” is D or E, “w” is S or N, “x” is any amino acid other than N, H and I, “y” is any amino acid or no amino acid, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAIwyzHG (SEQ ID NO: 55), wherein “v” is D or E, “w” is S or N, “y” is any amino acid other than G, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwIAzHG (SEQ ID NO: 66), wherein “v” is D or E, “w” is S or N, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwxyzHG (SEQ ID NO: 30), wherein “v” is D or E, “w” is S or N, “x” is S, T or Q, “y” is any amino acid or no amino acid, and “z” is GGRPALE (SEQ ID NO: 23), GGKQALE (SEQ ID NO: 23), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwxyzHG (SEQ ID NO: 30), wherein “v” is D or E, “w” is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwx (SEQ ID NO: 67), wherein “v” is D or E, “w” is S or N, and “x” is S, T or Q. In yet another embodiment, the repeat sequence comprises: LTPvQVVAIAwxy (SEQ ID NO: 16), wherein “v” is D or E, “w” is S or N, “x” is S, T or Q, and “y” is selected from: D, A, I, N, H, K, S, and G. In yet another embodiment, the repeat sequence comprises: LTPvQVVAIAwxyzGHGG (SEQ ID NO: 17), wherein “v” is Q, D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, “w” is S or N, “x” is any amino acid other than N, H and I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwyzGHGG (SEQ ID NO: 70), wherein “v” is Q, D or E, “w” is S or N, “y” is any amino acid other than G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwIAzGHGG, (SEQ ID NO: 71) wherein “v” is Q, D or E, “w” is S or N, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, “w” is S or N, “x” is S, T or Q, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, V is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwx (SEQ ID NO: 67), wherein “v” is Q, D or E, “w” is S or N, and “x” is S, T or Q. In yet another embodiment, the repeat sequence comprises: LTPvQVVAIAwxy (SEQ ID NO: 72), wherein “v” is Q, D or E, “w” is S or N, “x” is S, T or Q, and “y” is selected from: D, A, I, N, H, K, S, and G.

The above-mentioned gene-editing proteins comprise a repeat variable di-residue (RVD) at residue 12 or 13, e.g., at “x” and “y” in the various above-mentioned repeat sequences, e.g., LTPvQVVAIAwxyzα (SEQ ID NO: 22), which targets the DNA-binding domain to a target DNA molecule. In embodiments, the RVD recognizes one base pair in the nucleic acid molecule. In embodiments, the RVD recognizes a C residue in the nucleic acid molecule and is selected from HD, N(null), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from Nl and NS. In embodiments, the RVD recognizes a T residue in the nucleic acid molecule and is selected from NG, HG, H(null), and IG.

In some embodiments, the RVD recognizing a C residue in the nucleic acid molecule is HD. In some embodiments, the RVD recognizing a C residue in the nucleic acid molecule is N(null). In some embodiments, the RVD recognizing a C residue in the nucleic acid molecule is HA. In some embodiments, the RVD recognizing a C residue in the nucleic acid molecule is ND. In some embodiments, the RVD recognizing a C residue in the nucleic acid molecule is HI. In some embodiments, the RVD recognizing a G residue in the nucleic acid molecule is NN. In some embodiments, the RVD recognizing a G residue in the nucleic acid molecule is NH. In some embodiments, the RVD recognizing a G residue in the nucleic acid molecule is NK. In some embodiments, the RVD recognizing a G residue in the nucleic acid molecule is HN. In some embodiments, the RVD recognizing a G residue in the nucleic acid molecule is NA. In some embodiments, the RVD recognizing an A residue in the nucleic acid molecule is Nl. In some embodiments, the RVD recognizing an A residue in the nucleic acid molecule is NS. In some embodiments, the RVD recognizing a T residue in the nucleic acid molecule is NG. In some embodiments, the RVD recognizing a T residue in the nucleic acid molecule is HG. In some embodiments, the RVD recognizing a T residue in the nucleic acid molecule is H(null). In some embodiments, the RVD recognizing a T residue in the nucleic acid molecule is IG.

In some embodiments, alternative DNA binding domains are employed.

For example, the alternative DNA binding domains described herein are, in embodiments, paired with the novel engineered nuclease domains described herein.

For example, the alternative DNA binding domains described herein are, in embodiments, used in the conditional activity: temperature dependence methods described herein.

For example, the alternative DNA binding domains described herein are, in embodiments, used in the conditional activity: methylation status methods described herein.

In embodiments, the engineered gene-editing proteins do not require a thymine (T) in the zero position of the target site (“To”).

In embodiments, the engineered gene-editing proteins that comprise DNA-binding domains comprise alterations in the in the N-terminal region to remove the T₀ requirement.

In embodiments, there is provided a method of gene-editing a cell with one or more of the present gene-editing proteins, optionally with also using a linear DNA repair template, optionally also using conditional activity methods described herein, where the target site lacks a thymine (T) in the zero position.

Wild type N-terminal region is characterized by the sequence: Asp225-IVGVGKQWSGARAL-Glu240 (DIVGVGKQWSGARALE; SEQ ID NO: 73). In embodiments, there is provided the engineered N-terminal region of Asp225-IVGVGKQKRGARAL-Glu240 (underling showing the change WS->KR) (DIVGVGKQKRGARALE; SEQ ID NO: 74).

In embodiments, there is provided an engineered N-terminal region in which KQWS is replaced with one or more amino acids, e.g., about 2-10 amino acids, or about 4-10 amino acids, or about 6-10 amino acids, or about 8-10 amino acids, or about 4 amino acids, or about 6 amino acids, or about 8 amino acids, or about 10 amino acids.

In embodiments, there is provided the engineered N-terminal region of Asp225-IVGVGGSKRGAGSGARAL-Glu244 (underling showing the change KQWS->GSKRGAGS) (DIVGVGGSKRGAGSGARALE; SEQ ID NO: 75).

In some cases, a cell is contacted with a demethylating agent during the process of gene-editing. In embodiments, the demethylating agent is selected from 5-azacitidine and 5-aza-2′-deoxycitidine (decitabine).

In some embodiments, the gene-editing protein comprises: (a) the DNA-binding domain comprising a plurality of repeat sequences and at least one of the repeat sequences comprises a repeat variable di-residue (RVD) at residue 12 or 13; and (b) the nuclease domain comprising a catalytic domain, the catalytic domain comprising a hybrid of the catalytic domains of Fokl and Stsl, comprising the α1, α2, α3, α4, α5, α6, β1, β2, β, β4, β5, and β6 domains of Fokl with at least one of the domains of Fokl being substituted in whole or in part with the α1, α2, α3, α4, α5, α6, β1, β2, β3, β4, β5, and β6 domains of Stsl and optionally comprising at least one mutation. In embodiments, the nuclease domain is capable of forming a dimer with another nuclease domain.

In some embodiments, certain fragments of an endonuclease cleavage domain are used, including fragments that are truncated at the N-terminus, fragments that are truncated at the C-terminus, fragments that have internal deletions, and fragments that combine N-terminus, C-terminus, and/or internal deletions, which maintain part or all of the catalytic activity of the full endonuclease cleavage domain. Determining whether a fragment can maintain part, or all of the catalytic activity of the full domain can be accomplished by, for example, synthesizing a gene-editing protein that contains the fragment according to the methods of the present invention, inducing cells to express the gene-editing protein according to the methods of the present invention, and measuring the efficiency of gene editing. In some embodiments, a measurement of gene-editing efficiency is used to ascertain whether any specific fragment maintains part or all of the catalytic activity of the full endonuclease cleavage domain. Certain embodiments are therefore directed to a biologically active fragment of an endonuclease cleavage domain. In one embodiment, the endonuclease cleavage domain is selected from: FokI, StsI, StsI-HA, StsI-HA2, StsI-UHA, StsI-UHA2, StsI-HF, and StsI-UHF or a natural or engineered variant or biologically active fragment thereof, or a hybrid or chimera thereof.

In embodiments, the gene-editing protein comprises a linker. In another embodiment, the linker connects a DNA-binding domain to a nuclease domain. In a further embodiment, the linker is between about 1 and about 10 amino acids long. In some embodiments, the linker is about 1, about 2, or about 3, or about 4, or about 5, or about 6, or about 7, or about 8, or about 9, or about 10 amino acids long. In one embodiment, the gene-editing protein is capable of generating a nick or a double-strand break in a target DNA molecule.

In embodiments, the gene-editing protein is any of those described in International Patent Publication No. WO 2014/071219 or WO2021/231549, hereby incorporated by reference in their entireties.

In various embodiments, the cell is transfected (e.g., contacted) with a synthetic nucleic acid encoding the gene-editing protein at about 30° C. to about 35° C., e.g., without limitation about 33° C. In embodiments, the contacting occurs at about 30° C. In some embodiments, the contacting occurs at about 31° C. In some embodiments, the contacting occurs at about 32° C. In some embodiments, the contacting occurs at about 33° C. In some embodiments, the contacting occurs at about 34° C. In some embodiments, the contacting occurs at about 35° C. In embodiments, the gene-editing protein is functionally temperature-switchable. In embodiments, the method further comprises the step of (c) culturing the contacted cell at about 30° C. to about 35° C. In embodiments, the method further comprises the step of (c) culturing the contacted cell at about 30° C. In embodiments, the method further comprises the step of (c) culturing the contacted cell at about 31° C. In embodiments, the method further comprises the step of (c) culturing the contacted cell at about 32° C. In embodiments, the method further comprises the step of (c) culturing the contacted cell at about 33° C. In embodiments, the method further comprises the step of (c) culturing the contacted cell at about 34° C. In embodiments, the method further comprises the step of (c) culturing the contacted cell at about 35° C.

Further description of temperature-sensitive gene-editing is found in WO2021/231549. The entire contents of which are incorporated by reference in their entirety.

In embodiments, the synthetic nucleic acid encoding the gene-editing protein is transfected along with a repair template. In some cases, the repair template is a double stranded synthetic oligodeoxynucleotide (dsODNs). In embodiments, the dsODNs comprises a repair template and comprises the TTAGGG motif. In some cases, the dsODN comprises a repair template and lacks the TTAGGG motif and a separate dsODNs comprising the TTAGGG motif is transfected into the cell. In embodiments, the RNA is mRNA. In embodiments, the RNA is modified mRNA. In embodiments, the modified mRNA comprises one or more non-canonical nucleotides. In some embodiments, non-canonical nucleotides are incorporated into RNA to increase the efficiency with which the RNA can be translated into protein, and can decrease the toxicity of the RNA. In embodiments, the RNA molecule comprises one or more non-canonical nucleotides. In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 20 nucleotides to about 250 nucleotides.

In some embodiments, the synthetic RNA comprises about 200 nucleotides to about 5000 nucleotides. In some embodiments, the synthetic RNA comprises from about 500 to about 2000 nucleotides, or about 500 to about 1500 nucleotides, or about 500 to about 1000 nucleotides.

In various cases, transfection of a cell with synthetic nucleic acids for gene-editing the cell may be facilitated by use of the ToRNAdo™ Nucleic-Acid Delivery System. This system relates to new lipids that find use, inter alia, in improved delivery of biological payloads, e.g., nucleic acids, to cells. The system relates to use of a compound of Formula (IV)

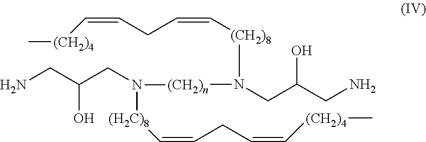

where n is 1, 2, 3, 4, 5, 6, 7, 8, 9, 10, 11, 12, 13, 14, or 15. Further description of ToRNAdo™ Nucleic-Acid Delivery System is found in one or both of U.S. Pat. No. 10,501,404 and WO2021003462. The entire contents of which are incorporated by reference in their entirety.

In any of the herein-disclosed aspects or embodiments, a synthetic RNA molecule encoding the gene-editing protein may be in the form of a circular RNA (circRNA). The circRNA are manufactured by methods do not require a linear oligonucleotide (splint) to pre-orient the two reacting ends of a linear RNA to assist in ligation to yield a circRNA, the circRNA are manufactured by methods that do not require ribozymes to yield a circRNA, and/or the circRNA are manufactured by methods that do not require HPLC-based purification, e.g., post-ligation. A nucleic acid that can be manufactured into a circRNA has the structure: 5′-X-Y-A-IRES-B-CDS-C-Y′-Z 3′. Here, Y and Y′ each independently comprise one or more nucleotides and Y and Y′ are substantially complementary; X and Z each independently comprise one or more nucleotides and X and Z are not substantially complementary; IRES comprises an internal ribosome entry site; CDS comprises a coding sequence; and A, B, and C are each independently a spacer comprising one or more nucleotides or null. The CDS of a circRNA encodes the gene-editing protein(s). Additional details regarding circRNAs useful in the present disclosure are described in PCT/US2022/026564, the contents of which are incorporated herein by reference in its entirety.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 20 nucleotides to about 250 nucleotides.

Certain embodiments are directed to a nucleic acid comprising a 5′-cap structure selected from Cap 0, Cap 1, Cap 2, and Cap 3 or a derivative thereof. In one embodiment, the nucleic acid comprises one or more UTRs. In another embodiment, the one or more UTRs increase the stability of the nucleic acid. In a further embodiment, the one or more UTRs comprise an alpha-globin or beta-globin 5′-UTR. In a still further embodiment, the one or more UTRs comprise an alpha-globin or beta-globin 3′-UTR. In a still further embodiment, the RNA molecule comprises an alpha-globin or beta-globin 5′-UTR and an alpha-globin or beta-globin 3′-UTR. In one embodiment, the 5′-UTR comprises a Kozak sequence that is substantially similar to the Kozak consensus sequence. In another embodiment, the nucleic acid comprises a 3′-poly(A) tail. In a further embodiment, the 3′-poly(A) tail is between about 20 nt and about 250 nt or between about 120 nt and about 150 nt long. In a further embodiment, the 3′-poly(A) tail is about 20 nt, or about 30 nt, or about 40 nt, or about 50 nt, or about 60 nt, or about 70 nt, or about 80 nt, or about 90 nt, or about 100 nt, or about 110 nt, or about 120 nt, or about 130 nt, or about 140 nt, or about 150 nt, or about 160 nt, or about 170 nt, or about 180 nt, or about 190 nt, or about 200 nt, or about 210 nt, or about 220 nt, or about 230 nt, or about 240 nt, or about 250 nt long.

In some embodiments, the RNA comprises a tail composed of a plurality of adenines with one or more guanines.

In embodiments, the RNA comprises (a) a sequence encoding a protein, and (b) a tail region comprising deoxyadenosine nucleotides and one or more other nucleotides.

In embodiments, the one or more other nucleotides comprises deoxyguanosine residues. In embodiments, the tail region comprises about 1%, about 2%, about 5%, about 10%, about 15%, about 20%, about 25%, about 30%, about 35%, about 40%, about 45%, or about 50% deoxyguanosine residues. In embodiments, the tail region comprises more than 50% deoxyguanosine residues.

In embodiments, the one or more other nucleotides comprises deoxycytidine residues. In embodiments, the tail region comprises about 1%, about 2%, about 5%, about 10%, about 15%, about 20%, about 25%, about 30%, about 35%, about 40%, about 45%, or about 50% deoxycytidine residues. In embodiments, the tail region comprises more than 50% deoxycytidine residues.

In embodiments, the one or more other nucleotides comprises deoxythymidine residues. In embodiments, the tail region comprises about 1%, about 2%, about 5%, about 10%, about 15%, about 20%, about 25%, about 30%, about 35%, about 40%, about 45%, or about 50% deoxythymidine residues. In embodiments, the tail region comprises more than 50% deoxythymidine residues.

In embodiments, the one or more other nucleotides comprise deoxyguanosine residues and deoxycytidine residues. In embodiments, the tail region comprises about 99%, about 98%, about 95%, about 90%, about 85%, about 80%, about 75%, about 70%, about 65%, about 60%, about 55%, or about 50% deoxyadenosine residues. In embodiments, the tail region comprises fewer than 50% deoxyadenosine residues.

In embodiments, the one or more other nucleotides comprises guanosine residues.

In embodiments, the tail region comprises about 1%, about 2%, about 5%, about 10%, about 15%, about 20%, about 25%, about 30%, about 35%, about 40%, about 45%, or about 50% guanosine residues. In embodiments, the tail region comprises more than 50% guanosine residues.

In embodiments, the one or more other nucleotides comprises cytidine residues. In embodiments, the tail region comprises about 1%, about 2%, about 5%, about 10%, about 15%, about 20%, about 25%, about 30%, about 35%, about 40%, about 45%, or about 50% cytidine residues. In embodiments, the tail region comprises more than 50% cytidine residues.

In embodiments, the one or more other nucleotides comprises uridine residues. In embodiments, the tail region comprises about 1%, about 2%, about 5%, about 10%, about 15%, about 20%, about 25%, about 30%, about 35%, about 40%, about 45%, or about 50% uridine residues. In embodiments, the tail region comprises more than 50% uridine residues.

In embodiments, the one or more other nucleotides comprise guanosine residues and cytidine residues. In embodiments, the tail region comprises about 99%, about 98%, about 95%, about 90%, about 85%, about 80%, about 75%, about 70%, about 65%, about 60%, about 55%, or about 50% adenosine residues.

In embodiments, the tail region comprises fewer than 50% adenosine residues.

In embodiments, the tail is (A)₁₅₀ (SEQ ID NO: 61). In embodiments, the tail is (A₃₉G)₃(A)₃₀ (SEQ ID NO: 62). In embodiments, the tail is (A₁₉G)₇(A)₁₀ (SEQ ID NO: 63). In embodiments, the tail is (A₉G)₁₅ (SEQ ID NO: 64).

In embodiments, the length of the tail region is between about 80 nucleotides and about 120 nucleotides, about 120 nucleotides and about 160 nucleotides, about 160 nucleotides and about 200 nucleotides, about 200 nucleotides and about 240 nucleotides, about 240 nucleotides and about 280 nucleotides, or about 280 nucleotides and about 320 nucleotides.

In embodiments, the length of the tail region is greater than 320 nucleotides.

In embodiments, the RNA comprises a 5′ cap structure. In embodiments, the RNA 5′-UTR comprises a Kozak consensus sequence. In embodiments, the RNA 5′-UTR comprises a sequence that increases RNA stability in vivo, and the 5′-UTR may comprise an alpha-globin or beta-globin 5′-UTR.

In embodiments, the RNA 3′-UTR comprises a sequence that increases RNA stability in vivo, and the 3′-UTR may comprise an alpha-globin or beta-globin 3′-UTR. In embodiments, the RNA comprises a 3′ poly(A) tail. In embodiments, the RNA 3′ poly(A) tail is from about 20 nucleotides to about 250 nucleotides in length.

In embodiments, the RNA is from about 200 nucleotides to about 5000 nucleotides in length.

In embodiments, the RNA is prepared by in vitro transcription. In embodiments, the RNA is synthetic. In some embodiments, the synthetic RNA comprises about 200 nucleotides to about 5000 nucleotides. In some embodiments, the synthetic RNA comprises from about 500 to about 2000 nucleotides, or about 500 to about 1500 nucleotides, or about 500 to about 1000 nucleotides.

Further description of gene-editing is found in one or more of WO/2013/086008, WO/2014/071219, WO/2015/117021, WO/2016/131052, WO/2018/035377, WO/2019/191341, WO/2021/003462, WO2021/231549, or WO2021/222389. The entire contents of which are incorporated by reference in their entirety.

### RNA Modifications

In embodiments, the present disclosure relates to RNA-based modifications, e.g., reprogramming and/or gene-editing. In some embodiments, an RNA molecule encodes a gene-editing protein. In some embodiments, a RNA molecule encodes a reprogramming factor.

In embodiments, the RNA is mRNA. In embodiments, the RNA is modified mRNA. In embodiments, the modified mRNA comprises one or more non-canonical nucleotides.

In some embodiments, non-canonical nucleotides are incorporated into RNA to increase the efficiency with which the RNA can be translated into protein, and can decrease the toxicity of the RNA. In embodiments, the RNA molecule comprises one or more non-canonical nucleotides.

In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In embodiments, the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In some embodiments, the one or more non-canonical nucleotides are selected from 5-hydroxycytidine, 5-methylcytidine, 5-hydroxymethylcytidine, 5-carboxycytidine, 5-formylcytidine, 5-methoxycytidine, 5-hydroxyuridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine. In some embodiments, at least 50%, or at least 55%, or at least 60%, or at least 65%, or at least 70%, or at least 75%, or at least 80%, or at least 85%, or at least 90%, or at least 95%, or 100% of the non-canonical nucleotides are one or more of 5-hydroxycytidine, 5-methylcytidine, 5-hydroxymethylcytidine, 5-carboxycytidine, 5-formylcytidine, 5-methoxycytidine, 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 50%, or at least about 55%, or at least 60%, or at least 65%, or at least 70%, or at least 75%, or at least 80%, or at least 85%, or at least 90%, or at least 95%, or 100% of cytidine residues are non-canonical nucleotides selected from 5-hydroxycytidine, 5-methylcytidine, 5-hydroxymethylcytidine, 5-carboxycytidine, 5-formylcytidine, 5-methoxycytidine.

In some embodiments, at least about 20%, or about 30%, or about 40%, or about 50%, or at least about 55%, or at least 60%, or at least 65%, or at least 70%, or at least 75%, or at least 80%, or at least 85%, or at least 90%, or at least 95%, or 100% of uridine residues are non-canonical nucleotides selected from 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 10% (e.g., 10%, or about 20%, or about 30%, or about 40%, or about 50%) of guanosine residues are non-canonical nucleotides, and the non-canonical nucleotide is optionally 7-deazaguanosine. In some embodiments, the RNA contains no more than about 50% 7-deazaguanosine in place of guanosine residues.

In some embodiments, the synthetic RNA molecule does not contain non-canonical nucleotides in place of adenosine residues.

Other non-canonical nucleotides that can be used in place of or in combination with 5-methyluridine include but are not limited to: pseudouridine and 5-methylpseudouridine (a.k.a. “1-methylpseudouridine”, a.k.a. “N1-methylpseudouridine”) or one or more derivatives thereof. Other non-canonical nucleotides that can be used in place of or in combination with 5-methylcytidine and/or 5-hydroxymethylcytidine include, but are not limited to: pseudoisocytidine, 5-methylpseudoisocytidine, 5-hydroxymethylcytidine, 5-formylcytidine, 5-carboxycytidine, N4-methylcytidine, N4-acetylcytidine or one or more derivatives thereof. In certain embodiments, for example, when performing only a single transfection or when the cells being transfected are not particularly sensitive to transfection-associated toxicity or innate-immune signaling, the fractions of non-canonical nucleotides can be reduced. Reducing the fraction of non-canonical nucleotides can be beneficial, in part, because reducing the fraction of non-canonical nucleotides can reduce the cost of the nucleic acid. In certain situations, for example, when minimal immunogenicity of the nucleic acid is desired, the fractions of non-canonical nucleotides can be increased.

Note that alternative naming schemes exist for certain non-canonical nucleotides. For example, in certain situations, 5-methylpseudouridine can be referred to as “3-methylpseudouridine” or “N3-methylpseudouridine” or “1-methylpseudouridine” or “N1-methylpseudouridine”. Nucleotides that contain the prefix “amino” can refer to any nucleotide that contains a nitrogen atom bound to the atom at the stated position of the nucleotide, for example, 5-aminocytidine can refer to 5-aminocytidine, 5-methylaminocytidine, and 5-nitrocytidine. Similarly, nucleotides that contain the prefix “methyl” can refer to any nucleotide that contains a carbon atom bound to the atom at the stated position of the nucleotide, for example, 5-methylcytidine can refer to 5-methylcytidine, 5-ethylcytidine, and 5-hydroxymethylcytidine, nucleotides that contain the prefix “thio” can refer to any nucleotide that contains a sulfur atom bound to the atom at the given position of the nucleotide, and nucleotides that contain the prefix “hydroxy” can refer to any nucleotide that contains an oxygen atom bound to the atom at the given position of the nucleotide, for example, 5-hydroxyuridine can refer to 5-hydroxyuridine and uridine with a methyl group bound to an oxygen atom, wherein the oxygen atom is bound to the atom at the 5C position of the uridine.

In some embodiments, non-canonical nucleotides are incorporated into RNA to increase the efficiency with which the RNA can be translated into protein and can decrease the toxicity of the RNA. In embodiments, the RNA molecule comprises one or more non-canonical nucleotides. In some embodiments, the nucleic acid comprises one or more non-canonical nucleotide members of the 5 methylcytidine de-methylation pathway. In some embodiments, the nucleic acid comprises at least one of: 5 methylcytidine, 5 hydroxymethylcytidine, 5 formylcytidine, and 5 carboxycytidine or a derivative thereof. In some embodiments, the nucleic acid comprises at least one of: pseudouridine, 5 methylpseudouridine, 5 methyluridine, 5 methylcytidine, 5 hydroxymethylcytidine, N4-methylcytidine, N4-acetylcytidine, and 7-deazaguanosine or a derivative thereof.

Certain non-canonical nucleotides can be incorporated more efficiently than other non-canonical nucleotides into RNA molecules by RNA polymerases that are commonly used for in vitro transcription, due in part to the tendency of these certain non-canonical nucleotides to participate in standard base-pairing interactions and base-stacking interactions, and to interact with the RNA polymerase in a manner similar to that in which the corresponding canonical nucleotide interacts with the RNA polymerase. As a result, certain nucleotide mixtures containing one or more non-canonical nucleotides can be beneficial in part because in vitro-transcription reactions containing these nucleotide mixtures can yield a large quantity of RNA. Certain embodiments are therefore directed to a nucleotide mixture containing one or more nucleotides that includes one or more substitutions at the 2C and/or 4C and/or 5C positions in the case of a pyrimidine or the 6C and/or 7N and/or 8C positions in the case of a purine. Nucleotide mixtures include, but are not limited to (numbers preceding each nucleotide indicate an exemplary fraction of the non-canonical nucleotide triphosphate in an in vitro-transcription reaction, for example, 0.2 pseudoisocytidine refers to a reaction containing adenosine-5′-triphosphate, guanosine-5′-triphosphate, uridine-5′-triphosphate, cytidine-5′-triphosphate, and pseudoisocytidine-5′-triphosphate, wherein pseudoisocytidine-5′-triphosphate is present in the reaction at an amount approximately equal to 0.2 times the total amount of pseudoisocytidine-5′-triphosphate+cytidine-5′-triphosphate that is present in the reaction, with amounts measured either on a molar or mass basis, and wherein more than one number preceding a nucleoside indicates a range of exemplary fractions): 1.0 pseudouridine, 0.1-0.8 2-thiouridine, 0.1-0.8 5-methyluridine, 0.2-1.0 5-hydroxyuridine, 0.2-1.0 5-methoxyuridine, 0.1-1.0 5-aminouridine, 0.1-1.0 4-thiouridine, 0.1-1.0 2-thiopseudouridine, 0.1-1.0 4-thiopseudouridine, 0.1-1.0 5-hydroxypseudouridine, 0.2-1 5-methylpseudouridine, 0.2-1.0 5-methoxypseudouridine, 0.1-1.0 5-aminopseudouridine, 0.2-1.0 2-thiocytidine, 0.1-0.8 pseudoisocytidine, 0.2-1.0 5-methylcytidine, 0.2-1.0 5-hydroxycytidine, 0.2-1.0 5-hydroxymethylcytidine, 0.2-1.0 5-methoxycytidine, 0.1-1.0 5-aminocytidine, 0.2-1.0 N4-methylcytidine, 0.2-1.0 5-methylpseudoisocytidine, 0.2-1.0 5-hydroxypseudoisocytidine, 0.2-1.0 5-aminopseudoisocytidine, 0.2-1.0 N4-methylpseudoisocytidine, 0.2-1.0 2-thiopseudoisocytidine, 0.2-1.0 7-deazaguanosine, 0.2-1.0 6-thioguanosine, 0.2-1.0 6-thio-7-deazaguanosine, 0.2-1.0 8-azaguanosine, 0.2-1.0 7-deaza-8-azaguanosine, 0.2-1.0 6-thio-8-azaguanosine, 0.1-0.5 7-deazaadenosine, and 0.1-0.5 N6-methyladenosine.

In some embodiments, the RNA comprising one or more non-canonical nucleotides composition or synthetic polynucleotide composition (e.g., which may be prepared by in vitro transcription) contains substantially or entirely the canonical nucleotide at positions having adenine or “A” in the genetic code. The term “substantially” in this context refers to at least 90%. In these embodiments, the RNA composition or synthetic polynucleotide composition may further contain (e.g., consist of) 7-deazaguanosine at positions with “G” in the genetic code as well as the corresponding canonical nucleotide “G”, and the canonical and non-canonical nucleotide at positions with G may be in the range of 5:1 to 1:5, or in some embodiments in the range of 2:1 to 1:2. In these embodiments, the RNA composition or synthetic polynucleotide composition may further contain (e.g., consist of) one or more (e.g., two, three or four) of 5-hydroxycytidine, 5-methylcytidine, 5-hydroxymethylcytidine, 5-carboxycytidine, 5-formylcytidine, 5-methoxycytidine at positions with “C” in the genetic code as well as the canonical nucleotide “C”, and the canonical and non-canonical nucleotide at positions with C may be in the range of 5:1 to 1:5, or in some embodiments in the range of 2:1 to 1:2. In some embodiments, the level of non-canonical nucleotide at positions of “C” are as described in the preceding paragraph. In these embodiments, the RNA composition or synthetic polynucleotide composition may further contain (e.g., consist of) one or more (e.g., two, three, or four) of 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine at positions with “U” in the genetic code as well as the canonical nucleotide “U”, and the canonical and non-canonical nucleotide at positions with “U” may be in the range of 5:1 to 1:5, or in some embodiments in the range of 2:1 to 1:2. In some embodiments, the level of non-canonical nucleotide at positions of “U” are as described in the preceding paragraph.

In embodiments, combining certain non-canonical nucleotides can be beneficial in part because the contribution of non-canonical nucleotides to lowering the toxicity of RNA molecules can be additive. Certain embodiments are therefore directed to a nucleotide mixture, wherein the nucleotide mixture contains more than one of the non-canonical nucleotides listed above, for example, the nucleotide mixture contains both pseudoisocytidine and 7-deazaguanosine or the nucleotide mixture contains both N4-methylcytidine and 7-deazaguanosine, etc. In one embodiment, the nucleotide mixture contains more than one of the non-canonical nucleotides listed above, and each of the non-canonical nucleotides is present in the mixture at the fraction listed above, for example, the nucleotide mixture contains 0.1-0.8 pseudoisocytidine and 0.2-1.0 7-deazaguanosine or the nucleotide mixture contains 0.2-1.0 N4-methylcytidine and 0.2-1.0 7-deazaguanosine, etc.

In certain situations, for example, when it may not be necessary or desirable to maximize the yield of an in vitro-transcription reaction, nucleotide fractions other than those given above may be used. The exemplary fractions and ranges of fractions listed above relate to nucleotide-triphosphate solutions of typical purity (greater than 90% purity). Larger fractions of these and other nucleotides can be used by using nucleotide-triphosphate solutions of greater purity, for example, greater than about 95% purity or greater than about 98% purity or greater than about 99% purity or greater than about 99.5% purity, which can be achieved, for example, by purifying the nucleotide triphosphate solution using existing chemical-purification technologies such as high-pressure liquid chromatography (HPLC) or by other means. In one embodiment, nucleotides with multiple isomers are purified to enrich the desired isomer.

In some embodiments, the one or more non-canonical nucleotides avoids substantial cellular toxicity.

In some embodiments, the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

In some embodiments, at least about 50% of cytidine residues are non-canonical nucleotides, and which are selected from 5-hydroxycytidine, 5-methylcytidine, 5-hydroxymethylcytidine, 5-carboxycytidine, 5-formylcytidine, and 5-methoxycytidine.

In some embodiments, at least about 75% or at least about 90% of cytidine residues are non-canonical nucleotides, and the non-canonical nucleotides are selected from 5-hydroxycytidine, 5-methylcytidine, 5-hydroxymethylcytidine, 5-carboxycytidine, 5-formylcytidine, and 5-methoxycytidine.

In some embodiments, at least about 20% of uridine, or at least about 40%, or at least about 50%, or at least about 75%, or at about least 90% of uridine residues are non-canonical nucleotides, and the non-canonical are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 40%, or at least about 50%, or at least about 75%, or at about least 90% of uridine residues are non-canonical nucleotides, and the non-canonical nucleotides are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 10% of guanine residues are non-canonical nucleotides, and the non-canonical nucleotide is optionally 7-deazaguanosine. In some embodiments, the synthetic RNA comprises no more than about 50% 7-deazaguanosine in place of guanosine residues. In some embodiments, the synthetic RNA does not comprise non-canonical nucleotides in place of adenosine residues.

### Methods of Treatment

An aspect of the present disclosure is a method for treating a cancer. The method comprising administering to a subject in need a therapeutically-effective amount of a first pharmaceutical composition comprising one or both of a population of isolated lymphoid lineage cells and a population of isolated myeloid lineage cells.

In various embodiments, the method further comprises administering to the subject in need a synthetic mRNA encoding a gene-editing protein (e.g., a temperature-sensitive gene-editing protein) and a single-stranded or double-stranded repair template which encodes a chimeric antigen receptor (CAR). In some cases, the gene-editing protein creates a single-stranded break or a double-stranded break in the genomic DNA of a cell in the subject and the single-stranded or double-stranded repair template which encodes the CAR inserts into the break. In these embodiments, the cell in the subject expresses the CAR.

In numerous embodiments, the method further comprises administering to the subject in need a synthetic mRNA encoding a gene-editing protein (e.g., a temperature-sensitive gene-editing protein) and a single-stranded or double-stranded repair template which encodes a cytokine. In some cases, the gene-editing protein creates a single-stranded break or a double-stranded break in the genomic DNA of a cell in the subject and the single-stranded or double-stranded repair template which encodes the cytokine inserts into the break. In these embodiments, the cell in the subject expresses or over expresses the cytokine.

When the synthetic mRNA and/or the repair template is administered to a subject, the synthetic mRNA and/or the repair is combined with a lipid system comprising a compound of Formula (IV).

In these embodiments, the cytotoxic lymphocyte targets and kills cancer cells and the isolated myeloid lineage cells kill cancer cells and/or promote cancer cell killing by cytotoxic lymphocytes.

In various embodiments, the present methods and compositions find use in methods of treating, preventing, or ameliorating a disease, disorder, and/or condition. For instance, in some embodiments, the described methods of in vivo delivery, including administration strategies, and formulations are used in a method of treatment. In some methods, the described methods reduce symptoms associated with a disease. In some embodiments, the methods eliminate the underlying cause of the disease. In some embodiments, the methods are used in the treatment of a disease requiring immunosuppression. In some embodiments, the methods reduce inflammation. In some embodiments, the methods reduce immune response.

In various embodiments, the present invention pertains to pharmaceutical compositions comprising the recombinantly engineered cells described herein and a pharmaceutically acceptable carrier or excipient. In some embodiments, the present invention pertains to pharmaceutical compositions comprising the iPSC-derived cells of the lymphoid lineage, including cytotoxic lymphocytes, iPSC-derived cells of the myeloid lineage, e.g., monocytes which can be differentiated into functional M1 and M2 macrophages having enhanced cytokine secretion and tumor cell-killing activity, and/or synthetic RNA molecules encoding the gene-editing protein or expression cassettes for expressing a protein of interest, e.g., a CAR or for expressing or overexpressing a cytokine.

In embodiments, the disclosed composition is suitable for use in the treatment of amyotrophic lateral sclerosis (ALS), spinal cord injury, degenerative disc disease, coronary artery disease, acute myocardial infarction, alcoholic liver cirrhosis, hepatitis C virus (HCV)-induced cirrhosis, multiple sclerosis (MS), osteoarthritis (OA), osteoarthritis of the knee, kidney allograft, critical limb ischemia, ischemic cardiomyopathy, Crohn's disease, idiopathic pulmonary fibrosis, anal fistula, spinal cord injury, systemic lupus erythematosus (SLE), acute respiratory distress syndrome (ARDS), acute graft-versus-host disease (aGvHD), preterm bronchopulmonary dysplasia (BPD), autism nonischemic heart failure, and/or Type 2 diabetes mellitus.

In embodiments, the present methods relate to therapeutic use in autoimmune diseases or disorders. Examples of autoimmune diseases or disorders that may be treated or prevented by the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre syndrome, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, stiff-man syndrome, systemic lupus erythematosus, lupus erythematosus, takayasu arteritis, temporal arteristis, giant cell arteritis, ulcerative colitis, uveitis, vitiligo and Wegener's granulomatosis. Preferably autoimmune disorders that may be treated or prevented by the present compositions include rheumatoid arthritis, type 1 diabetes mellitus, multiple sclerosis, systemic lupus erythematosus, and atopy.

In embodiments, the present methods relate to therapeutic use in degenerative diseases or disorders. A degenerative diseases or disorders is a disease in which the function or structure of the affected tissues or organs will progressively deteriorate overtime. Examples of degenerative diseases that can be treated or prevented with the present invention include Amyotrophic Lateral Sclerosis (ALS), Alzheimer's disease, Parkinson's Disease, Multiple system atrophy, Niemann Pick disease, Atherosclerosis, Progressive supranuclear palsy, Tay-Sachs Disease, Diabetes, Heart Disease, Keratoconus, Inflammatory Bowel Disease (IBD), Prostatitis, Osteoarthritis, Osteoporosis, Rheumatoid Arthritis, Huntington's Disease, Chronic traumatic encephalopathy, Epilepsy, Dementia, Renal failure, Multiple sclerosis, Malaria with CNS degeneration, Neuro-AIDS, Lysosomal storage diseases, Encephalitis of viral, bacterial or autoimmune origin.

In embodiments, the present methods relate to therapeutic use in a lung diseases or disorders.

In embodiments, the lung disease or disorder is a lung disease or disorder that would benefit therapeutically from suppression of immune responses in the lung. In some embodiments, inflammation is associated with the lung disease or disorder.

In some embodiments, the lung disease or disorder is selected from Asbestosis, Asthma, Bronchiectasis, Bronchitis, Chronic Cough, Chronic Obstructive Pulmonary Disease (COPD), Common Cold, Croup, Cystic Fibrosis, Hantavirus, Idiopathic Pulmonary Fibrosis, Influenza, Lung Cancer, Pandemic Flu, Pertussis, Pleurisy, Pneumonia, Pulmonary Embolism, Pulmonary Hypertension, Respiratory Syncytial Virus (RSV), Sarcoidosis, Sleep Apnea, Spirometry, Sudden Infant Death Syndrome (SIDS), and Tuberculosis.

In some embodiments, the lung disease or disorder is chronic obstructive pulmonary disease (COPD), reactive airway disease such as asthma, bronchiolitis, acute lung injury, lung allograft rejection (acute or chronic), pulmonary fibrosis, interstitial lung disease or hypersensitivity pneumonitis. In embodiments, the disease or disorder is an acute lung injury (ALI). In embodiments, the ALI is a pulmonary disorder that can be induced directly by inhalation of chemicals (chemical induced acute lung injury) or other means (e.g., infection) or can be induced indirectly by systemic injury (e.g., infection). Acute lung injury includes subcategories of respiratory distress syndromes including infant respiratory distress syndrome (IRDS), hyaline membrane disease (HMD), neonatal respiratory distress syndrome (NRDS), respiratory distress syndrome of newborn (RDSN), surfactant deficiency disorder (SDD), acute respiratory distress syndrome (ARDS), respiratory complication from systemic inflammatory response syndrome (SIRS), or severe acute respiratory syndrome (SARS).

In embodiments, the present invention relates to the therapeutic use of the present cells for the treatment of one or more symptoms associated with a viral infection.

In embodiments, the composition is suitable for use in the treatment of an infectious disease, optionally selected from an infection with a pathogen, optionally selected from a bacterium, virus, fungus, or parasite.

In embodiments, the pathogen is a virus. In embodiments, the virus is: (a) an influenza virus, optionally selected from Type A, Type B, Type C, and Type D influenza viruses, or (b) a member of the Coronaviridae family, optionally selected from (i) a betacoronavirus, optionally selected from Severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or (ii) an alphacoronavirus, optionally selected from HCoV-NL63 and HCoV-229E.

In embodiments, the virus is SARS-CoV-2. In embodiments, the virus is SARS-CoV-2, which has caused COVID-19. In embodiments, the COVID-19 is characterized by one or more of fever, cough, shortness of breath, diarrhea, upper respiratory symptoms, lower respiratory symptoms, pneumonia, and respiratory distress.

In some embodiments, the composition is suitable for use in the treatment of an infection, wherein the infection is a coronavirus infection. In some embodiments, the coronavirus infection is one or more of severe acute respiratory syndrome coronavirus 2 (SARS-CoV-2), SARS-CoV, Middle East Respiratory Syndrome-Corona Virus (MERS-CoV), HCoV-HKU1, HCoV-OC43, HCoV-NL63, and HCoV-229E. In various embodiments, the coronavirus infection is SARS or COVID-19. In further embodiments, the subject is infected by SARS-CoV-2.

In embodiments, the therapy prevents or mitigates development of acute respiratory distress syndrome (ARDS) in a patient when administered. In embodiments, the therapy improves oxygenation in a patient when administered. In embodiments, the therapy improves systemic blood pressure oxygenation in a patient when administered, e.g., reducing or mitigating shock, e.g., requiring less pressor support. In embodiments, the therapy improves lung and/or alveolar permeability in a patient when administered.

In embodiments, the therapy prevents or mitigates a transition from respiratory distress to cytokine imbalance in a patient when administered. In embodiments, the therapy reverses or prevents a cytokine storm in a patient when administered. In embodiments, the therapy reverses or prevents a cytokine storm in the lungs or systemically in a patient when administered. In embodiments, the cytokine storm is selected from one or more of systemic inflammatory response syndrome, cytokine release syndrome, macrophage activation syndrome, and hemophagocytic lymphohistiocytosis.

In embodiments, the therapy reverses or prevents excessive production of one or more inflammatory cytokines in a patient when administered. In embodiments, the inflammatory cytokine is one or more of IL-6, IL-1, IL-1 receptor antagonist (IL-1ra), IL-2ra, IL-10, IL-18, TNFα, interferon-γ, CXCL10, and CCL7.

In embodiments, the present invention relates to the therapeutic use of the present cells for the treatment of one or more symptoms associated with a coronavirus infection.

Coronaviruses (CoVs) are members of the family Coronaviridae, including betacoronavirus and alphacoronavirus—respiratory pathogens that have relatively recently become known to invade humans. The Coronaviridae family includes such betacoronavirus as severe acute respiratory syndrome coronavirus 2 (SARS-CoV-2), SARS-CoV, Middle East Respiratory Syndrome-Corona Virus (MERS-CoV), HCoV-HKU1, and HCoV-OC43. Alphacoronavirus includes, e.g., HCoV-NL63 and HCoV-229E. In embodiments, the present invention relates to the therapeutic use of the present cells for the treatment of one or more symptoms of infection with any of severe acute respiratory syndrome coronavirus 2 (SARS-CoV-2), SARS-CoV, Middle East Respiratory Syndrome-Corona Virus (MERS-CoV), HCoV-HKU1, and HCoV-OC43. Alphacoronavirus includes, e.g., HCoV-NL63 and HCoV-229E.

Without wishing to be bound by theory, coronaviruses invade cells through utilization of their “spike” surface glycoprotein that is responsible for viral recognition of Angiotensin Converting Enzyme 2 (ACE2), a transmembrane receptor on mammalian hosts that facilitate viral entrance into host cells. (Zhou et al., A pneumonia outbreak associated with anew coronavirus of probable bat origin, Nature 2020).

Symptoms associated with coronavirus infections include, but are not limited to, fever, tiredness, dry cough, aches and pains, shortness of breath and other breathing difficulties, diarrhea, upper respiratory symptoms (e.g., sneezing, runny nose, nasal congestion, cough, sore throat), and/or pneumonia. In embodiments, the present compositions and methods are useful in treating or mitigating any of these symptoms.

In embodiments, the present invention relates to the therapeutic use of the present cells for the treatment of one or more symptoms of infection with SARS-CoV-2, including Coronavirus infection 2019 (COVID-19), caused by SARS-CoV-2 (e.g., 2019-nCoV).

In some settings, including subjects afflicted with coronavirus infections, it is possible that the morbidity and mortality of pulmonary viral infection is related to an exaggerated or overwhelming inflammatory response. In varying clinical circumstances this response can be described as “cytokine response syndrome”, “cytokine storm”, or “secondary hemophagocytic lymphohistiocytosis” (sHLH). In embodiments, the present compositions and methods are useful in treating or mitigating any of these exaggerated or overwhelming inflammatory responses. Collectively it is surmised that these highly proinflammatory states can lead to death due to pulmonary collapse such as acute respiratory distress syndrome (ARDS) or systemic, multi-organ failure affecting organs such as liver, kidney, heart and brain. In embodiments, the present cells treat or mitigate a “cytokine response syndrome”, “cytokine storm”, or “secondary hemophagocytic lymphohistiocytosis” (sHLH).

In embodiments, COVID-19 is characterized, in part, by elevation of Interleukin-2 (IL-2), Interleukin-7 (IL-7), granulocyte colony stimulating factor (GCSF), interferon-gamma inducible protein 10, monocyte chemoattractant protein-1 (MCP-1), macrophage inflammatory protein 1-alpha (MIP1a), and tumor necrosis factor-alpha (TNFα). In embodiments, the present compositions and methods are useful in treating or mitigating increases of any of these factors.

In embodiments, the present cells prevent a COVID-19 patient from having a disease that develops from respiratory distress to cytokine storm.

In embodiments, the present cells treat or mitigate ARDS.

In some embodiments, a cytokine storm is associated with COVID-19 and is treated or mitigated via a method comprising administering to a subject in need thereof an effective amount of cells effective for the treatment of a coronavirus infection and/or a cytokine storm associated with a coronavirus infection, wherein the subject has abnormal (e.g. increased or decreased) expression or activity of one or more of IL-6, IL-1, TNF, interferon-γ, CXCL10, CCL7, IL-1 receptor antagonist (IL-1ra), IL-2ra, IL-10, IL-18, CCL2/MCP-1, CCL5/RANTES, CCL7/MCP-3, MCP-2, tumor necrosis factor-alpha (TNFα), interferon-γ (IFNγ), CXCL10, CXC3, Granulocyte colony stimulatory factor (GCSF), Macrophage inflammatory protein 1 alpha (MIP-1a), IL-22, and Interferon gamma induced protein 10 (IP-10).

In some embodiments, the subject has a modulated (e.g. decreased or increased) expression or activity of one or more of IL-6, IL-1, TNF, interferon-γ, CXCL10, CCL7, IL-1 receptor antagonist (IL-1ra), IL-2ra, IL-10, IL-18, CCL2/MCP-1, CCL5/RANTES, CCL7/MCP-3, MCP-2, tumor necrosis factor-alpha (TNFα), interferon-γ (IFNγ), CXCL10, CXC3, Granulocyte colony stimulatory factor (GCSF), Macrophage inflammatory protein 1 alpha (MIP-1a), IL-22, and Interferon gamma induced protein 10 (IP-10).

In embodiments, the disease/indication is associated with one or more cancers. The one or more cancers may comprise: adenoid cystic carcinoma, adrenal gland tumor, amyloidosis, anal cancer, appendix cancer, astrocytoma, ataxia-telangiectasia, Beckwith-Wiedemann Syndrome, bile duct caner (Cholangiocarcinoma), Birt-Hogg Dube Syndrome, bladder cancer, bone cancer (sarcoma of bone), brain stem glioma, brain tumor, breast cancer, breast cancer (inflammatory), breast cancer (metastatic), breast cancer in men, carney complex, central nervous system tumors (brain and spinal cord), cervical cancer, childhood cancer, colorectal cancer, Cowden Syndrome, craniopharyngioma, desmoid tumor, desmoplastic infantile ganglioglioma tumor, ependymoma, esophageal cancer, Ewing Sarcoma, eye cancer, eyelid cancer, familial adenomatous polyposis, familial GIST, familial malignant melanoma, familial pancreatic cancer, gallbladder cancer, gastrointestinal stromal tumor GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, HIV/AIDS related cancer, juvenile polyposis syndrome, kidney cancer, lacrimal gland tumor, laryngeal and hypopharyngeal cancer, leukemia—acute lymphoblastic—ALL, leukemia, acute lymphocytic—ALL, leukemia—acute myeloid—ALL, leukemia—acute myeloid—AML, leukemia—B-cell prolymphocytic leukemia and hairy cell leukemia, leukemia—chronic lymphocytic—CLL, leukemia—chronic myeloid—CML, leukemia—chronic t-cell lymphocytic, leukemia—eosinophilic, Li-Fraumeni Syndrome, liver cancer, lung cancer—non-small cell, lung cancer—small cell, lymphoma—Hodgkin, lymphoma—Non-Hodgkin, Lynch Syndrome, mastocytosis, medulloblastoma, melanoma, meningioma, mesothelioma, multiple endocrine neoplasia type 1, multiple endocrine neoplasia type 2, multiple myeloma, MUTYH/MYH—associated polyposis, myelodysplastic syndromes—MDS, nasal cavity and paranasal sinus cancer, nasopharyngeal cancer, neuroblastoma, neuroendocrine tumor of the gastrointestinal tract, neuroendocrine tumor of the lung, neuroendocrine tumor of the pancreas, neuroendocrine tumors, neurofibromatosis type 1, neurofibromatosis type 2, nevoid basal cell carcinoma syndrome, oral and oropharyngeal cancer, osteosarcoma, ovarian cancer, fallopian tube cancer, peritoneal cancer, pancreatic cancer, parathyroid cancer, penile cancer, Peutz-Jeghers Syndrome, pheochromocytoma and paraganglioma, pituitary gland tumor, pleuropulmonary blastoma, prostate cancer, retinoblastoma, rhabdomyosarcoma, salivary gland cancer, sarcoma—Kaposi, sarcomas of specific organs, sarcomas—soft tissue, skin cancer (non-melanoma), skin cancer (melanoma), small bowel cancer, stomach cancer, testicular cancer, thyoma and thymic carcinoma, thyroid cancer, tuberous sclerosis complex, unknown primary, uterine cancer, vaginal cancer, Von Hippel-Lindau Syndrome, vulvar cancer, Waldenstrom macroglobulinemia (lymphoplasmacytic lymphoma), Werner Syndrome, Wilms tumor, and xeroderma pigmentosum.

In an aspect, the present disclosure provides a method for treating a cancer in a patient in need thereof. The method comprising administering to the cancer patient a therapeutically-effective amounts of any herein-disclosed cytotoxic lymphocyte.

An aspect of the present disclosure is a method for killing a cancer cell. The method comprising steps of: (1) obtaining a herein-disclosed cytotoxic lymphocyte and (2) contacting cytotoxic lymphocyte with the cancer. In some cases, the cancer cell is in vivo.

Yet another aspect of the present disclosure is a method for treating a cancer patient in need thereof. The method comprising a step of administering to the cancer patient a therapeutically-effective amounts of a herein-disclosed cytotoxic lymphocyte.

In aspects, the present disclosure provides a method of treating cancer, comprising: (a) obtaining an isolated cytotoxic lymphocyte comprising a genetically engineered disruption in a beta-2-microglobulin (B2M) gene; and (b) administering the isolated cytotoxic lymphocyte to a subject in need thereof.

In some cases, the lymphoid lineage cell is a T cell, e.g., a cytotoxic T cell or gamma-delta T cell; an NK cell; or an NK-T cell.

In some cases, the myeloid lineage cell is a macrophage, e.g., an M1 macrophage or an M2 macrophage.

In embodiments, the cytotoxic lymphocyte is an NK cell.

In embodiments, the cancer is a blood cancer. In embodiments, the cancer is a solid tumor. In embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs' syndrome.

The cytotoxic lymphocyte of the present disclosure may be administered systemically (e.g., via a vein or artery) or may be introduced into a tumor or in the vicinity of the tumor.

### Administration and Formulations

In some embodiments, the present disclosure relates to compositions described herein in the form of a pharmaceutical composition.

An aspect of the present disclosure is a method for treating a cancer. The method comprising administering to a subject in need a therapeutically-effective amount of a first pharmaceutical composition comprising one or both of a population of isolated lymphoid lineage cells and a population of isolated myeloid lineage cells.

In embodiments, the first pharmaceutical composition comprises the population of isolated lymphoid lineage cells and wherein the subject in need is administered a therapeutically-effective amount of a second pharmaceutical composition comprising a population of isolated myeloid lineage cells.

In some embodiments, the first pharmaceutical composition comprises the population of isolated myeloid lineage cells and wherein the subject in need is administered a therapeutically-effective amount of a second pharmaceutical composition comprising a population of isolated lymphoid lineage cells. In some cases, the first pharmaceutical composition and the second pharmaceutical composition are administered simultaneously or sequentially. The first pharmaceutical composition and the second pharmaceutical composition may be administered sequentially with the first pharmaceutical composition administered before the second pharmaceutical composition or the first pharmaceutical composition and the second pharmaceutical composition may be administered sequentially with the second pharmaceutical composition administered before the first pharmaceutical composition.

In various embodiments, the first pharmaceutical composition comprises both the population of isolated lymphoid lineage cells and the population of isolated myeloid lineage cells.

In various embodiments, the present invention pertains to pharmaceutical compositions comprising the recombinantly engineered cells described herein and a pharmaceutically acceptable carrier or excipient. In some embodiments, the present invention pertains to pharmaceutical compositions comprising the iPSC-derived cells of the lymphoid lineage, including cytotoxic lymphocytes, iPSC-derived cells of the myeloid lineage, e.g., monocytes which can be differentiated into functional M1 and M2 macrophages having enhanced cytokine secretion and tumor cell-killing activity, and/or synthetic RNA molecules encoding the gene-editing protein or expression cassettes for expressing a protein of interest, e.g., a CAR or for expressing or overexpressing a cytokine.

Therapeutic treatments comprise the use of one or more routes of administration and of one or more formulations that are designed to achieve a therapeutic effect at an effective dose, while minimizing toxicity to the subject to which treatment is administered. Illustrative formulations/compositions of the present disclosure include engineered cells along with a suitable delivery reagent, e.g., a liquid carrier.

In various embodiments, the effective dose is an amount that substantially avoids cell toxicity in vivo. In various embodiments, the effective dose is an amount that substantially avoids an immune reaction in a human subject. For example, the immune reaction may be an immune response mediated by the innate immune system. Immune response can be monitored using markers known in the art (e.g., cytokines, interferons, TLRs). In some embodiments, the effective dose obviates the need for treatment of the human subject with immune suppressants agents (e.g., B18R) used to moderate the residual toxicity.

Upon formulation, solutions may be administered in a manner compatible with the dosage formulation and in such amount as is therapeutically effective, as described herein. The formulations may easily be administered in a variety of dosage forms such as injectable solutions and the like. For parenteral administration in an aqueous solution, for example, the solution generally is suitably buffered, and the liquid diluent first rendered isotonic with, for example, sufficient saline or glucose. Such aqueous solutions may be used, for example, for intravenous, intramuscular, subcutaneous and intraperitoneal administration. Preferably, sterile aqueous media are employed as is known to those of skill in the art. Pharmaceutical preparations may additionally comprise delivery reagents (a.k.a. “vehicles”, a.k.a. “delivery vehicles”) and/or excipients. Pharmaceutically acceptable delivery reagents, excipients, and methods of preparation and use thereof, including methods for preparing and administering pharmaceutical preparations to patients (a.k.a. “subjects”) are well known in the art, and are set forth in numerous publications, including, for example, in US Patent Appl. Pub. No. US 2008/0213377, the entirety of which is incorporated herein by reference.

The present pharmaceutical compositions can comprise excipients, including liquids such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the composition is formulated for one or more of intrathecal, intra-lesional, intra-coronary, intravenous (IV), intra-articular, intramuscular, and intra-endobronchial administration and administration via intrapancreatic endovascular injection, intra-nucleus pulposus, lumbar puncture, intra-myocardium, transendocardium, intra-fistula tract, intermedullary space, intradural space and leg injection.

In embodiments, the composition is formulated for infusion. In some embodiments, the composition is formulated for infusion, wherein the composition is delivered to the bloodstream of a subject or patient through a needle in a vein of the subject or patient through a peripheral line, a central line, a tunneled line, an implantable port, and/or a catheter. In some embodiments, the subject or patient may also receive supportive medications or treatments, such as hydration, by infusion. In some embodiments, the composition is formulated for intravenous infusion. In some embodiments, the infusion is continuous infusion, secondary intravenous therapy (IV), and/or IV push. In some embodiments, the infusion of the composition may be administered through the use of equipment selected from one or more of an infusion pump, hypodermic needle, drip chamber, peripheral cannula, and pressure bag.

In embodiments, the method of treating a subject comprises administering a cell of the present disclosure to a subject in need thereof. In embodiments, the cell is formulated for therapeutic use. In embodiments, the cell is suitable for administration to a human subject. In embodiments, the method is conducted in vivo.

In numerous embodiments, the administering is intravenous, intraarterial, intratumoral, or injected in the vicinity of a tumor.

In embodiments when a synthetic RNA molecule encoding a temperature-sensitive gene-editing protein is administered, the method comprises reducing the body temperature of a subject, optionally via whole-body hypothermia. In embodiments, the body temperature of the subject is reduced by from about 0.5° C. to about 1° C. In embodiments, the body temperature of the subject is reduced by from about 1° C. to about 1.5° C. In embodiments, the body temperature of the subject is reduced by from about 1.5° C. to about 2° C. In embodiments, the body temperature of the subject is reduced by from about 2° C. to about 2.5° C. In embodiments, the body temperature of the subject is reduced by from about 2.5° C. to about 3° C. In embodiments, the body temperature of the subject is reduced by from about 3° C. to about 3.5° C. In embodiments, the body temperature of the subject is reduced by from about 3.5° C. to about 4° C. In embodiments, the body temperature of the subject is reduced by from about 4° C. to about 4.5° C. In embodiments, the body temperature of the subject is reduced by from about 4.5° C. to about 5° C. In embodiments, the body temperature of the subject is reduced by from about 5° C. to about 5.5° C. In embodiments, the body temperature of the subject is reduced by from about 5.5° C. to about 6° C. In embodiments, the body temperature of the subject is reduced by from about 6° C. to about 6.5° C. In embodiments, the body temperature of the subject is reduced by from about 6.5° C. to about 7° C. In embodiments, the body temperature of the subject is reduced by from about 7° C. to about 7.5° C. In embodiments, the body temperature of the subject is reduced by from about 7.5° C. to about 8° C. In embodiments, the body temperature of the subject is reduced by from about 8° C. to about 8.5° C. In embodiments, the body temperature of the subject is reduced by from about 8.5° C. to about 9° C. In embodiments, the body temperature of the subject is reduced by from about 9° C. to about 9.5° C. In embodiments, the body temperature of the subject is reduced by from about 9.5° C. to about 10° C. In embodiments, the body temperature of the subject is reduced by from about 10° C. to about 10.5° C. In embodiments, the body temperature of the subject is reduced by from about 10.5° C. to about 11° C. In embodiments, the body temperature of the subject is reduced by from about 11° C. to about 11.5° C. In some embodiments, the reducing the body temperature of the subject is performed for a specific amount of time. In some embodiments, the specific amount of time is from about 15 minutes to about 30 minutes. In some embodiments, the specific amount of time is from about 30 minutes to about 45 minutes. In some embodiments, the specific amount of time is from about 45 minutes to about 60 minutes. In embodiments, the specific amount of time is from about 1 hour to about 1.5 hours. In embodiments, the specific amount of time is from about 1.5 hours to about 2 hours. In embodiments, the specific amount of time is from about 2 hours to about 2.5 hours. In embodiments, the specific amount of time is from about 2.5 hours to about 3 hours. In embodiments, the specific amount of time is from about 3 hours to about 3.5 hours. In embodiments, the specific amount of time is from about 3.5 hours to about 4 hours. In embodiments, the specific amount of time is from about 4 hours to about 4.5 hours. In embodiments, the specific amount of time is from about 4.5 hours to about 5 hours. In embodiments, the specific amount of time is from about 5 hours to about 5.5 hours. In embodiments, the specific amount of time is from about 5.5 hours to about 6 hours. In embodiments, the specific amount of time is from about 6 hours to about 6.5 hours.

In embodiments when a synthetic RNA molecule encoding a temperature-sensitive gene-editing protein is administered, the method comprises applying one or more cooling elements to a cell or tissue in vivo to reduce temperature, the cooling element optionally being a cryocompression device. In embodiments, the temperature is reduced by from about 0.5° C. to about 1° C. In embodiments, the temperature is reduced by from about 1° C. to about 1.5° C. In embodiments, the temperature is reduced by from about 1.5° C. to about 2° C. In embodiments, the temperature is reduced by from about 2° C. to about 2.5° C. In embodiments, the temperature is reduced by from about 2.5° C. to about 3° C. In embodiments, the temperature is reduced by from about 3° C. to about 3.5° C. In embodiments, the temperature is reduced by from about 3.5° C. to about 4° C. In embodiments, the temperature is reduced by from about 4° C. to about 4.5° C. In embodiments, the temperature is reduced by from about 4.5° C. to about 5° C. In embodiments, the temperature is reduced by from about 5° C. to about 5.5° C. In embodiments, the temperature is reduced by from about 5.5° C. to about 6° C. In embodiments, the temperature is reduced by from about 6° C. to about 6.5° C. In embodiments, the temperature is reduced by from about 6.5° C. to about 7° C. In embodiments, the temperature is reduced by from about 7° C. to about 7.5° C. In embodiments, the temperature is reduced by from about 7.5° C. to about 8° C. In embodiments, the temperature is reduced by from about 8° C. to about 8.5° C. In embodiments, the temperature is reduced by from about 8.5° C. to about 9° C. In embodiments, the temperature is reduced by from about 9° C. to about 9.5° C. In embodiments, the temperature is reduced by from about 9.5° C. to about 10° C. In embodiments, the temperature is reduced by from about 10° C. to about 10.5° C. In embodiments, the temperature is reduced by from about 10.5° C. to about 11° C. In embodiments, the temperature is reduced by from about 11° C. to about 11.5° C.

In embodiments when a synthetic RNA molecule encoding a temperature-sensitive gene-editing protein is administered, the applying one or more cooling elements to a cell or tissue in vivo to reduce temperature is performed for a specific amount of time. In some embodiments, the specific amount of time is from about 15 minutes to about 30 minutes. In some embodiments, the specific amount of time is from about 30 minutes to about 45 minutes. In some embodiments, the specific amount of time is from about 45 minutes to about 60 minutes. In some embodiments, the specific amount of time is from about 1 hour to about 1.5 hours. In some embodiments, the specific amount of time is from about 1.5 hours to about 2 hours. In some embodiments, the specific amount of time is from about 2 hours to about 2.5 hours. In some embodiments, the specific amount of time is from about 2.5 hours to about 3 hours. In some embodiments, the specific amount of time is from about 3 hours to about 3.5 hours. In some embodiments, the specific amount of time is from about 3.5 hours to about 4 hours. In some embodiments, the specific amount of time is from about 4 hours to about 4.5 hours. In some embodiments, the specific amount of time is from about 4.5 hours to about 5 hours. In some embodiments, the specific amount of time is from about 5 hours to about 5.5 hours. In some embodiments, the specific amount of time is from about 5.5 hours to about 6 hours. In some embodiments, the specific amount of time is from about 6 hours to about 6.5 hours.

Further description of temperature-sensitive cell administration is found in WO2021/231549. The entire contents of which are incorporated by reference in their entirety.

In some embodiments, the present invention relates to one or more administration techniques described in U.S. Pat. Nos. 5,711,964; 5,891,468; 6,316,260; 6,413,544; 6,770,291; and 7,390,780, the entire contents of which are hereby incorporated by reference in their entireties.

### Lipids Cell Contacting Transfection

In embodiments, the present invention relates delivery of the present synthetic RNA molecules via a lipid. In some embodiments, mRNAs encoding a gene-editing protein and/or a reprogramming factor are delivered via a lipid.

In embodiments, the lipid is a compound of Formula (I)

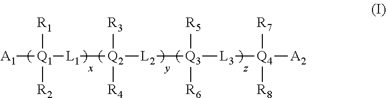

wherein: Q₁, Q₂, Q₃, and Q₄ are independently an atom or group capable of adopting a positive charge;
A₁ and A₂ are independently null, H, or optionally substituted C₁-C₆ alkyl;
L₁, L₂, and L₃ are independently null, a bond, (C₁-C₂₀)alkanediyl, (halo)(C₁-C₂₀)alkanediyl, (hydroxy)(C₁-C₂₀)alkanediyl, (alkoxy)(C₁-C₂₀)alkanediyl, arylene, heteroarylene, cycloalkanediyl, heterocycle-diyl, or any combination of the aforementioned optionally linked by one or more of an ether, an ester, an anhydride, an amide, a carbamate, a secondary amine, a tertiary amine, a quaternary ammonium, a thioether, a urea, a carbonyl, or an imine;
R₁, R₂, R₃, R₄, R₅, R₆, R₇, and R₈ are independently null, H, (C₁-C₆₀)alkyl, (halo)(C₁-C₆₀)alkyl, (hydroxy)(C₁-C₆₀)alkyl, (alkoxy)(C₁-C₆₀)alkyl, (C₂-C₆₀)alkenyl, (halo)(C₂-C₆₀)alkenyl, (hydroxy)(C₂-C₆₀)alkenyl, (alkoxy)(C₂-C₆₀)alkenyl, (C₂-C₆₀)alkynyl, (halo)(C₂-C₆₀)alkynyl, (hydroxy)(C₂-C₆₀)alkynyl, (alkoxy)(C₂-C₆₀)alkynyl, wherein at least one of R₁, R₂, R3, R4, R5, R6, R7, and R8 comprises at least two unsaturated bonds; and x, y, and z are independently 0, 1, 2, 3, 4, 5, 6, 7, 8, 9, 10, 11, 12, 13, 14, or 15.

In embodiments, the lipid is a compound of Formula (II):

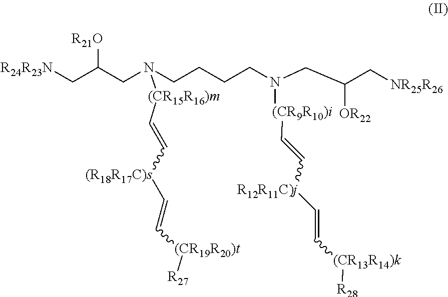

wherein: R9, R10, R11, R12, R13, R14, R15, R16, R17, R18, R19, R20, R21, R22, R23, R24, R25, R26, R27, and R28 are independently H, halo, OH, (C1-C6)alkyl, (halo)(C1-C6)alkyl, (hydroxy)(C1-C6)alkyl, (alkoxy)(C1-C₆)alkyl, aryl, heteroaryl, cycloalkyl, or heterocyclo; and
i, j, k, m, s, and t are independently 0, 1, 2, 3, 4, 5, 6, 7, 8, 9, 10, 11, 12, 13, 14, or 15.

In embodiments, the lipid is a compound of Formula (III):

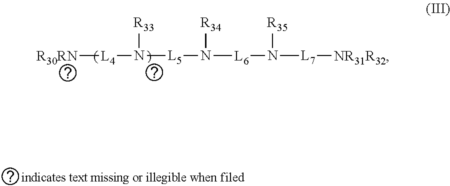

wherein L₄, L₅, L₆, and L₇ are independently a bond, (C₁-C₂₀)alkanediyl, (halo)(C₁-C₂₀)alkanediyl, (hydroxy)(C₁-C₂₀)alkanediyl, (alkoxy)(C₁-C₂₀)alkanediyl, arylene, heteroarylene, cycloalkanediyl, heterocycle-diyl, —(CH₂)ᵥ₁—C(O)—, —((CH₂)ᵥ₁—O)ᵥ₂—, or —((CH₂)ᵥ₁—C(O)—O)ᵥ₂—;
R₂₉, R₃₀, R₃₁, R₃₂, R₃₃, R₃₄, and R₃₅ are independently H, (C₁-C₆₀)alkyl, (halo)(C₁-C₆₀)alkyl, (hydroxy)(C₁-C₆₀)alkyl, (alkoxy)(C₁-C₆₀)alkyl, (C₂-C₆₀)alkenyl, (halo)(C₂-C₆₀)alkenyl, (hydroxy)(C₂-C₆₀)alkenyl, (alkoxy)(C₂-C₆₀)alkenyl, (C₂-C₆₀)alkynyl, (halo)(C₂-C₆₀)alkynyl, (hydroxy)(C₂-C₆₀)alkynyl, (alkoxy)(C₂-C₆₀)alkynyl, wherein at least one of R₂₉, R₃₀, R₃₁, R₃₂, R₃₃, R₃₄, and R₃₅ comprises at least two unsaturated bonds;
v, v₁ and v₂ are independently 0, 1, 2, 3, 4, 5, 6, 7, 8, 9, 10, 11, 12, 13, 14, or 15.

In embodiments, the lipid is a compound of Formula (IV):

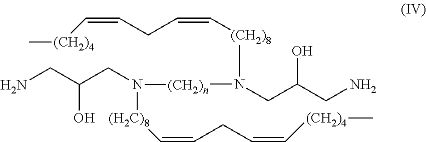

wherein n is 1, 2, 3, 4, 5, 6, 7, 8, 9, 10, 11, 12, 13, 14, or 15.

In embodiments, the lipid is a compound of Formula (V):

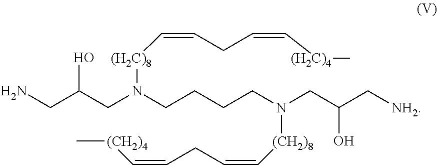

In embodiments, the lipid is a compound of Formula (VI):

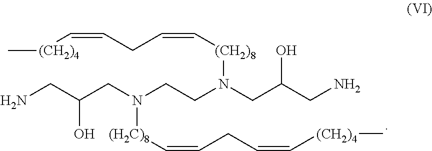

In embodiments, the lipid is a compound of Formula (VII):

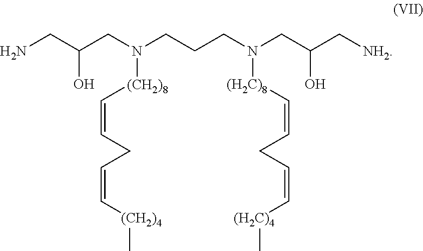

In embodiments, the lipid is a compound of Formula (VIII):

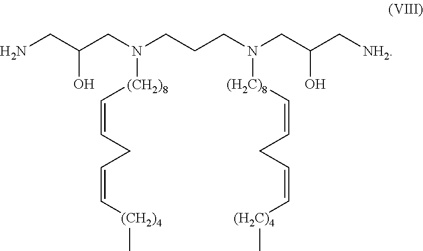

In embodiments, the lipid is a compound of Formula (IX):

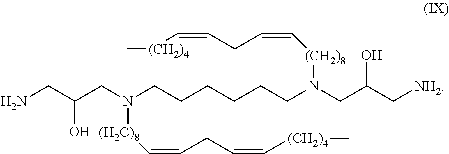

In embodiments, the lipid is a compound of Formula (X):

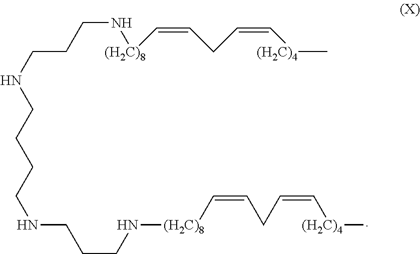

In embodiments, the lipid is a compound of Formula (XI):

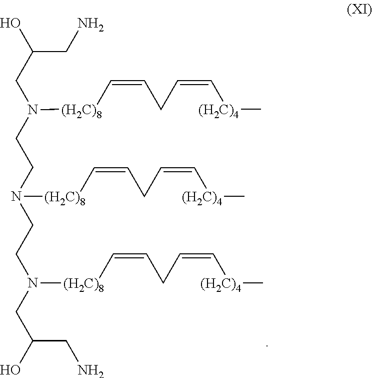

In embodiments, the lipid is a compound of Formula (XII):

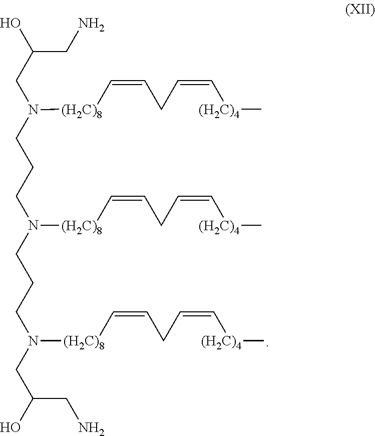

In embodiments, the lipid is a compound of Formula (XIII):

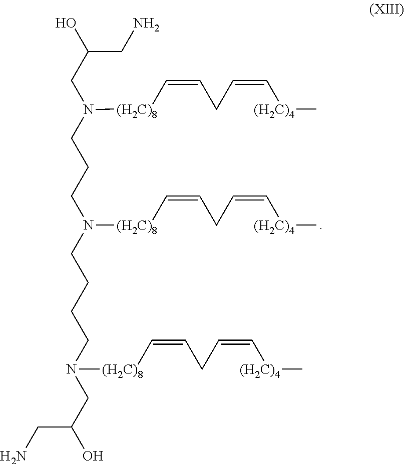

In embodiments, the lipid is a compound of Formula (XIV):

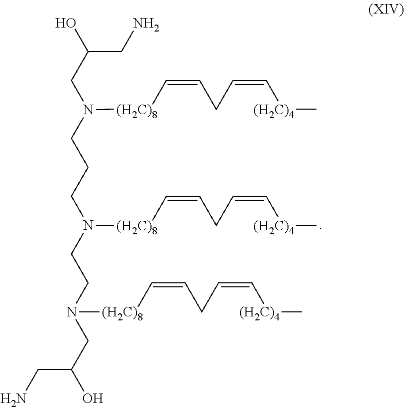

In embodiments, the lipid is a compound of Formula (XIV):

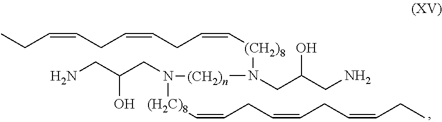

wherein n is 1, 2, 3, 4, 5, 6, 7, 8, 9, 10, 11, 12, 13, 14, or 15.

In embodiments, the lipid is a compound of Formula (XVI):

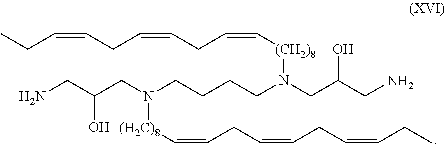

In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle.

In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle which does not require an additional or helper lipid. In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle that further comprises a neutral lipid (e.g., dioleoylphosphatidylethanolamine (DOPE), 1,2-Dioleoyl-sn-glycero-3-phosphocholine (DOPC), or cholesterol) and/or a further cationic lipid (e.g., N-[1-(2,3-dioleoyloxy)propyl]-N,N,N-trimethylammonium chloride (DOTMA), 1,2-bis(oleoyloxy)-3-3-(trimethylammonium) propane (DOTAP), or 1,2-dioleoyl-3-dimethylammonium-propane (DODAP)).

In embodiments, the lipid is any of those described in International Patent Publication No. WO 2021/003462, hereby incorporated by reference in its entirety.

In embodiments, the lipid is any of those of Table A.

TABLE A
Illustrative Biocompatible Lipids and Polymers

3β -[N-(N′,N′-dimethylaminoethane)-carbamoyl]cholesterol (DC-Cholesterol)
1,2-dioleoyl-3-trimethylammonium-propane (DOTAP/18:1 TAP)
N-(4-carboxybenzyl)-N,N-dimethyl-2,3-bis(oleoyloxy)propan-1-aminium (DOBAQ)
1,2-dimyristoyl-3-trimethylammonium-propane (14:0 TAP)
1,2-dipalmitoyl-3-trimethylammonium-propane (16:0 TAP)
1,2-stearoyl-3-trimethylammonium-propane (18:0 TAP)
1,2-dioleoyl-3-dimethylammonium-propane (DODAP/18:1 DAP)
1,2-dimyristoyl-3-dimethylammonium-propane (14:0 DAP)
1,2-dipalmitoyl-3-dimethylammonium-propane (16:0 DAP)
1,2-distearoyl-3-dimethylammonium-propane (18:0 DAP)
dimethyldioctadecylammonium (18:0 DDAB)
1,2-dilauroyl-sn-glycero-3-ethylphosphocholine (12:0 EthylPC)
1,2-dimyristoyl-sn-glycero-3-ethylphosphocholine (14:0 EthylPC)
1,2-dimyristoleoyl-sn-glycero-3-ethylphosphocholine (14:1 EthylPC)
1,2-dipalmitoyl-sn-glycero-3-ethylphosphocholine (16:0 EthylPC)
1,2-distearoyl-sn-glycero-3-ethylphosphocholine (18:0 EthylPC)
1,2-dioleoyl-sn-glycero-3-ethylphosphocholine (18:1 EthylPC)
1-palmitoyl-2-oleoyl-sn-glycero-3-ethylphosphocholine (16:1-18:1 EthylPC)
1,2-di-O-octadecenyl-3-trimethylammonium propane (DOTMA)
N1-[2-((1S)-1-[(3-aminopropyl)amino]-4-[di(3-amino-propyl)amino]butylcarboxamido)ethyl]-
3,4-di[oleyloxy]-benzamide (MVL5)
2,3-dioleyloxy-N-[2-spermine carboxamide]ethyl-N,N-dimethyl-1-propanammonium
trifluoroacetate (DOSPA)
1,3-di-oleoyloxy-2-(6-carboxy-spermyl)-propylamid (DOSPER)
N-[1-(2,3-dimyristyloxy)propyl]-N,N-dimethyl-N-(2-hydroxyethyl)ammonium bromide
(DMRIE)
LIPOFECTAMINE, LIPOFECTAMINE 2000, LIPOFECTAMINE RNAiMAX,
LIPOFECTAMINE 3000, LIPOFECTAMINE MessengerMAX, TransIT mRNA
dioctadecyl amidoglyceryl spermine (DOGS)
dioleoyl phosphatidyl ethanolamine (DOPE)
1,2-dilinoleyloxy-3-dimethylaminopropane (DLinDMA)
1,2-dilinoleyl-4-(2-dimethylaminoethyl)-[1,3]-dioxolane (DLin-KC2-DMA)
Heptatriaconta-6,9,28,31-tetraen-19-yl 4-(dimethylamino)butanoate (DLin-MC3-DMA)
N1,N4-dimyristyl-N1,N4-di-(2-hydroxy-3-aminopropyl)-diaminobutane (DHDMS)
N1,N4-dioleyl-N1,N4-di-(2-hydroxy-3-aminopropyl)-diaminobutane (DHDOS)
1,2-distearoyl-sn-glycero-3-phosphocholine (18:0 PC DSPC)
1,2-dioleyl-sn-glycero-3-phosphocholine (18:1 PC)
1,2-distearyl-sn-glycero-3-phosphatidyl ethanolamine (DSPE)
1,2-dilinoleyl-3-dimethylammonium-propane (18:2 DAP)
hexadimethrine bromide (Polybrene ™)
DEAE-Dextran
protamine
protamine sulfate
poly-L-lysine
poly-D-lysine
Poly(beta-amino-ester) polymer
polyethyleneimine
block co-polymer comprising one or more of: PEG, PLGA, PPG, PEI, PLL, PCL,
a PLURONIC

Protein Expression and/or Secretion Signature

Each type of cell expresses particular sets of proteins, within the cell, on the cell's surface, and secreted into the extracellular space. The particular sets of proteins that each type of cell expresses depends on the general and immediate function of the cell. Protein expression is correlated with mRNA levels and thus can be assayed by methods that analyze the distribution, amount, and identity of particular mRNAs within a cell. There are several methods of quantitatively measuring mRNA, including northern blotting and reverse transcription-quantitative PCR (RT-qPCR). Hybridization microarrays may also be used to generate expression profiles or high-throughput analyses of a range of genes within a cell. Further, ‘tag based’ technologies, such as Serial analysis of gene expression (SAGE) and RNA-Seq can be used to determine the relative measure of the cellular concentration of different mRNAs.

In some embodiments, protein expression of specific cells is determined by determining concentration of different mRNAs by one or more of northern blotting, RT-qPCR, hybridization microarrays, and tag-based technologies, such as SAGE and RNA-Seq.

There are generally two strategies used for detection of proteins in the extracellular milieu: direct methods and indirect methods. The direct method comprises a one-step staining, and may involve a labeled antibody (e.g., FITC conjugated antiserum) reacting directly with the protein in the extracellular milieu. The indirect method comprises an unlabeled primary antibody that reacts with the protein in the extracellular milieu, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, NY, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, CA). In some embodiments, proteins are detected in the extracellular milieu of monocytes or macrophages using detection methods comprising one or more antibodies. In some embodiments, the detection methods further comprise labels, including radioactive labels, fluorescent labels, hapten labels such as, biotin, or an enzyme such as horse radish peroxidase or alkaline phosphatase.

In some embodiments, flow cytometry is used to determine whether cells express certain sets of proteins that are on the surface or that are secreted. In some embodiments, antibodies specific to particular proteins are used in combination with proteomic approaches to determine, e.g., the protein secretion signature of a particular cell. In some embodiments, the supernatant of a purified set of cell types is assayed using a Western blot to determine the concentrations of an array of secreted proteins, to which antibodies are available. In some embodiments, the protein secretion signatures of specific cell derived from different sources, such as iPSCs, skin cells, or bone marrow are determined and compared.

In some embodiments, iPSC-derived monocytes are characterized for expression of key hematopoietic and myeloid-lineage markers CD11b, CD13, CD14, CD33, CD45, CD80, CD163, CD206, and SIRPα. The expression of these makers iPSC-derived monocytes may be compared peripheral blood mononuclear cell (PBMC)-derived monocytes.

In some embodiments, the iPSC-derived monocytes show similar expression of CD11b, CD13, CD14, CD33, CD45, and CD163 compared to PBMC-derived monocytes, and increased expression of markers indicative of an activated state: CD80 and CD206.

In some embodiments, the iPSC-derived macrophages are characterized for expression of CD11b, CD68, CD80, CD86, CD163, CD206, and SIRPα and for secretion of, at least, the cytokines TNFα, IL-12p70, and IL-10. In embodiments, M1 and M2 polarized iPSC-derived macrophages secrete similar levels of TNFα, IL-12p70, and IL-10 compared to PBMC-derived macrophages.

In some embodiments, the iMSCs are characterized for expression of CD34, CD44, CD45, CD73, and CD90.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. The terminology used herein is for the purpose of describing particular cases only and is not intended to be limiting.

As used herein, “a,” “an,” or “the” can mean one or more than one.

Herein the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Further, the term “about” when used in connection with a referenced numeric indication means the referenced numeric indication plus or minus up to 10% of that referenced numeric indication. For example, the language “about 50” covers the range of 45 to 55. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.” Although the open-ended term “comprising” is used herein to describe and claim the invention, the present invention, or embodiments thereof, may alternatively be described using alternative terms such as “consisting of” or “consisting essentially of.”

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

By preventing is meant, at least, avoiding the occurrence of a disease and/or reducing the likelihood of acquiring the disease.

By treating is meant, at least, ameliorating or avoiding the effects of a disease, including reducing a sign or symptom of the disease.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids. “Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

By “synthetic RNA molecule” is meant an RNA molecule that is produced outside of a cell or that is produced inside of a cell using bioengineering, by way of non-limiting example, an RNA molecule that is produced in an in vitro-transcription reaction, an RNA molecule that is produced by direct chemical synthesis or an RNA molecule that is produced in a genetically-engineered E. coli cell.

By “medium” is meant a solvent or a solution comprising a solvent and a solute, by way of non-limiting example, Dulbecco's Modified Eagle's Medium (DMEM), DMEM+10% fetal bovine serum (FBS), saline or water.

By “transfection medium” is meant a medium that can be used for transfection, by way of non-limiting example, Dulbecco's Modified Eagle's Medium (DMEM), DMEM/F12, saline or water.

By “Oct4 protein” is meant a protein that is encoded by the POU5F1 gene, or a natural or engineered variant, family-member, orthologue, fragment or fusion construct thereof, by way of non-limiting example, human Oct4 protein, mouse Oct4 protein, Oct1 protein, a protein encoded by POU5F1 pseudogene 2, a DNA-binding domain of Oct4 protein or an Oct4-GFP fusion protein. In some embodiments the Oct4 protein comprises an amino acid sequence that has at least 70% identity with SEQ ID NO: 76, or in other embodiments, at least 75%, 80%, 85%, 90%, or 95% identity with SEQ ID NO: 76. In some embodiments, the Oct4 protein comprises an amino acid sequence having from 1 to 20 amino acid insertions, deletions, or substitutions (collectively) with respect to SEQ ID NO: 76. Or in other embodiments, the Oct4 protein comprises an amino acid sequence having from 1 to 15 or from 1 to 10 amino acid insertions, deletions, or substitutions (collectively) with respect to SEQ ID NO: 76.

(SEQ ID NO: 76)
MAGHLASDFAFSPPPGGGGDGPGGPEPGWVDPRTWLSFQGPPGGPGIGPG
VGPGSEVWGIPPCPPPYEFCGGMAYCGPQVGVGLVPQGGLETSQPEGEAG
VGVESNSDGASPEPCTVTPGAVKLEKEKLEQNPEESQDIKALQKELEQFA
KLLKQKRITLGYTQADVGLTLGVLFGKVFSQTTICRFEALQLSFKNMCKL
RPLLQKWVEEADNNENLQEICKAETLVQARKRKRTSIENRVRGNLENLFL
QCPKPTLQQISHIAQQLGLEKDVVRVWFCNRRQKGKRSSSDYAQREDFEA
AGSPFSGGPVSFPLAPGPHFGTPGYGSPHFTALYSSVPFPEGEAFPPVSV
TTLGSPMHSN

By “Sox2 protein” is meant a protein that is encoded by the SOX2 gene, or a natural or engineered variant, family-member, orthologue, fragment or fusion construct thereof, by way of non-limiting example, human Sox2 protein, mouse Sox2 protein, a DNA-binding domain of Sox2 protein or a Sox2-GFP fusion protein. In some embodiments the Sox2 protein comprises an amino acid sequence that has at least 70% identity with SEQ ID NO: 77, or in other embodiments, at least 75%, 80%, 85%, 90%, or 95% identity with SEQ ID NO: 77. In some embodiments, the Sox2 protein comprises an amino acid sequence having from 1 to 20 amino acid insertions, deletions, or substitutions (collectively) with respect to SEQ ID NO: 77. Or in other embodiments, the Sox2 protein comprises an amino acid sequence having from 1 to 15 or from 1 to 10 amino acid insertions, deletions, or substitutions (collectively) with respect to SEQ ID NO: 77.

(SEQ ID NO: 77)
MYNMMETELKPPGPQQTSGGGGGNSTAAAAGGNQKNSPDRVKRPMNAFMV
WSRGQRRKMAQENPKMHNSEISKRLGAEWKLLSETEKRPFIDEAKRLRAL
HMKEHPDYKYRPRRKTKTLMKKDKYTLPGGLLAPGGNSMASGVGVGAGLG
AGVNQRMDSYAHMNGWSNGSYSMMQDQLGYPQHPGLNAHGAAQMQPMHRY
DVSALQYNSMTSSQTYMNGSPTYSMSYSQQGTPGMALGSMGSVVKSEASS
SPPVVTSSSHSRAPCQAGDLRDMISMYLPGAEVPEPAAPSRLHMSQHYQS
GPVPGTAINGTLPLSHM

By “Klf4 protein” is meant a protein that is encoded by the KLF4 gene, or a natural or engineered variant, family-member, orthologue, fragment or fusion construct thereof, by way of non-limiting example, human Klf4 protein, mouse Klf4 protein, a DNA-binding domain of Klf4 protein or a Klf4-GFP fusion protein. In some embodiments the Klf4 protein comprises an amino acid sequence that has at least 70% identity with SEQ ID NO: 78, or in other embodiments, at least 75%, 80%, 85%, 90%, or 95% identity with SEQ ID NO: 78. In some embodiments, the Klf4 protein comprises an amino acid sequence having from 1 to 20 amino acid insertions, deletions, or substitutions (collectively) with respect to SEQ ID NO: 78. Or in other embodiments, the Klf4 protein comprises an amino acid sequence having from 1 to 15 or from 1 to 10 amino acid insertions, deletions, or substitutions (collectively) with respect to SEQ ID NO: 78.

(SEQ ID NO: 78)
QPPGESDMAVSDALLPSFSTFASGPAGREKTLRQAGAPNNRWREELSHMK
RLPPVLPGRPYDLAAATVATDLESGGAGAACGGSNLAPLPRRETEEFNDL
LDLDFILSNSLTHPPESVAATVSSSASASSSSSPSSSGPASAPSTCSFTY
PIRAGNDPGVAPGGTGGGLLYGRESAPPPTAPFNLADINDVSPSGGFVAE
LLRPELDPVYIPPQQPQPPGGGLMGKFVLKASLSAPGSEYGSPSVISVSK
GSPDGSHPVVVAPYNGGPPRTCPKIKQEAVSSCTHLGAGPPLSNGHRPAA
HDFPLGRQLPSRTTPTLGLEEVLSSRDCHPALPLPPGFHPHPGPNYPSFL
PDQMQPQVPPLHYQELMPPGSCMPEEPKPKRGRRSWPRKRTATHTCDYAG
CGKTYTKSSHLKAHLRTHTGEKPYHCDWDGCGWKFARSDELTRHYRKHTG
HRPFQCQKCDRAFSRSDHLALHMKRHF

By “c-Myc protein” is meant a protein that is encoded by the MYC gene, or a natural or engineered variant, family-member, orthologue, fragment or fusion construct thereof, by way of non-limiting example, human c-Myc protein, mouse c-Myc protein, l-Myc protein, c-Myc (T58A) protein, a DNA-binding domain of c-Myc protein or a c-Myc-GFP fusion protein. In some embodiments the c-Myc protein comprises an amino acid sequence that has at least 70% identity with SEQ ID NO: 79, or in other embodiments, at least 75%, 80%, 85%, 90%, or 95% identity with SEQ ID NO: 79. In some embodiments, the c-Myc protein comprises an amino acid having from 1 to 20 amino acid insertions, deletions, or substitutions (collectively) with respect to SEQ ID NO: 79. Or in other embodiments, the c-Myc protein comprises an amino acid sequence having from 1 to 15 or from 1 to 10 amino acid insertions, deletions, or substitutions (collectively) with respect to SEQ ID NO: 79.

(SEQ ID NO: 79)
MDFFRVVENQQPPATMPLNVSFTNRNYDLDYDSVQPYFYCDEEENFYQQQ
QQSELQPPAPSEDIWKKFELLPTPPLSPSRRSGLCSPSYVAVTPFSLRGD
NDGGGGSFSTADQLEMVTELLGGDMVNQSFICDPDDETFIKNIIIQDCMW
SGFSAAAKLVSEKLASYQAARKDSGSPNPARGHSVCSTSSLYLQDLSAAA
SECIDPSVVFPYPLNDSSSPKSCASQDSSAFSPSSDSLLSSTESSPQGSP
EPLVLHEETPPTTSSDSEEEQEDEEEIDVVSVEKRQAPGKRSESGSPSAG
GHSKPPHSPLVLKRCHVSTHQHNYAAPPSTRKDYPAAKRVKLDSVRVLRQ
ISNNRKCTSPRSSDTEENVKRRTHNVLERQRRNELKRSFFALRDQIPELE
NNEKAPKVVILKKATAYILSVQAEEQKLISEEDLLRKRREQLKHKLEQLR
NSCA

Any aspect or embodiment described herein can be combined with any other aspect or embodiment as disclosed herein.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties. The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### ADDITIONAL EMBODIMENTS

Embodiment A1: A method for manufacturing a population of cells that is enriched for cytotoxic lymphocytes, the method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes; wherein steps (5) and (6) occur in an adherent culturing vessel. In this embodiment, when CD34+ cells are selected, the embryoid bodies may be first chemically and/or mechanically dissociated.

Embodiment A2: The method of Embodiment A1, wherein the stem cell is an induced pluripotent stem (iPSC).

Embodiment A3: The method of Embodiment A1 or Embodiment A2, wherein the stem cell has a wild-type genome or has a genetically engineered disruption in a beta-2-microglobulin (B2M) gene.

Embodiment A4: The method of Embodiment A3, wherein the stem cell has a biallelic disruption in a B2M gene.

Embodiment A5: The method of any one of Embodiments A1 to A4, wherein the bioreactor is suited for culturing shear-sensitive cells and/or does not require use of anti-foaming agents or shear protectants.

Embodiment A6: The method of any one of Embodiments A1 to A5, wherein the bioreactor is a vertical wheel bioreactor.

Embodiment A7: The method of any one of Embodiments A1 to A6, wherein the medium in step (2) is serum-free and feeder-free culture medium.

Embodiment A8: The method of Embodiment A7, wherein the serum-free and feeder-free culture medium is an mTeSR™ medium.

Embodiment A9: The method of any one of Embodiments A1 to A8, wherein the medium in step (6) is a serum-free and feeder-free culture medium.

Embodiment A10: The method of Embodiment A9, wherein the serum-free and feeder-free culture medium is a StemDiff™ NK medium.

Embodiment A11: The method of any one of Embodiments A1 to A10, wherein the adherent culturing vessel is a multi-well plate or a cell culturing flask.

Embodiment A12: The method of any one of Embodiments A1 to A11, wherein the method provides from about 10-fold to about 100-fold more cytotoxic lymphocytes than obtained by a method in which each of the culturing steps comprise adherent culturing vessels; obtained by a method in which step (2) comprises a spheroid suspension culture and steps (3), (5), and (6) occur in adherent culturing vessels; and/or obtained by a method in which steps (5) and (6) occur in bioreactor.

Embodiment A13: The method of any one of Embodiments A1 to A12, wherein the cytotoxic lymphocytes are enriched for CD56+ cells, for CD16+ cells, NKG2D+ cells, CD226+ Cells, NKp46+ cells, NKp44+ cells, CD244+ cells, and/or CD94+ cells.

Embodiment A14: The method of any one of Embodiments A1 to A13, wherein the method provides from about 5-fold to about 30-fold more AD16+ cells than obtained by a method in which step (2) comprises a spheroid suspension culture and steps (3), (5), and (6) occur in adherent culturing vessels and/or obtained by a method in which steps (5) and (6) occur in bioreactor.

Embodiment A15: The method of any one of Embodiments A1 to A14, wherein the method provides from about 5-fold to about 25-fold more NDG2D+ cells than obtained by a method in which step (2) comprises a spheroid suspension culture and steps (3), (5), and (6) occur in adherent culturing vessels and/or obtained by a method in which steps (5) and (6) occur in bioreactor.

Embodiment A16: The method of any one of Embodiments A1 to A15, wherein the method provides from about 2-fold to about 30-fold more NKp44+ cells than obtained by a method in which step (2) comprises a spheroid suspension culture and steps (3), (5), and (6) occur in adherent culturing vessels and/or obtained by a method in which steps (5) and (6) occur in bioreactor.

Embodiment A17: The method of any one of Embodiments A1 to A16, wherein the method provides from about 2-fold to about 8-fold more CD94+ cells than obtained by a method in which step (2) comprises a spheroid suspension culture and steps (3), (5), and (6) occur in adherent culturing vessels and/or obtained by a method in which steps (5) and (6) occur in bioreactor.

Embodiment A18: The method of any one of Embodiments A1 to A17, wherein the method provides from about 2-fold more NKp46 cells than obtained by a method in which step (2) comprises a spheroid suspension culture and steps (3), (5), and (6) occur in adherent culturing vessels.

Embodiment A19: The method of any one of Embodiments A1 to A18, wherein the cytotoxic lymphocyte targets and kills cancer cells.

Embodiment A20: The method of Embodiment A19, wherein the cancer cell is a K562 cancer cell.

Embodiment A21: The method of Embodiment A19 or Embodiment A20, wherein the cytotoxic lymphocyte targets and kills cancer cells without requiring IL-15 and/or without requiring IL-2 activation.

Embodiment A22: The method of any one of Embodiments A19 to A21, wherein the cytotoxic lymphocyte targets and kills at least 70% of cancer cells in a population within about 4 hours.

Embodiment A23: The method of any one of Embodiments A19 to A22, wherein the cytotoxic lymphocyte targets and kills at least 80% of cancer cells in a population within about 24 hours.

Embodiment A24: The method of any one of Embodiments A1 to A18, wherein the cytotoxic lymphocyte has reduced cytotoxicity to an NK-resistant cancer cell.

Embodiment A25: The method of Embodiment A24, wherein the NK-resistant cancer cell is a NAMALWA cell.

Embodiment A26: The method of any one of Embodiments A1 to A25, wherein the cytotoxic lymphocyte is a Natural Killer (NK) cell.

Embodiment A27: The method of Embodiment A26, wherein the NK cell is a mature NK cell.

Embodiment A28: The method of any one of Embodiments A1 to A25, wherein the cytotoxic lymphocyte is a Natural killer T (NKT) cell.

Embodiment A29: The method of any one of Embodiments A1 to A25, wherein the cytotoxic lymphocyte is a delta-gamma T cell.

Embodiment A30: The method of any one of Embodiments A1 to A29, wherein the iPSC was reprogrammed from a somatic cell comprising contacting the somatic cell with one or more ribonucleic acids (RNAs), wherein each RNA encodes one or more reprogramming factors.

Embodiment A31: The method of any one of Embodiments A1 to A30, wherein the cytotoxic lymphocyte is further engineered with chimeric antigen receptors (CARs).

Embodiment A32: A method for killing a cancer cell, the method comprising steps of: (1) obtaining a cytotoxic lymphocyte of any one of Embodiments A1 to A31 and (2) contacting cytotoxic lymphocyte with the cancer.

Embodiment A33: The method of Embodiment A32, wherein the cancer cell is in vivo.

Embodiment A34: A method for treating a cancer patient in need thereof, the method comprising administering to the cancer patient a therapeutically-effective amounts of the cytotoxic lymphocyte of any one of Embodiments A1 to A31.

Embodiment A35: The method of Embodiment A34, wherein the administering is intravenous, intraarterial, intratumoral, or injected in the vicinity of a tumor.

Embodiment A36: The method of any one of Embodiments A32 to A35, wherein the cancer is a blood cancer.

Embodiment A37: The method of any one of Embodiments A32 to A35, wherein the cancer is a solid tumor.

Embodiment A38: The method of any one of Embodiments A32 to A37, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Embodiment A39: A pharmaceutical composition comprising the cytotoxic lymphocyte of any one of Embodiments A1 to A31 and a pharmaceutically acceptable carrier or excipient.

Embodiment B1: A method for producing macrophages from an induced a pluripotent stem cell (iPSC), the method comprising steps of: (1) obtaining an iPSC; (2) culturing the iPSC in a first medium for about three days; (3) culturing the iPSC in a second for about four days; (4) culturing the iPSC in a monocyte differentiating medium for at least seven days, thereby obtaining monocytes; (5) isolating the monocytes; (6) culturing the monocytes for about four days; (7) culturing the monocytes in the presence of M-CSF for three to four days, thereby obtaining macrophages; and (8) harvesting the macrophages.

Embodiment B2: The method of Embodiment B1, wherein the macrophages are further contacted with interferon gamma (IFN-γ) and/or lipopolysaccharide (LPS) to obtain M1 macrophages and/or the macrophages are further contacted with IL-4 to obtain M2 macrophages.

Embodiment B3: The method of Embodiment B1 or Embodiment B2, wherein an average yield of 4.1×104 cells per cm2 of isolated monocytes are obtained in step (5).

Embodiment B4: The method of any one of Embodiments B1 to B3, wherein step (3) is repeated two times, three times, or four times, with each repeat occurring every three to four days.

Embodiment B5: The method of any one of Embodiments B1 to B4, wherein isolating the monocytes comprises a CD14+ antibody, optionally, adhered to a solid support, e.g., a bead including a magnetic bead.

Embodiment B6: The method of any one of Embodiments B1 to B5, wherein the monocytes are tested for a reduction in expression of pluripotent stem cell markers, e.g., TRA-1-60 and TRA-1-81.

Embodiment B7: The method of any one of Embodiments B1 to B6, wherein the monocytes are tested for expression of one or more of the following markers: CD3, CD11b, CD13, CD33, CD45, CD68, CD80, CD206, and SIRPa.

Embodiment B8: The method of any one of Embodiments B1 to B7, wherein the monocytes have similar expression of CD11b, CD14, CD33, CD45, and CD163 relative to peripheral blood mononuclear cell (PBMC)-derived monocytes.

Embodiment B9: The method of any one of Embodiments B1 to B8, wherein the monocytes have an increase in one or more of CD3, CD68, CD80, and CD206 relative to PBMC-derived monocytes.

Embodiment B10: The method of any one of Embodiments B1 to B9, wherein the monocytes have both higher viability in culture and superior recovery from cryopreservation relative to PBMC-derived monocytes.

Embodiment B11: The method of any one of Embodiments B2 to B10, wherein the IFN-γ is provided at 50 ng/mL and the LPS is provided at 10 ng/mL.

Embodiment B12: The method of any one of Embodiments B2 to B11, wherein the IL-4 is provided at 10 ng/mL.

Embodiment B13: The method of any one of Embodiments B1 to B12, wherein the macrophages are tested for expression of one or more of the following markers: CD11b, CD68, CD80, CD86, CD163, CD206, and SIRPa.

Embodiment B14: The method of any one of Embodiments B2 to B13, wherein the M1 and M2 macrophages secrete similar levels of TNFα, IL-12p70, and IL-10 compared to PBMC-derived macrophages.

Embodiment B15: The method of any one of Embodiments B1 to B14, wherein macrophages, e.g., the M1 and M2 macrophages, are capable of killing cancer cells.

Embodiment B16: The method of any one of Embodiments B1 to B15, wherein the iPSC was reprogrammed from a differentiated or non-pluripotent cell.

Embodiment B17: The method of Embodiment B16, wherein the from a differentiated or non-pluripotent cell was reprogramed by transfecting the cell with the one or more synthetic RNA molecules encoding one or more reprogramming factors selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

Embodiment B18: The method of Embodiment B16 or Embodiment B17, wherein the differentiated or non-pluripotent cell is selected from fibroblasts, keratinocytes, melanocyte blood cells, bone marrow cells, adipose cells, and tissue-resident progenitor cells.

Embodiment B19: The method of any one of Embodiments B1 to B18, wherein the iPSC or a progenitor cell was gene-edited.

Embodiment B20: The method of Embodiment B19, wherein the iPSC or a progenitor cell was gene-edited to knockout the beta-2 microglobulin (B2M) gene.

Embodiment B21: The method of Embodiment B20, wherein the iPSC or a progenitor cell was gene-edited to knockout both copies of the B2M gene, i.e., a biallelic knockout of B2M.

Embodiment B22: The method of any one of Embodiments B19 to B21, wherein gene-editing comprises transfection of a repair template.

Embodiment B23: The method of any one of Embodiments B19 to B22, wherein the repair template comprises a TTAGGG motif for decreasing synthetic oligodeoxynucleotides (ODNs)-related activation of pro-inflammatory responses.

Embodiment B24: The method of Embodiment B22 or Embodiment B23, wherein the cell is transfected with a TTAGGG motif separate from the repair template.

Embodiment B25: The method of any one of Embodiments B16 to B24, wherein the differentiated or non-pluripotent cell was contacted with resveratrol before reprogramming.

Embodiment B26: The method of any one of Embodiments B19 to B25, wherein the iPSC was contacted with resveratrol before gene-editing.

Embodiment B27: The method of any one of Embodiments B19 to B26, wherein the iPSC was contacted with resveratrol after gene-editing.

Embodiment B28: The method of any one of Embodiment B1 to B27, wherein rather than starting with an iPSC, another stem cell is obtained.

Embodiment B29: The method of Embodiment B28, wherein the other stem cell is an embryonic stem cell.

Embodiment B30. An isolated macrophage obtained by the method of any one of Embodiments B1 to B29.

Embodiment B31: A pharmaceutical composition comprising the isolated macrophage of Embodiment B30 and a pharmaceutically-acceptable excipient.

Embodiment B32. An isolated M1 macrophage and/or an isolated M2 macrophage obtained by the method of any one of Embodiments B2 to B29.

Embodiment B33: A pharmaceutical composition comprising the isolated M1 macrophage and/or an isolated M2 macrophage of Embodiment B32 and a pharmaceutically-acceptable excipient.

Embodiment B34: A method for treating a cancer comprising in vivo administering to a subject in need the pharmaceutical composition of Embodiment B31 or Embodiment B33.

Embodiment B35: A method for decreasing synthetic oligodeoxynucleotides (ODNs)-related activation of pro-inflammatory responses, the method comprising transfecting a cell with an ODN comprising a TTAGGG motif.

Embodiment B36: The method of Embodiment B35, wherein the ODN is a double stranded ODN (dsODN) and comprise a repair template.

Embodiment B37: The method of Embodiment B36, wherein the TTAGGG motif is attached to the 5′ and/or the 3′ end of the repair template.

Embodiment B38: The method of Embodiment B35, wherein the ODN is a single stranded ODN (ssODN) and does not comprise a repair template.

Embodiment B39: The method of any one of Embodiments B35 to B38, wherein transfecting a cell with a TTAGGG motif results in approximately 50% higher viability than when the motif is not transfected.

Embodiment B40: The method of any one of Embodiments B36 to B39, wherein transfecting a cell with a TTAGGG motif results in approximately 50% higher expression of a gene encoded by the repair template than when the motif is not transfected.

Embodiment B41: The method of any one of Embodiments B35 to B40, wherein the cell is transfected with a synthetic nucleic acid encoding a gene-editing protein along with a repair template.

Embodiment B42: The method of any one of Embodiments B35 to B41, wherein the ODN comprising one, two, three, four, five, six, seven, eight, nine, ten or more repeats of the TTAGGG motif.

Embodiment B43: The method of any one of Embodiments B35 to B42, wherein the cells that are transfected with a TTAGG-containing ODN (either as a repair template or as separate ODN) are skin cells, pluripotent stem cells, embryonic stem cells, iPSCs, MSCs (including iMSCs), mesenchymal stromal/stem cells, hematopoietic cells, hematopoietic stem cells, lymphocytes, β-cells, T-cells (including CAR-T), NK cell (including CAR-NK), monocytes, macrophages (including CAR-myeloid cells and CAR-mesenchymal stromal/stem cells), retinal pigmented epithelial cells, hematopoietic cells, a hematopoietic stem cells, myeloid cells, tumor-infiltrating lymphocytes, marrow-infiltrating lymphocytes, a peripheral blood lymphocytes, cardiac cells, airway epithelial cells, neural stem cells, neurons, glial cells, bone cells, blood cells, and dental pulp stem cells.

Embodiment B44: The method of any one of Embodiments B35 to B42, wherein the cell that is transfected with a TTAGG-containing ODN reduces the upregulation of IFIT1 and IFIT3 relative cells that are not transfected with a TTAGG-containing ODN.

Embodiment B45. An isolated cell obtained by the method of any one of Embodiments B35 to B44.

Embodiment B46: A method for enhancing the efficiency of gene-editing, the method comprising contacting a cell with resveratrol before gene-editing.

Embodiment B47: The method of Embodiment B46, wherein contacting the cell with resveratrol arrests the cell in S/G2 phase.

Embodiment B48: The method of Embodiment B46 or Embodiment B47, wherein the cell is further contacted with resveratrol after gene-editing.

Embodiment B49: The method of any one of Embodiments B46 to B48, wherein gene-editing comprise transfection of a synthetic nucleic acid encoding a gene-editing protein.

Embodiment B50: A method for enhancing the efficiency of gene-editing, the method comprising contacting a cell that has been gene-edited with resveratrol.

Embodiment B51: The method of Embodiment B50, wherein gene-editing comprise transfection of a synthetic nucleic acid encoding a gene-editing protein.

Embodiment C1: A composition comprising an engineered cell comprising a genetically engineered disruption in a beta-2-microglobulin (B2M) gene, wherein the engineered cell is a cytotoxic lymphocyte, e.g., a lymphoid cell lineage, or the engineered cell is a myeloid lineage cell, e.g., a macrophage, or a mesenchymal stromal/stem cell, or a hematopoietic stem cell.

Embodiment C2: The composition of Embodiment C1, wherein the cytotoxic lymphocyte comprises genetically engineered disruptions of all substantially all copies of the B2M gene.

Embodiment C3: The composition of Embodiment C1 or C2, wherein the cytotoxic lymphocyte has a loss of function of the B2M gene.

Embodiment C4: The composition of Embodiment C1-C3, wherein the cytotoxic lymphocyte has a loss of function of both alleles of the B2M gene, optionally caused by contacting the cytotoxic lymphocyte with RNA encoding one or more gene-editing proteins.

Embodiment C5: The composition of any one of Embodiments C1-C4, wherein the genetically engineered disruption of the B2M gene is in exon 3 of human B2M.

Embodiment C6: The composition of any one of Embodiments C1-C5, wherein the genetically engineered disruption of the B2M gene is a deletion.

Embodiment C7: The composition of Embodiment C6, wherein the deletion is about 10 to about 20 nucleotides.

Embodiment C8: The composition of Embodiment C7, wherein the deletion is about 14 nucleotides.

Embodiment C9: The composition of Embodiment C7 or Embodiment C8, wherein the deletion is near nucleotides 500 to 550 of the human B2M gene.

Embodiment C10: The composition of Embodiment C9, wherein the deletion is of the sequence TTGACTTACTGAAG (SEQ ID NO: 14), or a functional equivalent thereof.

Embodiment C11: The composition of any one of Embodiments C1-C10, wherein the cytotoxic lymphocyte has downregulated MHC class I expression and/or activity.

Embodiment C12: The composition of any one of Embodiments C1-C11, wherein the cytotoxic lymphocyte is not substantially recognized by a host immune system upon administration to a subject.

Embodiment C13: The composition of any one of Embodiments C1-C12, wherein the cytotoxic lymphocyte has reduced or eliminated susceptibility to cell killing by host T cells as compared to a cytotoxic lymphocyte which does not comprise a genetically engineered disruption in the B2M gene.

Embodiment C14: The composition of any one of Embodiments C1-C13, wherein the cytotoxic lymphocyte has reduced or eliminated susceptibility to cell killing by other host cytotoxic lymphocytes as compared to another cytotoxic lymphocyte which comprises a genetically engineered disruption in the B2M gene.

Embodiment C15: The composition of any one of Embodiments C1-C14, wherein the cytotoxic lymphocyte is a stealth cell.

Embodiment C16: The composition of any one of Embodiments C1-C15, wherein the cytotoxic lymphocyte has reduced or eliminated host cytotoxic lymphocyte fratricide, e.g, NK-cell fratricide.

Embodiment C17: The composition of any one of Embodiments C1-C16, wherein the cytotoxic lymphocyte is capable of self-activating.

Embodiment C18: The composition of Embodiment C17, wherein the cytotoxic lymphocyte is capable of self-activating in the absence of an interleukin, optionally selected from interleukin-2 (IL-2) and interleukin-15 (IL-15).

Embodiment C19: The composition of any one of Embodiments C1-C18, wherein the cytotoxic lymphocyte is capable of inducing tumor cell cytotoxicity.

Embodiment C20: The composition of any one of Embodiments C1-C19, wherein the cytotoxic lymphocyte is capable of inducing tumor cell cytotoxicity in the absence of an interleukin, optionally selected from IL-2 and IL-15.

Embodiment C21: The composition of any one of Embodiments C1-C20, wherein the cytotoxic lymphocyte further comprises a genetically engineered disruption in a MHC II transactivator (CIITA) gene.

Embodiment C22: The composition of Embodiment C21, wherein the cytotoxic lymphocyte has downregulated MHC class II expression and/or activity.

Embodiment C23: The composition of any one of Embodiments C1-C22, wherein the cytotoxic lymphocyte comprises a genetically engineered alteration in one or more genes selected from HLA-A, HLA-B, HLA-C, HLA-E, HLA-F and HLA-G.

Embodiment C24: The composition of any one of Embodiments C1-C23, wherein the cytotoxic lymphocyte expresses a fusion protein comprising a B2M polypeptide and a HLA-A, HLA-B, HLA-C, HLA-E, HLA-F and HLA-G polypeptide.

Embodiment C25: The composition of Embodiment C24, wherein the fusion protein expressed by insertion of a repair template into a single or double strand break of the B2M gene; wherein the repair template comprises the coding sequence for B2M and the HLA gene, e.g., the coding sequence for one or more of HLA class I histocompatibility antigen, alpha chains (HLAs).

Embodiment C26: The composition of Embodiment C24 and Embodiment C25, wherein the fusion protein replaces endogenous B2M and HLA pairs expressed by a cytotoxic lymphocyte; thereby reducing the likelihood that the cytotoxic lymphocyte will be reduced or eliminated by a host cytotoxic lymphocyte.

Embodiment C27: The composition of any one of Embodiments C1-C26, wherein the cytotoxic lymphocyte does not comprise a genetically engineered alteration in one or more genes selected from HLA-A, HLA-B, HLA-C, HLA-E, HLA-F and HLA-G.

Embodiment C28: The composition of any one of Embodiments C1-C27, wherein the genetically engineered alteration is a genetically engineered reduction or elimination in expression and/or activity of one or more genes selected from HLA-A, HLA-B, HLA-C, HLA-E, HLA-F and HLA-G.

Embodiment C29: The composition of any one of Embodiments C1-C27, wherein the genetically engineered alteration is a genetically engineered increase in expression and/or activity of one or more genes selected from HLA-A, HLA-B, HLA-C, HLA-E, HLA-F and HLA-G.

Embodiment C30: The composition of any one of Embodiments C1-C29, wherein the cytotoxic lymphocyte, optionally, an NK cell, is genetically modified to express a recombinant chimeric antigen receptor (CAR) comprising an intracellular signaling domain, a transmembrane domain, and an extracellular domain comprising an antigen binding region.

Embodiment C31: The composition of Embodiment C30, the intracellular signaling domain comprises at least one immunoreceptor tyrosine-based activation motif (ITAM)-containing domain.

Embodiment C32: The composition of any one of Embodiments C30 or C31, wherein the intracellular signaling domain is from one of CD3-zeta, CD28, CD27, CD134 (OX40), and CD137 (4-1BB).

Embodiment C33: The composition of any one of Embodiments C30-C32, wherein the transmembrane domain is from one of CD28 or a CD8.

Embodiment C34: The composition of any one of Embodiments C30-C33, wherein the antigen binding region binds one antigen.

Embodiment C35: The composition of any one of Embodiments C30-C33, wherein the antigen binding region binds two antigens.

Embodiment C36: The composition of any one of Embodiments C30-C35, wherein the extracellular domain comprising an antigen binding region comprises: (a) C natural ligand or receptor, or fragment thereof, or (b) an immunoglobulin domain, optionally a single-chain variable fragment (scFv).

Embodiment C37: The composition of any one of Embodiments C30-C35, wherein the extracellular domain comprising an antigen binding region comprises two of: (a) a natural ligand or receptor, or fragment thereof, or (b) an immunoglobulin domain, optionally a single-chain variable fragment (scFv).

Embodiment C38: The composition of any one of Embodiments C30-C35, wherein the extracellular domain comprising an antigen binding region comprises one of each of: (a) a natural ligand or receptor, or fragment thereof, and (b) an immunoglobulin domain, optionally a single-chain variable fragment (scFv).

Embodiment C39: The composition of any one of Embodiments C30-38, wherein the antigen binding region binds a tumor antigen.

Embodiment C40: The composition of any one of Embodiments C30-C39, wherein the antigen binding region comprises one or more of: a) CD94/NKG2a, which optionally binds HLA-E on a tumor cell; b) CD96, which optionally binds CD155 on a tumor cell; c) TIGIT, which optionally binds CD155 or CD112 on a tumor cell; d) DNAM-1, which optionally binds CD155 or CD112 on a tumor cell; e) KIR, which optionally binds HLA class I on a tumor cell; f) NKG2D, which optionally binds NKG2D-L on a tumor cell; g) CD16a, which optionally binds an antibody/antigen complex on a tumor cell and/or wherein the CD16a is optionally a high affinity variant, optionally homozygous or heterozygous for F158V; h) NKp30, which optionally binds B7-H6 on a tumor cell; i) NKp44; and j) NKp46.

Embodiment C41: The composition of any one of Embodiments C30-C40, wherein the antigen binding region comprises an immunoglobulin domain, optionally an scFv directed against HLA-E, CD155, CD112 HLA class I, NKG2D-L, or B7-H6.

Embodiment C42: The composition of any one of Embodiments C30-C41, wherein the antigen binding region binds an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2.

Embodiment C43: The composition of any one of Embodiments C30-C42, wherein the antigen binding region binds two antigens, the antigens being:
a. an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2 and
b. an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2.

Embodiment C44: The composition of any one of Embodiments C30-C43, wherein the extracellular domain of the recombinant CAR comprises the extracellular domain of an NK cell activating receptor or a scFv.

Embodiment C45: The composition of any one of Embodiments C30-C44, wherein the cytotoxic lymphocyte comprises a gene-edit in one or more of IL-7, CCL17, CCR4, IL-6, IL-6R, IL-12, IL-15, NKG2A, NKG2D, KIR, TRAIL, TRAC, PD1, and HPK1.

Embodiment C46: The composition of Embodiment C45, wherein the gene-edit in one or more of IL-7, CCL17, CCR4, IL-6, IL-6R, IL-12, IL-15, NKG2A, NKG2D, KIR, TRAIL, TRAC, PD1, and HPK1 is caused by contacting the cell with RNA encoding one or more gene-editing proteins.

Embodiment C47: The composition of Embodiment C46, wherein the gene-edit of causes a reduction or elimination of expression and/or activity of IL-6, NKG2A, NKG2D, KIR, TRAC, PD1, and/or HPK1.

Embodiment C48: The composition of Embodiment C46, wherein the gene-edit causes an increase of expression and/or activity of IL-7, CCL17, CCR4, IL-6R, IL-12, IL-15, and/or TRAIL.

Embodiment C49: The composition of any one of Embodiments C1-C48, wherein the lymphoid cell is a T cell.

Embodiment C50: The composition of Embodiment C49, wherein the T cell is a gamma-delta T cell.

Embodiment C51: The composition of any one of Embodiments C1-C48, wherein the lymphoid cell is an NK cell.

Embodiment C52: The composition of Embodiment C51, wherein the NK cell is an NK-T cell.

Embodiment C53: The composition of Embodiment C51, wherein the NK cell is a human cell.

Embodiment C54: The composition of any one of Embodiments C51-C53, wherein the NK cell is derived from somatic cell of a subject.

Embodiment C55: The composition of any one of Embodiments C51-C54, wherein the NK cell is derived from allogeneic or autologous cells.

Embodiment C56: The composition of any one of Embodiments C51-C55, wherein the NK cell is derived from an induced pluripotent stem (iPS) cell.

Embodiment C57: The composition of Embodiment C56, wherein the iPS is derived from reprogramming a somatic cell to an iPS cell, the reprogramming comprising contacting the iPS cell with a ribonucleic acid (RNA) encoding one or more reprogramming factors, optionally selected from Oct4, Sox2, cMyc, and Klf4.

Embodiment C58: The composition of Embodiment C57, wherein the reprogramming comprising contacting the iPS cell with one or more RNAs encoding each Oct4, Sox2, cMyc, and Klf4.

Embodiment C59: The composition of any one of Embodiments C56 or C57, wherein the iPS cell is derived from allogeneic or autologous cells.

Embodiment C60: The composition of any one of Embodiments C1-C59, wherein the genetically engineered disruption of the B2M comprises a gene-edit and the gene-edit is caused by contacting the cell with RNA encoding one or more gene-editing proteins.

Embodiment C61: The composition of any one of Embodiments C1-C60, wherein the NK cell expresses one or more of CD56 and CD16.

Embodiment C62: The composition of Embodiment C61, wherein the NK cell expresses CD16a, which optionally binds an antibody/antigen complex on a tumor cell and/or wherein the CD16a is optionally a high affinity variant, optionally homozygous or heterozygous for F158V.

Embodiment C63: The composition of any one of Embodiments C1-C62, wherein the NK cell does not express CD3.

Embodiment C64: The composition of any one of Embodiments C1-C63, wherein the NK cell is CD56bright CD16dim/−.

Embodiment C65: The composition of any one of Embodiments C1-C64, wherein the NK cell is CD56dim CD16+.

Embodiment C66: The composition of any one of Embodiments C1-C65, wherein the NK cell is a NKtolerant cell, optionally comprising CD56brightNK cells or CD27− CD11b− NK cells.

Embodiment C67: The composition of any one of Embodiments C1-C65, wherein the NK cell is a NKcytotoxic cell, optionally comprising CD56dimNK cells or CD11b+ CD27− NK cells.

Embodiment C68: The composition of any one of Embodiments C1-C65, wherein the NK cell is a NKregulatory cell, optionally comprising CD56bright NK cells or CD27+ NK cells.

Embodiment C69: The composition of any one of Embodiments C1-C65, wherein the NK cell is a natural killer T (NKT) cell.

Embodiment C70: The composition of any one of Embodiments C1-C69, wherein the NK cell secretes one or more cytokines selected from interferon-gamma (IFN-g), tumor necrosis factor-alpha (TNF-a), tumor necrosis factor-beta (TNF-b), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b).

Embodiment C71: The composition of any one of Embodiments C1-C70, wherein the NK cell further comprises one or more recombinant genes capable of encoding a suicide gene product.

Embodiment C72: The composition of Embodiment C71, wherein the suicide gene product comprises a protein selected from the group consisting of thymidine kinase and an apoptotic signaling protein.

Embodiment C73: The composition of any one of Embodiments C60-C72, wherein the gene-editing protein is selected from a nuclease, a transcription activator-like effector nuclease (TALEN), RiboSlice, a zinc-finger nuclease, a meganuclease, a nickase, a clustered regularly interspaced short palindromic repeat (CRISPR)-associated protein or a natural or engineered variant, family-member, orthologue, fragment or fusion construct thereof.

Embodiment C74: The composition of any one of Embodiments C2-C73, wherein the RNA is mRNA.

Embodiment C75: The composition of Embodiment C74, wherein the RNA is modified mRNA.

Embodiment C76: The composition of Embodiment C75, wherein the modified mRNA comprises one or more non-canonical nucleotides.

Embodiment C77: The composition of Embodiment C76, wherein the non-canonical nucleotides have one or more substitutions at positions selected from the 2C, 4C, and 5C positions for a pyrimidine, or selected from the 6C, 7N and 8C positions for a purine.

Embodiment C78: The composition of any one of Embodiments C76 or C77, wherein the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

Embodiment C79: The composition of any one of Embodiments C2-C78, wherein the RNA comprises a 5′ cap structure.

Embodiment C80: The composition of any one of Embodiments C2-C79, wherein the RNA 5′-UTR comprises a Kozak consensus sequence.

Embodiment C81: The composition of Embodiment C80, wherein the RNA 5′-UTR comprises a sequence that increases RNA stability in vivo, and the 5′-UTR may comprise an alpha-globin or beta-globin 5′-UTR.

Embodiment C82: The composition of any one of Embodiments C2-C81, wherein the RNA 3′-UTR comprises a sequence that increases RNA stability in vivo, and the 3′-UTR may comprise an alpha-globin or beta-globin 3′-UTR.

Embodiment C83: The composition of any one of Embodiments C2-C82, wherein the RNA comprises a 3′ poly(A) tail.

Embodiment C84: The composition of Embodiment C83, wherein the RNA 3′ poly(A) tail is from about 20 nucleotides to about 250 nucleotides in length.

Embodiment C85: The composition of any one of Embodiments C2-C84, wherein the RNA is from about 200 nucleotides to about 5000 nucleotides in length.

Embodiment C86: The composition of any one of Embodiments C2-C85, wherein the RNA is prepared by in vitro transcription.

Embodiment C87: The composition of any one of Embodiments C1-C86, wherein the myeloid cell is a macrophage.

Embodiment C88: The composition of Embodiment C87, wherein the macrophage is a M1 macrophage or a M2 macrophage.

Embodiment C89: C pharmaceutical composition comprising an isolated NK cell of any of the above Embodiments.

Embodiment C90: C method of making an engineered cytotoxic lymphocyte, comprising steps of (a) reprogramming a somatic cell to an iPS cell, the reprogramming comprising contacting the iPS cell with a ribonucleic acid (RNA) encoding one or more reprogramming factors; (b) disrupting a B2M gene in the iPS cell, the disrupting comprising gene-editing the cell by contacting the cell with RNA encoding one or more gene-editing proteins; and (c) differentiating the iPS cell into a cytotoxic lymphocyte; wherein the cytotoxic lymphocyte is selected from a lymphoid cell or myeloid cell.

Embodiment C91: The method of Embodiment C90, wherein the cytotoxic lymphocyte is an NK cell.

Embodiment C92: The method of Embodiment C91, wherein the NK cell is an NK-T cell.

Embodiment C93: The method of Embodiment C91 or 92, wherein the NK cell is a human cell.

Embodiment C94: The method of Embodiment C90, wherein the lymphoid cell is a T cell.

Embodiment C95: The method of Embodiment C94, wherein the T cell is a gamma-delta T cell.

Embodiment C96: The method of Embodiment C90, wherein the myeloid cell is a macrophage.

Embodiment C97: The method of Embodiment C96, wherein the macrophage is a M1 macrophage or a M2 macrophage.

Embodiment C98: The method of any one of Embodiments C90-C97, wherein the somatic cell is a fibroblast or keratinocyte.

Embodiment C99: The method of any one of Embodiments C90-C98, wherein the method provides an increased proliferation rate of iPS cells as compared to the rate of iPS cells without a disruption of the B2M gene.

Embodiment C100: The method of any one of Embodiments C90-C99, wherein the method provides an increased proliferation rate of differentiating cells along a lymphoid lineage cell as compared to the rate of iPS cells without a disruption of the B2M gene.

Embodiment C101: The method of any one of Embodiments C90-C100, wherein the method provides an increased expansion of differentiating cells along a lymphoid lineage cell as compared to the rate of iPS cells without a disruption of the B2M gene.

Embodiment C102: The method of any one of Embodiments C90-C101, wherein the differentiating comprises embryoid body-based hematopoietic commitment.

Embodiment C103: The method of any one of Embodiments C90-C102, wherein the differentiating comprises enrichment of CD34+ cells.

Embodiment C104: The method of any one of Embodiments C90-C103, wherein the differentiating comprises differentiating into CD5+/CD7+ common lymphoid progenitors.

Embodiment C105: The method of any one of Embodiments C90-C104, wherein the method yields CD56dim CD16+ NK cells.

Embodiment C106: The method of any one of Embodiments C90-C105, wherein the RNA is associated with one or more lipid selected from Table C and/or Formulae I-XVI.

Embodiment C107: The method of any one of Embodiments C90-C106, wherein the cytotoxic lymphocyte is the cell of any one of Embodiments C1-C86.

Embodiment C108: C method of treating cancer, comprising steps of obtaining an isolated cytotoxic lymphocyte comprising a genetically engineered disruption in a B2M gene and administering the isolated cytotoxic lymphocyte to a subject in need thereof; wherein the cytotoxic lymphocyte is a lymphoid cell or a CAR-myeloid cell or a CAR-mesenchymal stromal/stem cell.

Embodiment C109: The method of Embodiment C108, wherein the cytotoxic lymphocyte is a T cell, e.g., a cytotoxic T cell or gamma-delta T cell; NK cell, e.g., a NK-T cell; or a macrophage, e.g., M1 macrophage or M2 macrophages an NK cell.

Embodiment C110: The method of any one of Embodiments C108 or C109, wherein the cancer is a blood cancer.

Embodiment C111: The method of any one of Embodiments C108 or C109, wherein the cancer is a solid tumor.

Embodiment C112: The method of any one of Embodiments C108-C111, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Embodiment C113: The method of any one of Embodiments C108-C112, wherein the cytotoxic lymphocyte is the cell of any one of Embodiments C1-C86.

Embodiment C114: C composition comprising an isolated cytotoxic lymphocyte comprising a gene edit in a CD16a gene, wherein the cytotoxic lymphocyte is selected from a lymphoid cell or myeloid cell.

Embodiment C115: The composition of Embodiment C114, wherein the gene edit transforms the CD16a into a high affinity variant of CD16a.

Embodiment C116: The composition of Embodiment C114 or Embodiment C115, wherein the gene edit introduces a phenylalanine to valine substitution (F158V) at position 158.

Embodiment C117: The composition of Embodiment C116, wherein the cell is homozygous or heterozygous for F158V.

Embodiment D1: A method for screening constructs capable of being expressed in an in vivo cell and for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; and (3) identifying cells that have been transformed and capable of expressing the fusion protein for the ability of its fusion protein to recognize and/or bind to a cancer cell.

Embodiment D2: A method for screening constructs capable of being expressed in an ex vivo cell and for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) culturing the cell capable of expressing the fusion protein which recognizes and/or binds to a cancer cell until a therapeutic amount of the cell is manufactured.

Embodiment D3: A method for screening constructs capable of being expressed in an ex vivo cell and for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) contacting an ex vivo cell with the synthetic mRNA encoding the gene-editing protein and the repair template encoding the fusion protein which was identified in step (3) as having the ability recognize and/or bind to a cancer cell; and (5) culturing the cell of step (4) until a therapeutic amount of the cell is manufactured.

Embodiment D4: A method for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; and (4) administering to a subject in need the synthetic mRNA encoding the gene-editing protein and the repair template encoding the fusion protein which has the ability recognize and/or bind to a cancer cell.

Embodiment D5: A method for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) culturing the cell capable of expressing the fusion protein which recognizes and/or binds to a cancer cell until a therapeutic amount of the cell is manufactured; and (5) administering a therapeutically-effective amount of the cells of step (4) to a subject in need.

Embodiment D6: A method for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) contacting an ex vivo cell with the synthetic mRNA encoding the gene-editing protein and the repair template encoding the fusion protein which was identified in step (3) as having the ability recognize and/or bind to a cancer cell; (5) culturing the cell of step (4) until a therapeutic amount of the cell is manufactured; and (6) administering a therapeutically-effective amount of the cells of step (4) to a subject in need.

Embodiment D7: The method of any one of Embodiments D1 to D6, wherein the fusion protein that recognizes and/or binds to a cancer cell is a chimeric antigen receptor (CAR).

Embodiment D8: The method of Embodiment D7, wherein the CAR comprises an intracellular signaling domain, a transmembrane domain, and an extracellular domain comprising an antigen binding region.

Embodiment D9: The method of Embodiment D8, wherein the intracellular signaling domain comprises at least one immunoreceptor tyrosine-based activation motif (ITAM)-containing domain.

Embodiment D10: The method of Embodiment D8 or Embodiment D9, wherein the intracellular signaling domain is from one of CD3-zeta, CD28, CD27, CD134 (OX40), and CD137 (4-1BB).

Embodiment D11: The method of any one of Embodiments D8 to D10, wherein the transmembrane domain is from one of CD28 or a CD8.

Embodiment D12: The method of any one of Embodiments D8 to D11, wherein the antigen binding region binds one antigen.

Embodiment D13: The method of any one of Embodiments D8 to D12, wherein the antigen binding region binds two antigens.

Embodiment D14: The method of any one of Embodiments D8 to D13, wherein the extracellular domain comprising an antigen binding region comprises: (a) C natural ligand or receptor, or fragment thereof, or (b) an immunoglobulin domain, optionally a single-chain variable fragment (scFv).

Embodiment D15: The method of any one of Embodiments D8 to D14, wherein the extracellular domain comprising an antigen binding region comprises two of (a) a natural ligand or receptor, or fragment thereof, or (b) an immunoglobulin domain, optionally a single-chain variable fragment (scFv).

Embodiment D16: The method of any one of Embodiments D8 to D15, wherein the extracellular domain comprising an antigen binding region comprises one of each of: (a) a natural ligand or receptor, or fragment thereof, and (b) an immunoglobulin domain, optionally a single-chain variable fragment (scFv).

Embodiment D17: The method of any one of Embodiments D8 to D16, wherein the antigen binding region binds a tumor antigen.

Embodiment D18: The method of any one of Embodiments D8 to D17, wherein the antigen binding region comprises one or more of: a. CD94/NKG2a, which optionally binds HLA-E on a tumor cell; b. CD96, which optionally binds CD155 on a tumor cell; c. TIGIT, which optionally binds CD155 or CD112 on a tumor cell; d. DNAM-1, which optionally binds CD155 or CD112 on a tumor cell; e. KIR, which optionally binds HLA class I on a tumor cell; f. NKG2D, which optionally binds NKG2D-L on a tumor cell; g. CD16a, which optionally binds an antibody/antigen complex on a tumor cell and/or wherein the CD16a is optionally a high affinity variant, optionally homozygous or heterozygous for F158V; h. NKp30, which optionally binds B7-H6 on a tumor cell; i. NKp44; and j. NKp46.

Embodiment D19: The method of any one of Embodiments D8 to D18, wherein the antigen binding region comprises an immunoglobulin domain, optionally an scFv directed against HLA-E, CD155, CD112 HLA class I, NKG2D-L, or B7-H6.

Embodiment D20: The method of any one of Embodiments D8 to D19, wherein the antigen binding region binds an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2.

Embodiment D21: The method of any one of Embodiments D8 to D20, wherein the antigen binding region binds two antigens, the antigens being: a. an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2 and b. an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2.

Embodiment D22: The method of any one of Embodiments D8 to D21, wherein the extracellular domain of the recombinant CAR comprises the extracellular domain of an NK cell activating receptor or a scFv.

Embodiment D24: The method of any one of Embodiments D1 to D22, wherein the cell type is of the lymphoid cell lineage or the myeloid cell lineage.

Embodiment D25: The method of Embodiment D24, wherein the lymphoid lineage cell is a T cell, e.g., a cytotoxic T cell or gamma-delta T cell, or an NK cell, e.g., an NK-T cell.

Embodiment D26: The method of Embodiment D24, wherein the myeloid lineage cell is a macrophage, e.g., an M1 macrophage or an M2 macrophage.

Embodiment D27: The method of any one of Embodiments D1 to D26, wherein after gene editing, the cell is a CAR-T cell, CAR-NK cell, a CAR-myeloid cell, or a CAR-mesenchymal stromal/stem cell.

Embodiment E1: A method for treating a cancer, the method comprising administering to a subject in need a therapeutically-effective amount of a first pharmaceutical composition comprising one or both of a population of isolated lymphoid lineage cells and a population of isolated myeloid lineage cells.

Embodiment E2: The method of Embodiment E1, wherein the first pharmaceutical composition comprises the population of isolated lymphoid lineage cells and wherein the subject in need is administered a therapeutically-effective amount of a second pharmaceutical composition comprising a population of isolated myeloid lineage cells.

Embodiment E3: The method of Embodiment E1, wherein the first pharmaceutical composition comprises the population of isolated myeloid lineage cells and wherein the subject in need is administered a therapeutically-effective amount of a second pharmaceutical composition comprising a population of isolated lymphoid lineage cells.

Embodiment E4: The method of Embodiment E2 or Embodiment E3, wherein the first pharmaceutical composition and the second pharmaceutical composition are administered simultaneously or sequentially.

Embodiment E5: The method of Embodiment E4, wherein the first pharmaceutical composition and the second pharmaceutical composition are administered sequentially with the first pharmaceutical composition administered before the second pharmaceutical composition.

Embodiment E6: The method of Embodiment E4, wherein the first pharmaceutical composition and the second pharmaceutical composition are administered sequentially with the second pharmaceutical composition administered before the first pharmaceutical composition.

Embodiment E7: The method of Embodiment E1, wherein the first pharmaceutical composition comprises both the population of isolated lymphoid lineage cells and the population of isolated myeloid lineage cells.

Embodiment E8: The method of any one of Embodiments E1 to E7, wherein one or more of the isolated lymphoid lineage cells and/or one or more of the isolated myeloid lineage cells comprise a genomic modification that expresses a chimeric antigen receptor (CAR).

Embodiment E9: The method of Embodiment E8, wherein the CAR comprises an antigen binding region that binds to one or more antigens expressed by a cancer cell.

Embodiment E10: The method of Embodiment E9, wherein the antigen binding region binds to one or more tumor antigens.

Embodiment E11: The method of any one of Embodiments E8 to E10, wherein the CAR comprises an antigen binding region that binds to ROR1.

Embodiment E12: The method of any one of Embodiments E1 to E11, wherein one or more of the isolated lymphoid lineage cells and/or one or more of the isolated myeloid lineage cells comprise a genomic modification that expresses or over expresses a cytokine.

Embodiment E13: The method of any one of Embodiments E1 to E12, wherein one or more of the isolated lymphoid lineage cells and/or one or more of the isolated myeloid lineage cells comprise a genomic modification which disrupts the beta-2-microglobulin (B2M) gene, optionally, wherein the cells express a fusion protein comprising a B2M polypeptide and an HLA polypeptide (e.g., an HLA-A, HLA-B, HLA-C, HLA-E, HLA-F and HLA-G polypeptide), e.g., the coding sequence for one or more of HLA class I histocompatibility antigen, alpha chains (HLAs).

Embodiment E14: The method of any one of Embodiments E1 to E13, further comprising administering to the subject in need a synthetic mRNA encoding a gene-editing protein (e.g., a temperature-sensitive gene-editing protein) and a single-stranded or double-stranded repair template which encodes a chimeric antigen receptor (CAR).

Embodiment E15: The method of Embodiment E14, wherein the gene-editing protein creates a single-stranded break or a double-stranded break in the genomic DNA of a cell in the subject and the single-stranded or double-stranded repair template which encodes the CAR inserts into the break.

Embodiment E16: The method of Embodiment E15, wherein the cell in the subject expresses the CAR.

Embodiment E17: The method of any one of Embodiments E1 to E16, further comprising administering to the subject in need a synthetic mRNA encoding a gene-editing protein (e.g., a temperature-sensitive gene-editing protein) and a single-stranded or double-stranded repair template which encodes a cytokine.

Embodiment E18: The method of Embodiment E17, wherein the gene-editing protein creates a single-stranded break or a double-stranded break in the genomic DNA of a cell in the subject and the single-stranded or double-stranded repair template which encodes the cytokine inserts into the break.

Embodiment E19: The method of Embodiment E18, wherein the cell in the subject expresses or over expresses the cytokine.

Embodiment E20: The method of any one of Embodiments E14 to E19, wherein the synthetic mRNA and/or the repair template is combined with a lipid system comprising a compound of Formula (IV).

Embodiment E21: The method of any one of Embodiments E14 to E20, wherein the cell in the subject is of the lymphoid lineage or is of the myeloid lineage.

Embodiment E22: The method of any one of Embodiments E1 to E21, wherein the isolated lymphoid lineage cell and/or the isolated myeloid lineage cell is derived from an induced pluripotent stem cell (iPSC).

Embodiment E23: The method of Embodiment E22, wherein the isolated lymphoid lineage cell and the isolated myeloid lineage cell are derived from the same iPSC.

Embodiment E24: The method of Embodiment E22 or Embodiment E23, wherein the iPSC comprises a genomic modification that expresses a chimeric antigen receptor (CAR).

Embodiment E25: The method of any one of Embodiments E22 to E24, wherein the iPSC comprises a genomic modification that expresses or over expresses a cytokine.

Embodiment E26: The method of any one of Embodiments E22 to E25, wherein the iPSC comprises a genomic modification which disrupts the beta-2-microglobulin (B2M) gene.

Embodiment E27: The method of any one of Embodiments E1 to E26, wherein the isolated lymphoid lineage cells are manufactured by a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes; wherein steps (5) and (6) occur in an adherent culturing vessel.

Embodiment E28: The method of any one of Embodiments E1 to E27, wherein the isolated lymphoid lineage cells comprise cytotoxic lymphocytes.

Embodiment E29: The method of Embodiment E28, wherein the isolated lymphoid lineage cells comprising cytotoxic lymphocytes are enriched for CD56+ cells, for CD16+ cells, NKG2D+ cells, CD226+ Cells, NKp46+ cells, NKp44+ cells, CD244+ cells, and/or CD94+ cells.

Embodiment E30: The method of Embodiment E28 or Embodiment E29, wherein the cytotoxic lymphocyte targets and kills cancer cells.

Embodiment E31: The method of Embodiment E30, wherein the cytotoxic lymphocyte targets and kills cancer cells without requiring IL-15 and/or without requiring IL-2 activation.

Embodiment E32: The method of any one of Embodiments E28 to E31, wherein the cytotoxic lymphocyte has reduced cytotoxicity to an NK-resistant cancer cell.

Embodiment E33: The method of any one of Embodiments E28 to E32, wherein the cytotoxic lymphocyte is a Natural Killer (NK) cell or is a cytotoxic T cell.

Embodiment E34: The method of Embodiment E33, wherein the NK cell is a mature NK cell.

Embodiment E35: The method of any one of Embodiments E28 to E34, wherein the cytotoxic lymphocyte is a Natural killer T (NKT) cell.

Embodiment E36: The method of any one of Embodiments E33 to E35, wherein the NK cell expresses CD16a and/or the NK cell does not express CD3.

Embodiment E37: The method of any one of Embodiments E33 to E36, wherein the NK cell is CD56bright CD16dim/−.

Embodiment E38: The method of any one of Embodiments E33 to E37, wherein the NK cell secretes one or more cytokines selected from interferon-gamma (IFNγ), tumor necrosis factor-alpha (TNFα), tumor necrosis factor-beta (TNFβ), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b).

Embodiment E39: The method of any one of Embodiments E28 to E38, wherein the cytotoxic lymphocyte is a delta-gamma T cell.

Embodiment E40: The method of any one of Embodiments E28 to E39, wherein the cytotoxic lymphocyte is further engineered to express a chimeric antigen receptor (CAR).

Embodiment E41: The method of any one of Embodiments E28 to E40, wherein the cytotoxic lymphocyte is further engineered to express or overexpress a cytokine.

Embodiment E42: The method of any one of Embodiments E1 to E41, wherein the isolated myeloid lineage cells are manufactured by a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a myeloid progenitor medium; and (6) culturing the cells of step (5) in a macrophage cell medium under conditions to obtain a population of cells enriched for macrophages; wherein steps (5) and (6) occur in a bioreactor.

Embodiment E43: The method of any one of Embodiments E1 to E42, wherein the isolated myeloid lineage cells comprise a megakaryocyte, erythrocyte, mast cell, myeloblast, dendritic cell, basophil, neutrophil, eosinophil, monocyte, or macrophage.

Embodiment E44: The method of any one of Embodiments E1 to E43, wherein the isolated myeloid lineage cells express one or more of CD11b, CD13, CD14, CD33, CD45, CD80, CD163, CD206, and SIRPα, e.g., in amounts that are similar to amounts expressed by PBMC-derived cells.

Embodiment E45: The method of any one of Embodiments E1 to E44, wherein the isolated myeloid lineage cells have increased expression of CD80 and/or CD206, which is indicative of an activated state.

Embodiment E46: The method of any one of Embodiments E1 to E45, wherein the isolated myeloid lineage cell is a macrophage.

Embodiment E47: The method of Embodiment E46, wherein the macrophage expresses one or more of CD11b, CD68, CD80, CD86, CD163, CD206, and SIRPα in amounts that are similar to amounts expressed by PBMC-derived cells and/or secretes one or more of TNFα, IL-12p70, and IL-10 in amounts that are similar to amounts expressed by PBMC-derived cells.

Embodiment E48: The method of Embodiment E46 or Embodiment E47, wherein the macrophage expresses one or more of CD34, CD44, CD45, CD73, and CD90.

Embodiment E49: The method of any one of Embodiments E46 to E48, wherein the method further comprises a step of differentiating the macrophages into M1 and/or M2 macrophages, e.g., by exposure to MCSF.

Embodiment E50: The method of Embodiment E49, wherein the method further comprises a step of polarizing the M1 macrophages with interferon gamma (IFN-γ) and/or lipopolysaccharide (LPS) and/or treating the M2 macrophages with IL-4.

Embodiment E51: The method of any one of Embodiments E46 to E50, wherein the macrophages comprise M1 macrophages and/or M2 macrophages.

Embodiment E52: The method of Embodiment E51, wherein the M1 macrophages and/or M2 macrophages secrete one or more of TNFα, IL-12p70, and IL-10 in amounts that are similar to amounts expressed by PBMC-derived cells.

Embodiment E53: The method of any one of Embodiments E1 to E52, wherein the isolated myeloid lineage cells kill cancer cells and/or promote cancer cell killing by cytotoxic lymphocytes.

Embodiment E54: The method of any one of Embodiments E1 to E53, wherein the isolated myeloid lineage cell is further engineered to express a chimeric antigen receptor (CAR).

Embodiment E55: The method of any one of Embodiments E1 to E54, wherein the isolated myeloid lineage cell is further engineered to express or overexpress a cytokine.

Embodiment E56: The method of any one of Embodiments E27 to E55, when CD34+ cells are selected, the embryoid bodies are first chemically and/or mechanically dissociated.

Embodiment E57: The method of any one of Embodiments E27 to E56, wherein the stem cell is an induced pluripotent stem (iPSC).

Embodiment E58: The method of any one of Embodiments E27 to E57, wherein the stem cell stem has a wild-type genome or has a genetically engineered disruption in a beta-2-microglobulin (B2M) gene.

Embodiment E59: The method of Embodiment E58, wherein the stem cell has a biallelic disruption in a B2M gene.

Embodiment E60: The method of Embodiment E58 or Embodiment E59, wherein the stem cell expresses a fusion protein comprising a B2M polypeptide and an HLA polypeptide (e.g., an HLA-A, HLA-B, HLA-C, HLA-E, HLA-F and HLA-G polypeptide), e.g., the coding sequence for one or more of HLA class I histocompatibility antigen, alpha chains (HLAs).

Embodiment E61: The method of any one of Embodiments E57 to E60, wherein the iPSC was reprogrammed from a somatic cell comprising contacting the somatic cell with one or more ribonucleic acids (RNAs), wherein each RNA encodes one or more reprogramming factors.

Embodiment E62: The method of Embodiment E61, wherein the one or more reprogramming factors are selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

Embodiment E63: The method of Embodiment E61 or Embodiment E62, wherein the somatic cell is selected from fibroblasts, keratinocytes, melanocyte blood cells, bone marrow cells, adipose cells, and tissue-resident progenitor cells.

Embodiment E64: The method of any one of Embodiments E57 to E63, wherein the iPSC is further engineered to express a chimeric antigen receptor (CAR).

Embodiment E65: The method of any one of Embodiments E57 to E64, wherein the iPSC is further engineered to express or overexpress a cytokine.

Embodiment E66: The method of any one of Embodiments E1 to E65, wherein the isolated lymphoid lineage cells and the isolated myeloid lineage cells are manufactured by a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor.

Embodiment E67: The method of Embodiment E66, when CD34+ cells are selected, the embryoid bodies are first chemically and/or mechanically dissociated.

Embodiment E68: The method of Embodiment E66 or Embodiment E67, wherein the stem cell is an induced pluripotent stem (iPSC).

Embodiment E69: The method of any one of Embodiments E66 to E68, wherein the method of manufacturing provides at least 1×10⁶ myeloid lineage cells/ml and at least 3×10⁵ lymphoid lineage cells/ml.

Embodiment E70: The method of any one of Embodiments E66 to E69, wherein the method of manufacturing provides both CD14+ (>95% positive) macrophages and CD56^{bright}/CD16^{dim} NK cells.

Embodiment E71: The method of any one of Embodiments E66 to E70, wherein the method of manufacturing is amenable to scaling to clinically relevant doses.

Embodiment E72: The method of any one of Embodiments E1 to E71, wherein the population of isolated lymphoid lineage cells and the population of isolated myeloid lineage cells act synergistically to kill cancer cells.

Embodiment E73: The method of any one of Embodiments E1 to E72, wherein the administering is intravenous, intraarterial, intratumoral, or injected in the vicinity of a tumor.

Embodiment E74: The method of any one of Embodiments E1 to E73, wherein the cancer is a blood cancer.

Embodiment E75: The method of any one of Embodiments E1 to E74, wherein the cancer is a solid tumor.

Embodiment E76: The method of any one of Embodiments E1 to E75, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs' syndrome.

Embodiment E77: A plurality of compositions for use in the method of any one of Embodiments E1 to E76.

Embodiment E78: A method for killing a cancer cell or for inhibiting the proliferation of a cancer cell, the method comprising contacting the cancer cell with a population of isolated lymphoid lineage cells and a population of isolated myeloid lineage cells.

Embodiment E79: The method of Embodiment E78, wherein the cancer cell is contacted with the population of isolated lymphoid lineage cells and the population of isolated myeloid lineage cells simultaneously.

Embodiment E80: The method of Embodiment E78, wherein the cancer cell is contacted with the population of isolated lymphoid lineage cells before being contacted with the population of isolated myeloid lineage cells or the cancer cell is contacted with the population of isolated lymphoid lineage cells after being contacted with the population of isolated myeloid lineage cells.

Embodiment E81: The method of any one of Embodiments E78 to E80, wherein one or more of the isolated lymphoid lineage cells and/or one or more of the isolated myeloid lineage cells comprise a genomic modification that expresses a chimeric antigen receptor (CAR).

Embodiment E82: The method of Embodiment E81, wherein the CAR comprises an antigen binding region that binds to one or more antigens expressed by a cancer cell.

Embodiment E83: The method of Embodiment E82, wherein the antigen binding region binds to one or more tumor antigens.

Embodiment E84: The method of any one of Embodiments E81 to E83, wherein the CAR comprises an antigen binding region that binds to ROR1.

Embodiment E85: The method of any one of Embodiments E78 to E84, wherein one or more of the isolated lymphoid lineage cells and/or one or more of the isolated myeloid lineage cells comprise a genomic modification that expresses or over expresses a cytokine.

Embodiment E86: The method of any one of Embodiments E78 to E85, wherein one or more of the isolated lymphoid lineage cells and/or one or more of the isolated myeloid lineage cells comprise a genomic modification which disrupts the beta-2-microglobulin (B2M) gene, optionally, wherein the cells express a fusion protein comprising a B2M polypeptide and an HLA polypeptide (e.g., an HLA-A, HLA-B, HLA-C, HLA-E, HLA-F and HLA-G polypeptide), e.g., the coding sequence for one or more of HLA class I histocompatibility antigen, alpha chains (HLAs).

Embodiment E87: The method of any one of Embodiments E78 to E86, wherein the isolated lymphoid lineage cells comprise cytotoxic lymphocytes.

Embodiment E88: The method of Embodiment E87, wherein the isolated lymphoid lineage cells comprising cytotoxic lymphocytes are enriched for CD56+ cells, for CD16+ cells, NKG2D+ cells, CD226+ Cells, NKp46+ cells, NKp44+ cells, CD244+ cells, and/or CD94+ cells.

Embodiment E89: The method of Embodiment E87 or Embodiment E88, wherein the cytotoxic lymphocyte targets and kills cancer cells.

Embodiment E90: The method of Embodiment E89, wherein the cytotoxic lymphocyte targets and kills cancer cells without requiring IL-15 and/or without requiring IL-2 activation.

Embodiment E91: The method of any one of Embodiments E87 to E90, wherein the cytotoxic lymphocyte has reduced cytotoxicity to an NK-resistant cancer cell.

Embodiment E92: The method of any one of Embodiments E87 to E91, wherein the cytotoxic lymphocyte is a Natural Killer (NK) cell or is a cytotoxic T cell.

Embodiment E93: The method of Embodiment E92, wherein the NK cell is a mature NK cell.

Embodiment E94: The method of any one of Embodiments E87 to E90, wherein the cytotoxic lymphocyte is a Natural killer T (NKT) cell.

Embodiment E95: The method of any one of Embodiments E92 to E94, wherein the NK cell expresses CD16a and/or the NK cell does not express CD3.

Embodiment E96: The method of any one of Embodiments E92 to E95, wherein the NK cell is CD56bright CD16dim/−.

Embodiment E97: The method of any one of Embodiments E92 to E96, wherein the NK cell secretes one or more cytokines selected from interferon-gamma (IFNγ), tumor necrosis factor-alpha (TNFα), tumor necrosis factor-beta (TNFβ), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b).

Embodiment E98: The method of any one of Embodiments E87 to E97, wherein the cytotoxic lymphocyte is a delta-gamma T cell.

Embodiment E99: The method of any one of Embodiments E87 to E98, wherein the cytotoxic lymphocyte is further engineered to express a chimeric antigen receptor (CAR).

Embodiment E100: The method of any one of Embodiments E87 to E99, wherein the cytotoxic lymphocyte is further engineered to express or overexpress a cytokine.

Embodiment E101: The method of any one of Embodiments E78 to E100, wherein the isolated myeloid lineage cells comprise a megakaryocyte, erythrocyte, mast cell, myeloblast, dendritic cell, basophil, neutrophil, eosinophil, monocyte, or macrophage.

Embodiment E102: The method of any one of Embodiments E78 to E101, wherein the isolated myeloid lineage cells express one or more of CD11b, CD13, CD14, CD33, CD45, CD80, CD163, CD206, and SIRPα, e.g., in amounts that are similar to amounts expressed by PBMC-derived cells.

Embodiment E103: The method of any one of Embodiments E78 to E102, wherein the isolated myeloid lineage cells have increased expression of CD80 and/or CD206, which is indicative of an activated state.

Embodiment E104: The method of any one of Embodiments E78 to E103, wherein the isolated myeloid lineage cell is a macrophage.

Embodiment E105: The method of Embodiment E104, wherein the macrophage expresses one or more of CD11b, CD68, CD80, CD86, CD163, CD206, and SIRPα in amounts that are similar to amounts expressed by PBMC-derived cells and/or secretes one or more of TNFα, IL-12p70, and IL-10 in amounts that are similar to amounts expressed by PBMC-derived cells.

Embodiment E106: The method of Embodiment E104 or Embodiment E105, wherein the macrophage expresses one or more of CD34, CD44, CD45, CD73, and CD90.

Embodiment E107: The method of any one of Embodiments E104 to E106, wherein the macrophages comprise M1 macrophages and/or M2 macrophages.

Embodiment E108: The method of Embodiment E107, wherein the M1 macrophages and/or M2 macrophages secrete one or more of TNFα, IL-12p70, and IL-10 in amounts that are similar to amounts expressed by PBMC-derived cells.

Embodiment E109: The method of any one of Embodiments E78 to E108, wherein the isolated myeloid lineage cells kill cancer cells and/or promote cancer cell killing by cytotoxic lymphocytes.

Embodiment E110: The method of any one of Embodiments E78 to E109, wherein the isolated myeloid lineage cell is further engineered to express a chimeric antigen receptor (CAR).

Embodiment E111: The method of any one of Embodiments E78 to E110, wherein the isolated myeloid lineage cell is further engineered to express or overexpress a cytokine.

Embodiment E112: The method of any one of Embodiments E78 to E111, wherein the isolated lymphoid lineage cells and the isolated myeloid lineage cells are manufactured by a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor.

Embodiment E113: The method of Embodiment E112, when CD34+ cells are selected, the embryoid bodies are first chemically and/or mechanically dissociated.

Embodiment E114: The method of Embodiment E112 or Embodiment E113, wherein the stem cell is an induced pluripotent stem (iPSC).

Embodiment E115: The method of Embodiment E114, wherein the isolated lymphoid lineage cell and the isolated myeloid lineage cell are derived from the same iPSC.

Embodiment E116: The method of Embodiment E114 or Embodiment E115, wherein the iPSC comprises a genomic modification that expresses a chimeric antigen receptor (CAR).

Embodiment E117: The method of any one of Embodiments E114 to E116, wherein the iPSC comprises a genomic modification that expresses or over expresses a cytokine.

Embodiment E118: The method of any one of Embodiments E114 to E117, wherein the iPSC comprises a genomic modification which disrupts the beta-2-microglobulin (B2M) gene.

Embodiment E119: The method of Embodiment E118, wherein the iPSC comprises a biallelic disruption in a B2M gene.

Embodiment E120: The method of Embodiment E118 or Embodiment E119, wherein the iPSC expresses a fusion protein comprising a B2M polypeptide and an HLA polypeptide (e.g., an HLA-A, HLA-B, HLA-C, HLA-E, HLA-F and HLA-G polypeptide) e.g., the coding sequence for one or more of HLA class I histocompatibility antigen, alpha chains (HLAs).

Embodiment E121: The method of any one of Embodiments E114 to E120, wherein the iPSC was reprogrammed from a somatic cell comprising contacting the somatic cell with one or more ribonucleic acids (RNAs), wherein each RNA encodes one or more reprogramming factors.

Embodiment E122: The method of Embodiment E121, wherein the one or more reprogramming factors are selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

Embodiment E123: The method of Embodiment E121 or Embodiment E122, wherein the somatic cell is selected from fibroblasts, keratinocytes, melanocyte blood cells, bone marrow cells, adipose cells, and tissue-resident progenitor cells.

Embodiment E124: The method of any one of Embodiments E77 to E123, wherein the contacting is in vitro.

Embodiment E125: The method of any one of Embodiments E77 to E123, wherein the contacting is in vivo.

Embodiment E126: A plurality of compositions for use in the method of any one of Embodiments E78 to E125.

Embodiment E127: A method for manufacturing a plurality of population of cells comprising a population of isolated lymphoid lineage cells and a population of isolated myeloid lineage cells for treating a cancer, for killing a cancer cell, and/or for inhibiting the proliferation of a cancer cell, the method a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor.

Embodiment E128: The method of Embodiment E127, when CD34+ cells are selected, the embryoid bodies are first chemically and/or mechanically dissociated.

Embodiment E129: The method of Embodiment E127 or Embodiment E128, wherein the stem cell is an induced pluripotent stem (iPSC).

Embodiment E130: The method of Embodiment E129, wherein the isolated lymphoid lineage cell and the isolated myeloid lineage cell are derived from the same iPSC.

Embodiment E131: The method of Embodiment E129 or Embodiment E130, wherein the iPSC comprises a genomic modification that expresses a chimeric antigen receptor (CAR).

Embodiment E132: The method of any one of Embodiments E129 to E131, wherein the iPSC comprises a genomic modification that expresses or over expresses a cytokine.

Embodiment E133: The method of any one of Embodiments E129 to E132, wherein the iPSC comprises a genomic modification which disrupts the beta-2-microglobulin (B2M) gene.

Embodiment E134: The method of Embodiment E133, wherein the iPSC comprises a biallelic disruption in a B2M gene.

Embodiment E135: The method of Embodiment E133 or Embodiment E134, wherein the iPSC expresses a fusion protein comprising a B2M polypeptide and an HLA polypeptide (e.g., an HLA-A, HLA-B, HLA-C, HLA-E, HLA-F and HLA-G polypeptide) e.g., the coding sequence for one or more of HLA class I histocompatibility antigen, alpha chains (HLAs).

Embodiment E136: The method of any one of Embodiments E129 to E135, wherein the iPSC was reprogrammed from a somatic cell comprising contacting the somatic cell with one or more ribonucleic acids (RNAs), wherein each RNA encodes one or more reprogramming factors.

Embodiment E137: The method of Embodiment E136, wherein the one or more reprogramming factors are selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

Embodiment E138: The method of Embodiment E136 or Embodiment E137, wherein the somatic cell is selected from fibroblasts, keratinocytes, melanocyte blood cells, bone marrow cells, adipose cells, and tissue-resident progenitor cells.

Embodiment E139: The method of any one of Embodiments E127 to E138, wherein one or more of the isolated lymphoid lineage cells and/or one or more of the isolated myeloid lineage cells comprise a genomic modification that expresses a chimeric antigen receptor (CAR).

Embodiment E140: The method of Embodiment E139, wherein the CAR comprises an antigen binding region that binds to one or more antigens expressed by a cancer cell.

Embodiment E141: The method of Embodiment E140, wherein the antigen binding region binds to one or more tumor antigens.

Embodiment E142: The method of any one of Embodiments E139 to E141, wherein the CAR comprises an antigen binding region that binds to ROR1.

Embodiment E143: The method of any one of Embodiments E127 to E142, wherein one or more of the isolated lymphoid lineage cells and/or one or more of the isolated myeloid lineage cells comprise a genomic modification that expresses or over expresses a cytokine.

Embodiment E144: The method of any one of Embodiments E127 to E143, wherein one or more of the isolated lymphoid lineage cells and/or one or more of the isolated myeloid lineage cells comprise a genomic modification which disrupts the beta-2-microglobulin (B2M) gene.

Embodiment E145: The method of Embodiment E144, wherein the cells express a fusion protein comprising a B2M polypeptide and an HLA polypeptide (e.g., an HLA-A, HLA-B, HLA-C, HLA-E, HLA-F and HLA-G polypeptide) e.g., the coding sequence for one or more of HLA class I histocompatibility antigen, alpha chains (HLAs).

Embodiment E146: The method of any one of Embodiments E127 to E145, wherein the isolated lymphoid lineage cells comprise cytotoxic lymphocytes.

Embodiment E147: The method of Embodiment E146, wherein the isolated lymphoid lineage cells comprising cytotoxic lymphocytes are enriched for CD56+ cells, for CD16+ cells, NKG2D+ cells, CD226+ Cells, NKp46+ cells, NKp44+ cells, CD244+ cells, and/or CD94+ cells.

Embodiment E148: The method of Embodiment E146 or Embodiment E147, wherein the cytotoxic lymphocyte targets and kills cancer cells.

Embodiment E149: The method of Embodiment E148, wherein the cytotoxic lymphocyte targets and kills cancer cells without requiring IL-15 and/or without requiring IL-2 activation.

Embodiment E150: The method of any one of Embodiments E146 to E149, wherein the cytotoxic lymphocyte has reduced cytotoxicity to an NK-resistant cancer cell.

Embodiment E151: The method of any one of Embodiments E146 to E150, wherein the cytotoxic lymphocyte is a Natural Killer (NK) cell or is a cytotoxic T cell.

Embodiment E152: The method of Embodiment E151, wherein the NK cell is a mature NK cell.

Embodiment E153: The method of any one of Embodiments E146 to E152, wherein the cytotoxic lymphocyte is a Natural killer T (NKT) cell.

Embodiment E154: The method of any one of Embodiments E151 to E153, wherein the NK cell expresses CD16a and/or the NK cell does not express CD3.

Embodiment E155: The method of any one of Embodiments E151 to E154, wherein the NK cell is CD56bright CD16dim/−.

Embodiment E156: The method of any one of Embodiments E151 to E155, wherein the NK cell secretes one or more cytokines selected from interferon-gamma (IFNγ), tumor necrosis factor-alpha (TNFα), tumor necrosis factor-beta (TNFβ), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b).

Embodiment E157: The method of any one of Embodiments E146 to E156, wherein the cytotoxic lymphocyte is a delta-gamma T cell.

Embodiment E158: The method of any one of Embodiments E146 to E157, wherein the cytotoxic lymphocyte is further engineered to express a chimeric antigen receptor (CAR).

Embodiment E159: The method of any one of Embodiments E146 to E158, wherein the cytotoxic lymphocyte is further engineered to express or overexpress a cytokine.

Embodiment E160: The method of any one of Embodiments E127 to E159, wherein the isolated myeloid lineage cells comprise a megakaryocyte, erythrocyte, mast cell, myeloblast, dendritic cell, basophil, neutrophil, eosinophil, monocyte, or macrophage.

Embodiment E161: The method of any one of Embodiments E127 to E160, wherein the isolated myeloid lineage cells express one or more of CD11b, CD13, CD14, CD33, CD45, CD80, CD163, CD206, and SIRPα, e.g., in amounts that are similar to amounts expressed by PBMC-derived cells.

Embodiment E162: The method of any one of Embodiments E127 to E161, wherein the isolated myeloid lineage cells have increased expression of CD80 and/or CD206, which is indicative of an activated state.

Embodiment E163: The method of any one of Embodiments E127 to E162, wherein the isolated myeloid lineage cell is a macrophage.

Embodiment E164: The method of Embodiment E163, wherein the macrophage expresses one or more of CD11b, CD68, CD80, CD86, CD163, CD206, and SIRPα in amounts that are similar to amounts expressed by PBMC-derived cells and/or secretes one or more of TNFα, IL-12p70, and IL-10 in amounts that are similar to amounts expressed by PBMC-derived cells.

Embodiment E165: The method of Embodiment E163 or Embodiment E164, wherein the macrophage expresses one or more of CD34, CD44, CD45, CD73, and CD90.

Embodiment E166: The method of any one of Embodiments E163 to E165, wherein the method further comprises a step of differentiating the macrophages into M1 and/or M2 macrophages, e.g., by exposure to MCSF.

Embodiment E167: The method of Embodiment E166, wherein the method further comprises a step of polarizing the M1 macrophages with interferon gamma (IFN-γ) and/or lipopolysaccharide (LPS) and/or treating the M2 macrophages with IL-4.

Embodiment E168: The method of any one of Embodiments E163 to E167, wherein the macrophages comprise M1 macrophages and/or M2 macrophages.

Embodiment E169: The method of Embodiment E168, wherein the M1 macrophages and/or M2 macrophages secrete one or more of TNFα, IL-12p70, and IL-10 in amounts that are similar to amounts expressed by PBMC-derived cells.

Embodiment E170: The method of any one of Embodiments E127 to E169, wherein the isolated myeloid lineage cells kill cancer cells and/or promote cancer cell killing by cytotoxic lymphocytes.

Embodiment E171: The method of any one of Embodiments E127 to E170, wherein the isolated myeloid lineage cell is further engineered to express a chimeric antigen receptor (CAR).

Embodiment E172: The method of any one of Embodiments E127 to E171, wherein the isolated myeloid lineage cell is further engineered to express or overexpress a cytokine.

Embodiment E173: The method of any one of Embodiments E136 to E172, wherein the iPSC was contacted with resveratrol before reprogramming.

Embodiment E174: The method of any one of Embodiments E127 to E173, one or more culturing steps comprise a medium which is serum-free culture medium and/or feeder-free culture medium, e.g., an mTeSR™ medium or a StemDiff™ NK medium.

Embodiment E175: The method of any one of Embodiments E127 to E174, wherein the adherent culturing vessel is a multi-well plate or a cell culturing flask.

Embodiment E176: The method of any one of Embodiments E127 to E175, wherein the wherein the method of manufacturing provides both CD14+ (>95% positive) macrophages and CD56^{bright}/CD16^{dim} NK cells.

Embodiment E177: The method of any one of Embodiments E127 to E176, wherein the method of manufacturing provides at least 1×10⁶ myeloid lineage cells/ml and at least 3×10⁵ lymphoid lineage cells/ml.

Embodiment E178: The method of any one of Embodiments E127 to E177, wherein the method of manufacturing is amenable to scaling to clinically relevant doses.

Embodiment E179: The method of any one of Embodiments E127 to E178, wherein the cancer is a blood cancer or is a solid tumor.

Embodiment E180: The method of any one of Embodiments E127 to E179, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs' syndrome.

Embodiment E181: A plurality of compositions manufactured by the method of any one of Embodiments E127 to E180.

Embodiment E182: A method for manufacturing a population of cells that is enriched for cytotoxic lymphocytes, the method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes; wherein steps (5) and (6) occur in an adherent culturing vessel.

Embodiment E183: A method for producing macrophages from an induced a pluripotent stem cell (iPSC), the method comprising steps of: (1) obtaining an iPSC; (2) culturing the iPSC in a first medium for about three days; (3) culturing the iPSC in a second for about four days; (4) culturing the iPSC in a monocyte differentiating medium for at least seven days, thereby obtaining monocytes; (5) isolating the monocytes; (6) culturing the monocytes for about four days; (7) culturing the monocytes in the presence of M-CSF for three to four days, thereby obtaining macrophages; and (8) harvesting the macrophages.

Embodiment E184: A method for decreasing synthetic oligodeoxynucleotides (ODNs)-related activation of pro-inflammatory responses, the method comprising transfecting a cell with an ODN comprising a TTAGGG motif.

Embodiment E185: A method for enhancing the efficiency of gene-editing, the method comprising contacting a cell with resveratrol before gene-editing.

Embodiment E186: A method for enhancing the efficiency of gene-editing, the method comprising contacting a cell that has been gene-edited with resveratrol.

Embodiment E187: A composition comprising an isolated cytotoxic lymphocyte comprising a genetically engineered disruption in a beta-2-microglobulin (B2M) gene, wherein the cytotoxic lymphocyte is selected from a lymphoid cell or myeloid cell or a mesenchymal stromal/stem cell, or a hematopoietic stem cell.

Embodiment E188: A method of making an engineered cytotoxic lymphocyte, comprising steps of: (1) reprogramming a somatic cell to an iPS cell, the reprogramming comprising contacting the iPS cell with a ribonucleic acid (RNA) encoding one or more reprogramming factors; (2) disrupting a B2M gene in the iPS cell, the disrupting comprising gene-editing the cell by contacting the cell with RNA encoding one or more gene-editing proteins (e.g., a temperature-sensitive gene-editing protein); and (3) differentiating the iPS cell into a cytotoxic lymphocyte; wherein the cytotoxic lymphocyte is selected from a lymphoid cell or myeloid cell.

Embodiment E189: A method of treating cancer comprising steps of: obtaining an isolated cytotoxic lymphocyte comprising a genetically engineered disruption in a B2M gene and administering the isolated cytotoxic lymphocyte to a subject in need thereof; wherein the cytotoxic lymphocyte is a lymphoid cell or a CAR-myeloid cell or CAR-mesenchymal stromal/stem cell.

Embodiment E190: A composition comprising an isolated cytotoxic lymphocyte comprising a gene edit in a CD16a gene, wherein the cytotoxic lymphocyte is selected from a lymphoid cell or myeloid cell.

Embodiment E191: A method for screening constructs capable of being expressed in an in vivo cell and for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; and (3) identifying cells that have been transformed and capable of expressing the fusion protein for the ability of its fusion protein to recognize and/or bind to a cancer cell.

Embodiment E192: A method for screening constructs capable of being expressed in an ex vivo cell and for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) culturing the cell capable of expressing the fusion protein which recognizes and/or binds to a cancer cell until a therapeutic amount of the cell is manufactured.

Embodiment E193: A method for screening constructs capable of being expressed in an ex vivo cell and for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) contacting an ex vivo cell with the synthetic mRNA encoding the gene-editing protein and the repair template encoding the fusion protein which was identified in step (3) as having the ability recognize and/or bind to a cancer cell; and (5) culturing the cell of step (4) until a therapeutic amount of the cell is manufactured.

Embodiment E194: A method for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; and (4) administering to a subject in need the synthetic mRNA encoding the gene-editing protein and the repair template encoding the fusion protein which has the ability recognize and/or bind to a cancer cell.

Embodiment E195: A method for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) culturing the cell capable of expressing the fusion protein which recognizes and/or binds to a cancer cell until a therapeutic amount of the cell is manufactured; and (5) administering a therapeutically-effective amount of the cells of step (4) to a subject in need.

Embodiment E196: A method for treating a cancer, the method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) contacting an ex vivo cell with the synthetic mRNA encoding the gene-editing protein and the repair template encoding the fusion protein which was identified in step (3) as having the ability recognize and/or bind to a cancer cell; (5) culturing the cell of step (4) until a therapeutic amount of the cell is manufactured; and (6) administering a therapeutically-effective amount of the cells of step (4) to a subject in need.

Any aspect or embodiment herein may be combined with any other aspect or embodiment as disclosed herein.

Embodiment E197: A pharmaceutical composition comprising an isolated myeloid cell of any herein-disclosed aspect or embodiment, e.g., a macrophage.

Embodiment E198: A pharmaceutical composition comprising an isolated mesenchymal stromal/stem cell of any herein-disclosed aspect or embodiment.

This invention is further illustrated by the following non-limiting examples.

### EXAMPLES

Autologous engineered cell therapies such as autologous chimeric antigen receptor T-cell (CAR-T) therapies have revolutionized the treatment of hematologic cancers, however they are limited by manufacturing time and variability, the requirement for lymphodepletion, and side effects related to cytokine release. Allogeneic cell therapies derived from gene-edited induced pluripotent stem cells (iPSCs) are being developed to address the challenges associated with autologous engineered cell therapies. These “off-the-shelf” cell therapies contain specific edits designed to reduce immune rejection and to confer enhanced therapeutic properties and greater safety. However, efficient, footprint-free, biallelic targeting of defined loci in iPSCs remains technically challenging with current gene-editing approaches.

Further, while induced pluripotent stem cells (iPSCs) readily differentiate into a wide variety of cell types both in vitro and in vivo, the development of directed differentiation protocols that reliably yield pure populations of functional cells has proved challenging, in particular when differentiating into cell of the lymphoid or myeloid lineage. Generating functional cytotoxic lymphocytes from iPSCs is of particular interest to support the development of off-the-shelf engineered cell therapies for immune-oncology applications.

What is needed is improved compositions and methods for generating cellular therapies that can be engineered and produced in a practical manner.

### Example 1: Cell Preparation Methods and Results

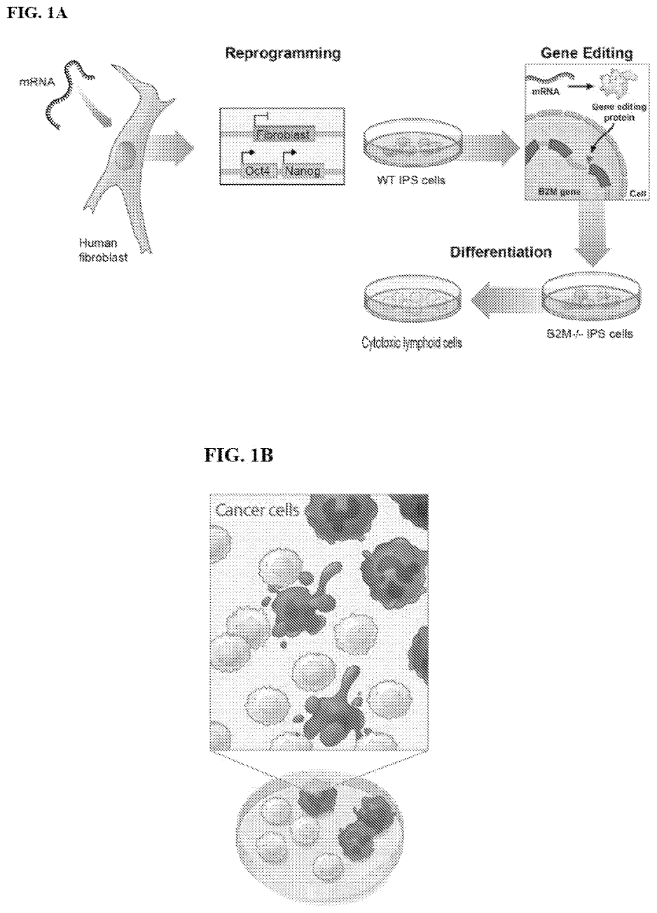

FIG. 1A shows schematic of cellular production methods used in this Example. mRNA-based cellular reprogramming (fibroblast to iPS cell) and gene-editing (beta-2-microglobulin (B2M) knockout) were employed. Further, edited cells were differentiated into cytotoxic lymphoid cells. FIG. 1B illustrates the differentiated cytotoxic lymphoid cells killing cancer cells.

Fibroblast cells were obtained from a human subject and reprogrammed to iPS cells using an mRNA-based reprogramming.

Efficient targeting of defined loci in iPSCs using messenger RNA (mRNA)-encoded gene-editing endonucleases comprising DNA-binding domains containing novel linker region was undertaken (e.g., a gene-editing protein comprising a DNA binding domain having at least one repeat of LTPEQVVAIAS*RVD*GGKQALETVQRLLPVLCQAGHGG (SEQ ID NO: 65; the “*RVD*” corresponds to the dinucleotide “xy” of SEQ ID NO: 22). Exon 3 of B2M, a key component of MHC class I molecules was targeted, and confirmed targeted editing in 10/12 lines, with 6/12 lines containing a desired biallelic deletion. Gene knockout in iPSCs was confirmed via RT-PCR and immunofluorescence in the context of B2M upregulation following exposure to interferon-γ.

More specifically, the following beta-2-microglobulin (B2M) gene was targeted:

(SEQ ID NO: 80)
AGAAATGAACTTTGAAAAGTATCTTGGGGCCAAATCATGTAGACTCTTGA
GTGATGTGTTAAGGAATGCTATGAGTGCTGAGAGGGCATCAGAAGTCCTT
GAGAGCCTCCAGAGAAAGGCTCTTAAAAATGCAGCGCAATCTCCAGTGA
CAGAAGATACTGCTAGAAATCTGCTAGAAAAAAAACAAAAAAGGCATGT
ATAGAGGAATTATGAGGGAAAGATACCAAGTCACGGTTTATTCTTCAAAA
TGGAGGTGGCTTGTTGGGAAGGTGGAAGCTCATTTGGCCAGAGTGGAA
ATGGAATTGGGAGAAATCGATGACCAAATGTAAACACTTGGTGCCTGATA
TAGCTTGACACCAAGTTAGCCCCAAGTGAAATACCCTGGCAATATTAATG
TGTCTTTTCCCGATATTCCTCAGGTACTCCAAAGATTCAGGTTTACTCA
CGTCATCCAGCAGAGAATGGAAAGTCAAATTTCCTGAATTGCTATGT
GTCTGGGTTTCATCCATCCGACATTGAAGTTGACTTACTGAAGAATGGA
GAGAGAATTGAAAAAGTGGAGCATTCAGACTTGTCTTTCAGCAAGGA
CTGGTCTTTCTATCTCTTGTACTACACTGAATTCACCCCCACTGAAA

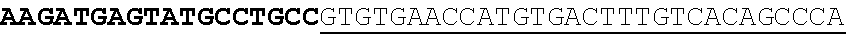

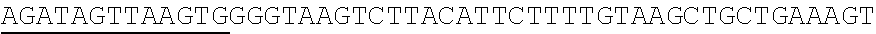

TGTGTATGAGTAGTCATATCATAAAGCTGCTTTGATATAAAAAAGGTCTAT
GGCCATACTACCCTGAATGAGTCCCA

and the following primers used: B2M_Fwd: CAGAGAAAGGCTCTTAAAAATGCAGCGCAATCTCCAG (SEQ ID NO: 81) and B2M_Rev: CACTTAACTATCTTGGGCTGTGACAAAGTCACATGGTTCACAC (SEQ ID NO: 82) and B2M_Rev_RC: GTGTGAACCATGTGACTTTGTCACAGCCCAAGATAGTTAAGTG (SEQ ID NO: 83). See FIG. 2.

In the above sequence, from top to bottom, the sequence features:
Bold: Exon.
Unmarked: Intron
Underlined (single): Left gene-editing protein binding site.
Underlined (double): Right gene-editing-protein binding site.
Large letters. Separation region between gene-editing-protein binding sites/cut region.
Underlined (dotted): Amplification primer binding sites.

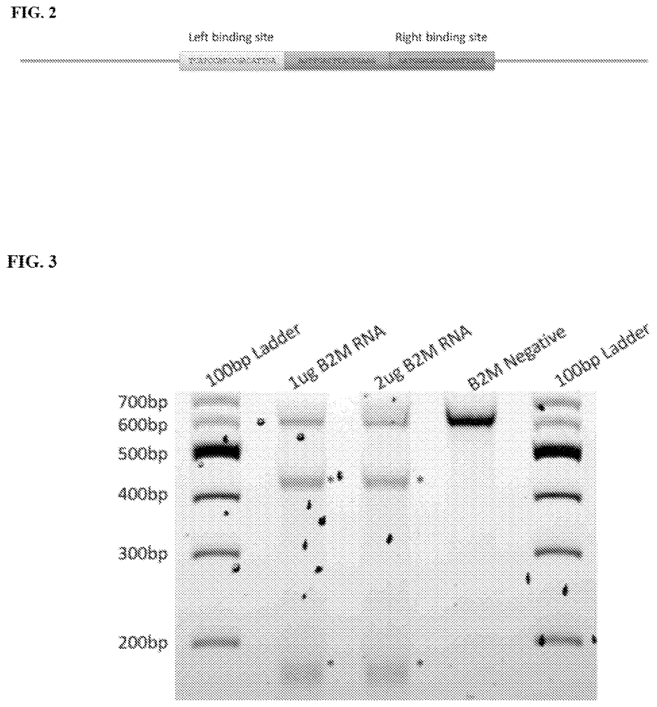

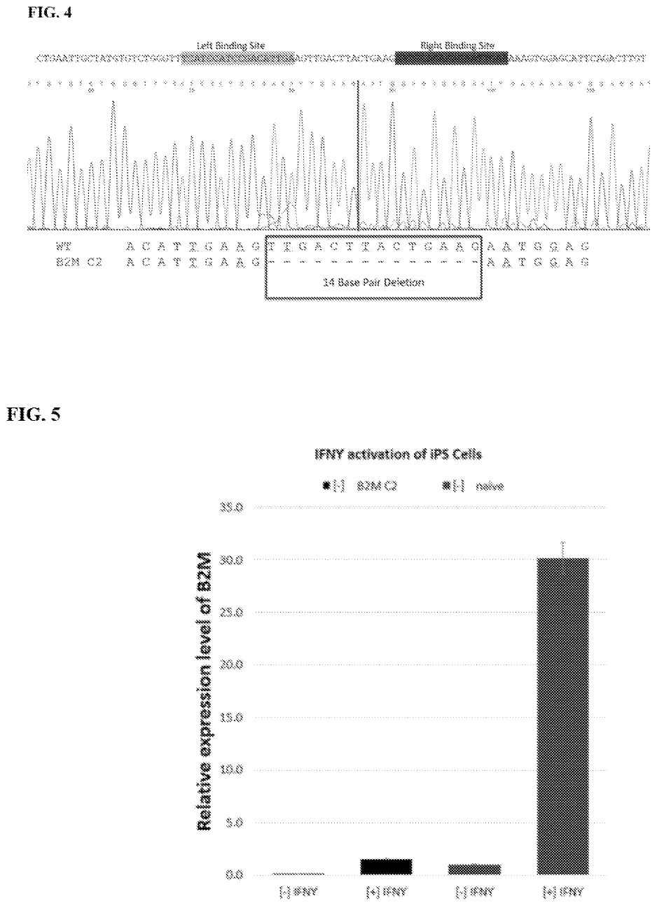

FIG. 3 shows successful gene-editing. 1.2×10⁵ iPSCs were electroporated and plated in conditioned media on a 24-well plate coated with rhLaminin-521 and grown for 48 hours. Cells were passaged into a 6-well plate coated with rhLaminin-521. Cells were cultured for an additional 4 days. Cells were split between genomic DNA extraction and 2.0×10⁴ cells were seeded into a well of a 6-well plate coated with rhLaminin-521. Amplicon length of B2M is 587 bp and edited band 1 was 416 bp (see “*”) and edited band 2 was 171 bp (see “*”). Sequencing confirmed a 14 base pair deletion in B2M (see FIG. 4).

FIG. 5 shows RNA levels of B2M with or without IFN gamma activation (“IFNY”; two left bars are the B2M knockout, and the two right bars are naïve cells). 1.5×10⁴ iPSCs were plated in conditioned media on a 24-well plate coated with rhLaminin-521 and grown for 24 hours. The media was then replaced with conditioned media with or without IFN-gamma at a concentration of 25 ng/mL (t=0). Daily media changes were performed until cells were harvested at t=72 hours. RNA was extracted, quantified, and normalized for RT-qPCR analysis. The housekeeping gene was GAPDH and the tested gene was B2M.

A scalable 3D culture system for directed differentiation of human iPSCs into functional NK cells was developed. The process involved a short, embryoid body-bases hematopoietic commitment step performed either in micro-patterned wells or, in a scaled-version of the process, in multi-layer culture vessels (StemDiff™ T/NK Cell Kit->StemSpan T/NK differentiation kit). Hematopoietic commitment was followed by outgrowth, enrichment of CD34+ cells, and differentiation into a CD5+/CD7+ common lymphoid progenitor. The process then proceeded through a either a 14-day NK cell differentiation phase to yield functional CD56dim/CD16+ NK cells or along a 22-day T cell differentiation phase to yield CD8+ T cells.

The following table outlines the cells generated:

| Culture Format | | Number of Cells into | Cell Counts |
|---|---|---|---|
| At CD34+ HSCP | Type | NK Differentiation | harvested |
| 3D | wild type | 1.25 × 10⁵ | 1.0 × 10³ |
| | B2M−/− | 6.25 × 10⁵ | 5.77 × 10⁵ |

This table also shows that, compared to wild type, B2M knockout cells proliferated robustly. The B2M−/−iPSC line with a 14-bp deletion at the target site (shown in FIG. 4) exhibited an increased proliferation rate both as iPSCs and during differentiation along a lymphoid lineage when compared to a wild-type iPSC line.

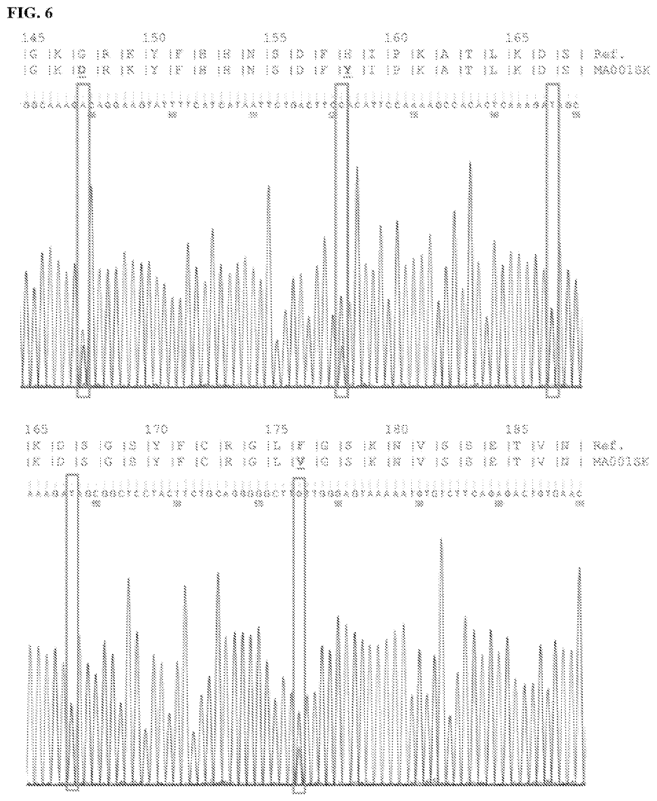

Further, resultant NK cells, as an illustrative differentiated cell type, were characterized for CD16a (UniProtKB P08637 (FCG3A HUMAN)) and determined to be heterozygous at G147D dbSNP:rs443082, Y158H dbSNP:rs396716, and F176V dbSNP:rs396991. F176V dbSNP:rs396991 shows a higher binding capacity of IgG1, IgG3 and IgG4. See FIG. 6. gDNA was amplified with 2 step PCR: Kapa HiFi HotStart (35×/64° C. extension), primers were F: CTGATCTAGAACTTACTGTGAATCCTTGTCACCTGCCAC (SEQ ID NO: 84) and R: GATAAGAAGGAGGCCAGCACGATAGGAACATATGACAC (SEQ ID NO: 85).

This Example, inter alia, demonstrates a scalable 3D process for the differentiation of both wild-types and engineered iPSCs into functional NK cells, as an illustrative differentiated cell type. The 3D process described herein is also useful for differentiation of both wild-types and engineered iPSCs into other functional cytotoxic lymphocytes of the lymphoid lineage, including but not limited to T cells, e.g., a cytotoxic T cells or gamma-delta T cells; NK-T cells; and other functional immune cells of the myeloid lineage, including macrophages, e.g., M1 macrophage or M2 macrophages. This process supports the development of next-generation cell therapies for immuno-oncology applications.

The methods disclosed herein are enhanced when transfected RNA is associated with one or more lipid selected from Table A and/or Formulae I-XVI.

### Example 2: Cells Characterization Methods and Results

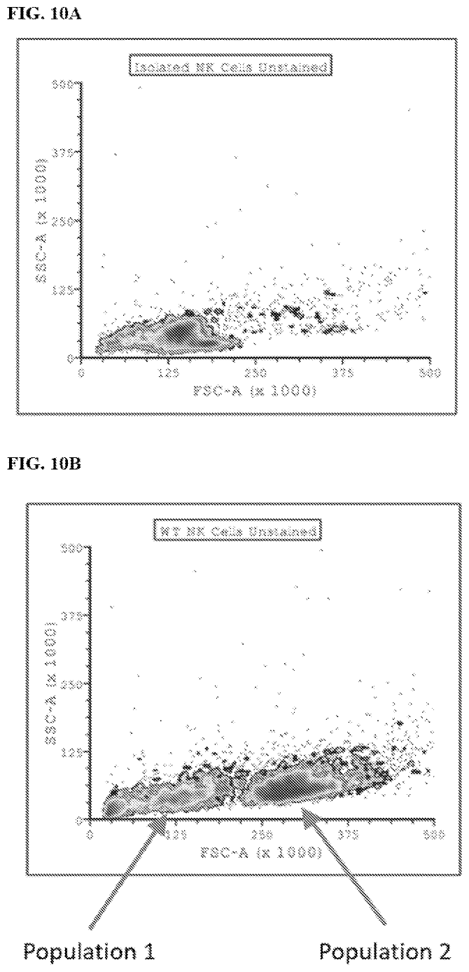

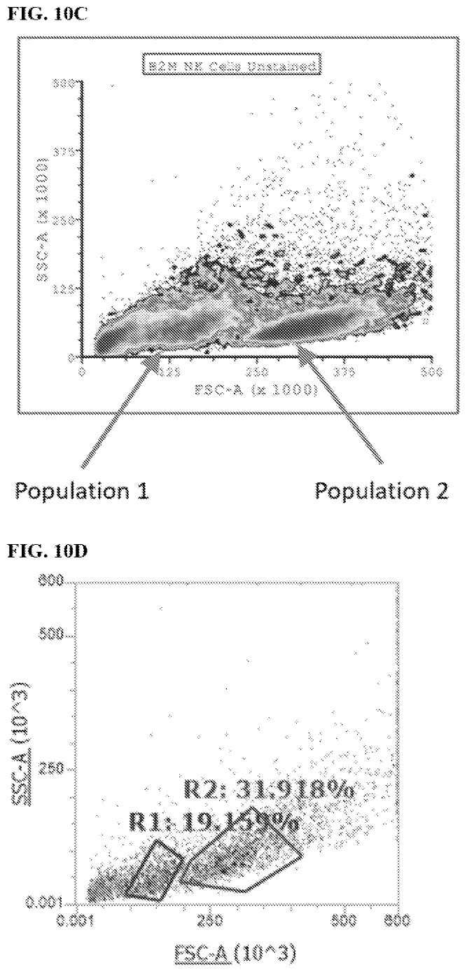

Phenotypical and functional characterization assays were used to evaluate cells of Example 1. Phenotypical characterization used flow cytometry staining of surface markers, specifically CD56/CD16 (e.g., gated on CD56). CD56/NKG2D, CD56/CD45, CD56/CD3, CD56/CD244, CD56/CD94/NKG2A, CD56/NKp46, CD56/NKp44, CD56/KIRs, CD56/TRAIL, and CD56/FASL were also assessed (e.g., gated on CD56). See, FIG. 10A to FIG. 10C and the below tables:

Data in this first table characterize PBMC-Isolated NK Cells vs. iPSC-Derived NK Cells from suspension round 1:

| | | WT | | B2M^{-/-} | |
|---|---|---|---|---|---|
| | Isolated | Population | Population | Population | Population |
| | NK Cells | 1 | 2 | 1 | 2 |
| % of | | 25.6% | 74.4% | 32.9% | 67.1% |
| Population | | | | | |
| CD56 | 77.5% | 82.1% | 22.3% | 84.9% | 68.2% |
| CD16 | 16.3% | 2.06% | 0.25% | 8.23% | 21.3% |
| CD3 | 28.0% | 76.1% | 20.0% | 61.5% | 22.9% |
| CD45 | 97.8% | 99.1% | 99.4% | 99.8% | 99.8% |
| NKG2D | 9.17% | 20.9% | 59.3% | 17.0% | 43.8% |

Data in this second table characterize iPSC-Derived NK Cells—AggreWell™ vs. iPSC-Derived NK Cells from suspension round 2

| | AggreWell ™ | | | | Suspension | | | |
|---|---|---|---|---|---|---|---|---|
| | WT | | B2M^{-/-} | | WT | | B2M^{-/-} | |
| | Pop 1 | Pop 2 | Pop 1 | Pop 2 | Pop 1 | Pop 2 | Pop 1 | Pop 2 |
| CD244 | 63% | 94% | 77% | 94% | 50% | 92% | 62% | 89% |
| CD336 | 61% | 77% | 55% | 32% | 42% | 33% | 4% | 5% |
| CD3 | 70% | 89% | 65% | 14% | 65% | 44% | 46% | 14% |
| CD4 | 30% | 51% | 25% | 24% | 50% | 48% | 59% | 56% |
| TCRαβ | 1% | 9% | 49% | 9% | 6% | 3% | 55% | 49% |
| TCRγδ | 0% | 0% | 75% | 11% | 82% | 49% | 77% | 13% |

The illustrative B2M-edited, differentiated cell type, i.e., NK cells were CD45+, CD56+, CD16−, NKG2D−, KIR2DL4−, KIR2DL1−, and CD8−. See also, FIG. 10D.

Functional characterization involved measurement of cytotoxicity, activation, and evaluation of cytokine release assay, as well as a proliferation assay for an illustrative differentiated cell type, i.e., NK cells.

NK Cell cytotoxicity was measured using target cells loaded with calcein AM (a cell-permeant dye that is used to determine cell viability in most eukaryotic cells. In live cells the nonfluorescent calcein AM is converted to a green-fluorescent calcein after acetoxymethyl ester hydrolysis by intracellular esterase). Various effector (NK cell)-to-target (K-562 cell) ratios (E:T ratio) were tested to observe tumor killing. K-562 cells are a cancer cell line derived from a 53-year-old female with chronic myelogenous leukemia (CML) in terminal blast crises (greater than 30% immature cells in the bone marrow (BM), peripheral blood, a large focus of blasts in the BM, or presence of extramedullary infiltration with blast cells). These cells are commonly used for cytotoxicity assays as they lack the MHC complex required to inhibit NK activity. The experiments also include effector cells incubated with and without a cytokine cocktail (IL-15 and IL-2).

K-562 cells were loaded with calcein AM for 1 hour and washed with complete RPMI-1640 with 10% FBS prior to co-culture with NK cells. Cells were co-cultured for 18 hours with images taken every 30 minutes using the Operetta high content imager. Once a run had finished, the cell suspension was centrifuged, and the media harvested. The conditioned media was tested on a Luminex MAGPIX to detect and measure the concentration of IFNγ and TNFα. Cells were resuspended and stained for CD56/CD16 and CD56/CD1074a.

Activation was assessed by measuring activation marker CD107a via flow cytometry using methods as described herein and/or as well-known in the art.

For the cytokine release assay, after cytotoxicity measurements, media was harvested, and cytokine release was assayed (IFNγ and TNFα) using methods as described herein and/or as well-known in the art.

For proliferation, cells were incubated in the presence of activating cytokine IL-15 with media replacements occurring every three days. Every three days samples were pelleted washed, reseeded in fresh media containing the activating cytokine, and counted to trace the number of cells as well as viability over time. Cells were tracked over 28 days. During this experiment, media was saved for cytokine evaluation through a Luminex immune panel.

The remaining cells from this handling were seeded onto on untreated 96-well plate at a known cell count and images, cell counts and media changes with IL-15 occurred every 3 days for 28 days. NK-92 cells are an interleukin-2 (IL-2) dependent natural killer cell line derived from peripheral blood mononuclear cells (PBMCs) from a 50-year-old Caucasian male with rapidly progressive non-Hodgkin's lymphoma. NK-92 cells are used as a control cell for NK cytotoxicity experiments to demonstrate the functional killing of tumor cells. NK-92 cells are used herein in the cytotoxicity assay with calcein AM. When NK cells engage, they secrete cytokines and histone into the media. Histones are highly involved in inflammation and coagulation mechanisms known as “immunothrombosis” occur, which are observable as cell “clumping”.

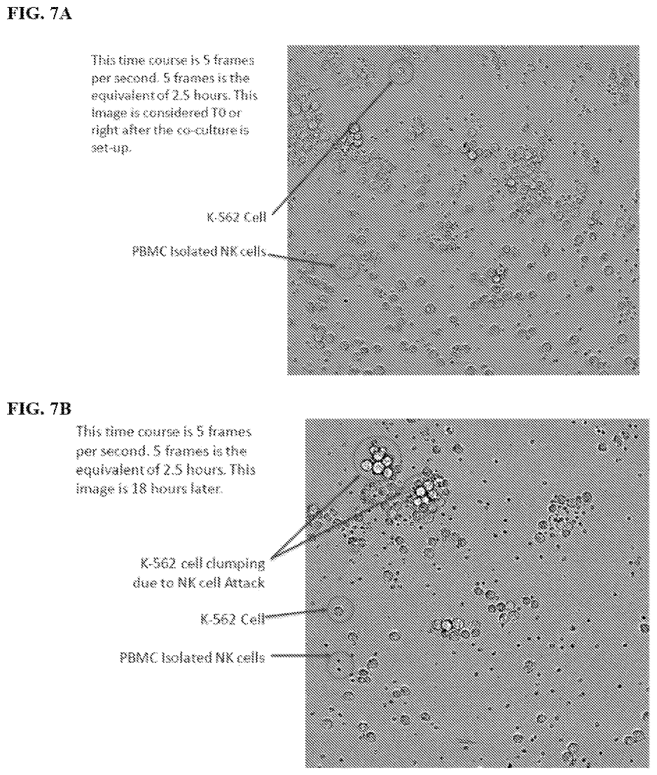

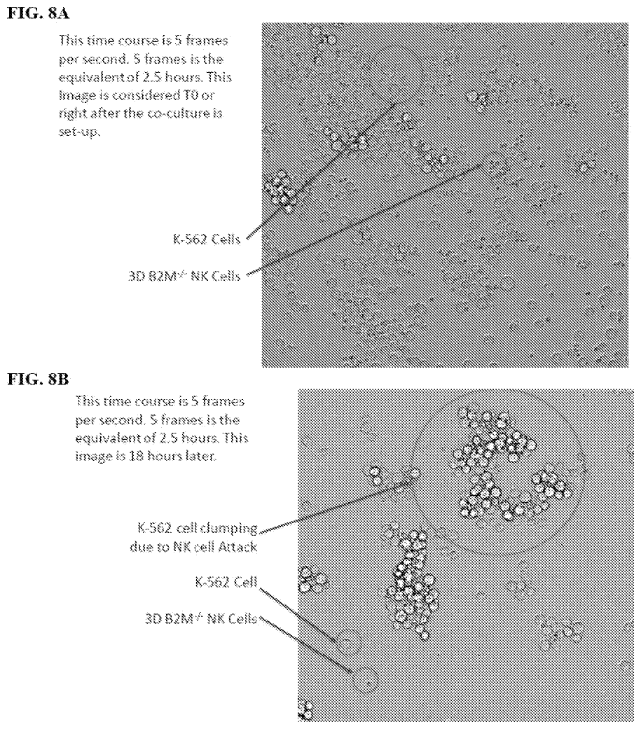

For cytotoxicity assay, activation, and cytokine release plate layout, samples were run in triplicate; cell mixtures were tested with and without a cytokine cocktail (IL-15+IL-2); PBMC isolated NK cells from a single donor were used as the control; PBMC isolated NK cells and 3D B2M−/− NK Cells (manufactured by methods of the present disclosure) were tested at two different E:T Ratios (20 k NK cells to 20 k K-562 Cells (1:1 E:T ratio) and 30 k NK cells to 20 k K-562 Cells (3:1 E:T ratio); 2D Wild Type and 2D B2M−/− NK Cells (manufactured by methods of the present disclosure) were tested at a 1:1 E:T ratio; and 3D Wild Type did not undergo the above testing and was plated for proliferation. NK Cell cytotoxicity assays are shown in FIG. 7A-B and FIG. 8A-B (time course is 5 frames per second. 5 frames are the equivalent of 2.5 hours. For orientation, it is noted that K-562 cells are larger in the images herein than NK cells. FIG. 7A-B shows PBMC Isolated NK cells (i.e., control cells) co-cultured with K-562 (3:1 E:T) without cytokine cocktail at time 0 and 18 hours later. K-562 cell clumping due to NK cell attack was observed, indicating that the assay is performing as expected. FIG. 8A-B shows the 3D B2M−/− NK cells co-cultured with K-562 (3:1 E:T) without cytokine cocktail. K-562 cell clumping due to NK cell attack is observed, and at levels that are surprisingly far greater than that of the PMBC-isolated NK cells (compare FIG. 7B and FIG. 8B and note more clumping and less unengaged NK cells.

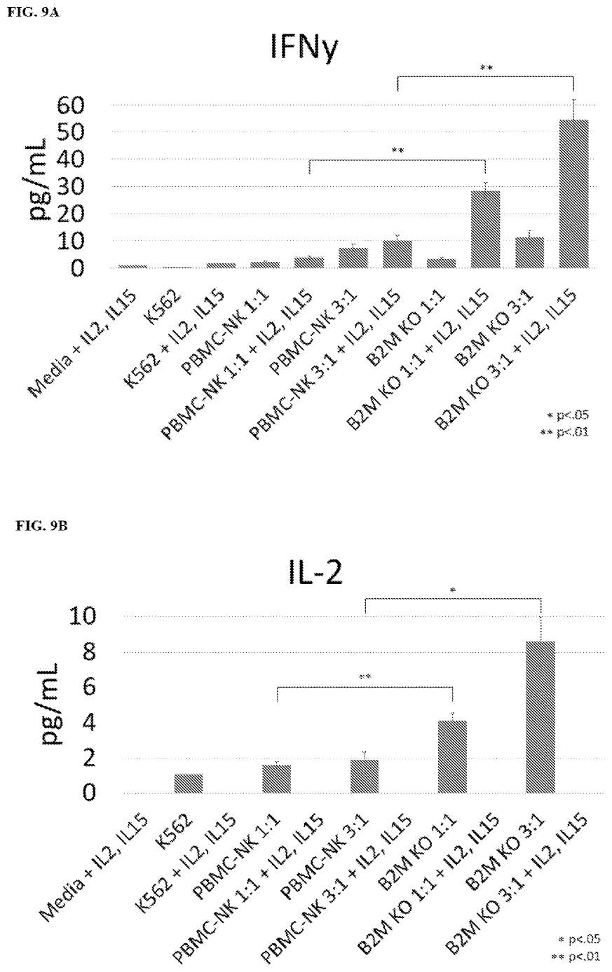

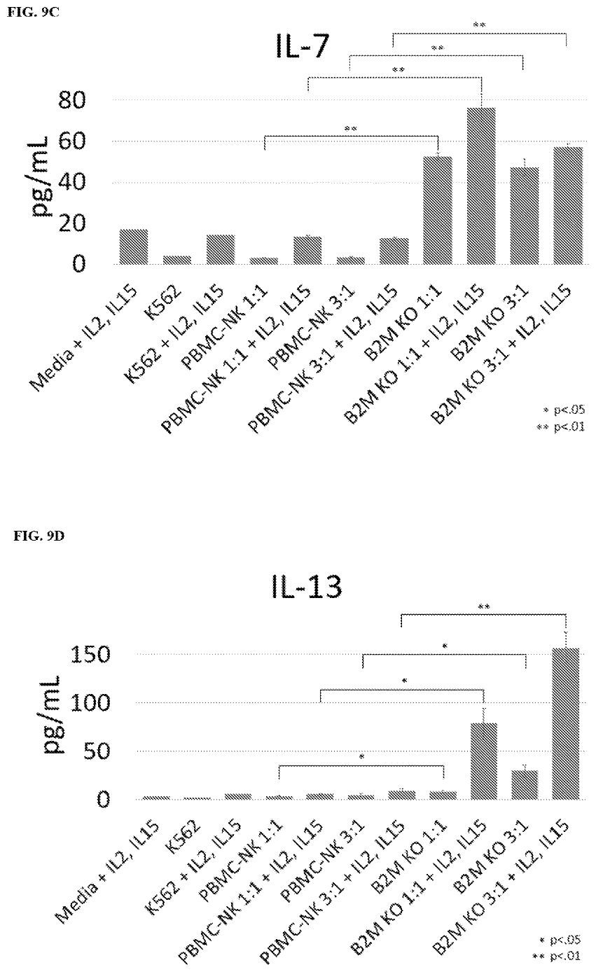

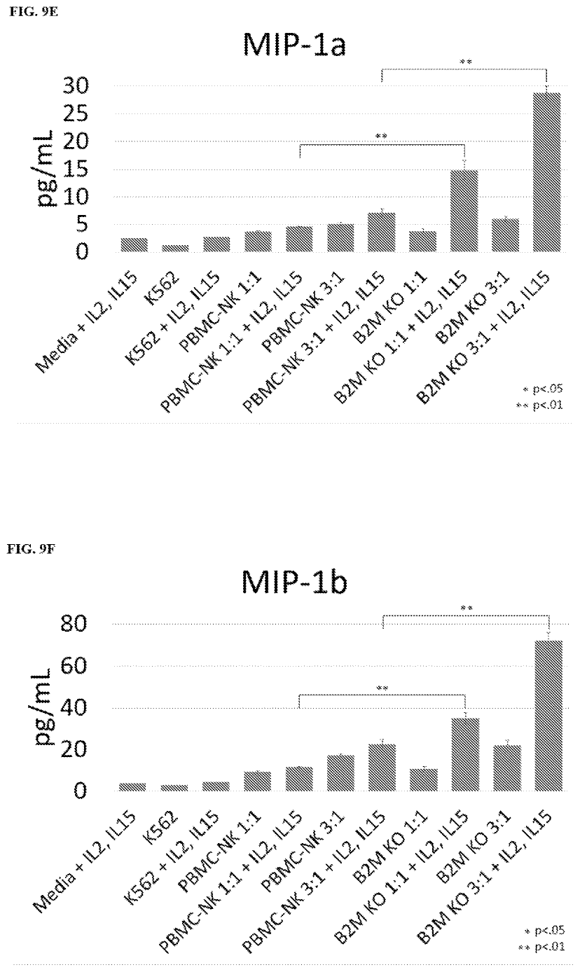

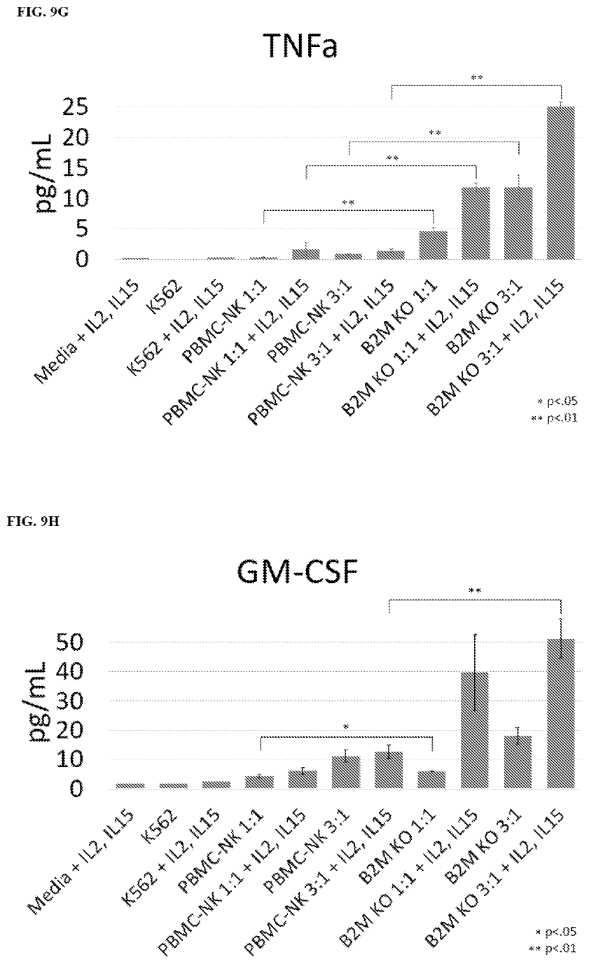

Results of the cytokine release assay with the Luminex MAGPIX are shown in FIG. 9A-FIG. 9H. Unless indicated (i.e., “+IL2, IL15”), conditions are without added IL-2 or IL-15. Further, ratio of cells is indicated (1:1 or 3:1). As elsewhere herein, wild-type PBMC-derived NK are control NK cells. FIG. 9A shows interferon gamma. FIG. 9B shows IL-2. FIG. 9C shows IL-7. FIG. 9D shows IL-13. FIG. 9E shows MIP-1a. FIG. 9F shows MIP-1b. FIG. 9G shows TNFα. FIG. 9H shows GM-CSF. In short, and without limitation, the data herein demonstrates the generation if B2M knockout cytotoxic lymphocytes that do not self-kill but, rather, self-activate (even in the absence of cytokines like IL-2 and IL-15). Further, these B2M knockout cells can kill tumor cells (even in the absence of cytokines like IL-2 and IL-15) and have unexpected expansion and proliferation properties.

Any cell type disclosed herein may have its B2M gene modified as described here, e.g., a cell of the lymphoid lineage, a cell of the myeloid lineage, a mesenchymal stromal/stem cell, or a hematopoietic stem cell.

### Example 3: B2M-HLA-E Insertion

In this example, repair template (the B2M-HLA-E repair template) comprising the B2M coding sequence, and the HLA-E (Major Histocompatibility Complex, Class I, E) coding sequence was inserted into a beta-2-microglobulin (B2M) edit. Here, iPSCs having their B2M gene edited, as disclosed herein, are contacted with a repair template comprising the coding sequence for HLA-E. Alternately, un-edited iPSCs are contacted with the gene-editing components to edit the B2M gene along with a repair template comprising the coding sequence for HLA-E. In both cases, the resulting cell (either as in iPSC or when differentiated into a cytotoxic lymphocyte of the lymphoid or differentiated into cells of the myeloid lineage, e.g., macrophages, or mesenchymal stromal/stem cells, or hematopoietic stem cells) will have an edited B2M gene and will express, in its place, HLA-E.

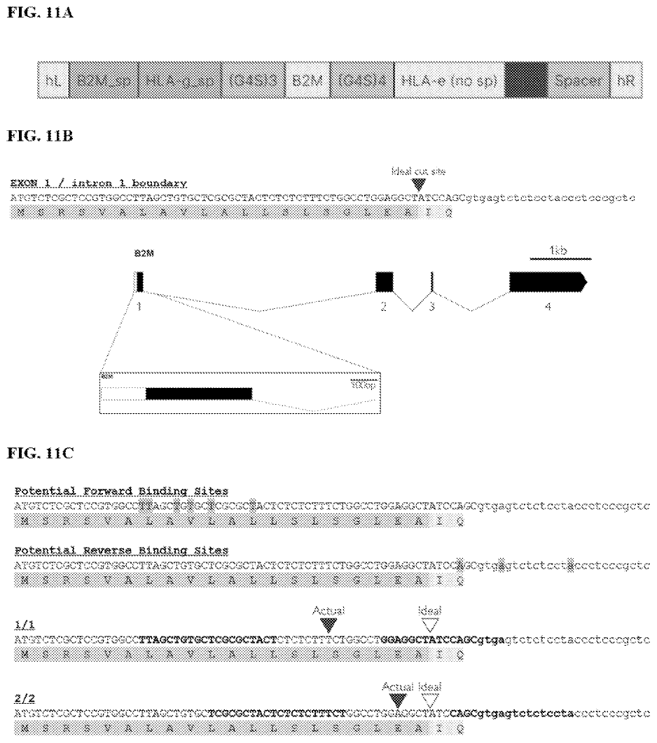

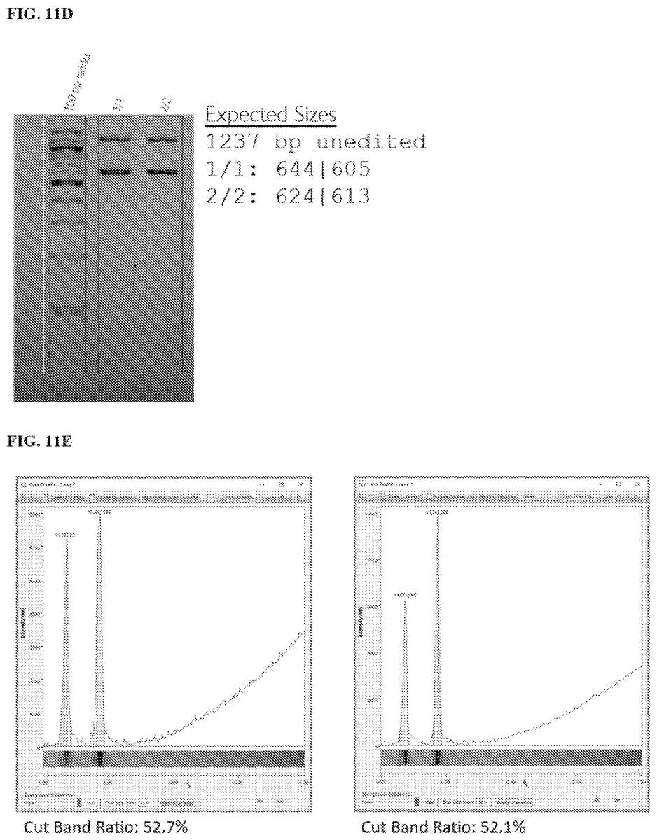

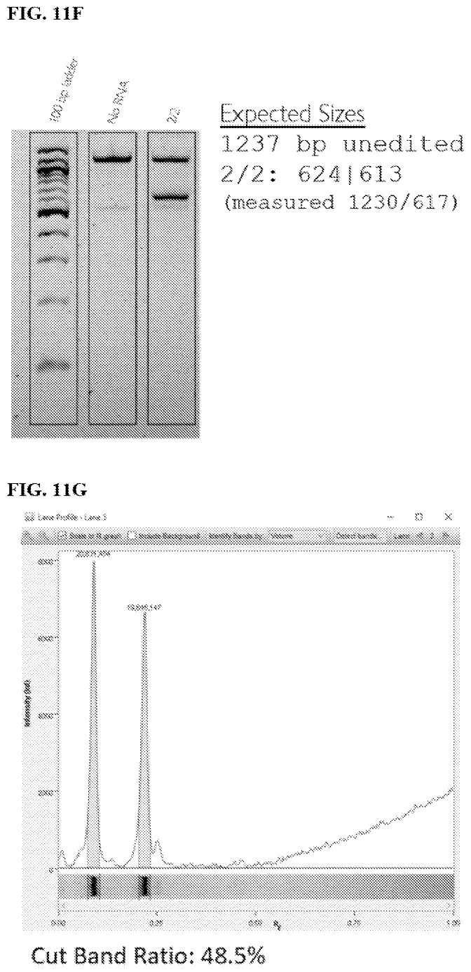

As shown in FIG. 11A the B2M signal peptide sequence (B2M_sp), which is contained entirely within Exon 1 of B2M, is included in a B2M-HLA-E repair template. Without wishing to be bound by theory, editing B2M Exon 1 and including entire B2M CDS offers the most direct path to generating the fusion.

An ideal insertion of the B2M-HLA-E repair template is located at B2M's Exon 1-Intron 1 boundary, as shown in FIG. 11B. Additional binding sites are shown in FIG. 11C, including actual lines 1/1 and 2/2 that were gene edited and which incorporated the B2M-HLA-E repair template into their genome. Using methods disclosed herein, cells were gene edited to insert the repair template into their genome. FIG. 11D shows a gel with sizes of two lines having the B2M-HLA-E repair template (of about 1.5 kb) inserted repair template into their genome at positions 1/1 and 2/2 of FIG. 11C. In this instance, mesenchymal stem cells (MSCs) cells were gene edited. FIG. 11E shows the intensity of signal and ratios thereof from the bands shown in FIG. 11D.

Using methods disclosed herein, cells were gene edited to insert the repair template into their genome. FIG. 11F shows a gel with sizes of one line having the B2M-HLA-E repair template (of about 1.5 kb) inserted repair template into their genome at position 2/2 of FIG. 11C. In this instance, iPSCs were gene edited. FIG. 11G shows the intensity of signal and ratios thereof from the bands shown in FIG. 11F.

FIG. 11H shows relevant sequences in the B2M-HLA-E repair template.

Notably, other cells, e.g., differentiated cells as described herein, could have been gene edited and inserted with a repair template comprising a coding sequence of interest, e.g., a HLA-E coding sequence.

Through the methods of this example, the repair template causes the cell to express a B2M, e.g., as a fusion protein, with HLA-E, which needs B2M to function. Thus, this method disrupts the native, endogenous B2M gene, to prevent other HLAs from functioning, thereby “stealthing” the cells.

The methods disclosed herein are enhanced when transfected RNA is associated with one or more lipid selected from Table A and/or Formulae I-XVI.

### Example 4: High Affinity CD16a Insertion

In this example, the C16a gene is edited and replaced with the coding sequence of a high affinity CD16a variant. As shown in FIG. 12A and FIG. 12B, the phenylalanine (F) at position 158 of CD16a is targeted for gene editing such that the F is replaced with a valine (V). Relevant sequences are shown in these figures.

As shown in FIG. 12B, no NheI site appears in the amplicons for CD16a, thus with CD16_NheI_ssODN_81 and CD16_NheI_ssODN_81_PT successful correction would be demonstrated by the presence of bands at ≈2127/912 bp following NheI digestion.

The methods disclosed herein are enhanced when transfected RNA is associated with one or more lipid selected from Table A and/or Formulae I-XVI.

### Example 5: Methods for Manufacturing a Population of Cells that are Enriched for Cytotoxic Lymphocytes

In this example, a population of cells that are enriched for cytotoxic lymphocytes are manufactured. The cytotoxic lymphocytes are useful in each method and pharmaceutical composition disclosed herein.

The a method for manufacturing a population of cells that are enriched for cytotoxic lymphocytes comprises steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes. In this aspect, steps (5) and (6) occur in an adherent culturing vessel. When CD34+ cells are selected, the embryoid bodies may be first chemically and/or mechanically dissociated.

Alternately, in step (5) the cells may be cultured in a myeloid progenitor medium and in step (6) the cells may be cultured in a macrophage progenitor medium under conditions to obtain a population of cells enriched for macrophages, e.g., M1 and M2 macrophages.

In embodiments, the stem cell is an induced pluripotent stem (iPSC). In some cases, the iPSC was reprogrammed from a somatic cell comprising contacting the somatic cell with one or more ribonucleic acids (RNAs), wherein each RNA encodes one or more reprogramming factors.

In some embodiments, the stem cell has a wild-type genome or has a genetically engineered disruption in a beta-2-microglobulin (B2M) gene. In some cases, the stem cell has a biallelic disruption in a B2M gene.

In various embodiments, the bioreactor is suited for culturing shear-sensitive cells and/or does not require use of anti-foaming agents or shear protectants, e.g., a vertical wheel bioreactor such as a PBS Biotech vertical-wheel bioreactor.

In embodiments, the medium in step (2) is serum-free and feeder-free culture medium, e.g., an mTeSR™ medium.

In some embodiments, the medium in step (6) is a serum-free and feeder-free culture medium, e.g., a StemDiff™ NK medium.

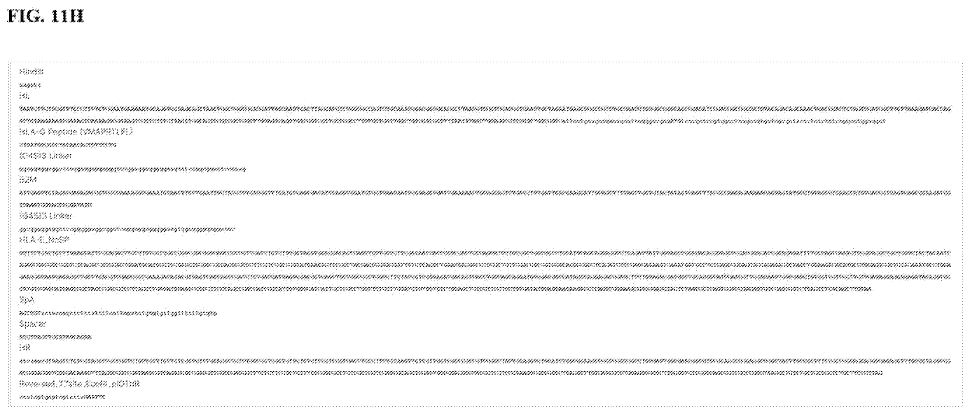

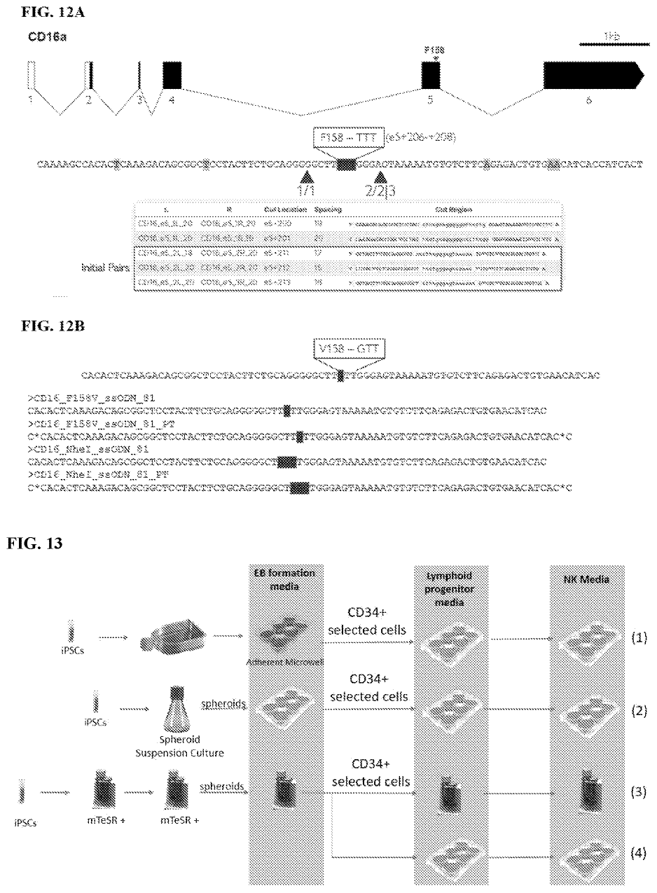

In various embodiments, the adherent culturing vessel is a multi-well plate or a cell culturing flask. FIG. 13 is a graphical representation of different protocols in the differentiation of cytotoxic lymphocytes. Protocol 1 uses a specialized micro-well cultureware to produce embryoid bodies. Protocol 2 uses a suspension pre-culture to create spheroids removes the need for the specialized culture vessel. Protocol 3 and Protocol 4 use a PBS Biotech mini-bioreactor with the Stem Cell Technologies StemDiff NK Media to create a fully suspension process.

In embodiments, the method provides from about 10-fold to about 100-fold more cytotoxic lymphocytes than obtained by a method in which each of the culturing steps comprise adherent culturing vessels; obtained by a method in which step (2) comprises a spheroid suspension culture and steps (3), (5), and (6) occur in adherent culturing vessels; and/or obtained by a method in which steps (5) and (6) occur in bioreactor.

The following table shows that using a bio-reactor in the differentiation process (shown in FIG. 13) leads to increased yields of cytotoxic lymphocytes and shows that the process can be scalable. Several different protocols were tested to see if the specialized micro-well could be removed to increase yields as well as increasing the ability to scale the process. Protocol 1 used adherent iPSCs to seed a microwell to create spheroids and then followed a fully static process in 6 well plates. Protocol 2 seeded iPSCs into a spheroid suspension culture and then took the spheroids from this culture into a fully static process in 6 well plates. Protocol 3 and protocol 4 used a Bioreactor in both the pre-culturing of the iPSCs and then the use of the embryoid body medias to create the highest yields of CD34+ cells/mL. The theoretical total fold expansion was calculated by multiplying the #of iPSCs in the beginning by each of the fold expansions of each step.

| | | Fold | Fold | |
|---|---|---|---|---|
| | | Expansion | Expansion | Theoretical |
| | # of CD34+ | in Lymphoid | in NK | Total Fold |
| | (cells/mL of | Progenitor | Differentiation | Expansion |
| | media) | Stage | State | from iPSCs |
| Protocol 1: | 12 × 10⁴ | 26x | 14x | 65x |
| Adherent | | | | |
| Microwell | | | | |
| Protocol 2: | 0.48 × 10⁴ | 8.0x | 1.6x | 35x |
| Suspension | | | | |
| Spheroid | | | | |
| Culture | | | | |
| Protocol 3: | 20 × 10⁴ | 2.5x | 4x | 29x |
| Bioreactor | | | | |
| toBioreactor | | | | |
| Protocol 4: | | 450x | 4x | 5200x |
| Bioreactor to | | | | |
| Static | | | | |

In some embodiments, the cytotoxic lymphocyte targets and kills cancer cells, e.g., a K562 cancer cell. In various embodiments, the cytotoxic lymphocyte targets and kills cancer cells without requiring IL-15 and/or without requiring IL-2 activation. In embodiments, the cytotoxic lymphocyte targets and kills at least 70% of cancer cells in a population within about 4 hours. In some embodiments, the cytotoxic lymphocyte targets and kills at least 80% of cancer cells in a population within about 24 hours. In various embodiments, the cytotoxic lymphocyte has reduced cytotoxicity to an NK-resistant cancer cell, e.g., a NAMALWA cell.

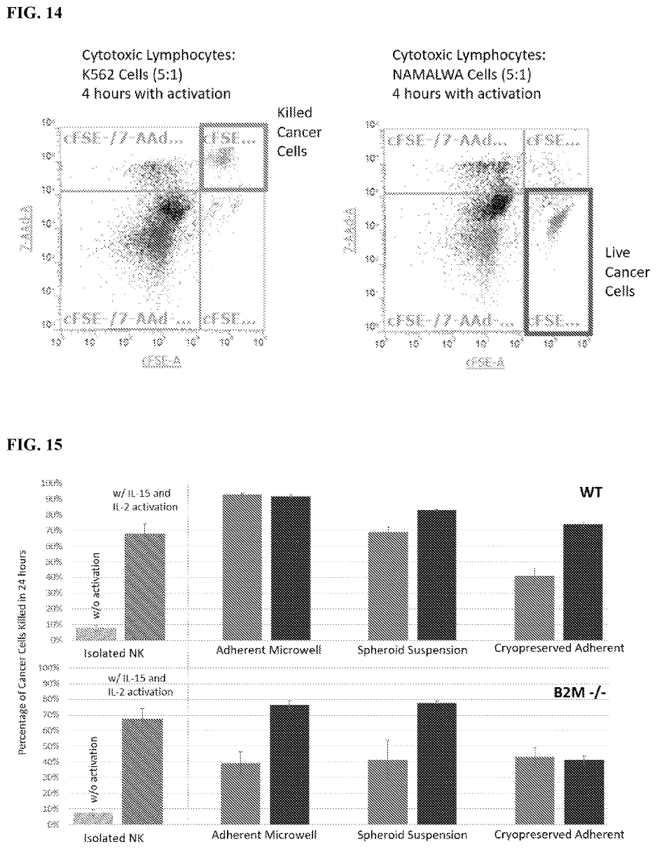

FIG. 14 is an illustrative flow cytometry fluorescence map used in data analysis of cytotoxicity assays for cytotoxic lymphocytes manufactured by protocol 4. A comparison of the left panel and the right panel shows the difference between the higher percentage of killed K562 cancer cells (left panel) and the living NAMALWA cells (an NK-resistant cancer cell; right panel). On the x axis is cFSE which is the stain used to identify the target cells, on the y axis is 7AAD which identifies dead cells. The percent of cancer cells killed is determined by the percentage of cFSE stained cells that are also 7AAD positive.

FIG. 15 shows the cytotoxic ability of differentiated cells were determined with K562 cells over a 24-hour period. While isolated NK cells (CD45+CD56+CD3−; left most pair of graphs) required activation with cytokines to become cytotoxic, wild-type cytotoxic lymphocytes generally showed un-controlled cytotoxicity (top row, middle two graphs). On the other hand, the B2M knockout cytotoxic lymphocytes (bottom row, middle two graphs) show cytokine-controlled cytotoxicity as compared to wild-type cells. Additionally, cryopreserved cells from the adherent method show a slight decrease in cytotoxic ability (right graphs). Errors bars represent standard deviation over the three different samples. The left data for each graph are cells that were not activated and the right data for each graph are cells that were activated with IL-15 and IL-2.

In some embodiments, the cytotoxic lymphocyte targets and kills cancer cells, e.g., a K562 cancer cell. In various embodiments, the cytotoxic lymphocyte targets and kills cancer cells without requiring IL-15 and/or without requiring IL-2 activation. In embodiments, the cytotoxic lymphocyte targets and kills at least 70% of cancer cells in a population within about 4 hours. In some embodiments, the cytotoxic lymphocyte targets and kills at least 80% of cancer cells in a population within about 24 hours.

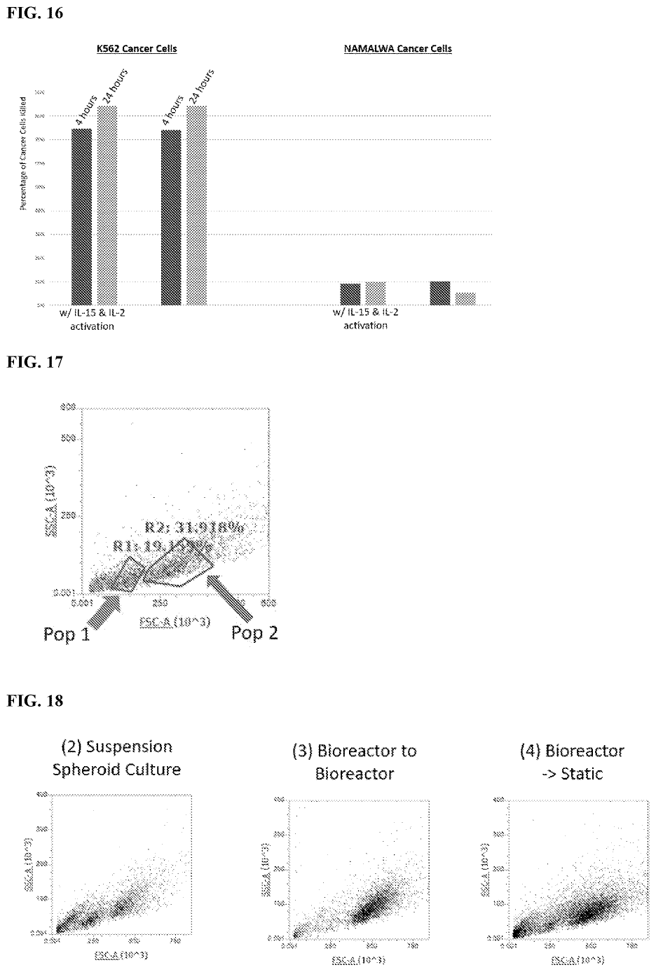

FIG. 16 shows that lower cytotoxicity is observed from an NK-resistant cancer cell lines by cytotoxic lymphocytes manufactured by protocol 4. Cytotoxicity Data was collected at 4 hours and 24 hours, with and without simulation with IL-15 & IL-2 activation, and for both K562 cancer cells and with NAMALWA cells (an NK-resistant cancer cell). The data shown was using cytotoxic lymphocytes created using Protocol 4: WT Bioreactor to Static Culture, with wild-type iPSCs. The ability of these cells to kill NAMALWA cells was significantly lower than their ability to kill K562 cells. Additionally, with these cells, most of the cytotoxicity occurred by 4 hours, which points to a NK or delta-gamma T cell identity. Thus, the cells manufactured by protocol 4 are efficient cancer cell killers. In embodiments, the cytotoxic lymphocyte is a Natural Killer (NK) cell. In some cases, the NK cell is a mature NK cell.

In some embodiments, the cytotoxic lymphocyte is a Natural killer T (NKT) cell.

In various embodiments, the cytotoxic lymphocyte is a delta-gamma T cell.

Flow cytometry reveals a mixed population which differs in regard to certain NK as well as certain T cell markers. Protocol 1 and 2 were tested with both wild-type and B2M KO cells and flow FSC vs. SSC revealed two distinct population in terms of size. Additionally, when looking at the difference in surface markers between the second population seems to have expression of NK markers (CD244, CD336). See the below Table. All cells were: CD45+, CD56+, CD16−, NKG2D−, KIR2DL4−, KIR2DL1−, and CD8−.

| | Adherent Microwell (Protocol 1) | | | | Suspension Spheroid (Protocol 2) | | | |
|---|---|---|---|---|---|---|---|---|
| | WT | | B2M KO | | WT | | B2M KO | |
| | Population | Population | Population | Population | Population | Population | Population | Population |
| | 1 | 2 | 1 | 2 | 1 | 2 | 1 | 2 |
| CD244 | 63% | 94% | 77% | 94% | 50% | 92% | 62% | 89% |
| CD336 | 61% | 77% | 55% | 32% | 42% | 33% | 4% | 5% |
| CD4 | 30% | 51% | 25% | 24% | 50% | 48% | 59% | 56% |
| TCRαβ | 1% | 9% | 49% | 9% | 6% | 3% | 55% | 49% |
| TCRγδ | 0% | 0% | 75% | 11% | 82% | 49% | 77% | 13% |

As shown in FIG. 17, flow FSC vs. SSC revealed two distinct population in terms of size.

The use of suspension pre-cultures does not predict final NK morphology or purity. Protocols 2 and 4, which both switch from a suspension iPSC pre-culture into a static lymphoid and NK culture, show two distinct populations whereas protocol 3 has one distinct population. See FIG. 18. Without wishing to be bound by theory, it appears that Population 1 cells possess monocyte characteristics and Population 2 cells possess NK or NKT (including delta gamma T cells) characteristics.

In some embodiments, the cytotoxic lymphocytes are enriched for CD56+ cells, for CD16+ cells, NKG2D+ cells, CD226+ Cells, NKp46+ cells, NKp44+ cells, CD244+ cells, and/or CD94+ cells.

In various embodiments, the method provides from about 5-fold to about 30-fold more CD16+ cells than obtained by a method in which step (2) comprises a spheroid suspension culture and steps (3), (5), and (6) occur in adherent culturing vessels and/or obtained by a method in which steps (5) and (6) occur in bioreactor.

In embodiments, the method provides from about 5-fold to about 25-fold more NDG2D+ cells than obtained by a method in which step (2) comprises a spheroid suspension culture and steps (3), (5), and (6) occur in adherent culturing vessels and/or obtained by a method in which steps (5) and (6) occur in bioreactor.

In some embodiments, the method provides from about 2-fold to about 30-fold more NKp44+ cells than obtained by a method in which step (2) comprises a spheroid suspension culture and steps (3), (5), and (6) occur in adherent culturing vessels and/or obtained by a method in which steps (5) and (6) occur in bioreactor.

In various embodiments, the method provides from about 2-fold to about 8-fold more CD94+ cells than obtained by a method in which step (2) comprises a spheroid suspension culture and steps (3), (5), and (6) occur in adherent culturing vessels and/or obtained by a method in which steps (5) and (6) occur in bioreactor.

In embodiments, the method provides from about 2-fold more NKp46 cells than obtained by a method in which step (2) comprises a spheroid suspension culture and steps (3), (5), and (6) occur in adherent culturing vessels.

These features are illustrated in the below table:

| | | Protocol (2) | Protocol (3) | Protocol (4) |
|---|---|---|---|---|
| | | Spheroid | Bioreactor to | Bioreactor |
| | | Suspension | Bioreactor | to Static |
| CD56 | NK Cells | 96% | 99% | 96% |
| CD16 | Mature NK Cells, ADCC | 1% | 2% | 31% |
| NKG2D | NK Activating Receptor | 4% | 6% | 83% |
| CD226 | DNAM-1 Receptor | 80% | 80% | 91% |
| NKp46 | Activating Receptor | 33% | 56% | 58% |
| | | Pop 1 > Pop 2 | | |
| NKp44 | Activating Receptor | 10% | 1% | 28% |
| | | Pop 2 > Pop 1 | | |
| CD244 | MHC Class I binding NK | 80% | 98% | 98% |
| | Cells | Pop 2 > Pop 1 | | |
| CD94 | Works with NKG2 | 24% | 11% | 79% |
| CD158a/h/g | KIR Family | 95% | 88% | 68% |
| | Inhibitory Receptor | | | |

### Example 6: Genetically Modifying a Gene-Edited and Differentiated Cell into a Chimeric Antigen Receptor (CAR)

In this example, cells of the present disclosure, e.g., myeloid lineage cells, e.g., macrophages, and a cytotoxic lymphocyte of the present disclosure, e.g., a T cell or NK cell, that was generated via methods of the present disclosure (e.g., for manufacturing a population of cells that are enriched for cytotoxic lymphocytes, enriched for macrophages, and/or by gene editing to disrupt the B2M gene and differentiation the cell from a stem cell into a cytotoxic lymphocyte of the lymphoid lineage or myeloid lineage), is genetically modified to express a recombinant chimeric antigen receptor (CAR). In some cases, the cytotoxic lymphocyte of the lymphoid lineage or cells of the myeloid lineage are derived from iPSC cells (as disclosed herein). The CAR comprises an intracellular signaling domain, a transmembrane domain, and an extracellular domain comprising an antigen binding region. In embodiments, the cells of the present disclosure is engineered to be directed to ROR1 and/or CD19. The methods for genetically modifying a cell to express a recombinant CAR are well known in the art and any such well-known method may be utilized with the cytotoxic lymphocytes of the present disclosure or cells of myeloid lineage of the present disclosure.

In some cases, the intracellular signaling domain of the CAR comprises at least one immunoreceptor tyrosine-based activation motif (ITAM)-containing domain.

In some cases, the intracellular signaling domain of the CAR is from one of CD3-zeta, CD28, CD27, CD134 (OX40), and CD137 (4-1BB).

In some cases, the transmembrane domain of the CAR is from one of CD28 or a CD8.

In some cases, the antigen binding region binds one antigen. In embodiments, the binding region binds two antigens.

In some cases, the extracellular domain comprising an antigen binding region comprises: (a) a natural ligand or receptor, or fragment thereof, or (b) an immunoglobulin domain, optionally a single-chain variable fragment (scFv). In embodiments, the extracellular domain comprising an antigen binding region comprises two of (a) a natural ligand or receptor, or fragment thereof, or (b) an immunoglobulin domain, optionally a single-chain variable fragment (scFv). In embodiments, the extracellular domain comprising an antigen binding region comprises one of each of: (a) a natural ligand or receptor, or fragment thereof, and (b) an immunoglobulin domain, optionally a single-chain variable fragment (scFv).

In some case, the antigen binding region comprises one or more of CD94/NKG2a, which optionally binds HLA-E on a tumor cell; CD96, which optionally binds CD155 on a tumor cell; TIGIT, which optionally binds CD155 or CD112 on a tumor cell; DNAM-1, which optionally binds CD155 or CD112 on a tumor cell; KIR, which optionally binds HLA class I on a tumor cell; NKG2D, which optionally binds NKG2D-L on a tumor cell; CD16a, which optionally binds an antibody/antigen complex on a tumor cell and/or wherein the CD16a is optionally a high affinity variant, optionally homozygous or heterozygous for F158V; NKp30, which optionally binds B7-H6 on a tumor cell; NKp44; and NKp46.

### Example 7: Treating Cancer

In this example, a cytotoxic lymphocyte of the present disclosure is used to treat a cancer.

The method for treating a cancer patient in need thereof comprises a step of administering to the cancer patient a therapeutically-effective amounts of a herein-disclosed cytotoxic lymphocyte, e.g., manufactured by a method that produces a population of cells that are enriched for cytotoxic lymphocytes. The cytotoxic lymphocyte may be a Natural Killer (NK) cell. In some cases, the NK cell is a mature NK cell. The cytotoxic lymphocyte may be a Natural killer T (NKT) cell. In some cases, the cytotoxic lymphocyte is a delta-gamma T cell.

In some case, the method for treating cancer comprises steps of obtaining an isolated cytotoxic lymphocyte comprising a genetically engineered disruption in a B2M gene; and administering the isolated cytotoxic lymphocyte to a subject in need thereof. In this method the cytotoxic lymphocyte is a lymphoid cell lineage and administered with or without a cell of the myeloid cell lineage. In some cases, the cytotoxic lymphocyte is a T cell, e.g., a cytotoxic T cell or gamma-delta T cell; NK cell, e.g., a NK-T cell; the cell of the myeloid lineage is a macrophage, e.g., M1 macrophage or M2 macrophages, or mesenchymal stromal/stem cells, or hematopoietic stem cells.

In some cases, additionally or alternately, the cytotoxic lymphocyte expresses a high affinity CD16a receptor.

In some cases, the cytotoxic lymphocyte expresses a fusion protein comprising a B2M polypeptide and a HLA-A, HLA-B, HLA-C, HLA-E, HLA-F and HLA-G polypeptide. The fusion protein may be expressed by insertion of a repair template into a single or double strand break of the B2M gene; in some cases, the repair template comprises the coding sequence for B2M and the HLA gene. In embodiments, the repair template includes the coding sequence for one or more of HLA class I histocompatibility antigen, alpha chains (HLAs). Notably, the fusion protein replaces endogenous B2M and HLA pairs expressed by a cytotoxic lymphocyte, thereby reducing the likelihood that the cytotoxic lymphocyte will be reduced or eliminated by a host cytotoxic lymphocyte.

In some cases, the cytotoxic lymphocyte is further genetically engineered to express a chimeric antigen receptor (CAR).

In some cases, the cytotoxic lymphocyte is further genetically engineered to express or overexpress a cytokine.

The cancer may be a blood cancer.

The cancer may be a solid tumor.

The cancer may be selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

### Example 8: Scalable, Mixed Population iPS-Cell Derived Lymphoid Cells and Myeloid Cells for Enhanced Tumor Cell Killing

Induced pluripotent stem (iPS) cell therapies have the potential to treat a wide variety of devastating diseases. iPS cell-derived lymphocytes (e.g., T cells and NK cells) engineered to express targeting molecules such as chimeric antigen receptors (CARs) have shown clinical promise to treat hematological malignancies. More recently, iPS cell-derived myeloid cells are being developed to treat both hematological malignancies and solid tumors due to the ability of these cells to infiltrate and modulate the tumor microenvironment. Despite preliminary success, several challenges still remain, including poor infiltration of cytotoxic lymphocytes into solid tumors and insufficient cytotoxicity of myeloid cells. We hypothesized that a multi-cell-type therapy comprising both lymphoid lineage cells and myeloid lineage cells may work synergistically, enhancing cytotoxicity and efficacy. In above examples, directed differentiation of mRNA reprogrammed iPS cells into functional cytotoxic lymphocytes (e.g., NK cells) and monocyte-derived M1 and M2 polarized macrophages was disclosed.

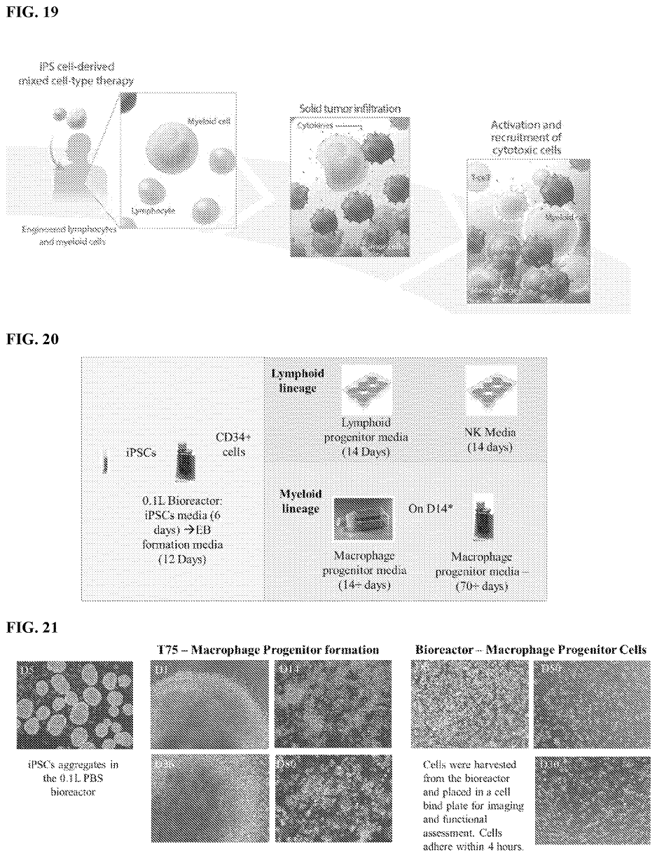

In this example, advances in the generation of a scalable bioreactor-based process for parallel differentiation of mRNA reprogrammed iPS cells into both CD14+ (>95% positive) macrophages and CD56bright/CD16dim NK cells is disclosed. Further, a plurality of populations of iPS-cell derived lymphoid lineage cells and myeloid lineage cells are combined to form a multi-cell type therapy for treating cancer. FIG. 19 illustrates usefulness of the plurality of populations in treating cancer. Disclosed herein is a method for manufacturing a plurality of population of cells that are enriched for lymphoid lineage cells, e.g., cytotoxic lymphocytes including NK cells, and are enriched for myeloid lineage cells, including macrophages. The method comprises steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for NK cells; and (6b) culturing the cells of step (5b) in a macrophage cell medium under conditions to obtain a population of cells enriched for macrophages.

In this illustrative method, steps (5a) and (6a) occur in an adherent culturing vessel and steps (5b) and (5c) occur in a bioreactor.

In embodiments, the stem cell is an induced pluripotent stem (iPSC). In some cases, the iPSC was reprogrammed from a somatic cell comprising contacting the somatic cell with one or more ribonucleic acids (RNAs), wherein each RNA encodes one or more reprogramming factors.

In some embodiments, the stem cell has a wild-type genome or has a genetically engineered disruption in a beta-2-microglobulin (B2M) gene. In some cases, the stem cell has a biallelic disruption in a B2M gene.

In various embodiments, the bioreactors are suited for culturing shear-sensitive cells and/or does not require use of anti-foaming agents or shear protectants, e.g., a vertical wheel bioreactor such as a PBS Biotech vertical-wheel bioreactor.

In embodiments, the medium in step (2) is serum-free and feeder-free culture medium, e.g., an mTeSR™ medium.

In some embodiments, the medium in step (6a) and/or step (6b) is a serum-free and feeder-free culture medium, e.g., a StemDiff™ NK medium.

In various embodiments, the adherent culturing vessel is a multi-well plate or a cell culturing flask. FIG. 20 is a graphical representation of an illustrative protocol for manufacturing the plurality of populations of cells that are enriched for lymphoid lineage cells, e.g., cytotoxic lymphocytes, including NK cells, and enriched for myeloid lineage cells, including macrophages. On day zero, iPSCs were thawed directly into a PBS 0.1 L vertical wheel bioreactor at 40,000 cells/mL and allowed to recover in mTeSR+. After the cells had formed aggregates (e.g., of >50 μm), a 100% media change was performed, and all aggregates were placed in EB formation media to generate CD34+ embryoid bodies (EBs). FIG. 21 (left image) shows iPSCs formed uniform EBs. After twelve days in EB media (now day 18), EBs were harvested from the bioreactor and divided into two subsets with one subset being used for lymphoid differentiation and the second subset begin used for myeloid differentiation.

For lymphoid lineage differentiation, EBs were dissociated and placed into static six-well plates. Cells remained in lymphoid progenitor media for two weeks followed by NK media for two weeks (46 days total).

For myeloid lineage differentiation, EBs, were plated into a coated T75 flask at 1 EB per cm² in macrophage progenitor media. EB's plated into the T75 adhered after 24 hours, and remained highly confluent over the culture period. See, FIG. 21, middle images.

After ten days, CD14+ cells began to bud off from the EBs. After fourteen days in the T75, (now day 32), cells in suspension were harvested from static and seeded into a new PBS 0.1 L bioreactor. Cells were maintained in both the T75 and bioreactor for greater than seventy days (now after day 88) and maintained a high viability (>95%). As shown, in FIG. 21, right images, CD14+ cells in the bioreactor maintained their morphology. Cells harvested from suspension culture would adhere quickly to cell bind plates when removed.

Macrophage progenitor cell growth and characteristics were assayed.

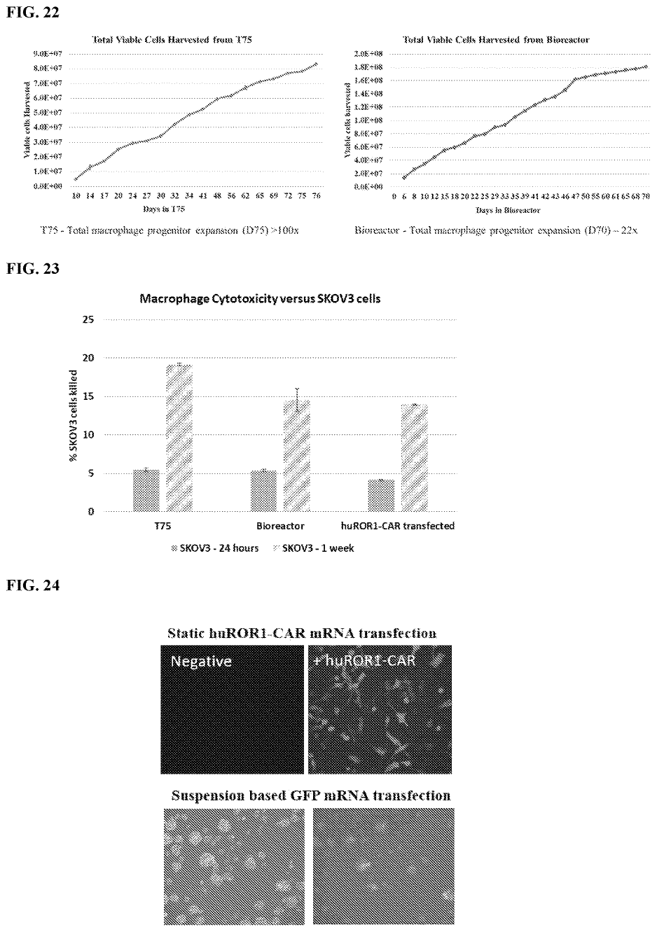

FIG. 22 shows the sum of viable macrophage progenitor cells harvested from the T75 flasks (left) and bioreactor (right) throughout the culture period. Cells were harvested throughout the duration of the culture period while being maintained in macrophage progenitor media. Cells were characterized via flow cytometry and cytokine secretion (see below table). Functional assessment via cytotoxicity and transfection were performed. The T75 was seeded at ˜1 EB/cm², <1M cells. Throughout the culture period of more than eighty days, over 80M CD14+ cells were harvested from the T75. On day zero of the bioreactor culture, 8M cells were seeded. Over 70 days, 180M CD14+ cells were harvested. Cells maintained high viability (>95%) while in culture.

Flow cytometry data from macrophage progenitor cells is shown below in the following table

| Surface | | T75 | | | Bioreactor | | |
|---|---|---|---|---|---|---|---|
| Marker | Expected | D 14 | D 37 | D 85 | D 8 | D 22 | D 70 |
| CD5 | − | − | − | − | − | − | − |
| CD11b | + | 82 | 99 | 98.9 | 96 | 75 | 99 |
| CD14 | + | 31 | 99 | 99 | 97 | 99 | 99 |
| CD19 | − | 18 | 0 | 0 | 26 | 0 | 0 |
| CD33 | + | 72 | 94 | 97 | 97 | 88 | 52 |
| CD45 | + | 63 | 98 | 98 | 91 | 98 | 98 |
| CD56 | − | − | 0 | 2 | 3.6 | 0 | 1 |
| HLA-DR | − | 0 | 0 | 7 | 0 | 0 | 1 |
| TLR4 | + | 0 | 84 | 94 | 98 | 85 | 55 |

The above flow data was performed with the addition of a human Fc blocker. AttuneNXT was used for data collection. Cells in the T75 had improved surface marker expression after day 14, and maintained high levels of expression for key myeloid and macrophage markers (>90% for CD14, CD33, and CD45) and were negative for key lymphoid markers (<10% CD5, CD19, CD56, and HLA-DR).

FIG. 23 shows an assessment of baseline macrophage cytotoxicity. The cytotoxicity of macrophage progenitor cells was measured after 24 hours and one week of co-incubation period with an ovarian cancer cell line (SKOV3) at a 5:1 E:T ratio. The percent dead cells was determined via a live/dead stain (7-AAD). After 24 hours or one week, cells were harvested and stained with CD45 to distinguish between the effector cells (CD45+) and the target cancer cells (CD45−). Cytotoxicity was determined by the percentage CD45− cells (x axis) that were also 7-AAD positive (y axis). Baseline cancer cell death was accounted for by subtracting the average spontaneous cancer cell death from the percent killed in the presence of the macrophages. Macrophages were more effective at killing SKOV3 cells over a longer timepoint. Increased clustering was seen after 72 hours.

FIG. 24 shows the results from transfection of macrophages with a mRNA encoding a chimeric antigen receptor (CAR). Cells were transfected with 1 μg/1M cells of mRNA encoding a GFP protein or encoding a humanized ROR1-CAR protein. Cells either were removed and plated in static and allowed to recover for 48 hours prior to transfection or were maintained in suspension culture and the mRNA was delivered via ToRNAdo™. The ROR1-CAR construct was stained with RPE-labeled ROR1 recombinant protein.

NK cell cryopreservation dynamics and other characteristics were assayed.

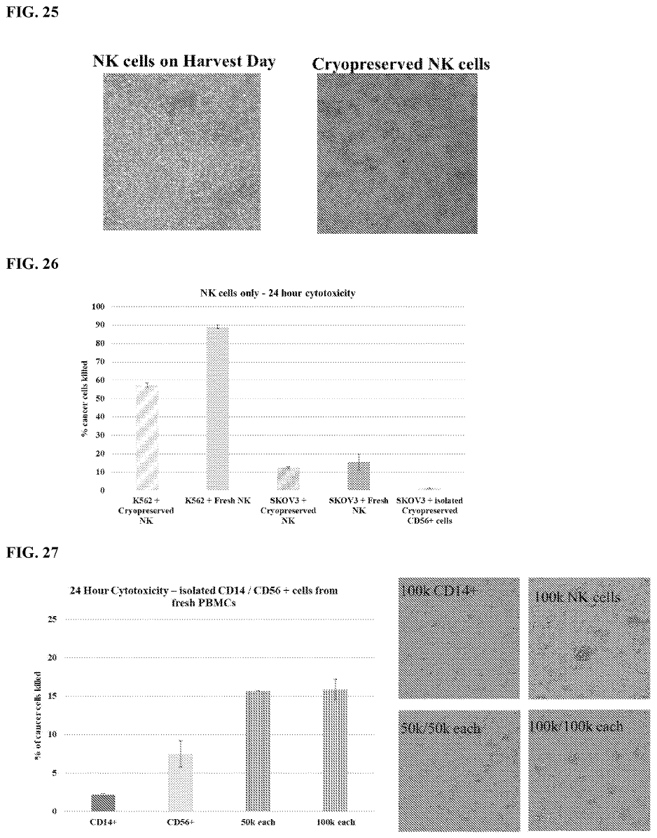

FIG. 25 shows the morphology of NK cells on the final day of the differentiation protocol versus 24 hours post thaw. NK cells maintained their uniform, round cell shape and size after being cryopreserved in CryoStor10. Cells were cryopreserved using Cryostor10 (6 M/mL). During thaw, cells had 88% viability and a 70% recovery per vial of about 4M cells.

The below table shows lymphoid cell expansion throughout differentiation. As shown, cells had the highest expansion after seeding the dissociated EB's into static six-well plates. Cells continued to expand during the static NK stage.

| | Lymphoid Cell Expansion |
|---|---|
| Thaw into Bioreactor | 5.7 × 10\hat{ }6 |
| # of CD34+ cells | 2.5 × 10\hat{ }6 |
| Fold Expansion Lymphoid | 33x |
| Fold Expansion NK Cells | 5.5x |

FIG. 26 shows effect of cryopreservation on the cytotoxicity of NK cells. Cytotoxicity of NK cells was measured after a 24 hour co-incubation period with the target cancer cell lines (K562 or SKOV3) at a 5:1 E:T ratio. The percent dead cells was determined via a live/dead stain (7-AAD). K562 cells were stained with CFSE prior to incubation. NK cells and SKOV3 cells were stained with CD45. Cytotoxicity was determined by the percentage of either CFSE stained target K562 cells or CD45-SKOV3 cells (x axis) that were also 7-AAD positive (y axis). Baseline cancer cell death was accounted for by subtracting the average spontaneous cancer cell death from the percent killed in the presence of the NK cells. Cryopreservation decreased the cytotoxicity of NK cells, however they were much more cytotoxic than isolated frozen CD56+ cells.

The below table shows data from flow cytometry data from fresh versus cryopreserved cytotoxic lymphocytes.

| Surface Marker | Expected | Cryopreserved | Fresh |
|---|---|---|---|
| CD56 | + | 76 | 74 |
| CD16 | − | 19 | 13 |
| CD3 | − | 18 | 16 |
| CD62L | + | 3 | 70 |
| CD226 | + | 2 | 6 |
| CD4 | − | 19 | 39 |
| CD8 | − | 4 | 5 |
| TCRgd | − | 38 | 32 |
| CD19 | − | 3 | 2 |
| CD34 | − | 2 | 3 |
| CD7 | + | 57 | 51 |
| CD11b | − | 9 | 24 |
| CD14 | − | 18 | 27 |

The flow data was performed with the addition of a human Fc blocker. AttuneNXT was used for data collection. Desired NKI characterization (CD56bright/CD16dim) was maintained after cryopreservation. TCR and CD7 remained unchanged, as well as maintenance of key negative markers (CD11b, CD14, CD19).

Assays characterizing effects of plurality of populations iPS-cell derived lymphoid lineage cells, e.g., cytotoxic lymphocytes, including NK cells, and iPS-cell derived myeloid lineage cells, including macrophages, was performed.

FIG. 27 illustrates the surprisingly synergistic effects of administration of a plurality of populations lymphoid lineage cells and myeloid lineage cells on killing cancer cells. CD14+ (including macrophages) and CD56+ cells (including NK cells) were isolated from fresh huPBMC's and immediately co-incubated with SKOV3 cancer cells. Cells were activated using IL2 and MCSF. After 24 hours, cells were harvested and flowed on the AttuneNXT. The CD14+/CD56+ cells showed synergistic tumor cell killing of SKOV3 ovarian cancer cells (combined: 15.6%; macrophage alone=2.2% (p<0.01); NK alone=7.5% (p<0.05); E:T=5:1). The combined populations of cells showed increased clustering and tumor cell engagement.

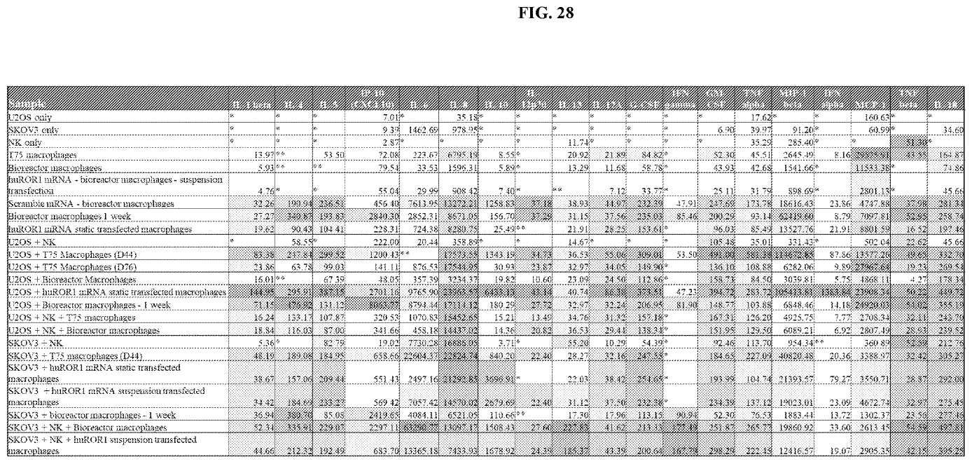

FIG. 28 shows data from cytotoxicity assay cytokine release heat map with iPS derived cells of the myeloid lineage (which includes macrophages) cells of the lymphoid lineage cells (which includes NK cells). In these experiments, media was sampled after a 24-hour incubation with SKOV3 and U2OS cells with activating cytokines IL-2 and MCSF. This data was processed on the Luminex MAGPIX. Transfection of macrophages with mRNA encoding a huROR1-CAR increased macrophage specific cytokine secretion (IL-8, IL-12p70, MIP-1beta) in the presence of cancer cells. When macrophages and NK cell were combined with SKOV3 cell, there was an increase in cytokine release (Il-6, IL-13, IFN-gamma, TNF-beta). Macrophages incubated for one week with cancer cells had increased expression in cytokines.

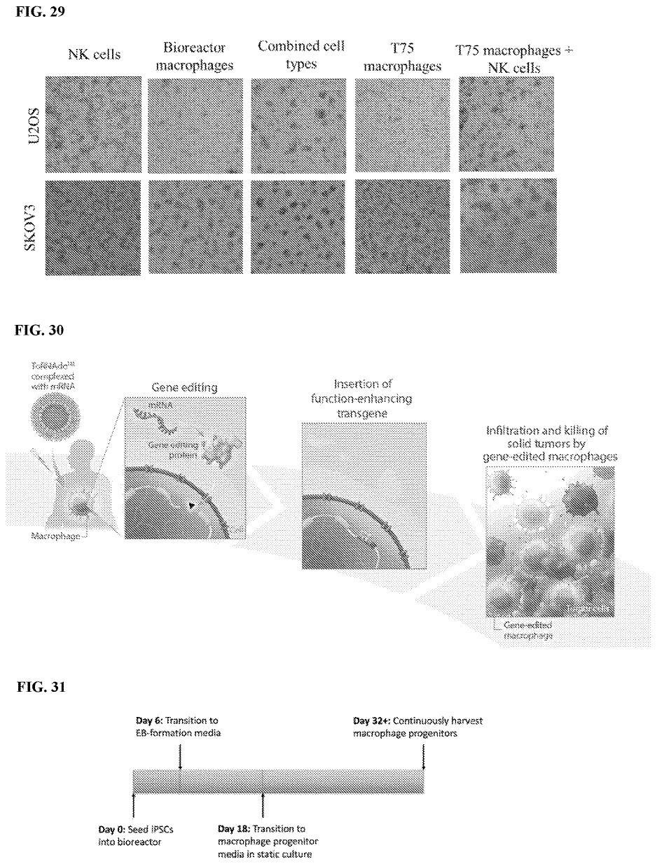

FIG. 29 shows iPSC derived immune cell clustering during a cytotoxicity assay after 24 hours. iPSC-derived NK cells and iPSC-derived macrophages (as disclosed above) were seeded with U2OS and SKOV3 cells at a 5:1 E:T ratio. After 24 hours, there was increased cell clustering when the two cell types were combined. NK cells were thawed and seeded directly into the assay. Cells were harvested fresh from the T75 (D83) and bioreactor (D68). When the two cell populations were combined, there was increased cell clustering in both U2OS and SKOV3 cells. Thus, these iPSC-derived cell populations, much like the natural cellular immune response, act synergistically to kill tumor cells in vitro. By more closely mimicking natural cellular immunity, multi-cell-type cell therapies, such as disclosed herein, represent a new class of cell therapies that plays an important role in the development of new medicines to treat cancer.

In summary, disclosed in this example, is a scalable platform for generating iPS cell-derived multi-cell-type therapies comprising both lymphoid lineage cells and myeloid lineage cells. Much like the natural cellular immune response, these iPSC-derived cells act synergistically to kill tumor cells. By more closely mimicking natural cellular immunity, multi-cell-type cell therapies represent a new class of cell therapies that may play an important role in the development of new medicines to treat cancer.

It should be noted that the present disclosure is a method for killing a cancer cell or for inhibiting the proliferation of a cancer cell. The method comprising contacting the cancer cell with a population of isolated lymphoid lineage cells and a population of isolated myeloid lineage cells.

In numerous embodiments, the cancer cell is contacted with the population of isolated lymphoid lineage cells and the population of isolated myeloid lineage cells simultaneously.

In embodiments, the cancer cell is contacted with the population of isolated lymphoid lineage cells before being contacted with the population of isolated myeloid lineage cells or the cancer cell is contacted with the population of isolated lymphoid lineage cells after being contacted with the population of isolated myeloid lineage cells.

In embodiments, the isolated lymphoid lineage cells and the isolated myeloid lineage cells are manufactured by a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor. In cases when CD34+ cells are selected, the embryoid bodies are first chemically and/or mechanically dissociated. In these embodiments, the stem cell may be an induced pluripotent stem (iPSC). In some cases, the isolated lymphoid lineage cell and the isolated myeloid lineage cell are derived from the same iPSC. In various cases, the iPSC comprises a genomic modification that expresses a chimeric antigen receptor (CAR) and/or comprises a genomic modification that expresses or over expresses a cytokine. The iPSC may comprise a genomic modification which disrupts the beta-2-microglobulin (B2M) gene, e.g., a biallelic disruption in a B2M gene; in these cases, the iPSC expresses a fusion protein comprising a B2M polypeptide and an HLA polypeptide (e.g., an HLA-A, HLA-B, HLA-C, HLA-E, HLA-F and HLA-G polypeptide).

In some embodiments, contacting the cancer cell with a population of isolated lymphoid lineage cells and a population of isolated myeloid lineage cells occurs in vitro.

In various embodiments, contacting the cancer cell with a population of isolated lymphoid lineage cells and a population of isolated myeloid lineage cells occurs in vivo.

### Example 9: Treating Cancer with a Plurality of iPSC-Derived Cells

In this example, a lymphoid lineage cells and myeloid lineage cells of the present disclosure are together used to treat a cancer.

An animal's immune system comprise a wide variety of immune cell types capable of contributing to an anti-cancer effect. And, in vivo, one type of immune cell promotes the cancer-killing ability of a second type of immune cell. Notably, macrophages are not adept at killing cancer cells but expert in infiltrating solid tumors and secreting cytokines that recruit cancer killing cells; on the other hand, NK cells are expert in killing cancer cells but rarely not infiltrate solid tumors alone and require recruitment by macrophages which have already infiltrated the solid tumor. Thus, each type of immune cell has its function which work in cooperation with the other cell types to attack and kill cancer cells. Nonetheless, many cell-based cancer therapeutics in clinical trials employ one type of immune cell rather than a plurality of immune cell types as existent in vivo.

This present invention provides a plurality cell types that includes (1) lymphoid lineage cells, e.g., cytotoxic lymphocytes including NK cells, which are iPSC derived and/or are genetically engineered to express a chimeric antigen receptor (CAR) and/or genetically engineered to express or overexpress a cytokine and (2) myeloid lineage cells, including macrophages, which are iPSC derived and/or are genetically engineered to express a chimeric antigen receptor (CAR) and/or genetically engineered to express or overexpress a cytokine.

An aspect of the present disclosure is a method for treating a cancer. The method comprising administering to a subject in need a therapeutically-effective amount of a first pharmaceutical composition comprising one or both of a population of isolated lymphoid lineage cells and a population of isolated myeloid lineage cells.

In embodiments, the first pharmaceutical composition comprises the population of isolated lymphoid lineage cells and wherein the subject in need is administered a therapeutically-effective amount of a second pharmaceutical composition comprising a population of isolated myeloid lineage cells.

In some embodiments, the first pharmaceutical composition comprises the population of isolated myeloid lineage cells and wherein the subject in need is administered a therapeutically-effective amount of a second pharmaceutical composition comprising a population of isolated lymphoid lineage cells. In some cases, the first pharmaceutical composition and the second pharmaceutical composition are administered simultaneously or sequentially. The first pharmaceutical composition and the second pharmaceutical composition may be administered sequentially with the first pharmaceutical composition administered before the second pharmaceutical composition or the first pharmaceutical composition and the second pharmaceutical composition may be administered sequentially with the second pharmaceutical composition administered before the first pharmaceutical composition.

The method for treating a cancer patient in need thereof comprises a step of administering to the cancer patient a therapeutically-effective amounts of a herein-disclosed lymphoid lineage cells (e.g., cytotoxic lymphocytes, including NK cells) and myeloid lineage cells (including macrophages), which are manufactured by a method disclosed herein.

The lymphoid lineage cells may be a Natural Killer (NK) cell. In some cases, the NK cell is a mature NK cell. The cytotoxic lymphocyte may be a Natural killer T (NKT) cell. In some cases, the cytotoxic lymphocyte is a delta-gamma T cell.

In some case, the method for treating cancer comprises steps of obtaining an isolated cytotoxic lymphocyte comprising a genetically engineered disruption in a B2M gene; and administering the isolated cytotoxic lymphocyte to a subject in need thereof. In this method the cytotoxic lymphocyte is a lymphoid cell lineage or myeloid cell. In some cases, the cytotoxic lymphocyte is a T cell, e.g., a cytotoxic T cell or gamma-delta T cell; NK cell, e.g., a NK-T cell, a CAR-myeloid cell, or a CAR-mesenchymal stromal/stem cell.

In some cases, additionally or alternately, the cytotoxic lymphocyte expresses a high affinity CD16a receptor.

In some cases, the cytotoxic lymphocyte expresses a fusion protein comprising a B2M polypeptide and an HLA-A, HLA-B, HLA-C, HLA-E, HLA-F and HLA-G polypeptide. The fusion protein may be expressed by insertion of a repair template into a single or double strand break of the B2M gene; in some cases, the repair template comprises the coding sequence for B2M and the HLA gene. In embodiments, the repair template includes the coding sequence for one or more of HLA class I histocompatibility antigen, alpha chains (HLAs). Notably, the fusion protein replaces endogenous B2M and HLA pairs expressed by a cytotoxic lymphocyte, thereby reducing the likelihood that the cytotoxic lymphocyte will be reduced or eliminated by a host cytotoxic lymphocyte.

In some cases, the myeloid lineage cell is a macrophage, e.g., an M1 macrophage or an M2 macrophage.

In some cases, the myeloid lineage cell is a cell derived from, or derivable from, a common myeloid progenitor cell. In embodiments, the myeloid cell is a megakaryocyte, erythrocyte, mast cell, or myeloblast. In embodiments, the myeloid cell is a cell derived from, or derivable from, a myeloblast. In embodiments, the myeloid cell is a basophil, neutrophil, eosinophil, or monocyte. In embodiments, the myeloid cell is a cell derived from, or derivable from a monocyte. In embodiments, the myeloid cell is a macrophage. In embodiments, the myeloid cell is a dendritic cell.

In some cases, the cytotoxic lymphocyte and/or the myeloid lineage cell is further genetically engineered to express a chimeric antigen receptor (CAR).

In some cases, the cytotoxic lymphocyte and/or the myeloid lineage cell is further genetically engineered to express or overexpress a cytokine.

When a subject in need lacks lymphoid lineage cells, the subject is administered iPSC-derived lymphoid lineage cells, e.g., cytotoxic lymphocytes including NK cells. The iPSC-derived lymphoid lineage cells may or may not be further engineered to express a CAR or to express or overexpress a cytokine.

When a subject in need lacks myeloid lineage cells, the subject is administered iPSC-derived myeloid lineage cells, including macrophages. The iPSC-derived myeloid lineage cells may or may not be further engineered to express a CAR or to express or overexpress a cytokine.

When a subject in need lacks lymphoid lineage cells and myeloid lineage cells, the subject is administered iPSC-derived lymphoid lineage cells, e.g., cytotoxic lymphocytes including NK cells, and is administered iPSC-derived myeloid lineage cells, including macrophages. The iPSC-derived myeloid lineage cells and/or iPSC-derived myeloid lineage cells may or may not be further engineered to express a CAR or to express or overexpress a cytokine.

In cases when the subject possesses natural lymphoid lineage cells, the subject is provided a synthetic mRNA encoding a gene-editing protein and a single-stranded or double-stranded repair template which encodes a chimeric antigen receptor (CAR). The gene-editing protein creates a single-stranded break or a double-stranded break in the genomic DNA of a cell in the subject and the single-stranded or double-stranded repair template which encodes the CAR inserts into the break. Consequently, the natural lymphoid lineage cells in the subject expresses the CAR. Alternately or additionally, the subject is provided a synthetic mRNA encoding a gene-editing protein and a single-stranded or double-stranded repair template which encodes a cytokine. The gene-editing protein creates a single-stranded break or a double-stranded break in the genomic DNA of a cell in the subject and the single-stranded or double-stranded repair template which encodes the cytokine inserts into the break. Consequently, the natural lymphoid lineage cells in the subject expresses or over expresses the cytokine. When the synthetic mRNA and/or the repair template is administered to a subject, the synthetic mRNA and/or the repair is combined with a lipid system comprising a compound of Formula (IV).

In cases when the subject possesses natural myeloid lineage cells, the subject is provided a synthetic mRNA encoding a gene-editing protein and a single-stranded or double-stranded repair template which encodes a chimeric antigen receptor (CAR). The gene-editing protein creates a single-stranded break or a double-stranded break in the genomic DNA of a cell in the subject and the single-stranded or double-stranded repair template which encodes the CAR inserts into the break. Consequently, the natural myeloid lineage cells in the subject expresses the CAR. Alternately or additionally, the subject is provided a synthetic mRNA encoding a gene-editing protein and a single-stranded or double-stranded repair template which encodes a cytokine. The gene-editing protein creates a single-stranded break or a double-stranded break in the genomic DNA of a cell in the subject and the single-stranded or double-stranded repair template which encodes the cytokine inserts into the break. Consequently, the natural myeloid lineage cells in the subject expresses or over expresses the cytokine. When the synthetic mRNA and/or the repair template is administered to a subject, the synthetic mRNA and/or the repair is combined with a lipid system comprising a compound of Formula (IV).

Any combination of iPSC-derived myeloid lineage cells, iPSC-derived lymphoid lineage cells, synthetic RNA encoding a CAR, and/or synthetic RNA encoding a cytokine may be administered to a subject in need.

The isolated lymphoid lineage cells may be manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes; wherein steps (5) and (6) occur in an adherent culturing vessel. In cases when CD34+ cells are selected, the embryoid bodies are first chemically and/or mechanically dissociated. In various cases, the stem cell is an induced pluripotent stem (iPSC).

The isolated myeloid lineage cells may be manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a myeloid progenitor medium; and (6) culturing the cells of step (5) in a macrophage cell medium under conditions to obtain a population of cells enriched for macrophages; wherein steps (5) and (6) occur in a bioreactor. In cases when CD34+ cells are selected, the embryoid bodies are first chemically and/or mechanically dissociated. In various cases, the stem cell is an induced pluripotent stem (iPSC). The isolated lymphoid lineage cells and the isolated myeloid lineage cells may be manufactured by a method comprising steps of: (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor. In cases when CD34+ cells are selected, the embryoid bodies are first chemically and/or mechanically dissociated. In various cases, the stem cell is an induced pluripotent stem (iPSC).

Other methods for manufacturing lymphoid lineage cells and myeloid lineage cells as disclosed herein may be used this method for treating a cancer.

In numerous embodiments, the administering is intravenous, intraarterial, intratumoral, or injected in the vicinity of a tumor.

The cancer may be a blood cancer.

The cancer may be a solid tumor.

The cancer may be selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

### Example 10: mRNA Cell Engineering Enables Rapid Prototyping of Macrophage Gene-Editing Strategies for Cancer Immunotherapies

In this example, advances in the generation of a gene-edited macrophages for use in cancer treatment is described.

Macrophages are drawing increased attention in cell therapy applications for their innate functionality in many organ tissues and high infiltration into tumors. However, much of the focus for macrophage cellular engineer is on taking a CAR designed for T-cells and using it in macrophages, which are fundamentally different immune cells. In order to address these problems, we aimed to create a rapid prototyping procedure to assess novel CAR design functionality in macrophages.

We use our in-house protocol for differentiating iPSCs into macrophages to generate a large, homogenous cell population. These cells are then transfected with mRNA encoding an ROR1-targeting CAR, displaying high expression and quantifiable functionality. FIG. 30 is a cartoon showing methods for rapid prototyping of macrophage gene-editing strategies for cancer immunotherapies.

FIG. 31 is a timeline showing efficient differentiation of iPSCs into macrophages. iPSCs were differentiated into macrophages using a three-stage process. The iPSCs were initially recovered from thaw in a bioreactor with mTeSR™ media, then embryoid bodies (EBs) were formed using EB-formation media. The EBs were transferred to a static culture plate for macrophage progenitor formation in X-VIVO 15 with IL-3 and M-CSF. Later, when a preferred construct is identified, scaling up macrophage production, after macrophage progenitor formation has occurred, the cells may be transferred to a bioreactor for expansion.

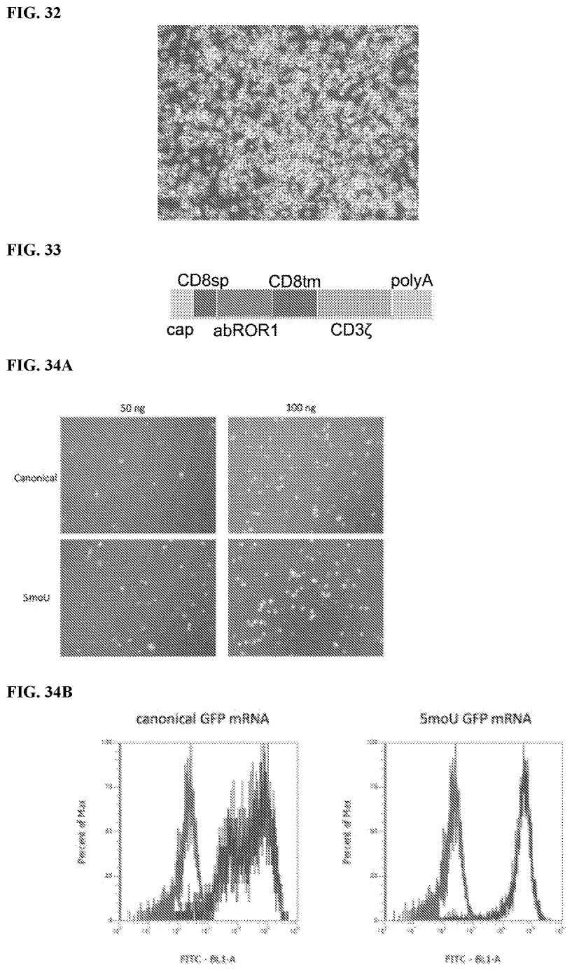

FIG. 32 is a photomicrograph showing iPSC-macrophages displaying macrophage-like morphology following differentiation. Following the the-stage differentiation over the course of 32 days, the harvested macrophages were plated into IMDM media with M-CSF to terminally differentiate into mature macrophages. The macrophage progenitors efficiently adhered within 4 hours and were imaged on an EVOS M5000 microscope.

FIG. 33 is a schematic of an mRNA encoding a ROR1-CAR. The coding sequence of an ROR1-CAR was designed with a CD3zeta activation domain (CD3ζ), CD8 signal peptide and transmembrane domains (CD8sp and CD8tm), ROR1 antibody domain (abROR1), a ^{m7}GpppN_{2′Ome}N cap and poly adenosine tail (polyA). Other domains known to be useful in the art, including those disclosed elsewhere herein, may be substituted for the domains mentioned here.

FIG. 34A and FIG. 34B relate to macrophages transfected with mRNA encoding GFP. While macrophages are known to efficiently uptake nucleotides including mRNA, they can elicit an immune response and potentially impair translation. The expression of mRNA encoding GFP was assayed using either canonical RNA nucleotides or 5-Methoxy-UTP (5moU). FIG. 34A includes florescent photomicrographs showing efficient expression of mRNA GFP in macrophages, with the 5moU nucleotides showing brighter expression compared to canonical. Images taken with EVOS M5000 microscope. FIG. 34B includes graphs of data from GFP-encoding mRNA transfected into macrophages which further validates these claims with flow cytometry data showing more consistent and efficient expression of GFP in 5moU transfected cells, collected on the Attune NxT flow cytometer.

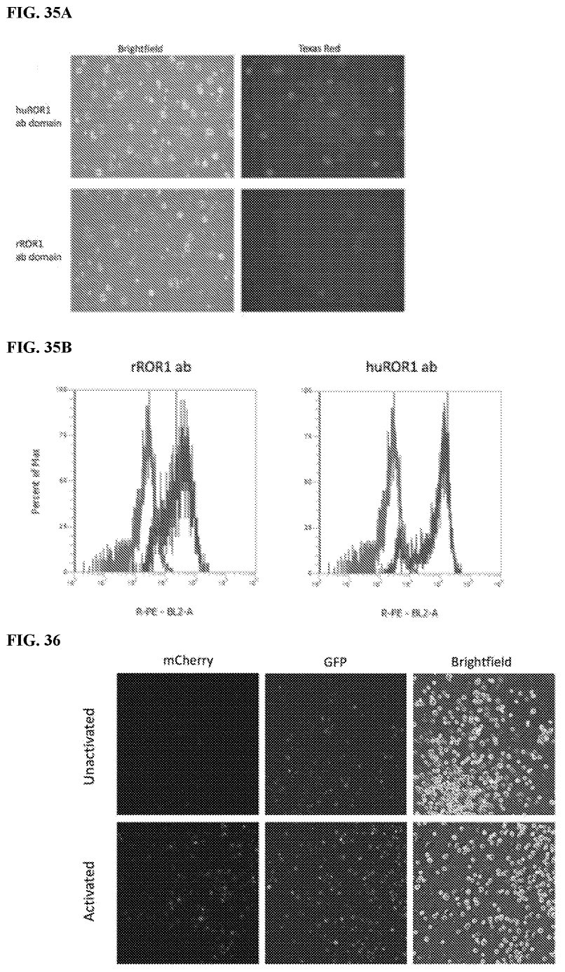

FIG. 35A and FIG. 35B relate to macrophages transfected with mRNA encoding an ROR1-CAR. Here, constructs with one of two different ROR1 antibody domains, the rabbit R12 (rROR1) and human (huROR1) domains, were tested to assess their ability to bind to PE-labelled ROR1 recombinant protein. Macrophages described in FIG. 32 were transfected mRNA encoding one of the two CARs. 24 hours post transfection, the wells were washed twice with DPBS and stained with PE-ROR1 for 30 minutes. The cells were washed again in DPBS and imaged in flow cytometry staining buffer on a Nikon Inverted microscope. FIG. 35A includes florescent photomicrographs following ROR1-PE staining of mRNA transfected macrophages with the ROR1-CAR. The cells were lifted, fixed with 4% paraformaldehyde, and assessed on the Attune NxT flow cytometer. FIG. 35B includes graphs of data from macrophages transfected with mRNA encoding an ROR1-CAR. Here, cells transfected with the huROR1 antibody domain showed greater binding affinity to the recombinant ROR1-PE protein compared to the rROR1 domain.

FIG. 36 includes photomicrographs of data from a Zymosan bead phagocytosis assay. huROR1-CAR transfected macrophages were split into un-activated and activated wells. The activated macrophages had ROR1-PE added to their culture, then both wells were incubated with GFP-labelled pHrodo Zymosan particles for four hours. ROR1 activation increased the intensity of Zymosan bead fluorescence caused by acidity in the phagosomes and lysosomes of macrophages.

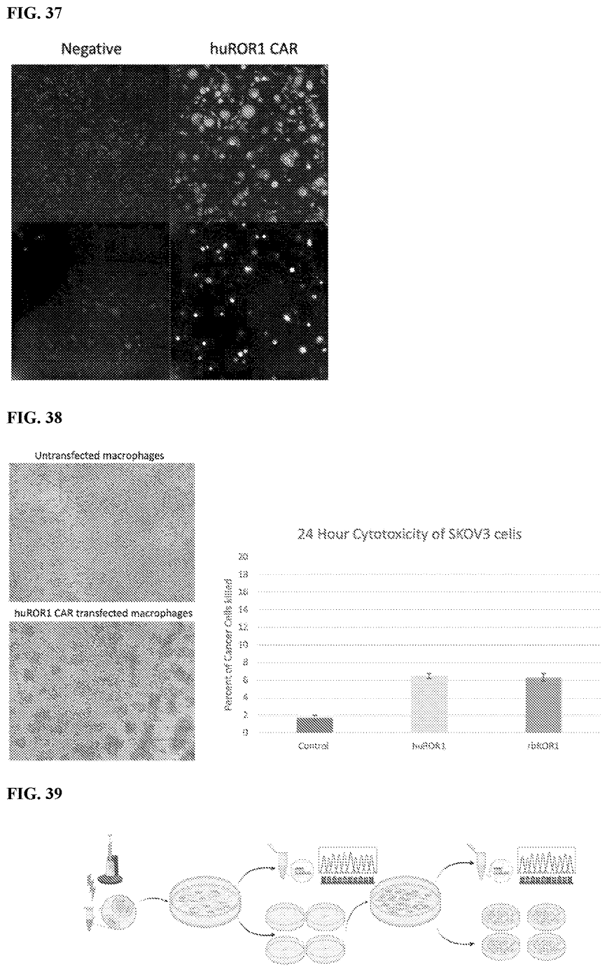

FIG. 37 includes photomicrographs of data from a CD3ζ phosphorylation assay. Macrophages were activated with ROR1-PE protein (orange, top right). Following activation, they were permeabilized with Triton X-100 and stained from intracellular CD3ζ phosphorylation (green, bottom right). Imaged on Operetta.

FIG. 38 includes photomicrographs (left) and a graph (right) of data from an SKOV3 cytotoxicity assay. Macrophages were co-cultured with SKOV-3 cancer cells known to express ROR1. Co-culture at 5:1 E:T. Both constructs provided statistically significant cancer cell killing.

FIG. 39 is a cartoon showing a protocol for insertion of transgene. Once a ROR1-CAR structure had been validated using mRNA transfection, the next step was into insert it into an iPSC line for large-scale production and characterization. This five-step protocol describes the procedure from electroporating iPSCs, recovering the heterogonous population, single-cell sorting and verifying insertion in the bulk population, recovering the single-cell wells, and verifying insertion in the homogenous isolated cell lines.

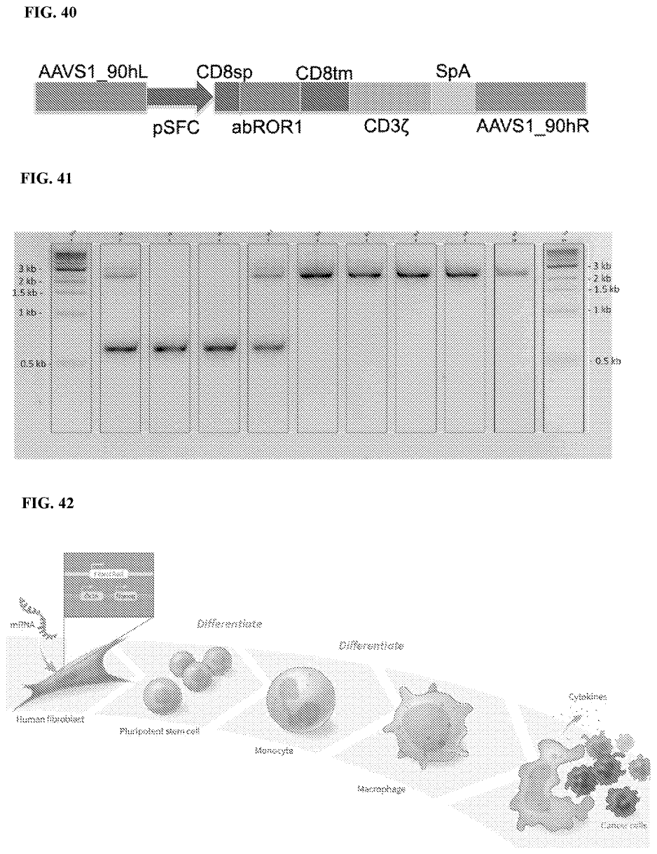

FIG. 40 is a schematic of the structure of a ROR1-CAR transgene. The transgene sequence of an ROR1-CAR was designed with 90 nucleotide AAVS1 homology arms (AAVS1_90hL and AAVS1_90hR) under the SFC promoter (pSFC), with the CAR sequence: CD3zeta activation domain (CD3ζ), CD8 signal peptide and transmembrane domains (CD8sp and CD8tm), ROR1 antibody domain (abROR1), and synthetic polyadenylation sequence (SpA).

FIG. 41 is a gel showing insertion of a ROR1-CAR transgene into iPSC lines. Following the protocol outlined in FIG. 39 and using the transgene described in FIG. 40, the transgene into 7 of 9 picked iPSC colonies was successfully inserted into iMSCs, with five cell lines displaying biallelic insertion. Colonies were passaged and split, with a portion of cells were seeded for expansion and the remainder isolated for gDNA extraction. The gDNA was then used to amplify the AAVS1 locus to verify insertion via PCR. Expected amplicon size was 600 bases long uninserted, 2.4 kb with transgene insertion. Columns 1 and 11 show a 1 kilobase ladder, while the remaining show isolated iPSC colonies after electroporation and single-cell sorting. kb: kilobase pair.

These data show efficient expression of mRNA transfected into iPSC- and PBMC-derived macrophages. It was discovered that 5-Methoxy-UTP (5moU) mRNA showed more consistent and brighter expression in macrophages than canonical mRNA nucleotides. mRNA encoding ROR1-CAR allowed rapid ideation and prototyping of CAR domains, including antigen binding domains, for functional optimization. ROR1-CAR macrophages display increased functionality following activation with target antigen, displaying quantifiable CD3ζ phosphorylation, Zymosan bead phagocytosis, and ROR1-expressing cancer cell cytotoxicity. And, mRNA gene editing enabled insertion of target transgene following optimization with efficient insertion and isolation for homogenous iPSC line development.

An aspect of the present disclosure is a method for screening constructs capable of being expressed in an in vivo cell and for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; and (3) identifying cells that have been transformed and capable of expressing the fusion protein for the ability of its fusion protein to recognize and/or bind to a cancer cell. Another aspect of the present disclosure is method for screening constructs capable of being expressed in an ex vivo cell and for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) culturing the cell capable of expressing the fusion protein which recognizes and/or binds to a cancer cell until a therapeutic amount of the cell is manufactured.

A further aspect of the present disclosure is a method for screening constructs capable of being expressed in an ex vivo cell and for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) contacting an ex vivo cell with the synthetic mRNA encoding the gene-editing protein and the repair template encoding the fusion protein which was identified in step (3) as having the ability recognize and/or bind to a cancer cell; and (5) culturing the cell of step (4) until a therapeutic amount of the cell is manufactured.

An additional aspect of the present disclosure is a method for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; and (4) administering to a subject in need the synthetic mRNA encoding the gene-editing protein and the repair template encoding the fusion protein which has the ability recognize and/or bind to a cancer cell.

In an aspect, the present disclosure provides a method for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) culturing the cell capable of expressing the fusion protein which recognizes and/or binds to a cancer cell until a therapeutic amount of the cell is manufactured; and (5) administering a therapeutically-effective amount of the cells of step (4) to a subject in need. In another aspect, the present disclosure provides a method for treating a cancer. The method comprising: (1) obtaining a cultured cell which corresponds to cell type present in a subject; (2) transfecting the cultured cell with a synthetic mRNA encoding a gene-editing protein and a repair template encoding a fusion protein that recognizes and/or binds to a cancer cell; (3) identifying cells that have been transformed and capable of expressing the fusion protein for the ability of its fusion protein to recognize and/or bind to a cancer cell; (4) contacting an ex vivo cell with the synthetic mRNA encoding the gene-editing protein and the repair template encoding the fusion protein which was identified in step (3) as having the ability recognize and/or bind to a cancer cell; (5) culturing the cell of step (4) until a therapeutic amount of the cell is manufactured; and (6) administering a therapeutically-effective amount of the cells of step (4) to a subject in need.

### Example 11: Induced Pluripotent Stem Cell (iPSC)-Derived Monocytes are Differentiated into Functional M1 and M2 Macrophages with Enhanced Cytokine Secretion and Tumor Cell-Killing Activity

In this example, iPSCs are which are then differentiated into monocytes which are further differentiated into cancer killing macrophages, i.e., M1 and M2 macrophages.

FIG. 42 is a cartoon showing methods for reprogramming fibroblasts into induced pluripotent stem cells (iPSCs), which are then differentiated into monocytes which are further differentiated into cancer killing macrophages.

Notably, the fibroblasts may be initially reprogrammed into iPSCs by transfecting the cells with suitable reprogramming factors. Shown in FIG. 42 are increased expression of Oct4 and Nanog; however, a cell can be reprogrammed with any combination of herein-disclosed reprogramming factors, e.g., Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

Alternately, the iPSC may be obtained from a stable iPSC cell line made by any method in the art. The general steps for the process of differentiating iPSCs into monocytes which are further differentiated into cancer killing macrophages, i.e., M1 and M2 macrophages comprises: (1) iPSC to monocyte differentiation, (2) CD14+ magnetic bead positive selection, and (3) monocyte to macrophage differentiation. These general steps are, respectively, shown in FIG. 43A, FIG. 43B, and FIG. 43C.

As shown in FIG. 43A, embryonic stem cells (ES cells) may be used as starting material for the process rather than iPSCs.

Beginning on day 14, monocyte cells can be harvested and continue to be harvested every 3-4 days. Additional details regarding differentiation of iPSCs to monocytes is found at the World Wide Web (www) stemcell.com/stemdiff-monocyte-kit.html, the contents of which are incorporated herein by reference in its entirety.

As shown in FIG. 43B, CD14+ cells, which comprise monocytes and macrophages, are separated using suitable reagents.

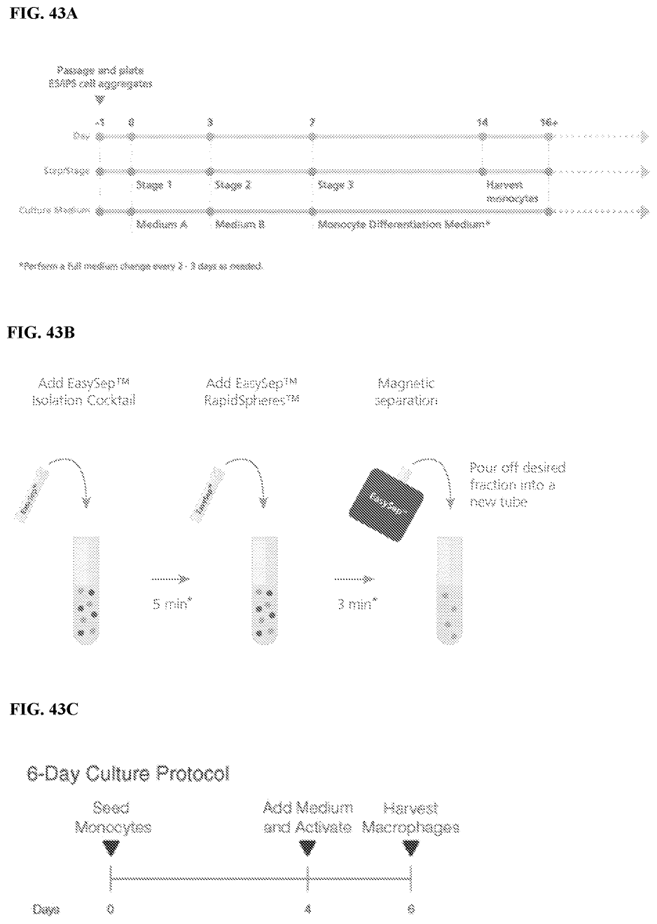

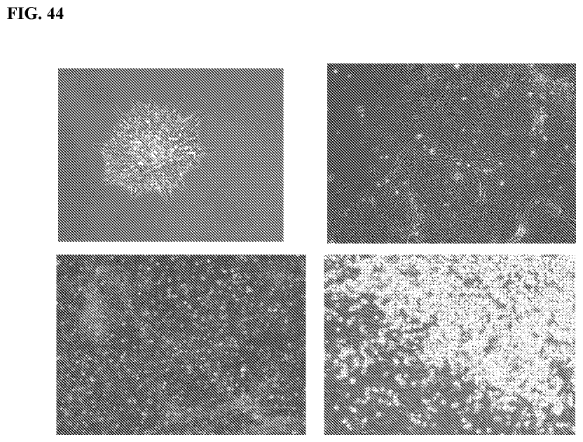

FIG. 44 shows progressing of cells from an iPSC colony (top left), day 3 mesoderm (top right), day 7 hematopoietic stem cell (bottom left), and finally day 14 monocyte (bottom right).

Beginning on day 14, cells were harvested every 3-4 days. CD14+ isolation yielded >95% CD14+ cells with an average yield of 4.1×10⁴ cells per cm² per harvest.

In the below tables, additional data of monocyte cell counts is shown and separated for hypoxic iPSCs and normoxic iPSCs.

| | | |
|---|---|---|
| Live Cell Counts | Pre-selection | Post-selection (CD14+) |

Hypoxic iPSCs

| | | |
|---|---|---|
| 1st harvest (Day 14) | 2.3 × 10⁶ | 1.035 × 10⁶ |
| 2nd harvest (Day 21) | 4.8 × 10⁶ | 1.2 × 10⁶ |

Normoxic iPSCs

| | | |
|---|---|---|
| 1st harvest (Day 14) | 12.9 × 10⁶ | 2.95 × 10⁶ |
| 2nd harvest (Day 21) | 16.9 × 10⁶ | 3.15 × 10⁶ |

Monocytes were tested for their loss of human pluripotent stem cell markers TRA-1-60 and TRA-1-81 and preservation of the hematopoietic lineage marker CD56. See below table.

| | iPSC (Day 0) | Monocyte (Day 14) |
|---|---|---|
| Tra-1-60 | 96.9% | 4.1% |
| Tra-1-81 | 87.5% | 0.4% |
| CD56 | 14.9% | 14.1% |

iPSC-derived monocytes were compared, using flow cytometry, to peripheral blood mononuclear cell (PBMC)-derived monocytes for expression of key hematopoietic and myeloid-lineage markers CD11b, CD14, CD33, CD45, CD80, CD163, CD206, and SIRPα. iPSC-derived monocytes showed similar expression of CD11b, CD14, CD33, CD45, and CD163 compared to peripheral blood mononuclear cell (PBMC)-derived monocytes, and increased expression of markers indicative of an activated state: CD80 and CD206. See the below table:

| | PBMC-Monocytes | iPSC-Monocytes |
|---|---|---|
| CD3 | 0.4% | 98.6% |
| CD11b | 80.5% | 93.4% |
| CD13 | 99.8% | 99.4% |
| CD33 | 99.5% | 82.3% |
| CD45 | 99.7% | 97.8% |
| CD68 | 0.0% | 6.5% |
| CD80 | 0.0% | 98.7 |
| CD206 | 0.8% | 93.2 |
| SIRPa | 99.6% | 62.0% |

Recovery of cryopreserved monocytes were then tested. Vials of 5×10⁶ live cells/mL were frozen in 20% DMSO/80% RPMI1640 and later thawed. The PBMC-monocytes (n=1) resulted in a thawed live cell count of 3.41×10⁶, which represents a 68.2% yield and the iPSC-monocytes (n=2) resulted in a thawed live cell count of 3.00×10⁶, which represents a 60.0% yield. The thawed cells were then characterized using flow cytometry and data is shown in the below table:

| | PBMC-Mono | iPSC-Mono |
|---|---|---|
| Live/Dead | 99%/1% | 94%/6% |
| CD14 | 83.2% | 78.9% |
| CD45 | 99.8% | 95.0% |
| CD163 | 12.5% | 3.0% |

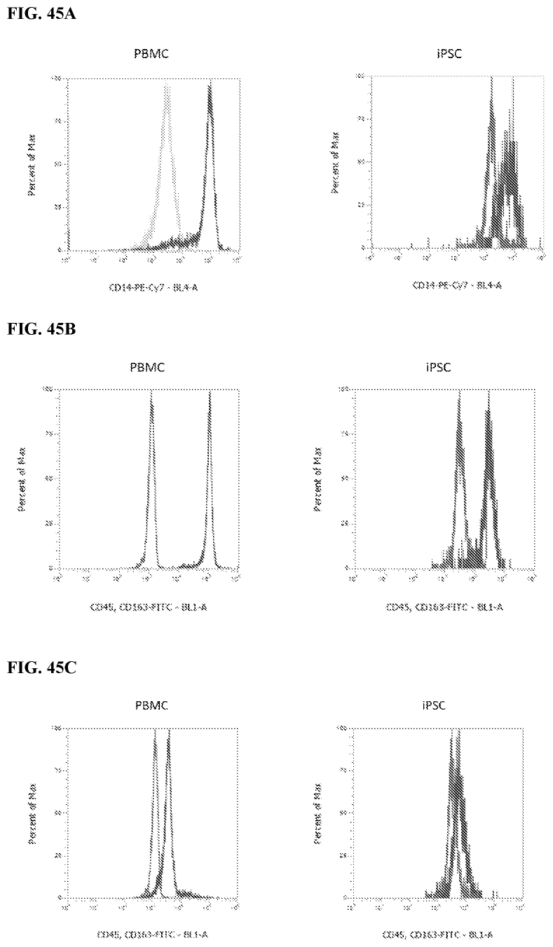

Flow cytograms for the data in the above table are shown in FIG. 45A to FIG. 45C.

Compared to PBMC-derived monocytes, iPSC-derived monocytes showed both higher viability in culture and superior recovery from cryopreservation.

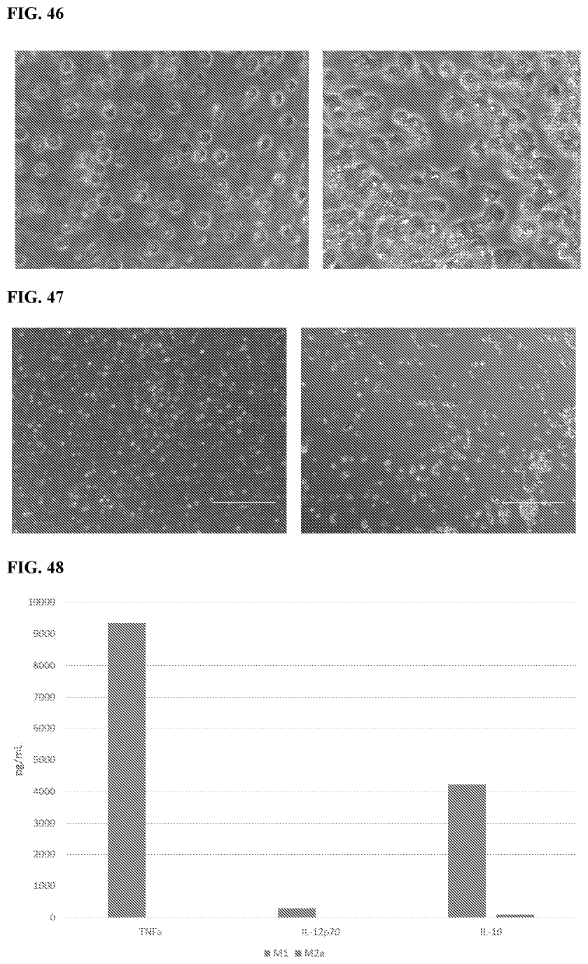

As shown in FIG. 43C, monocytes are cultured under conditions such that they differentiate into macrophages, e.g., by activation with macrophage colony-stimulating factor (M-CSF). Additional details regarding differentiation of monocytes to macrophage is found at the World Wide Web (www) stemcell.com/immunocult-sf-macrophage-medium.html, the contents of which are incorporated herein by reference in its entirety. FIG. 46 shows monocyte cultures at day zero (left image) and at day 4 and upon activation with M-CSF (right image).

iPSC-derived monocytes were further differentiated into macrophages by exposure to MCSF for 3-4 days, and were assessed for their ability to polarize, secrete pro- and anti-inflammatory cytokines, and for cytotoxic activity when co-cultured with cancer cells. M1 macrophages were polarized with interferon gamma (IFN-γ, 50 ng/mL) and lipopolysaccharide (LPS, 10 ng/mL) for 48 hours, while M2 macrophages were treated with IL-4 (10 ng/mL) for 48 hours. FIG. 47 shows macrophage cultures at day zero (left image) and at day 3 after (right image).

The below table shows macrophage yields from plating 500 k cells per well of a 24-well plate with 50 ng/mL of M-CSF for both fresh iPSC-monocytes and for cryopreserved monocytes.

| | Harvest/well | Yield |
|---|---|---|
| Fresh iPSC-Monocyte Harvest | | |
| M0 Stimulation | 4.17 × 10⁵ | 83.4% |
| M1 Stimulation | 5.04 × 10⁵ | 100.8% |
| M2a Stimulation | 5.43 × 10⁵ | 108.6% |
| Cryopreserved Monocytes | | |
| iPSC-Monocytes | 0.94 × 105 | 18.8% |
| PBMC-Monocytes | 0.56 × 105 | 11.2% |

iPSC-derived monocytes differentiated into macrophages with >90% efficacy, as assessed by cell adherence, morphology, and surface marker expression (CD14, CD45, CD163). See also the below table:

| | Primary M1 | iPSC-M1 | Primary M2a | iPSC-M2a |
|---|---|---|---|---|
| CD11b | 12.4 | 33.7 | 84.3 | 63.5 |
| CD68 | 2.6 | 0.0 | 0.5 | 0.2 |
| CD80 | 92.8 | 79.5 | 98.7 | 97.0 |
| CD86 | | 73.1 | | 70.9 |
| CD163 | | 52.6 | | 59.8 |
| CD206 | 24.9 | 17.5 | 95.7 | 18.7 |
| SIRPa | | 41.2 | | 16.9 |

M1 and M2 polarized iPSC-derived macrophages secreted similar levels of TNFα, IL-12p70, and IL-10 compared to PBMC-derived macrophages. See FIG. 48.

The ability of the iPSC-macrophages to kill cancer cells was then assayed.

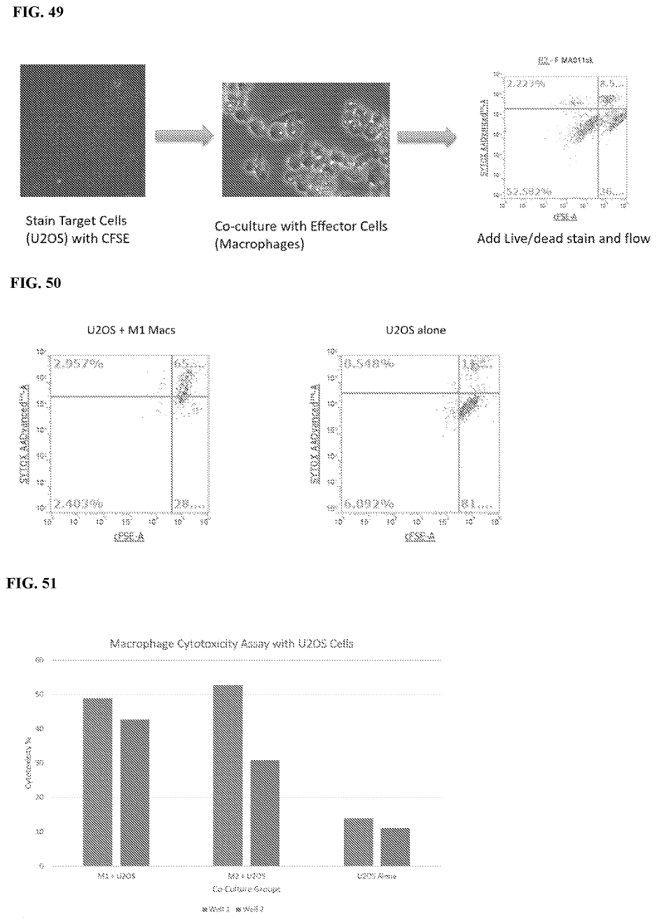

The method is illustrated in FIG. 49. As shown, cancer cells (here illustrated with U2OS, a sarcoma cell line) are stained with carboxyfluorescein succinimidyl ester (CFSE). The U2OS cells are co-cultured with effector cells, i.e., iPSC-macrophages of the present disclosure. Then the cells are stained to distinguish live and dead cells and the number of live and dead cells were qualified, here, shown in a flow cytometry scatter plot.

As shown in FIG. 50 and FIG. 51, iPSC-derived macrophages killed 45% of U2OS cancer cells in vitro after 24 hours at an E:T ratio of 5:1.

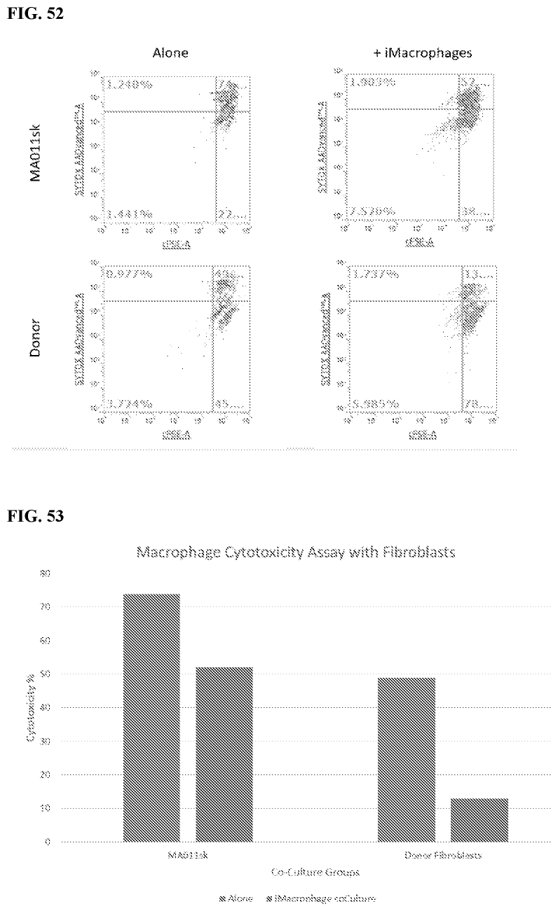

As shown in FIG. 52 and FIG. 53, iPSC-derived macrophages do not kill MA011sk cells or donor fibroblasts.

In summary, disclosed herein is a process for differentiating mRNA-reprogrammed iPSCs into cytotoxic macrophages. The mRNA reprogramming and differentiation processes are virus-free and DNA-free, avoiding any potential risk of vector integration. This disclosure provides proof of concept that mRNA-reprogrammed iPSCs represent a viable source of macrophages for the development of therapies to treat various indications, including solid tumors.

### Example 12: The Immunosuppressive TTAGGG Motif Improves Homology-Directed Insertion of DNA Sequences in Cells

In this example, dsODNs comprising the TTAGGG motif for decreased dsODN-related activation of a pro-inflammatory response in human cells, with decreasing the pro-inflammatory responses leading to higher transgene insertion efficiency. Notably, the TTAGGG motif is recognized by, and competitively binds to Pattern Recognition receptors in the cytoplasm of the cells to lessen the immune response mounted against the double stranded repair templates.

FIG. 54 is a graphical illustration of the mechanism of action that the inclusion of the TTAGGG motif has on cells during electroporation. Cells were electroporated with and without the addition of a gene editing protein, UltraSlice. All electroporation's were performed using the Invitrogen NEON.

In this example, the TTAGGG motif was incorporated onto the 5′ end of a double stranded repair template or co-electroporated as a short oligo fragment in primary fibroblasts, iMSC, or iPSC.

The sequence of the TTAGGG motif used in this example was: TTAGGGTTAGGGTTAGGGTTAGGG (SEQ ID NO: 15). This motif was incorporated at the 5′ and 3′ end of dsODN via PCR amplification using the primer pair A151-AAVS1, Forward primer: TTAGGGTTAGGGTTAGGGTTAGGGCCGAGCTGGGACCACCTTATATTCCCAGG (SEQ ID NO: 86) and A151-AAVS1, Reverse primer: TTAGGGTTAGGGTTAGGGTTAGGGAGGAGGTGGGGGTTAGACCCAATATCAGG (SEQ ID NO: 87). Control primers without the motif had the following sequences: AAVS1, Forward primer: CCGAGCTGGGACCACCTTATATTCCCAGG (SEQ ID NO: 88) and AAVS1, Reverse primer: AGGAGGTGGGGGTTAGACCCAATATCAGG (SEQ ID NO: 89). The full sequence of the repair template that human primary fibroblasts, iMSCs and iPSCs was: AAVS1-h90-pJeT-GFP-SpA-h90. This repair template included a dsODN encoding a GFP reporter and containing an SfoI restriction site targeting the AAVS1 site with 90 base pairs of homology. The A151 sequence was added to the end of a double stranded repair template using PCR amplification. Repair templates were then purified using the Qiagen QIAquick PCR Purification kit prior to electroporation. Note that AAV1 is a safe-harbor locus that was targeted for illustration purposes only.

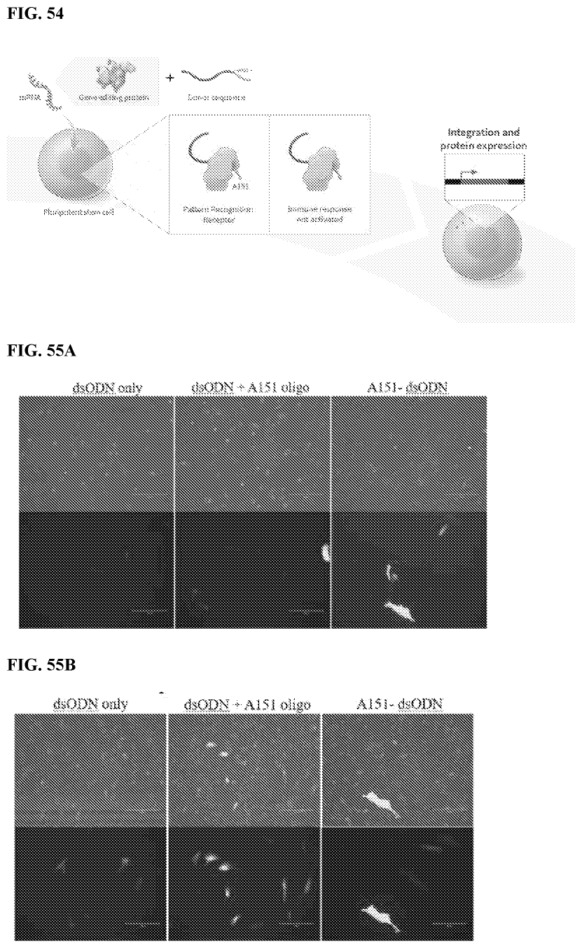

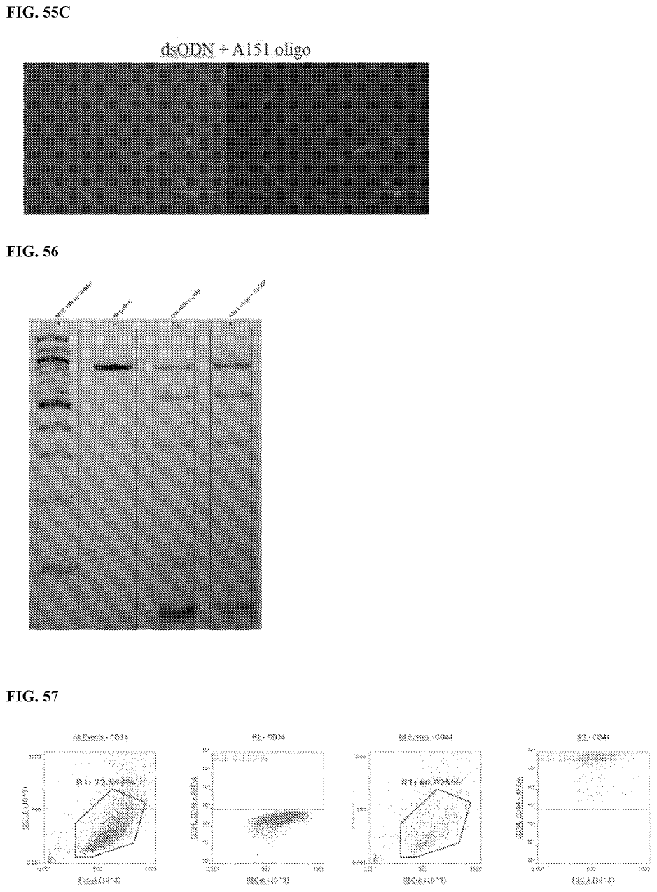

FIG. 55A shows that iMSC electroporated with the mRNA UltraSlice gene editing protein (e.g., having the general structure of LTPvQVVAIAwxyzα (SEQ ID NO: 22) and having features disclosed elsewhere herein) and with the repair template comprising the code for GFP, expressed GFP 24 hours after electroporation. FIG. 55B shows that iMSCs maintained GFP expression 72 hours after electroporation. FIG. 55C shows that iMSCs maintained GFP expression 28 days hours after electroporation and by passage 4. There was increased intensity of GFP in the presence of the TTAGGG motif. Further, cell count and recovery were higher compared to the electroporation with the control template only. Cells were electroporated with a dsODN that comprised the TTAGGG motif (right panel in FIG. 55A and FIG. 55B) or were electroporated with a dsODN that lacked the TTAGGG motif and the motif was in separate ODN (middle panel in FIG. 55A and FIG. 55B and right panel in FIG. 55C). Surprisingly, the iMSCs were frozen and resuspended in Synth-a-freeze, and after thawing regained similar GFP expression levels.

Importantly, in these gene-editing experiments with a GFP coding repair template directed to the AAV1 safe harbor site, the TTAGGG motif (whether included in the dsODN or co-transfected in the form of the A151 ODN) resulted in approximately 50% higher viability and approximately 50% more GFP-positive cells than when the motif was not present.

The presence of the A151 sequence decreased gene expression of immune response markers (IFIT1-3). iMSCs electroporated with the repair templates and A151 oligo were harvested and an RNA extraction was performed 24 hours after electroporation. The RT-PCR was performed on the QuantStudio 6 Flex by Life Technologies. The RT-PCR showed decreased expression of the immune markers IFIT1-3 when cells were exposed to the TTAGGG motif. This was found in conjunction with increased cell count after electroporation. All conditions were run with n=3 samples. The RQ values were normalized to the GAPDH probe in the electroporation only, negative sample. A 29-fold higher expression of IFIT1 and IFIT3 was observed in cells electroporated with dsODNs than in untreated controls. Interestingly, including TTAGGG motifs at the 5′-ends of the dsODNs limited the upregulation of IFIT1 and IFIT3 to 10- and 15-fold, respectively, while co-delivery of the TTAGGG motif prevented their upregulation altogether. The data is shown in the below table:

| | | | dsODN + A151 | A151 − |
|---|---|---|---|---|
| Gene | Negative | dsODN only | Oligo | dsODN |
| IFIT1 | 1.00 | 28.1 | 0.0590 | 16.8 |
| IFIT2 | 1.00 | 6.32 | 1.26 | 4.36 |
| IFIT3 | 1.00 | 29.4 | 1.96 | 11.0 |

iMSCs electroporated with the A151 oligo showed similar signs of gene editing than the UltraSlice only control, indicating that the prescience of this repeat sequence does not impede gene editing in primary cell lines. See FIG. 56.

iMSCs displayed the expected surface markers after electroporation with the A151 sequence present. The iMSCs were stained, fixed, and flowed on the AttuneNx for CD34, CD44, CD45, CD73, and CD90. Compared to a control population of iMSCs that were not electroporated, the populations displayed the same flow results. Briefly, both populations were positive for CD44, CD73, and CD90. Cells were negative for CD34 and CD45. FIG. 57 shows the spread of the population of iMSCs flowed. The populations showed clear signs of surface marker expression if they were positive (CD44) or negative (CD34). See the below table:

| | | | dsODN + oligo |
|---|---|---|---|
| Markers | Expected results | EDP iMSCs (%) | iMSCs (%) |
| CD34 | Negative | 8 | 0 |
| CD44 | Positive | 99 | 100 |
| CD45 | Negative | 6 | 10 |
| CD73 | Positive | 99 | 100 |
| CD90 | Positive | 98 | 99 |

The flow data indicates that the inclusion of the A151 sequence did not alter the surface markers, nor the viability of the cells after electroporation. During the harvesting process, cells had a viability of >97% for all populations and had a similar expansion timeline compared to the control iMSCs. See FIG. 57.

In summary, the data indicates that the incorporation of the telomeric repeat sequence TTAGGG motif can be incorporated at an end of a repair template to decrease the immune response of cells to the double stranded foreign DNA in the cytoplasm. This leads to increased integration into the overall cell population by improving cell viability after electroporation. Incorporating the TTAGGG motif as a separate oligo also provides the same benefits by competitively binding to TLRs in the cells during electroporation. This disclosure demonstrates for the first time that this sequence can be used to improve gene editing in, at least, primary fibroblasts, iMSCs, and iPSCs.

### Example 13: Contacting Resveratrol with a Cell in Advance of Gene Editing Arrests the Cell in S or G2 Phase and Enhances the Efficiency of Subsequent Gene-Editing

In this example, a cell that is to be gene-edited is pre-treated with Resveratrol.

The process of contacting Resveratrol with a cell in advance of gene editing is illustrated in FIG. 58. As shown in FIG. 58, Resveratrol arrests the cell in S or G2 phase and enhances the efficiency of subsequent gene-editing.

Fibroblast, as an illustrative cell type, was treated with 25 uM Resveratrol for 48 h followed by 8 h incubation in DMEM/10% FBS showed that there is approximately 40% increase in S/G2 population. See FIG. 59.

The following general protocol was used for an Sfol assay to quantify insertion of repair template.

### Cell Culture

1. Fibroblasts were cultured using DMEM+10% FBS media in 37° Celsius with 5% O₂ and 5% CO₂
2. iPSC was cultured using mTeSR+Y27632 in 37° Celsius with 5% O₂ and 5% CO₂

### Electroporation

3. After detaching cells with TrypleE and DTI, standard electroporation protocol was performed
4. 0.8 ug˜1 ug of AAVS1 Ultraslice RNA and 600 ng of SfoI ssDNA repair template was delivered by electroporation. Ultraslice is a temperature sensitive gene-editing protein. The SfoI base change ssDNA repair template was a single stranded DNA piece with homology arms that is used as template for targeted DNA repair
5. Fibroblasts were electroporated with 700 V, 20 ms, 1 pulse
6. iPSCs were electroporated with 1100 V, 20 ms, 2 pulse)

### Genomic DNA Extraction

7. Cells were detached 48 h˜72 h post electroporation and centrifuged to obtain cell pellets
8. Perform genomic DNA extraction was performed using ZYMO Research Quick DNA Miniprep Kit (Catalog #: D3024)

### AAVS1 Surveyor PCR

9. AAVS1 surveyor PCR was performed using KAPA Hifi Hotstart PCR kit

### SfoI/EheI Assay

10. 250 ng˜400 ng of AAVS1 PCR amplicon was obtained
11. Amplicon was mixed with 2 μl of 10× Tango Buffer and 1 μl of EheI enzyme
12. The mixture was incubated for 1 h in 37° C., followed by 20 mins at 65° C.

### Egel Loading/Band Quantification

13. After diluting SfoI assay product with 40 ul of H₂O, 20 ul of product was loaded to 2% Egel
14. After running 2% Egel for 10 minutes, the gel was visualized and SfoI band intensity was quantified using Chemidoc and ImageJ software

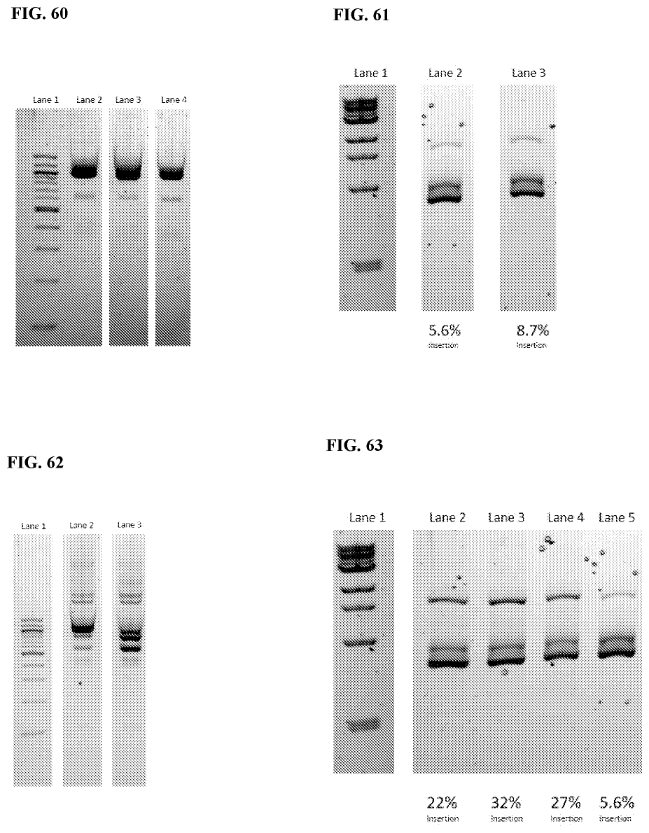

As shown in FIG. 60, Resveratrol pretreated fibroblasts have increased gene-editing efficiency. Following treatment with resveratrol, fibroblasts was co-transfected with mRNA encoding a chromatin context-sensitive gene-editing protein targeting the AAVS1 safe-harbor locus and a single-stranded DNA repair template designed to introduce a SfoI restriction-enzyme site through a G-to-C mutation. Single-base editing efficiency was determined by restriction fragment length polymorphism (RFLP) analysis. Notably, Lane 2 (Resveratrol 25 uM pretreated) showed a 2.2-fold increase in base change efficiency compared to Lane 3 (Untreated) condition. Lane 4 (Resveratrol 25 μM 48 h incubation) also shows approximately 2.3-fold increase in base change efficiency compared to Lane 3 (Untreated). In the gel, Lane 1: 100 base ladder; Lane 2: Ultraslice and SfoI base change ssDNA repair template (Resveratrol 25 μM 48 h pretreatment); Lane 3: Ultraslice and SfoI base change ssDNA repair template (Untreated); and Lane 4: Ultraslice and SfoI base change ssDNA repair template (Resveratrol 25 μM treatment 48 post electroporation).

FIG. 61 shows that post-electroporation Resveratrol treatment increased 1 kb insertion 1.6-fold in iPSC. iPSC was treated with 25 uM Resveratrol for 24 h post electroporation (Lane 3). Lane 3 shows that Resveratrol treatment increased 1 kb insertion rate from 5.6% to 8.7%. Thus, application of resveratrol after transfection (i.e., no cell cycle synchronization) yielded further improvement in single-base editing efficiency (>2-fold), suggesting that the effects of resveratrol on HDR are not confined to cell-cycle control. In this experiment, the gel image is AAVS1 sPCR amplicon and not Sfol assay product. In the gel, Lane 1: 1000 base ladder; Lane 2: Ultraslice (Clean cap) and SfoI 1 kb insertion ssDNA repair template with 90b homology arm (Reverse transcription); and Lane 3: Ultraslice (Clean cap) and SfoI 1 kb insertion ssDNA repair template with 90b homology arm (Reverse transcription)—Resveratrol 25 uM 24 h incubation.

As shown in FIG. 62, NU7441 (a DNA-PKs inhibitor that is known to inhibit NHEJ mediated DNA repair pathway) treated fibroblasts show significant increase in 300b insertion. Lane 3 (NU7 9 uM 72 h incubation) showed 7-fold increase in 300b insertion SfoI cut band intensity compared to Lane 2 (Untreated). In the gel, Lane 1: 100 base ladder; Lane 2: Ultraslice and SfoI 300 base insertion dsDNA repair template (Untreated); and Lane 3: Ultraslice and SfoI 300 base insertion dsDNA repair template (NU7441 9 uM 72 h treated).

FIG. 63 shows that a maximum 32% of 1 kb insertion rate was observed using ssDNA repair template in iPSCs. Lane 2 to 4 show that ssDNA synthesized with Lambda exonuclease methodology has 1 kb insertion rate ranging from 22% to 32% and lane 5 shows that ssDNA synthesized with Reverse transcription method has 1 kb insertion rate of 5.6%. In this experiment, the gel image is AAVS1 sPCR amplicon and not Sfol assay product. In this gel, Lane 1: 1000 base ladder; Lane 2: Ultraslice (Clean cap) and SfoI 1 kb insertion ssDNA repair template with 90b homology arm (Lambda exonuclease with single stranded decoy); Lane 3: Ultraslice (Clean cap) and SfoI 1 kb insertion ssDNA repair template with 90b homology arm (Lambda exonuclease); Lane 4: Ultraslice (Clean cap) and SfoI 1 kb insertion ssDNA repair template with 220b homology arm (Lambda exonuclease); and Lane 4: Ultraslice (Clean cap) and GFP 1 kb insertion ssDNA repair template with 90b homology arm (Reverse transcription).

In summary, the data indicates that Resveratrol treatment provides a straightforward method for improving HDR efficiency in cells. This treatment may be before transfecting with a gene-editing machinery or may be after transfecting with a gene-editing machinery. Thus, use of Resveratrol serves as a useful tool in the development of HDR-based gene-editing therapies.

1.-59. (canceled)

60. A method of engineering a cell, the method comprising
(a) contacting a cell with a synthetic RNA encoding a gene-editing protein, wherein the gene-editing protein generates a break in a target site of a beta-2-microglobulin (B2M) gene, and
(b) delivering to the cell a repair template comprising a nucleic acid sequence, wherein the nucleic acid sequence encodes an HLA-E polypeptide, wherein the nucleic acid sequence is inserted at the break in the target site of the B2M gene,
thereby generating an engineered cell.

61. The method of claim 60, wherein the nucleic acid sequence encodes a fusion polypeptide comprising the HLA-E polypeptide and a B2M polypeptide.

62. The method of claim 61, wherein the repair template comprises a nucleic acid sequence encoding a signal peptide.

63. The method of claim 60, wherein the break is a double strand break.

64. The method of claim 60, wherein the target site of the B2M gene comprises a sequence of SEQ ID NO: 12.

65. The method of claim 60, wherein the target site of the B2M gene is in Exon 1 of the B2M gene.

66. The method of claim 60, wherein the cell is an immune cell selected from the group consisting of a Natural Killer (NK) cell, a Natural Killer T (NKT) cell, a T cell, or a macrophage.

67. The method of claim 60, wherein the cell is a stem cell.

68. The method of claim 67, wherein the method further comprises generating the stem cell from a somatic cell.

69. The method of claim 60, wherein the method results in reduced surface expression of HLA-A, HLA-B, or HLA-C by the engineered cell.

70. The method of claim 60, wherein the method further comprises (c) contacting the cell with a nucleic acid sequence encoding a chimeric antigen receptor (CAR).

71. The method of claim 60, wherein the cell is further engineered to express a cytokine.

72. The method of claim 71, wherein the cytokine is selected from the group consisting of Interleukin (IL)-2, IL-15, IL-12, IL-7, IL-10, and IL-21.

73. The method of claim 60, wherein the gene-editing protein is selected from the group consisting of a transcription activator-like (TAL) effector nuclease, RiboSlice, a zinc-finger nuclease, a meganuclease, a nickase, or a clustered regularly interspaced short palindromic repeat (CRISPR)-associated protein.

74. The method of claim 60, wherein the synthetic RNA comprises one or more non-canonical nucleotides.

75. The method of claim 74, wherein the one or more non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

76. The method of claim 60, wherein the synthetic RNA, the repair template, or both the synthetic RNA and the repair template are delivered to the cell in combination with a lipid system.

77. The method of claim 76, wherein the lipid system comprises a compound of Formula (IV): 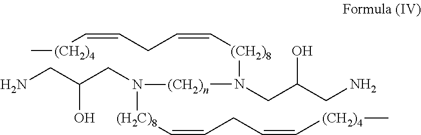
wherein n is an integer from 1 to 15.

78. The method of claim 77, wherein n is 4.

79. An engineered cell comprising a disruption in a B2M gene, wherein the engineered cell expresses a fusion protein comprising a fragment of a B2M polypeptide and a HLA-E polypeptide.


SEQUENCE LISTING (89 sequences)

SEQ ID NO: 1 - DNA, 18 bp, synthetic construct
  source 1..18: mol_type = other DNA
tcatccatccgacattga

SEQ ID NO: 2 - DNA, 16 bp, synthetic construct
  source 1..16: mol_type = other DNA
agttgacttactgaag

SEQ ID NO: 3 - DNA, 18 bp, synthetic construct
  source 1..18: mol_type = other DNA
aatggagagagaattgaa

SEQ ID NO: 4 - DNA, 16 bp, synthetic construct
  source 1..16: mol_type = other DNA
acattgaagaatggag

SEQ ID NO: 5 - DNA, 30 bp, synthetic construct
  source 1..30: mol_type = other DNA
acattgaagttgacttactgaagaatggag

SEQ ID NO: 6 - DNA, 96 bp, synthetic construct
  source 1..96: mol_type = other DNA
tgaattgctatgtgtctgggtttcatccatccgacattgaagttgacttactgaagaatggagagagaattgaaaaagtggagcattcagacttgt

SEQ ID NO: 7 - protein, 23 aa, synthetic construct
GKGRKYFHHNSDFHIPKATLKDS

SEQ ID NO: 8 - protein, 23 aa, synthetic construct
GKDRKYFHHNSDFYIPKATLKDS

SEQ ID NO: 9 - protein, 23 aa, synthetic construct
KDSGSYFCRGLFGSKNVSSETVN

SEQ ID NO: 10 - protein, 23 aa, synthetic construct
KDSGSYFCRGLVGSKNVSSETVN

SEQ ID NO: 11 - protein, 22 aa, synthetic construct
MSRSVALAVLALLSLSGLEAIQ

SEQ ID NO: 12 - DNA, 92 bp, synthetic construct
  source 1..92: mol_type = other DNA
atgtctcgctccgtggccttagctgtgctcgcgctactctctctttctggcctggaggctatccagcgtgagtctctcctaccctcccgctc

SEQ ID NO: 13 - protein, 119 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 14 - DNA, 14 bp, synthetic construct
  source 1..14: mol_type = other DNA
ttgacttactgaag

SEQ ID NO: 15 - DNA, 24 bp, synthetic construct
  source 1..24: mol_type = other DNA
ttagggttagggttagggttaggg

SEQ ID NO: 16 - protein, 14 aa, synthetic construct
LTPXQVVAIAXXXX

SEQ ID NO: 17 - protein, 18 aa, synthetic construct
LTPXQVVAIAXXXXGHGG

SEQ ID NO: 18 - protein, 18 aa, synthetic construct
LTPXQVVAIAXXXXGTHG

SEQ ID NO: 19 - protein, 19 aa, synthetic construct
GGKQALETVQRLLPVLCQD

SEQ ID NO: 20 - protein, 19 aa, synthetic construct
GGKQALETVQRLLPVLCQA

SEQ ID NO: 21 - protein, 4 aa, synthetic construct
GTHG

SEQ ID NO: 22 - protein, 15 aa, synthetic construct
LTPXQVVAIAXXXXX

SEQ ID NO: 23 - protein, 7 aa, synthetic construct
GGRPALE

SEQ ID NO: 24 - protein, 7 aa, synthetic construct
GGKQALE

SEQ ID NO: 25 - protein, 21 aa, synthetic construct
GGKQALETVQRLLPVLCQDHG

SEQ ID NO: 26 - protein, 21 aa, synthetic construct
GGKQALETVQRLLPVLCQAHG

SEQ ID NO: 27 - protein, 20 aa, synthetic construct
GKQALETVQRLLPVLCQDHG

SEQ ID NO: 28 - protein, 20 aa, synthetic construct
GKQALETVQRLLPVLCQAHG

SEQ ID NO: 29 - protein, 11 aa, synthetic construct
LTPXQVVAIAS

SEQ ID NO: 30 - protein, 16 aa, synthetic construct
LTPXQVVAIAXXXXHG

SEQ ID NO: 31 - protein, 4 aa, synthetic construct
GHGG

SEQ ID NO: 32 - protein, 4 aa, synthetic construct
HGSG

SEQ ID NO: 33 - protein, 4 aa, synthetic construct
HGGG

SEQ ID NO: 34 - protein, 4 aa, synthetic construct
GGHD

SEQ ID NO: 35 - protein, 4 aa, synthetic construct
GAHD

SEQ ID NO: 36 - protein, 4 aa, synthetic construct
AHDG

SEQ ID NO: 37 - protein, 4 aa, synthetic construct
PHDG

SEQ ID NO: 38 - protein, 4 aa, synthetic construct
GPHD

SEQ ID NO: 39 - protein, 4 aa, synthetic construct
GHGP

SEQ ID NO: 40 - protein, 4 aa, synthetic construct
PHGG

SEQ ID NO: 41 - protein, 4 aa, synthetic construct
PHGP

SEQ ID NO: 42 - protein, 4 aa, synthetic construct
AHGA

SEQ ID NO: 43 - protein, 4 aa, synthetic construct
LHGA

SEQ ID NO: 44 - protein, 4 aa, synthetic construct
VHGA

SEQ ID NO: 45 - protein, 4 aa, synthetic construct
IVHG

SEQ ID NO: 46 - protein, 4 aa, synthetic construct
IHGM

SEQ ID NO: 47 - protein, 4 aa, synthetic construct
RHGD

SEQ ID NO: 48 - protein, 4 aa, synthetic construct
RDHG

SEQ ID NO: 49 - protein, 4 aa, synthetic construct
RHGE

SEQ ID NO: 50 - protein, 4 aa, synthetic construct
HRGE

SEQ ID NO: 51 - protein, 4 aa, synthetic construct
HRGD

SEQ ID NO: 52 - protein, 4 aa, synthetic construct
GPYE

SEQ ID NO: 53 - protein, 4 aa, synthetic construct
NHGG

SEQ ID NO: 54 - protein, 4 aa, synthetic construct
THGG

SEQ ID NO: 55 - protein, 16 aa, synthetic construct
LTPXQVVAIAIXXXHG

SEQ ID NO: 56 - protein, 4 aa, synthetic construct
GSGS

SEQ ID NO: 57 - protein, 4 aa, synthetic construct
GSGG

SEQ ID NO: 58 - protein, 4 aa, synthetic construct
GGGG

SEQ ID NO: 59 - protein, 4 aa, synthetic construct
GRGG

SEQ ID NO: 60 - protein, 4 aa, synthetic construct
GKGG

SEQ ID NO: 61 - DNA, 150 bp, synthetic construct
  source 1..150: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - DNA, 150 bp, synthetic construct
  source 1..150: mol_type = other DNA
aaaaaaaaaaaaaaaaaaaaaaaaaaaaaaaaaaaaaaagaaaaaaaaaaaaaaaaaaaaaaaaaaaaaaaaaaaaaaagaaaaaaaaaaaaaaaaaaaaaaaaaaaaaaaaaaaaaaagaaaaaaaaaaaaaaaaaaaaaaaaaaaaaa

SEQ ID NO: 63 - DNA, 150 bp, synthetic construct
  source 1..150: mol_type = other DNA
aaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaa

SEQ ID NO: 64 - DNA, 150 bp, synthetic construct
  source 1..150: mol_type = other DNA
aaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaag

SEQ ID NO: 65 - protein, 37 aa, synthetic construct
LTPEQVVAIASRVDGGKQALETVQRLLPVLCQAGHGG

SEQ ID NO: 66 - protein, 16 aa, synthetic construct
LTPXQVVAIAXLAXHG

SEQ ID NO: 67 - protein, 12 aa, synthetic construct
LTPXQVVAIAXX

SEQ ID NO: 68 - protein, 18 aa, synthetic construct
GKQALETVQRLLPVLCQA

SEQ ID NO: 69 - protein, 18 aa, synthetic construct
GKQALETVQRLLPVLCQD

SEQ ID NO: 70 - protein, 17 aa, synthetic construct
LTPXQVVAIAXXXGHGG

SEQ ID NO: 71 - protein, 18 aa, synthetic construct
LTPXQVVAIAXLAXGHGG

SEQ ID NO: 72 - protein, 13 aa, synthetic construct
LTPXQVVAIAXXX

SEQ ID NO: 73 - protein, 16 aa, synthetic construct
DIVGVGKQWSGARALE

SEQ ID NO: 74 - protein, 16 aa, synthetic construct
DIVGVGKQKRGARALE

SEQ ID NO: 75 - protein, 20 aa, synthetic construct
DIVGVGGSKRGAGSGARALE

SEQ ID NO: 76 - protein, 360 aa, synthetic construct
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 77 - protein, 317 aa, synthetic construct
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 78 - protein, 477 aa, synthetic construct
QPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 79 - protein, 454 aa, synthetic construct
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 80 - DNA, 810 bp, synthetic construct
  source 1..810: mol_type = other DNA
agaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtccca

SEQ ID NO: 81 - DNA, 37 bp, synthetic construct
  source 1..37: mol_type = other DNA
cagagaaaggctcttaaaaatgcagcgcaatctccag

SEQ ID NO: 82 - DNA, 43 bp, synthetic construct
  source 1..43: mol_type = other DNA
cacttaactatcttgggctgtgacaaagtcacatggttcacac

SEQ ID NO: 83 - DNA, 43 bp, synthetic construct
  source 1..43: mol_type = other DNA
gtgtgaaccatgtgactttgtcacagcccaagatagttaagtg

SEQ ID NO: 84 - DNA, 39 bp, synthetic construct
  source 1..39: mol_type = other DNA
ctgatctagaacttactgtgaatccttgtcacctgccac

SEQ ID NO: 85 - DNA, 38 bp, synthetic construct
  source 1..38: mol_type = other DNA
gataagaaggaggccagcacgataggaacatatgacac

SEQ ID NO: 86 - DNA, 53 bp, synthetic construct
  source 1..53: mol_type = other DNA
ttagggttagggttagggttagggccgagctgggaccaccttatattcccagg

SEQ ID NO: 87 - DNA, 53 bp, synthetic construct
  source 1..53: mol_type = other DNA
ttagggttagggttagggttagggaggaggtgggggttagacccaatatcagg

SEQ ID NO: 88 - DNA, 29 bp, synthetic construct
  source 1..29: mol_type = other DNA
ccgagctgggaccaccttatattcccagg

SEQ ID NO: 89 - DNA, 29 bp, synthetic construct
  source 1..29: mol_type = other DNA
aggaggtgggggttagacccaatatcagg